(19) United States
(12) Patent Application Publication
(10) Pub. No.: US 2024/0164206 A1
(43) Pub. Date: May 16, 2024

(54) ORGANIC LIGHT-EMITTING COMPOUND AND ORGANIC LIGHT-EMITTING DEVICE COMPRISING SAME

(71) Applicant: SFC CO., LTD., Chungcheongbuk-do (KR)

(72) Inventors: Se-jin LEE, Chungcheongbuk-do (KR); Si-in KIM, Chungcheongbuk-do (KR); Seok-bae PARK, Chungcheongbuk-do (KR); Hee-dae KIM, Chungcheongbuk-do (KR); Yeong-tae CHOI, Chungcheongbuk-do (KR); Seung-soo LEE, Chungcheongbuk-do (KR); Ji-yung KIM, Chungcheongbuk-do (KR); Kyeong-hyeon KIM, Chungcheongbuk-do (KR); Kyung-tae KIM, Chungcheongbuk-do (KR); Myeong-jun KIM, Chungcheongbuk-do (KR); Tae-gyun LEE, Chungcheongbuk-do (KR); Joon-ho KIM, Chungcheongbuk-do (KR)

(21) Appl. No.: 18/276,999

(22) PCT Filed: Feb. 15, 2022
(86) PCT No.: PCT/KR2022/002196

§ 371 Date: Aug. 11, 2023

(30) Foreign Application Priority Data
Feb. 15, 2021 (KR) 10-2021-0019907

(51) Int. Cl. H10K 85/60; C07D 491/048; C07D 493/04; C09K 11/06

(52) U.S. Cl. CPC H10K 85/657; C07D 491/048; C07D 493/04; C09K 11/06; H10K 85/622; H10K 85/6574; H10K 50/12

(57) ABSTRACT

The present invention relates to: a pyrene derivative compound having a specific structure; and a high efficiency organic light-emitting device employing the pyrene derivative compound in a light emitting layer and thus having excellent light-emitting characteristics. The organic light-emitting device according to the present invention can be configured as a high efficiency organic light-emitting device having excellent light-emitting characteristics by employing the pyrene derivative compound having the specific structure as a host in the light emitting layer, and thus can be usefully applied industrially in lighting devices, as well as various display devices such as flat, flexible, and wearable displays.

### TECHNICAL FIELD

The present invention relates to an organic light emitting compound and an organic light emitting device including the same. More specifically, the present invention relates to a pyrene derivative having a specific structure and a highly efficient organic light emitting device including a light emitting layer employing the pyrene derivative as a host compound.

### BACKGROUND ART

Organic light emitting devices are self-luminous devices in which electrons injected from an electron injecting electrode (cathode) recombine with holes injected from a hole injecting electrode (anode) in a light emitting layer to form excitons, which emit light while releasing energy. Such organic light emitting devices have the advantages of low driving voltage, high luminance, large viewing angle, and short response time and can be applied to full-color light emitting flat panel displays. Due to these advantages, organic light emitting devices have received attention as next-generation light sources.

The above characteristics of organic light emitting devices are achieved by structural optimization of organic layers of the devices and are supported by stable and efficient materials for the organic layers, such as hole injecting materials, hole transport materials, light emitting materials, electron transport materials, electron injecting materials, and electron blocking materials. However, more research still needs to be done to develop structurally optimized structures of organic layers for organic light emitting devices and stable and efficient materials for organic layers of organic light emitting devices.

Particularly, for maximum efficiency in a light emitting layer, an appropriate combination of energy band gaps of a host and a dopant is required such that holes and electrons migrate to the dopant through stable electrochemical paths to form excitons. Thus, there is a continuing need to develop host and dopant materials.

### DETAILED DESCRIPTION OF THE INVENTION

### Problems to be Solved by the Invention

Accordingly, the present invention is intended to provide a host material having a specific structure that is employed in a light emitting layer of an organic light emitting device. The present invention is also intended to provide a highly efficient organic light emitting device that employs the host material to achieve improved luminescent properties.

### Means for Solving the Problems

One aspect of the present invention provides a pyrene derivative which is employed as a host compound in an organic layer, preferably a light emitting layer of a device and which is represented by Formula I:

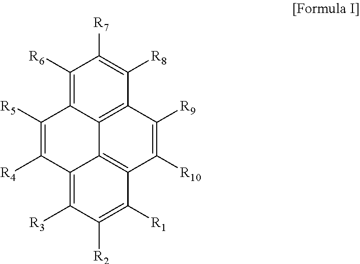

with the proviso that at least one specific structure selected from those represented by Structural Formulas 1 to 4:

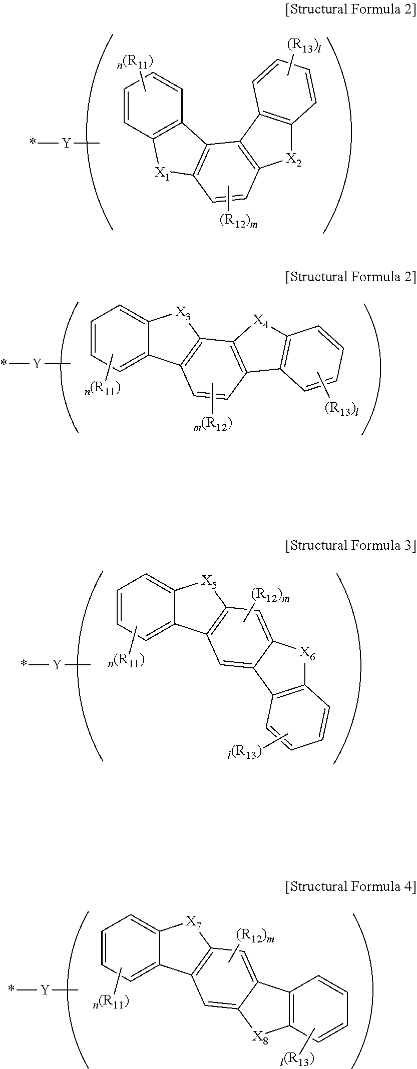

is introduced to the pyrene skeleton.

The specific structures of Formula I and Structural Formulas 1 to 4, definitions of the substituents in Formula I and Structural Formulas 1 to 4, and specific compounds that can be represented by Formula I and Structural Formulas 1 to 4 are described below.

The present invention also provides an organic light emitting device including a first electrode, a second electrode opposite to the first electrode, and a light emitting layer interposed between the first and second electrodes wherein the light emitting layer includes the compound represented by Formula I.

### Effects of the Invention

The organic light emitting device of the present invention includes a light emitting layer employing the pyrene derivative with a specific structure as a host. The use of the host ensures excellent luminescent properties and high efficiency of the device. Due to these advantages, the organic light emitting device of the present invention can find useful applications in not only lighting systems but also a variety of displays, including flat panel displays, flexible displays, and wearable displays.

### BEST MODE FOR CARRYING OUT THE INVENTION

The present invention will now be described in more detail.

One aspect of the present invention is directed to a compound represented by Formula I:

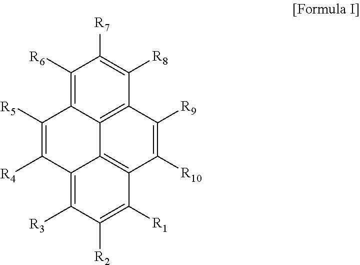

wherein R₁ to R₁₀ are the same as or different from each other and are each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₁-C₃₀ alkylamine, substituted or unsubstituted C₆-C₅₀ arylamine, substituted or unsubstituted C₁-C₃₀ alkylsilyl, substituted or unsubstituted C₆-C₆₀ arylsilyl, substituted or unsubstituted C₁-C₃₀ alkoxy, and substituted or unsubstituted C₆-C₅₀ aryloxy, with the proviso that at least one specific structure selected from those represented by Structural Formulas 1 to 4:

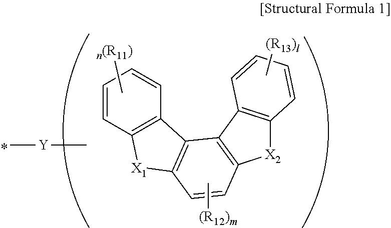

wherein X₁ and X₂ are each independently S, O or NR₁₄, R₁₄ is selected from hydrogen, deuterium, substituted or unsubstituted C₁-C₃₀ alkyl, substituted or unsubstituted C₃-C₃₀ cycloalkyl, substituted or unsubstituted C₆-C₅₀ aryl, and substituted or unsubstituted C₂-C₅₀ heteroaryl, R₁₁ to R₁₃ are the same as or different from each other and are each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₁-C₃₀ alkylamine, substituted or unsubstituted C₆-C₅₀ arylamine, substituted or unsubstituted C₁-C₃₀ alkylsilyl, substituted or unsubstituted C₆-C₅₀ arylsilyl, substituted or unsubstituted C₁-C₃₀ alkoxy, and substituted or unsubstituted C₆-C₅₀ aryloxy, the asterisk (*) represents a site where one of R₁ to R₁₀ is bonded, Y is a single bond or is selected from substituted or unsubstituted C₆-C₃₀ arylene and substituted or unsubstituted C₂-C₃₀ heteroarylene, with the proviso that one of R₁₁ to R₁₃ is linked to Y, each of l and n is an integer from 1 to 4, and m is an integer of 1 or 2, provided that when l is 2 or greater, the groups R₁₃ are the same as or different from each other, when m is 2 or greater, the groups R₁₂ are the same as or different from each other, and when n is 2 or greater, the groups R₁₁ are the same as or different from each other;

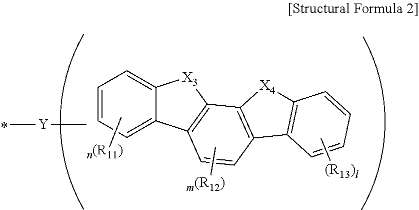

wherein X₃ to X₄ are as defined for X₁ and X₂ in Structural Formula 1 and R₁₁ to R₁₃, the asterisk (*), Y, l, n, and m are as defined in Structural Formula 1;

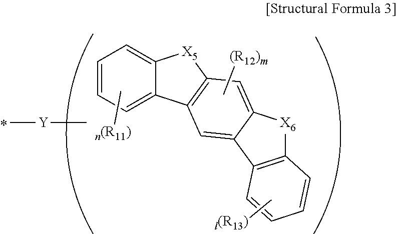

wherein X₅ to X₆ are as defined for X₁ and X₂ in Structural Formula 1 and R₁₁ to R₁₃, the asterisk (*), Y, l, n, and m are as defined in Structural Formula 1; and

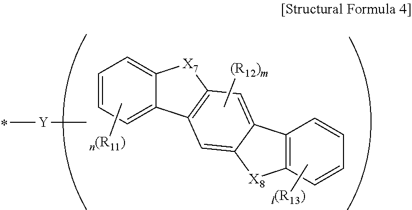

wherein X₇ to X₈ are as defined for X₁ and X₂ in Structural Formula 1 and R₁₁ to R₁₃, the asterisk (*), Y, l, n, and m are as defined in Structural Formula 1, is introduced to the pyrene skeleton.

Specifically, at least one of R₁ to R₁₀ is linked to one of Structural Formulas 1 to 4.

The compound of Formula I is employed as a host compound in a light emitting layer of an organic light emitting device to achieve high efficiency of the device.

According to one embodiment of the present invention, at least one of R₁, R₃, R₆, and R₈ may be linked to the structure represented by one of Structural Formulas 1 to 4 and at least one of R₁ to R₁₀ may be substituted or unsubstituted C₆-C₁₈ aryl.

According to one embodiment of the present invention, the structure represented by one of Structural Formulas 1 to 4 may be introduced to R₈ and a further substituent may be introduced to R₃.

As used herein, the term “substituted” in the definition of the substituents in Formula I and Structural Formulas 1 to 4 indicates substitution with one or more substituents selected from deuterium, cyano, halogen, hydroxyl, nitro, C₁-C₂₄ straight, branched or cyclic alkyl, C₃-C₂₄ cycloalkyl, C₁-C₂₄ straight, branched or cyclic haloalkyl, C₁-C₂₄ alkenyl, C₁-C₂₄ alkynyl, C₁-C₂₄ heteroalkyl, C₁-C₂₄ heterocycloalkyl, C₆-C₂₄ aryl, C₆-C₂₄ arylalkyl, C₂-C₂₄ heteroaryl, C₂-C₂₄ heteroarylalkyl, C₁-C₂₄ alkoxy, C₁-C₂₄ alkylamino, C₁-C₂₄ arylamino, C₁-C₂₄ heteroarylamino, C₁-C₂₄ alkylsilyl, C₁-C₂₄ arylsilyl, and C₁-C₂₄ aryloxy, or a combination thereof. As used herein, the term “unsubstituted” indicates having no substituent.

In the “substituted or unsubstituted C₁-C₁₀ alkyl”, “substituted or unsubstituted C₆-C₃₀ aryl”, etc., the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the expression “form a ring with an adjacent substituent” means that the corresponding substituent combines with an adjacent substituent to form a substituted or unsubstituted alicyclic or aromatic ring and the term “adjacent substituent” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other.

The alkyl groups may be straight or branched. The number of carbon atoms in the alkyl groups is not particularly limited but is preferably 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

The cycloalkyl group is intended to include monocyclic and polycyclic ones and may be optionally substituted with one or more other substituents. As used herein, the term “polycyclic” means that the cycloalkyl group may be directly attached or fused to one or more other cyclic groups. The other cyclic groups may be cycloalkyl groups and other examples thereof include heterocycloalkyl, aryl, and heteroaryl groups. The cycloalkyl group may be specifically a cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl or cyclooctyl group but is not limited thereto.

The heterocycloalkyl group is intended to include monocyclic and polycyclic ones interrupted by a heteroatom such as O, S, Se, N or Si and may be optionally substituted with one or more other substituents. As used herein, the term “polycyclic” means that the heterocycloalkyl group may be directly attached or fused to one or more other cyclic groups. The other cyclic groups may be heterocycloalkyl groups and other examples thereof include cycloalkyl, aryl, and heteroaryl groups.

The aryl groups may be monocyclic or polycyclic ones. Examples of the monocyclic aryl groups include, but are not limited to, phenyl, biphenyl, terphenyl, and stilbenyl groups. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

The heteroaryl groups refer to heterocyclic groups interrupted by one or more heteroatoms. Examples of the heteroaryl groups include, but are not limited to, thiophene, furan, pyrrole, imidazole, triazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

The alkoxy group may be specifically a methoxy, ethoxy, propoxy, isobutyloxy, sec-butyloxy, pentyloxy, iso-amyloxy or hexyloxy group but is not limited thereto.

The silyl group is intended to include alkyl-substituted silyl groups and aryl-substituted silyl groups. Specific examples of such silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl.

The amine group may be, for example, —NH₂, alkylamine, arylamine or arylheteroarylamine. The arylamine refers to an aryl-substituted amine group, the alkylamine refers to an alkyl-substituted amine group, and the arylheteroarylamine refers to an aryl- and heteroaryl-substituted amine group. The arylamine may be, for example, substituted or unsubstituted monoarylamine, substituted or unsubstituted diarylamine, or substituted or unsubstituted triarylamine. The aryl and/or heteroaryl groups in the arylamine and arylheteroarylamine groups may be monocyclic or polycyclic ones. The arylamine and arylheteroarylamine groups may include two or more aryl and/or heteroaryl groups. In this case, the aryl groups may be monocyclic and/or polycyclic ones and the heteroaryl groups may be monocyclic and/or polycyclic ones. The aryl and/or heteroaryl groups in the arylamine and arylheteroarylamine groups may be selected from those exemplified above.

The aryl groups in the aryloxy and arylthioxy groups are the same as those exemplified above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. Specific examples of the arylthioxy groups include, but are not limited to, phenylthioxy, 2-methylphenylthioxy, and 4-tert-butylphenylthioxy groups.

The halogen group may be, for example, fluorine, chlorine, bromine or iodine.

According to one embodiment of the present invention, the compound represented by Formula I may be selected from the following compounds 1 to 102:

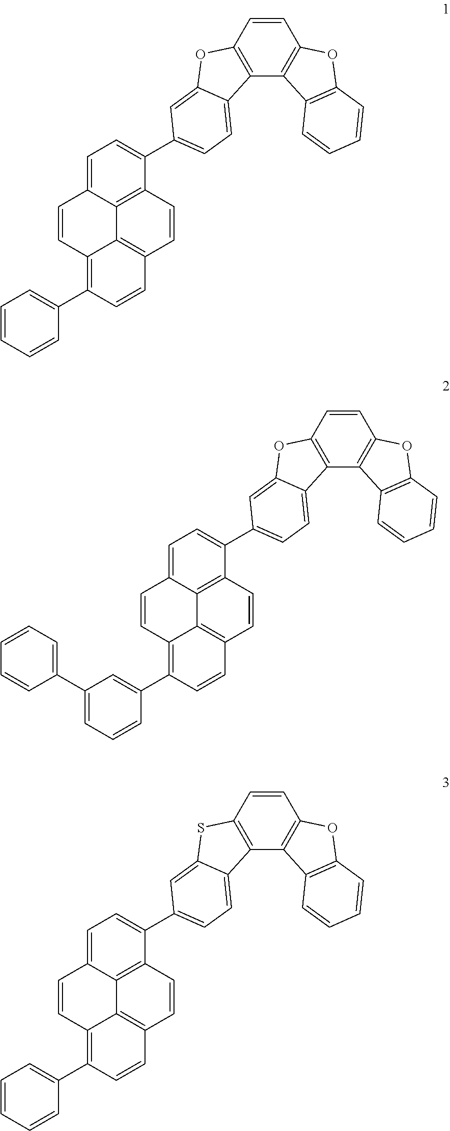 [SMILES: c1ccc(-c2ccc3ccc4c(-c5ccc6c(c5)oc5ccc7oc8ccccc8c7c56)ccc5ccc2c3c54)cc1 ; c1ccc(-c2cccc(-c3ccc4ccc5c(-c6ccc7c(c6)oc6ccc8oc9ccccc9c8c67)ccc6ccc3c4c65)c2)cc1 ; c1ccc(-c2ccc3ccc4c(-c5ccc6c(c5)sc5ccc7oc8ccccc8c7c56)ccc5ccc2c3c54)cc1]

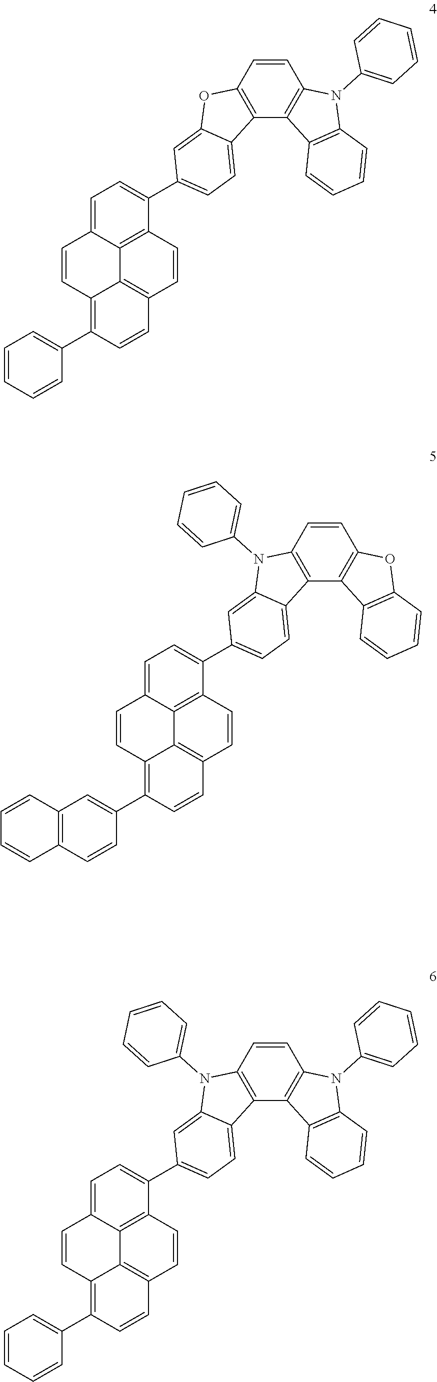 [SMILES: c1ccc(-c2ccc3ccc4c(-c5ccc6c(c5)oc5ccc7c(c8ccccc8n7-c7ccccc7)c56)ccc5ccc2c3c54)cc1 ; c1ccc(-n2c3cc(-c4ccc5ccc6c(-c7ccc8ccccc8c7)ccc7ccc4c5c76)ccc3c3c4c(ccc32)oc2ccccc24)cc1 ; c1ccc(-c2ccc3ccc4c(-c5ccc6c7c8c9ccccc9n(-c9ccccc9)c8ccc7n(-c7ccccc7)c6c5)ccc5ccc2c3c54)cc1]

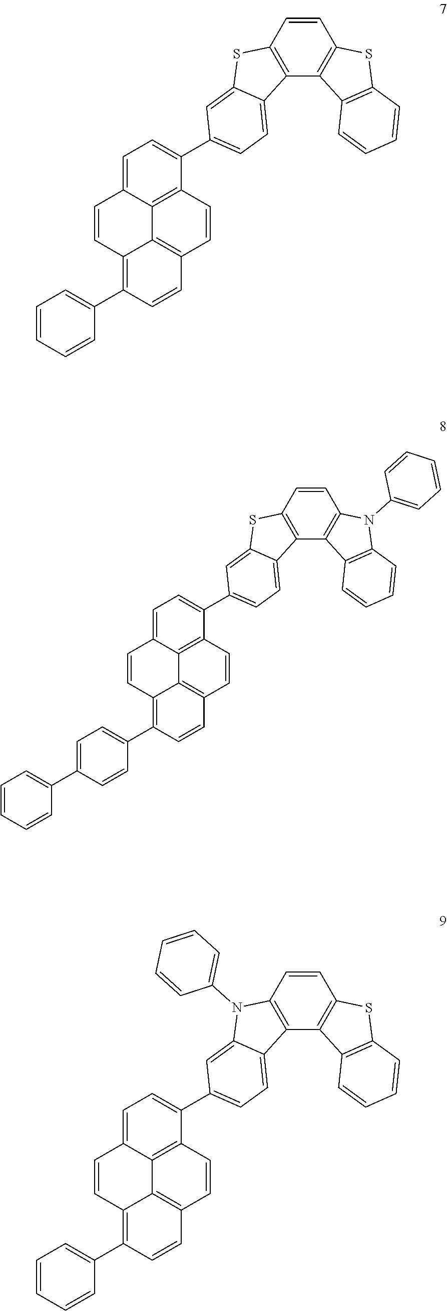 [SMILES: c1ccc(-c2ccc3ccc4c(-c5ccc6c(c5)sc5ccc7sc8ccccc8c7c56)ccc5ccc2c3c54)cc1 ; c1ccc(-c2ccc(-c3ccc4ccc5c(-c6ccc7c(c6)sc6ccc8c(c9ccccc9n8-c8ccccc8)c67)ccc6ccc3c4c65)cc2)cc1 ; c1ccc(-c2ccc3ccc4c(-c5ccc6c7c8c(ccc7n(-c7ccccc7)c6c5)sc5ccccc58)ccc5ccc2c3c54)cc1]

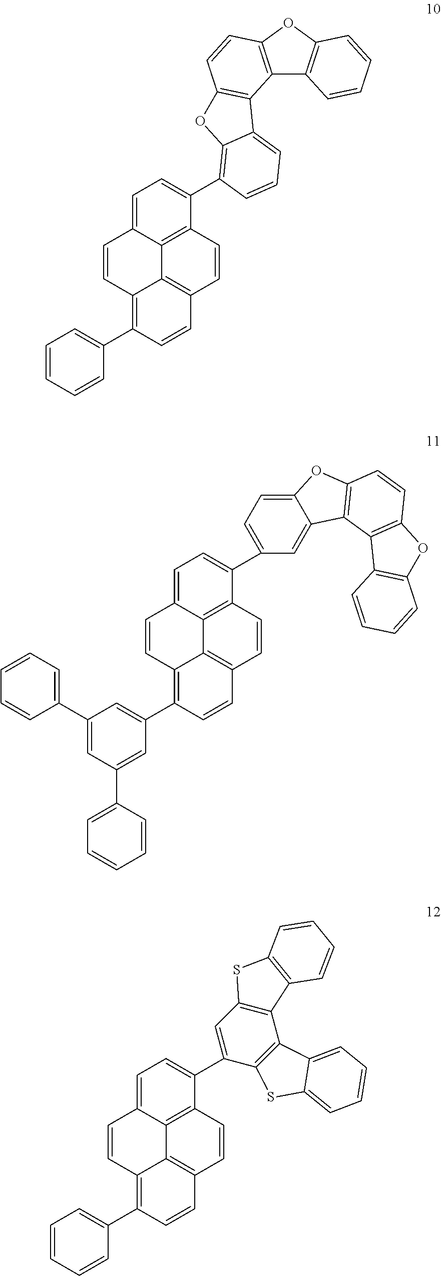 [SMILES: c1ccc(-c2ccc3ccc4c(-c5cccc6c5oc5ccc7oc8ccccc8c7c56)ccc5ccc2c3c54)cc1 ; c1ccc(-c2cc(-c3ccccc3)cc(-c3ccc4ccc5c(-c6ccc7oc8ccc9oc%10ccccc%10c9c8c7c6)ccc6ccc3c4c65)c2)cc1 ; c1ccc(-c2ccc3ccc4c(-c5cc6sc7ccccc7c6c6c5sc5ccccc56)ccc5ccc2c3c54)cc1]

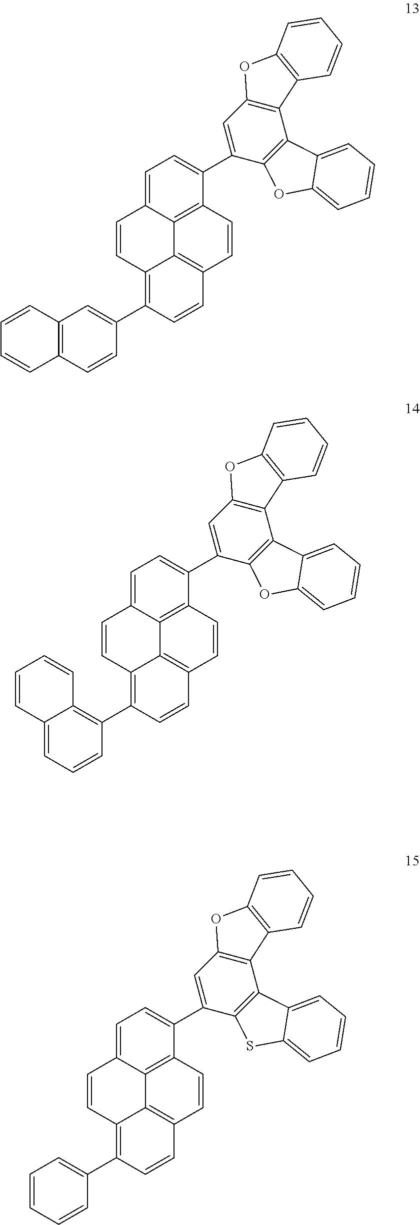 [SMILES: c1ccc2cc(-c3ccc4ccc5c(-c6cc7oc8ccccc8c7c7c6oc6ccccc67)ccc6ccc3c4c65)ccc2c1 ; c1ccc2c(-c3ccc4ccc5c(-c6cc7oc8ccccc8c7c7c6oc6ccccc67)ccc6ccc3c4c65)cccc2c1 ; c1ccc(-c2ccc3ccc4c(-c5cc6oc7ccccc7c6c6c5sc5ccccc56)ccc5ccc2c3c54)cc1]

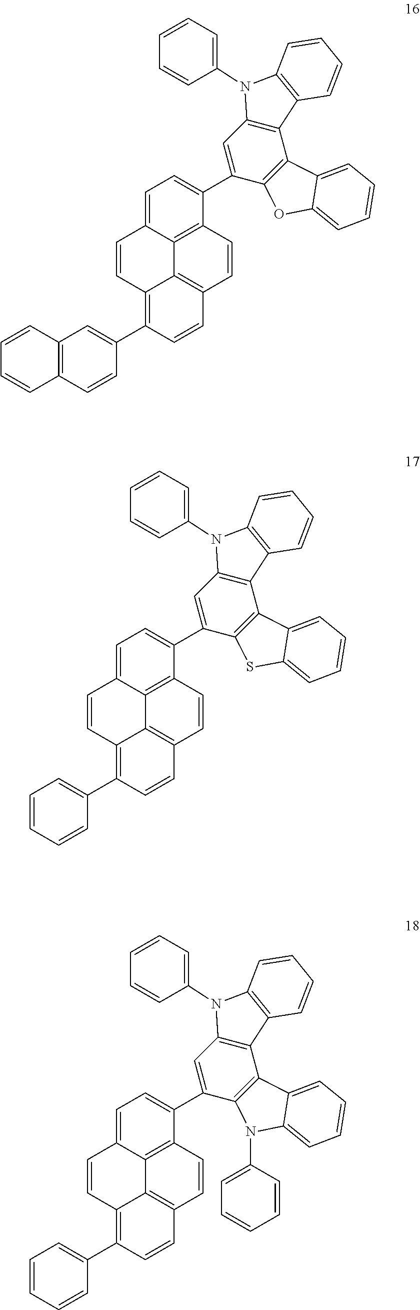 [SMILES: c1ccc(-n2c3ccccc3c3c4c(oc5ccccc54)c(-c4ccc5ccc6c(-c7ccc8ccccc8c7)ccc7ccc4c5c76)cc32)cc1 ; c1ccc(-c2ccc3ccc4c(-c5cc6c(c7ccccc7n6-c6ccccc6)c6c5sc5ccccc56)ccc5ccc2c3c54)cc1 ; c1ccc(-c2ccc3ccc4c(-c5cc6c(c7ccccc7n6-c6ccccc6)c6c7ccccc7n(-c7ccccc7)c56)ccc5ccc2c3c54)cc1]

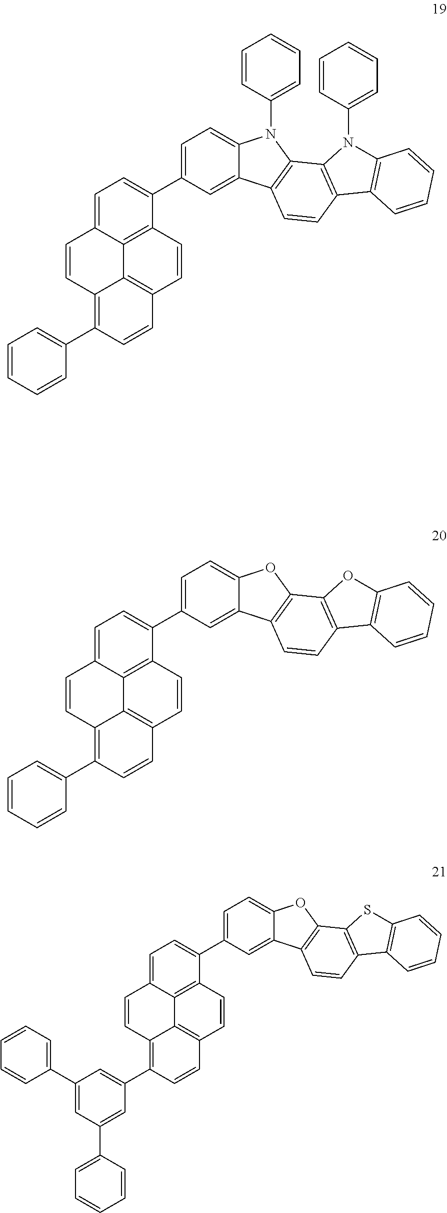 [SMILES: c1ccc(-c2ccc3ccc4c(-c5ccc6c(c5)c5ccc7c8ccccc8n(-c8ccccc8)c7c5n6-c5ccccc5)ccc5ccc2c3c54)cc1 ; c1ccc(-c2ccc3ccc4c(-c5ccc6oc7c(ccc8c9ccccc9oc87)c6c5)ccc5ccc2c3c54)cc1 ; c1ccc(-c2cc(-c3ccccc3)cc(-c3ccc4ccc5c(-c6ccc7oc8c(ccc9c%10ccccc%10sc98)c7c6)ccc6ccc3c4c65)c2)cc1]

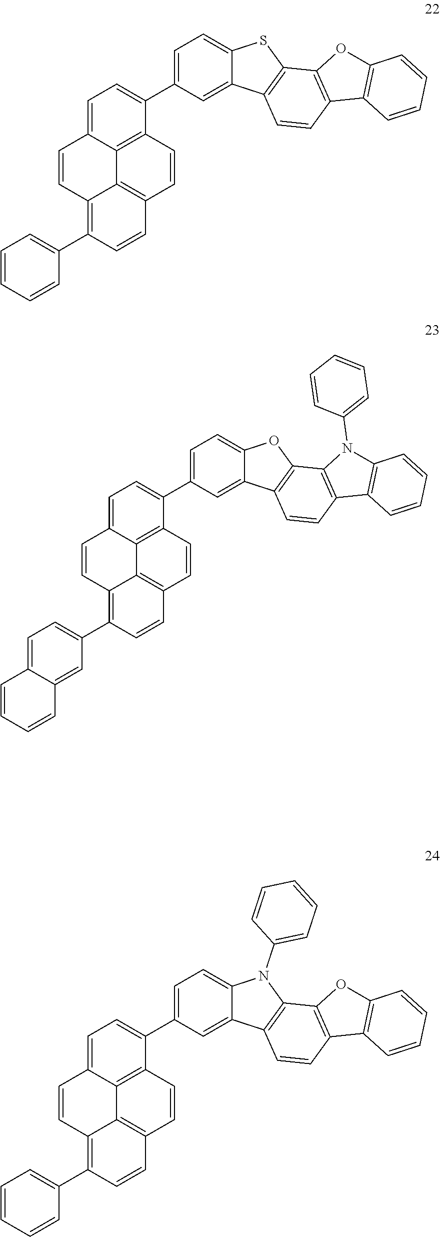 [SMILES: c1ccc(-c2ccc3ccc4c(-c5ccc6sc7c(ccc8c9ccccc9oc87)c6c5)ccc5ccc2c3c54)cc1 ; c1ccc(-n2c3ccccc3c3ccc4c5cc(-c6ccc7ccc8c(-c9ccc%10ccccc%10c9)ccc9ccc6c7c98)ccc5oc4c32)cc1 ; c1ccc(-c2ccc3ccc4c(-c5ccc6c(c5)c5ccc7c8ccccc8oc7c5n6-c5ccccc5)ccc5ccc2c3c54)cc1]

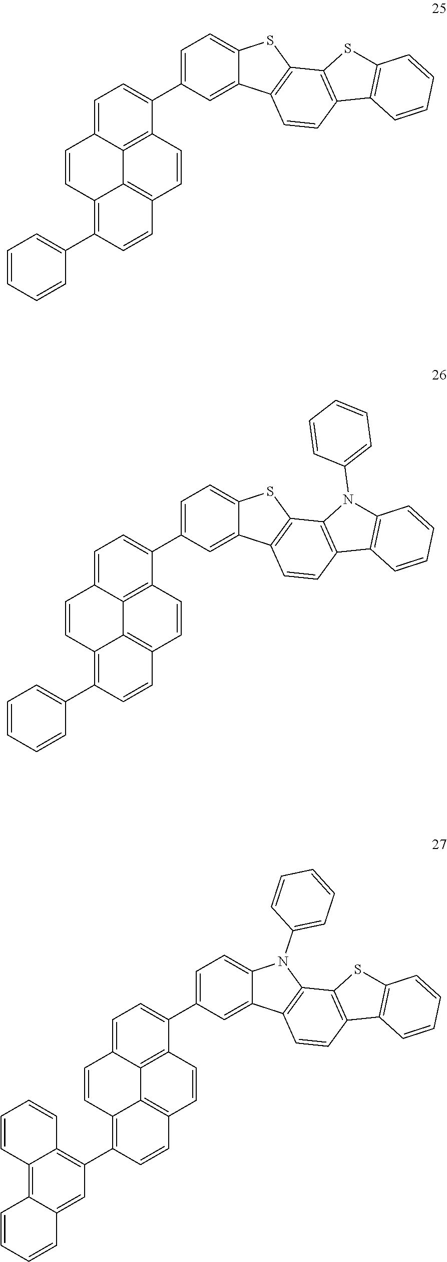 [SMILES: c1ccc(-c2ccc3ccc4c(-c5ccc6sc7c(ccc8c9ccccc9sc87)c6c5)ccc5ccc2c3c54)cc1 ; c1ccc(-c2ccc3ccc4c(-c5ccc6sc7c(ccc8c9ccccc9n(-c9ccccc9)c87)c6c5)ccc5ccc2c3c54)cc1 ; c1ccc(-n2c3ccc(-c4ccc5ccc6c(-c7cc8ccccc8c8ccccc78)ccc7ccc4c5c76)cc3c3ccc4c5ccccc5sc4c32)cc1]

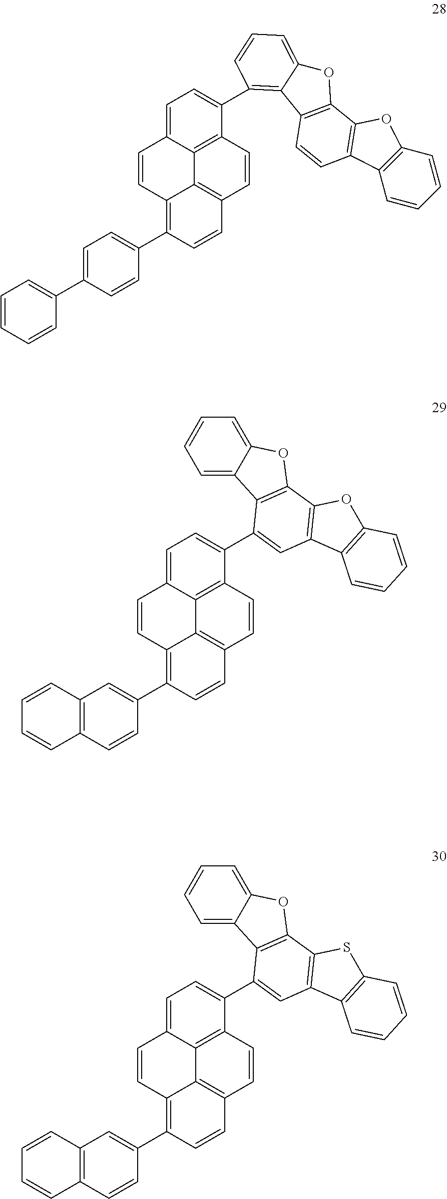 [SMILES: c1ccc(-c2ccc(-c3ccc4ccc5c(-c6cccc7oc8c(ccc9c%10ccccc%10oc98)c67)ccc6ccc3c4c65)cc2)cc1 ; c1ccc2cc(-c3ccc4ccc5c(-c6cc7c8ccccc8oc7c7oc8ccccc8c67)ccc6ccc3c4c65)ccc2c1 ; c1ccc2cc(-c3ccc4ccc5c(-c6cc7c8ccccc8sc7c7oc8ccccc8c67)ccc6ccc3c4c65)ccc2c1]

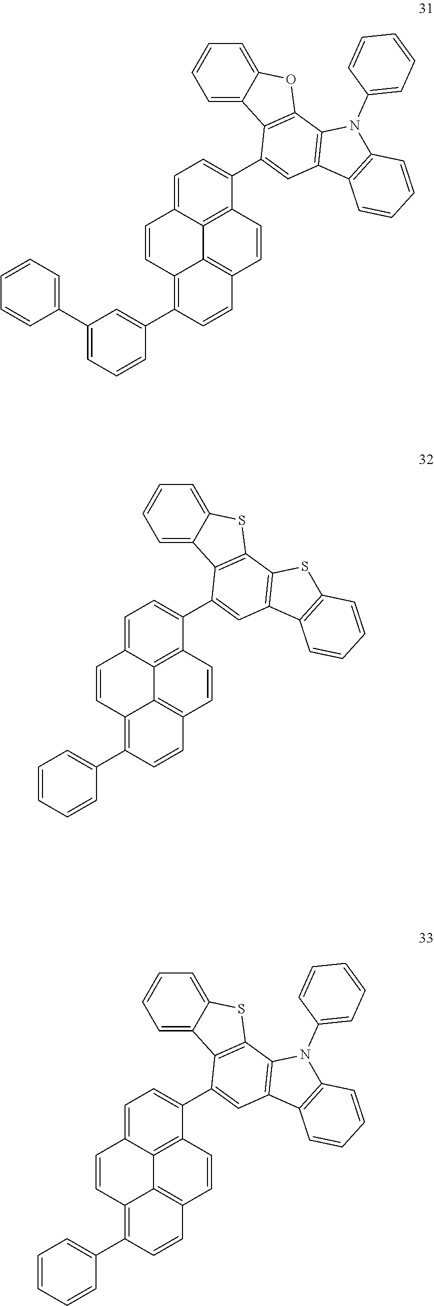 [SMILES: c1ccc(-c2cccc(-c3ccc4ccc5c(-c6cc7c8ccccc8n(-c8ccccc8)c7c7oc8ccccc8c67)ccc6ccc3c4c65)c2)cc1 ; c1ccc(-c2ccc3ccc4c(-c5cc6c7ccccc7sc6c6sc7ccccc7c56)ccc5ccc2c3c54)cc1 ; c1ccc(-c2ccc3ccc4c(-c5cc6c7ccccc7n(-c7ccccc7)c6c6sc7ccccc7c56)ccc5ccc2c3c54)cc1]

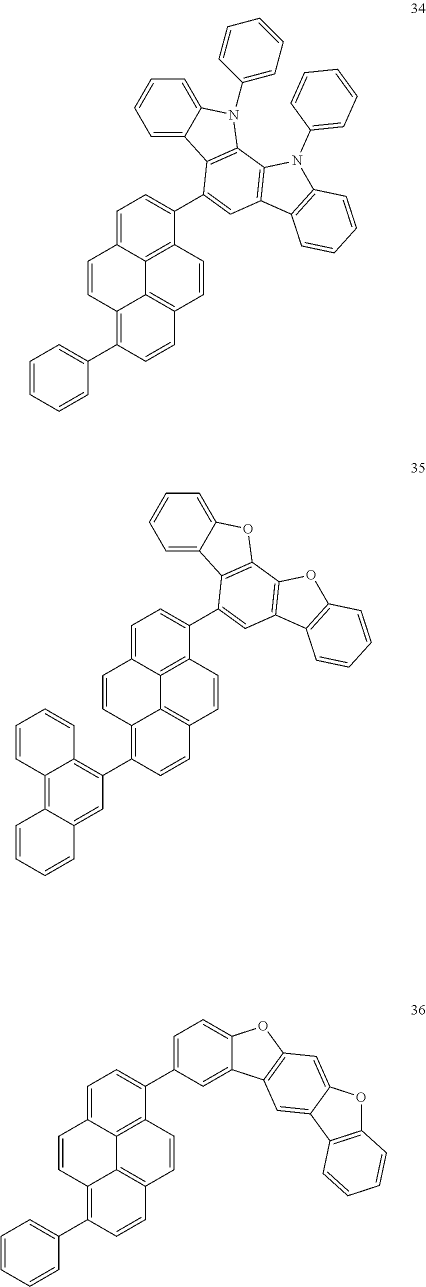 [SMILES: c1ccc(-c2ccc3ccc4c(-c5cc6c7ccccc7n(-c7ccccc7)c6c6c5c5ccccc5n6-c5ccccc5)ccc5ccc2c3c54)cc1 ; c1ccc2c(c1)cc(-c1ccc3ccc4c(-c5cc6c7ccccc7oc6c6oc7ccccc7c56)ccc5ccc1c3c54)c1ccccc12 ; c1ccc(-c2ccc3ccc4c(-c5ccc6oc7cc8oc9ccccc9c8cc7c6c5)ccc5ccc2c3c54)cc1]

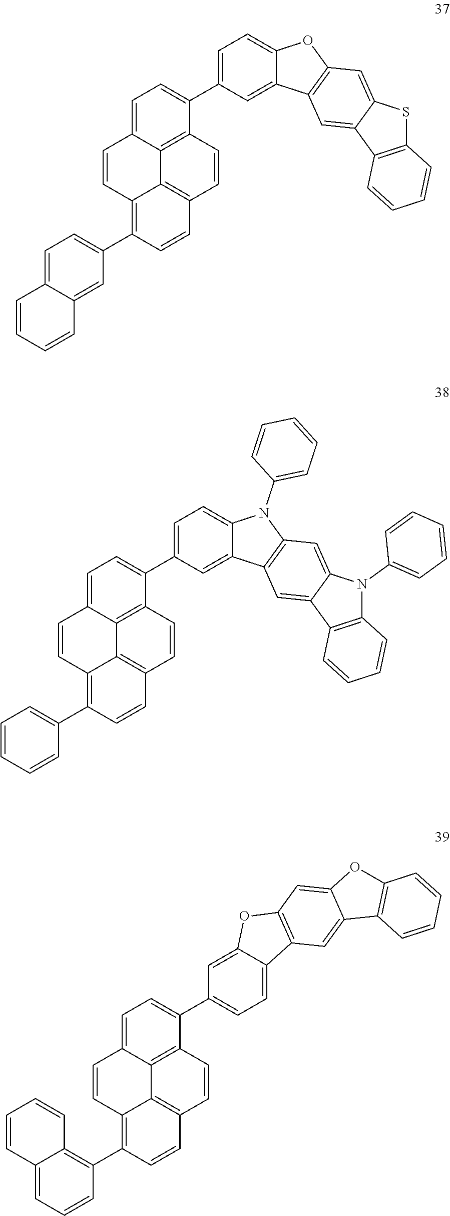 [SMILES: c1ccc2cc(-c3ccc4ccc5c(-c6ccc7oc8cc9sc%10ccccc%10c9cc8c7c6)ccc6ccc3c4c65)ccc2c1 ; c1ccc(-c2ccc3ccc4c(-c5ccc6c(c5)c5cc7c8ccccc8n(-c8ccccc8)c7cc5n6-c5ccccc5)ccc5ccc2c3c54)cc1 ; c1ccc2c(-c3ccc4ccc5c(-c6ccc7c(c6)oc6cc8oc9ccccc9c8cc67)ccc6ccc3c4c65)cccc2c1]

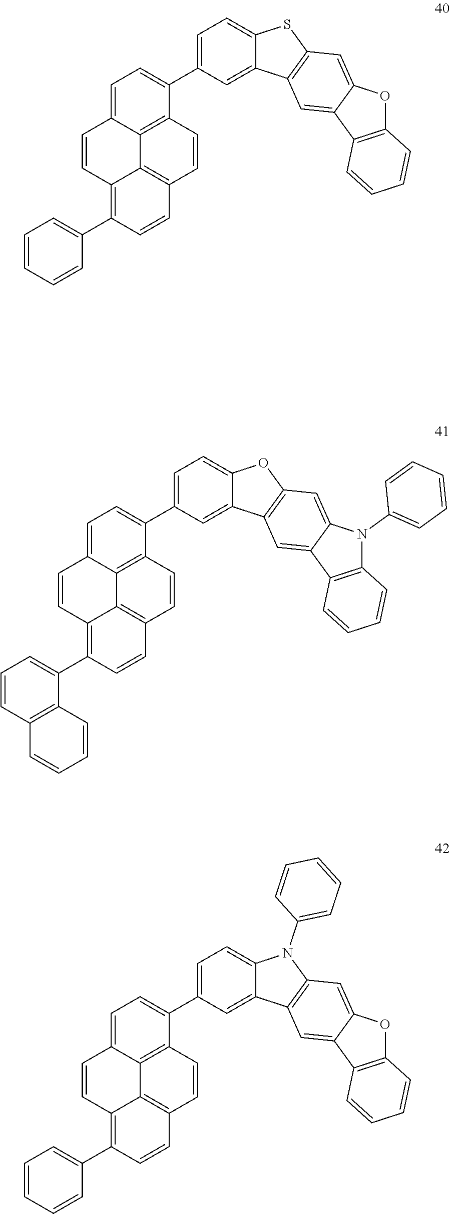 [SMILES: c1ccc(-c2ccc3ccc4c(-c5ccc6sc7cc8oc9ccccc9c8cc7c6c5)ccc5ccc2c3c54)cc1 ; c1ccc(-n2c3ccccc3c3cc4c(cc32)oc2ccc(-c3ccc5ccc6c(-c7cccc8ccccc78)ccc7ccc3c5c76)cc24)cc1 ; c1ccc(-c2ccc3ccc4c(-c5ccc6c(c5)c5cc7c(cc5n6-c5ccccc5)oc5ccccc57)ccc5ccc2c3c54)cc1]

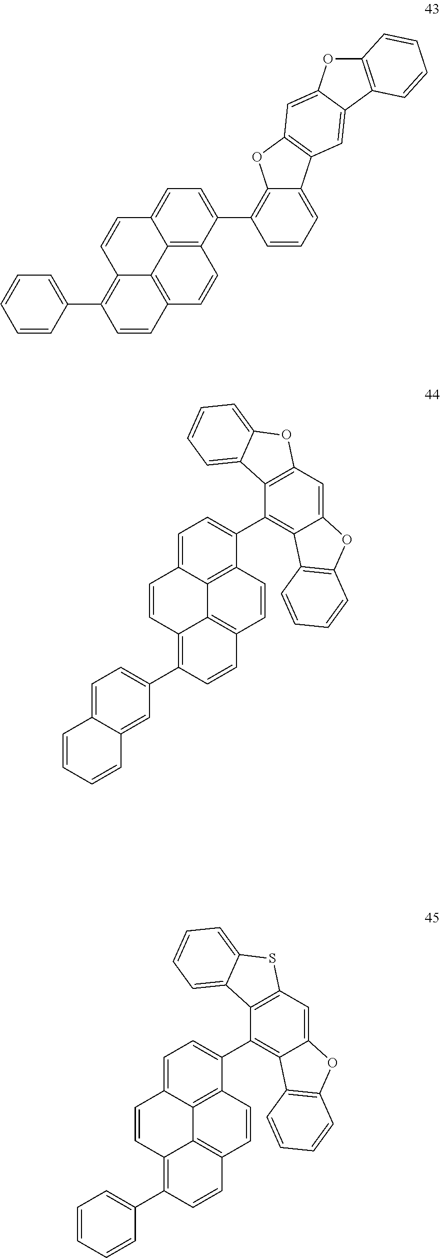 [SMILES: c1ccc(-c2ccc3ccc4c(-c5cccc6c5oc5cc7oc8ccccc8c7cc56)ccc5ccc2c3c54)cc1 ; c1ccc2cc(-c3ccc4ccc5c(-c6c7c(cc8oc9ccccc9c68)oc6ccccc67)ccc6ccc3c4c65)ccc2c1 ; c1ccc(-c2ccc3ccc4c(-c5c6c(cc7sc8ccccc8c57)oc5ccccc56)ccc5ccc2c3c54)cc1]

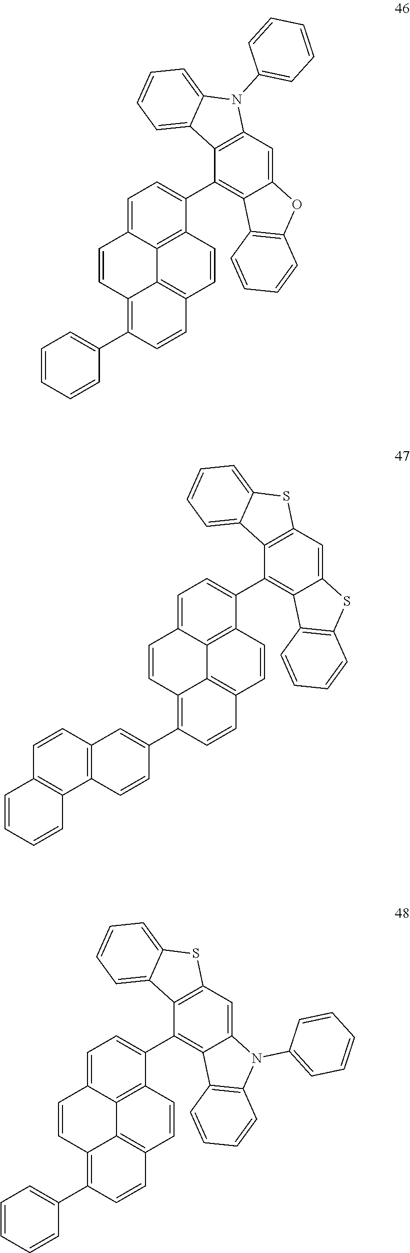 [SMILES: c1ccc(-c2ccc3ccc4c(-c5c6c(cc7c5c5ccccc5n7-c5ccccc5)oc5ccccc56)ccc5ccc2c3c54)cc1 ; c1ccc2c(c1)ccc1cc(-c3ccc4ccc5c(-c6c7c(cc8sc9ccccc9c68)sc6ccccc67)ccc6ccc3c4c65)ccc12 ; c1ccc(-c2ccc3ccc4c(-c5c6c(cc7c5c5ccccc5n7-c5ccccc5)sc5ccccc56)ccc5ccc2c3c54)cc1]

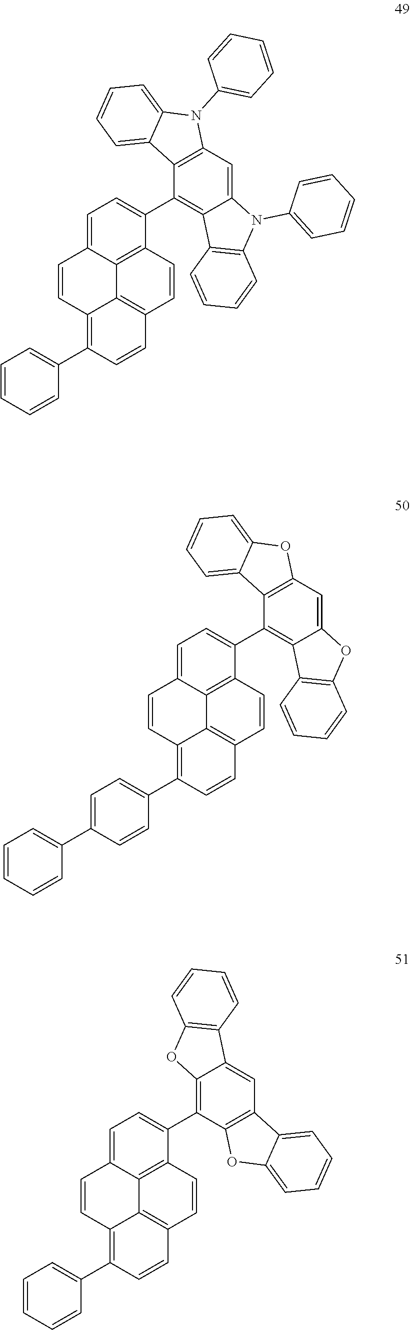 [SMILES: c1ccc(-c2ccc3ccc4c(-c5c6c7ccccc7n(-c7ccccc7)c6cc6c5c5ccccc5n6-c5ccccc5)ccc5ccc2c3c54)cc1 ; c1ccc(-c2ccc(-c3ccc4ccc5c(-c6c7c(cc8oc9ccccc9c68)oc6ccccc67)ccc6ccc3c4c65)cc2)cc1 ; c1ccc(-c2ccc3ccc4c(-c5c6oc7ccccc7c6cc6c5oc5ccccc56)ccc5ccc2c3c54)cc1]

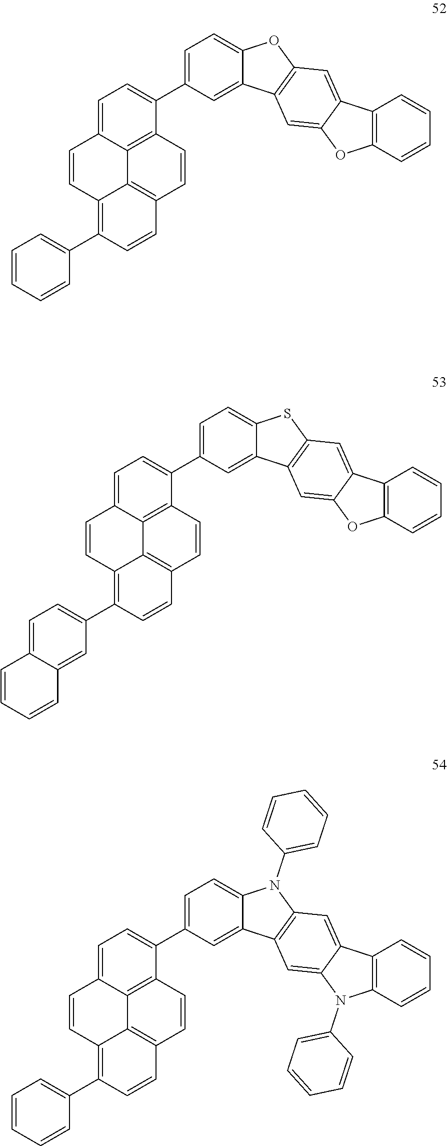 [SMILES: c1ccc(-c2ccc3ccc4c(-c5ccc6oc7cc8c(cc7c6c5)oc5ccccc58)ccc5ccc2c3c54)cc1 ; c1ccc2cc(-c3ccc4ccc5c(-c6ccc7sc8cc9c(cc8c7c6)oc6ccccc69)ccc6ccc3c4c65)ccc2c1 ; c1ccc(-c2ccc3ccc4c(-c5ccc6c(c5)c5cc7c(cc5n6-c5ccccc5)c5ccccc5n7-c5ccccc5)ccc5ccc2c3c54)cc1]

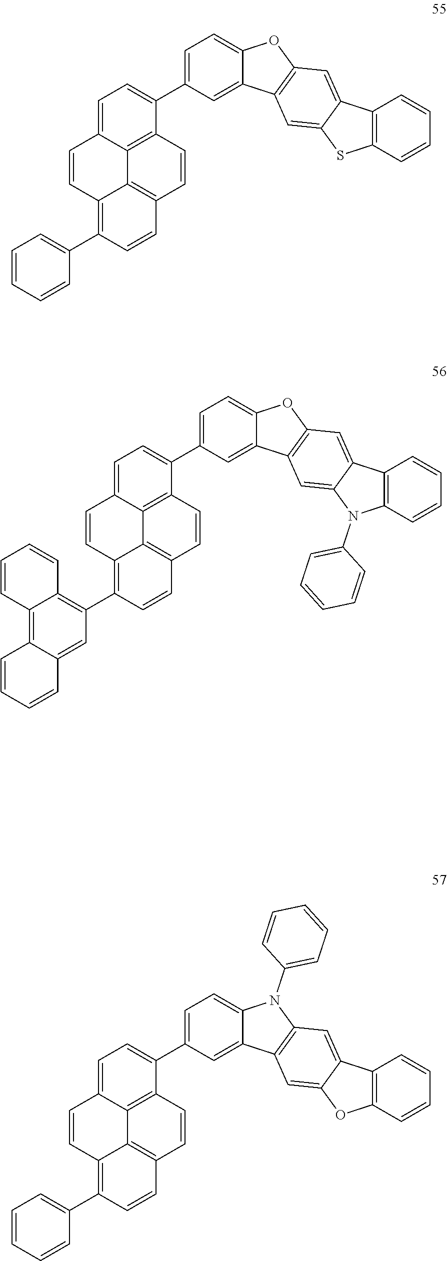 [SMILES: c1ccc(-c2ccc3ccc4c(-c5ccc6oc7cc8c(cc7c6c5)sc5ccccc58)ccc5ccc2c3c54)cc1 ; c1ccc(-n2c3ccccc3c3cc4oc5ccc(-c6ccc7ccc8c(-c9cc%10ccccc%10c%10ccccc9%10)ccc9ccc6c7c98)cc5c4cc32)cc1 ; c1ccc(-c2ccc3ccc4c(-c5ccc6c(c5)c5cc7oc8ccccc8c7cc5n6-c5ccccc5)ccc5ccc2c3c54)cc1]

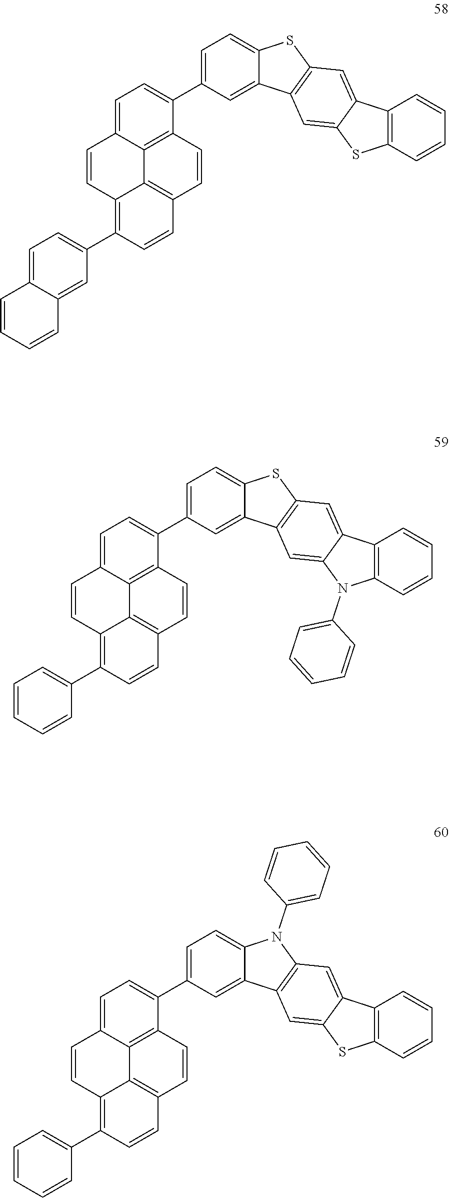 [SMILES: c1ccc2cc(-c3ccc4ccc5c(-c6ccc7sc8cc9c(cc8c7c6)sc6ccccc69)ccc6ccc3c4c65)ccc2c1 ; c1ccc(-c2ccc3ccc4c(-c5ccc6sc7cc8c9ccccc9n(-c9ccccc9)c8cc7c6c5)ccc5ccc2c3c54)cc1 ; c1ccc(-c2ccc3ccc4c(-c5ccc6c(c5)c5cc7sc8ccccc8c7cc5n6-c5ccccc5)ccc5ccc2c3c54)cc1]

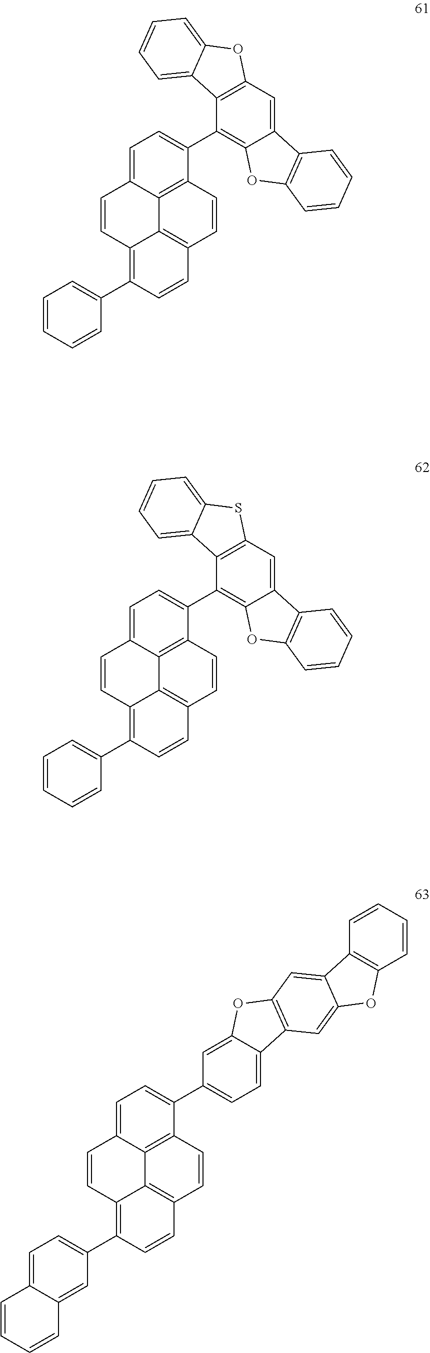 [SMILES: c1ccc(-c2ccc3ccc4c(-c5c6oc7ccccc7c6cc6oc7ccccc7c56)ccc5ccc2c3c54)cc1 ; c1ccc(-c2ccc3ccc4c(-c5c6oc7ccccc7c6cc6sc7ccccc7c56)ccc5ccc2c3c54)cc1 ; c1ccc2cc(-c3ccc4ccc5c(-c6ccc7c(c6)oc6cc8c(cc67)oc6ccccc68)ccc6ccc3c4c65)ccc2c1]

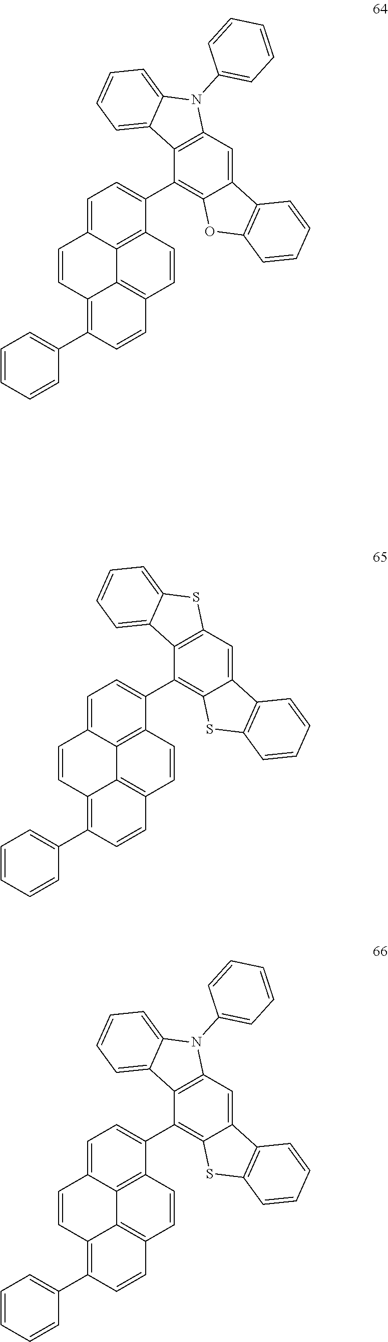 [SMILES: c1ccc(-c2ccc3ccc4c(-c5c6oc7ccccc7c6cc6c5c5ccccc5n6-c5ccccc5)ccc5ccc2c3c54)cc1 ; c1ccc(-c2ccc3ccc4c(-c5c6sc7ccccc7c6cc6sc7ccccc7c56)ccc5ccc2c3c54)cc1 ; c1ccc(-c2ccc3ccc4c(-c5c6sc7ccccc7c6cc6c5c5ccccc5n6-c5ccccc5)ccc5ccc2c3c54)cc1]

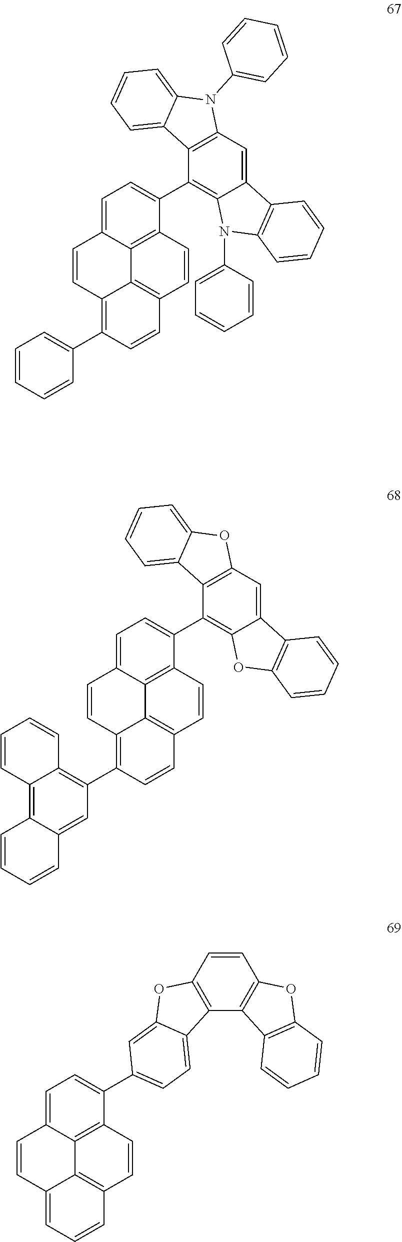 [SMILES: c1ccc(-c2ccc3ccc4c(-c5c6c7ccccc7n(-c7ccccc7)c6cc6c7ccccc7n(-c7ccccc7)c56)ccc5ccc2c3c54)cc1 ; c1ccc2c(c1)cc(-c1ccc3ccc4c(-c5c6oc7ccccc7c6cc6oc7ccccc7c56)ccc5ccc1c3c54)c1ccccc12 ; c1cc2ccc3ccc(-c4ccc5c(c4)oc4ccc6oc7ccccc7c6c45)c4ccc(c1)c2c34]

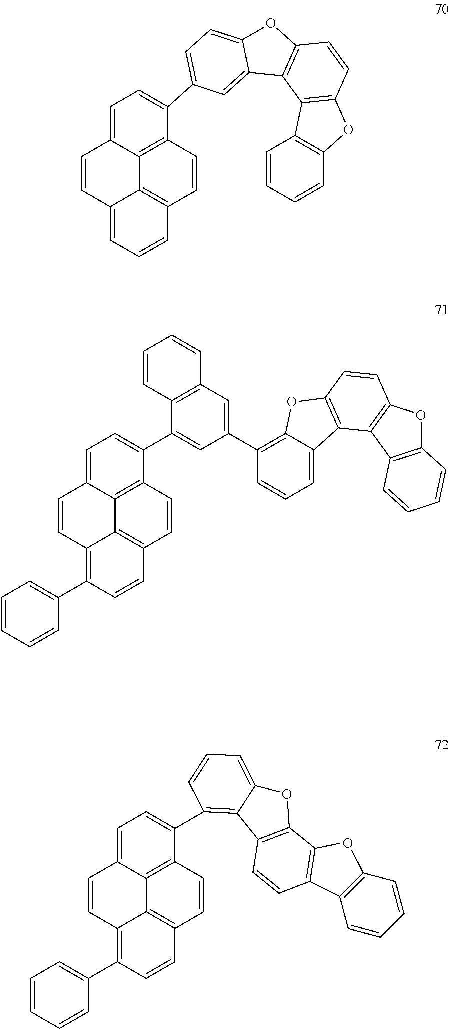 [SMILES: c1cc2ccc3ccc(-c4ccc5oc6ccc7oc8ccccc8c7c6c5c4)c4ccc(c1)c2c34 ; c1ccc(-c2ccc3ccc4c(-c5cc(-c6cccc7c6oc6ccc8oc9ccccc9c8c67)cc6ccccc56)ccc5ccc2c3c54)cc1 ; c1ccc(-c2ccc3ccc4c(-c5cccc6oc7c(ccc8c9ccccc9oc87)c56)ccc5ccc2c3c54)cc1]

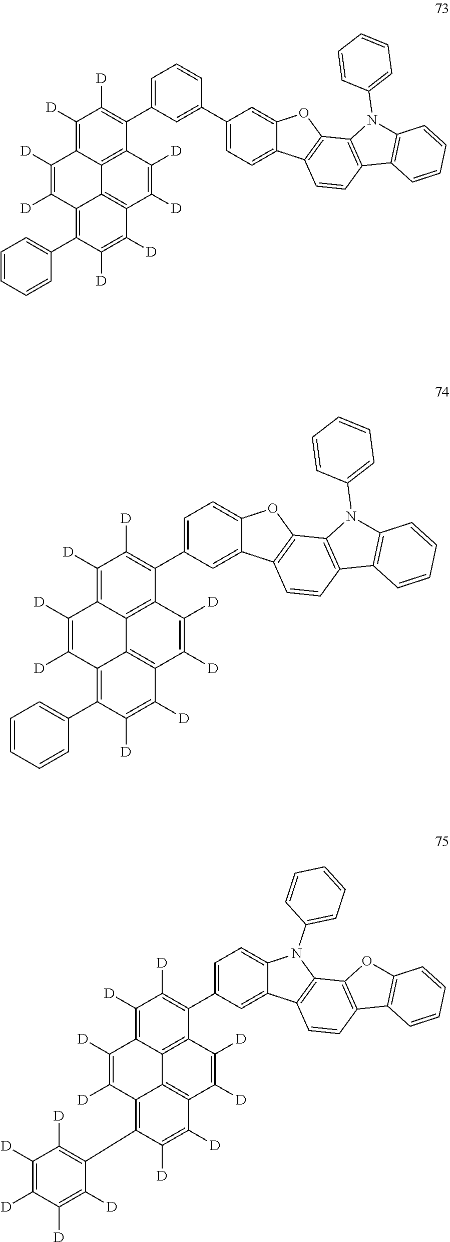 [SMILES: [2H]c1c([2H])c2c([2H])c([2H])c3c(-c4cccc(-c5ccc6c(c5)oc5c6ccc6c7ccccc7n(-c7ccccc7)c65)c4)c([2H])c([2H])c4c([2H])c([2H])c(c1-c1ccccc1)c2c43 ; [2H]c1c([2H])c2c([2H])c([2H])c3c(-c4ccc5oc6c(ccc7c8ccccc8n(-c8ccccc8)c76)c5c4)c([2H])c([2H])c4c([2H])c([2H])c(c1-c1ccccc1)c2c43 ; [2H]c1c([2H])c([2H])c(-c2c([2H])c([2H])c3c([2H])c([2H])c4c(-c5ccc6c(c5)c5ccc7c8ccccc8oc7c5n6-c5ccccc5)c([2H])c([2H])c5c([2H])c([2H])c2c3c54)c([2H])c1[2H]]

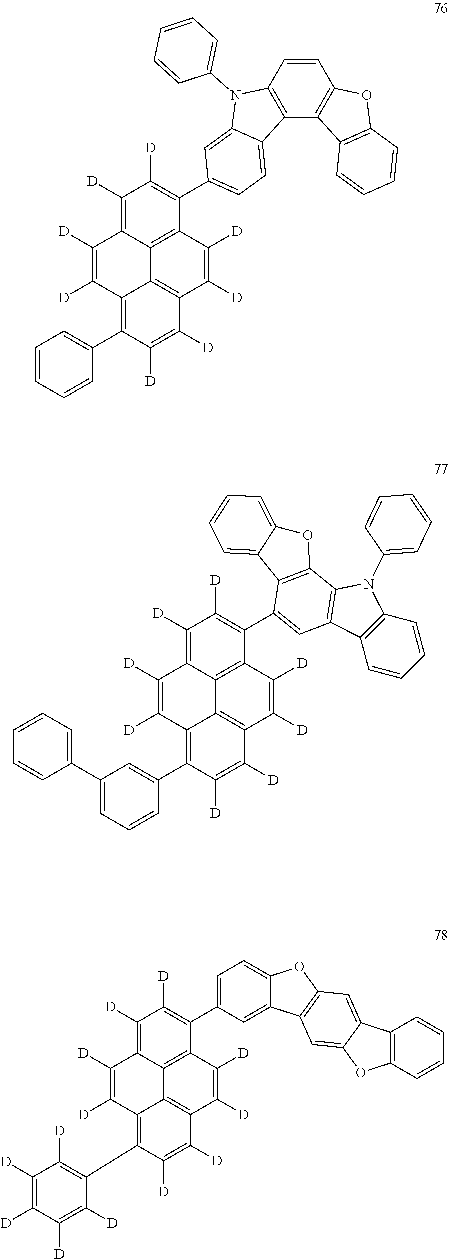 [SMILES: [2H]c1c([2H])c2c([2H])c([2H])c3c(-c4ccc5c6c7c(ccc6n(-c6ccccc6)c5c4)oc4ccccc47)c([2H])c([2H])c4c([2H])c([2H])c(c1-c1ccccc1)c2c43 ; [2H]c1c([2H])c2c([2H])c([2H])c3c(-c4cc5c6ccccc6n(-c6ccccc6)c5c5oc6ccccc6c45)c([2H])c([2H])c4c([2H])c([2H])c(c1-c1cccc(-c5ccccc5)c1)c2c43 ; [2H]c1c([2H])c([2H])c(-c2c([2H])c([2H])c3c([2H])c([2H])c4c(-c5ccc6oc7cc8c(cc7c6c5)oc5ccccc58)c([2H])c([2H])c5c([2H])c([2H])c2c3c54)c([2H])c1[2H]]

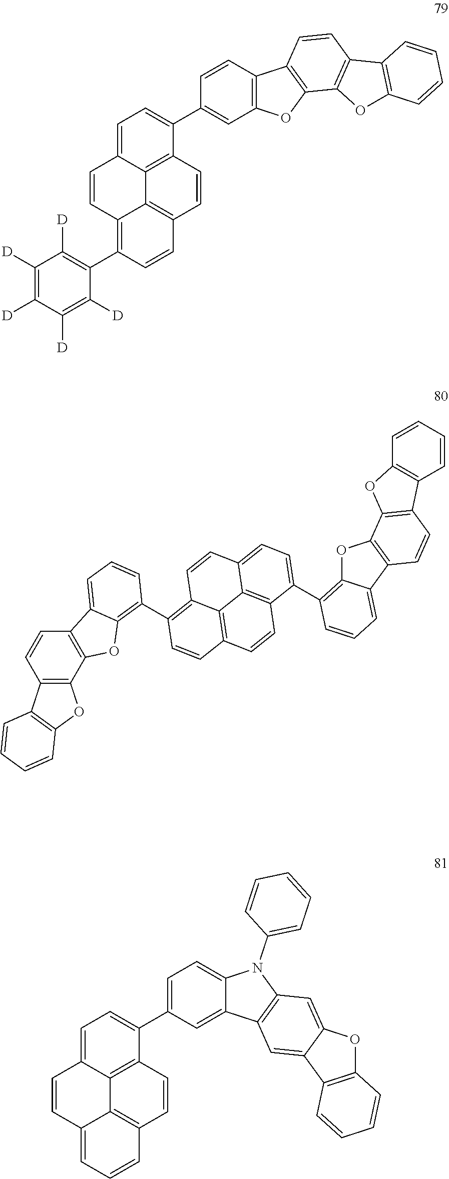 [SMILES: [2H]c1c([2H])c([2H])c(-c2ccc3ccc4c(-c5ccc6c(c5)oc5c6ccc6c7ccccc7oc65)ccc5ccc2c3c54)c([2H])c1[2H] ; c1ccc2c(c1)oc1c2ccc2c3cccc(-c4ccc5ccc6c(-c7cccc8c7oc7c8ccc8c9ccccc9oc87)ccc7ccc4c5c76)c3oc21 ; c1ccc(-n2c3ccc(-c4ccc5ccc6cccc7ccc4c5c67)cc3c3cc4c(cc32)oc2ccccc24)cc1]

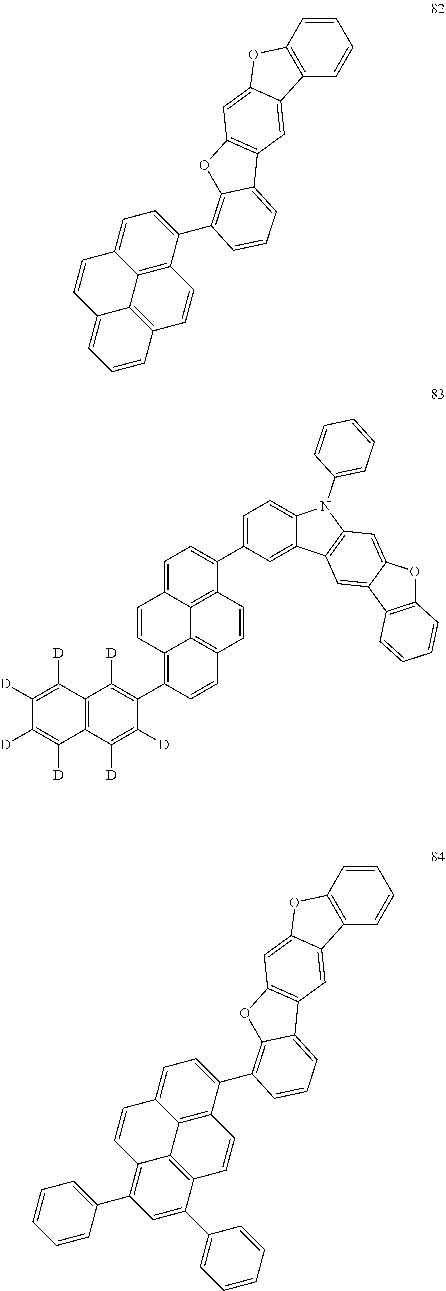 [SMILES: c1cc2ccc3ccc(-c4cccc5c4oc4cc6oc7ccccc7c6cc45)c4ccc(c1)c2c34 ; [2H]c1c([2H])c([2H])c2c([2H])c(-c3ccc4ccc5c(-c6ccc7c(c6)c6cc8c(cc6n7-c6ccccc6)oc6ccccc68)ccc6ccc3c4c65)c([2H])c([2H])c2c1[2H] ; c1ccc(-c2cc(-c3ccccc3)c3ccc4c(-c5cccc6c5oc5cc7oc8ccccc8c7cc56)ccc5ccc2c3c54)cc1]

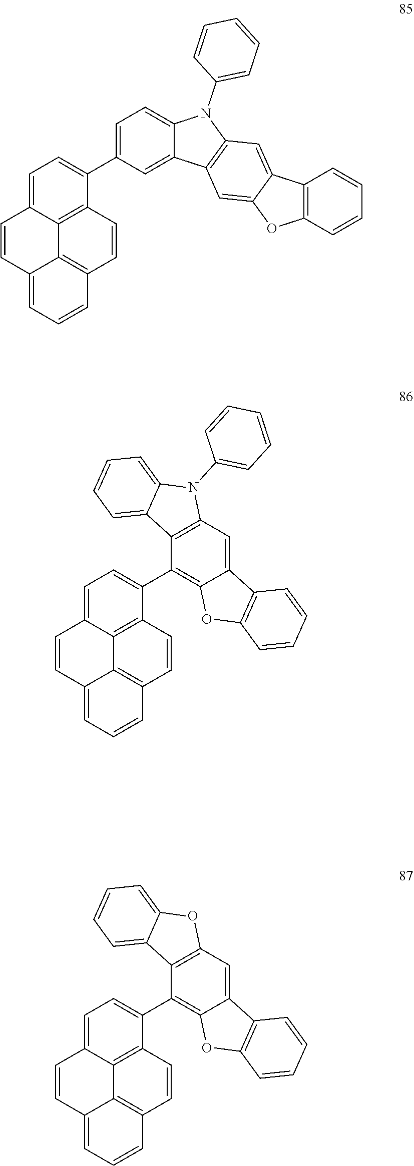 [SMILES: c1ccc(-n2c3ccc(-c4ccc5ccc6cccc7ccc4c5c67)cc3c3cc4oc5ccccc5c4cc32)cc1 ; c1ccc(-n2c3ccccc3c3c(-c4ccc5ccc6cccc7ccc4c5c67)c4oc5ccccc5c4cc32)cc1 ; c1cc2ccc3ccc(-c4c5oc6ccccc6c5cc5oc6ccccc6c45)c4ccc(c1)c2c34]

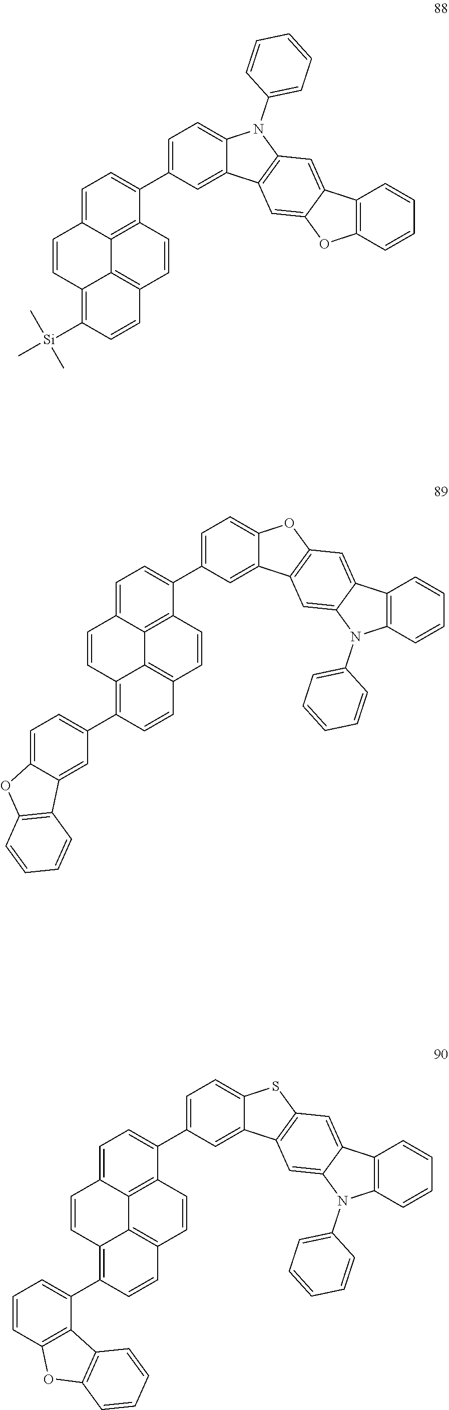 [SMILES: C[Si](C)(C)c1ccc2ccc3c(-c4ccc5c(c4)c4cc6oc7ccccc7c6cc4n5-c4ccccc4)ccc4ccc1c2c43 ; c1ccc(-n2c3ccccc3c3cc4oc5ccc(-c6ccc7ccc8c(-c9ccc%10oc%11ccccc%11c%10c9)ccc9ccc6c7c98)cc5c4cc32)cc1 ; c1ccc(-n2c3ccccc3c3cc4sc5ccc(-c6ccc7ccc8c(-c9cccc%10oc%11ccccc%11c9%10)ccc9ccc6c7c98)cc5c4cc32)cc1]

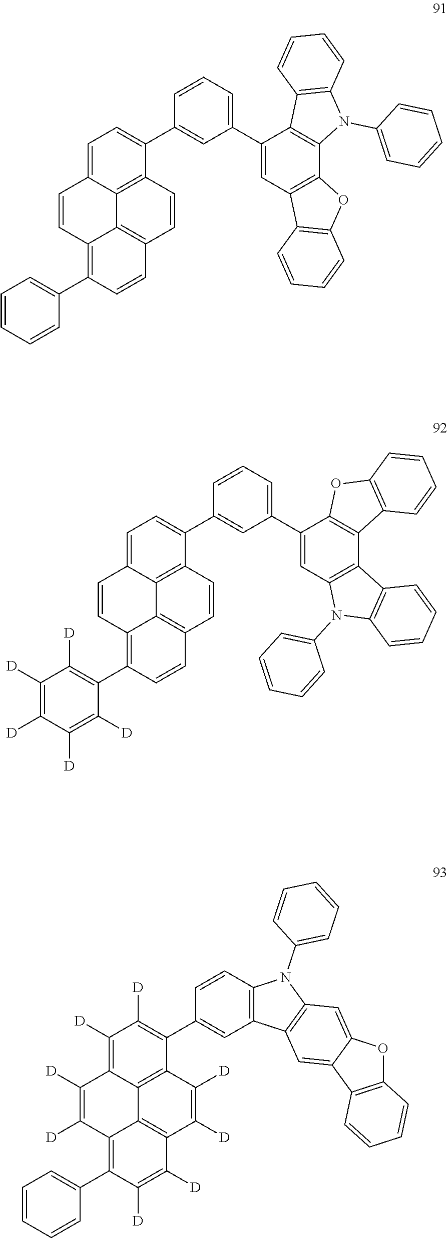 [SMILES: c1ccc(-c2ccc3ccc4c(-c5cccc(-c6cc7c8ccccc8oc7c7c6c6ccccc6n7-c6ccccc6)c5)ccc5ccc2c3c54)cc1 ; [2H]c1c([2H])c([2H])c(-c2ccc3ccc4c(-c5cccc(-c6cc7c(c8ccccc8n7-c7ccccc7)c7c6oc6ccccc67)c5)ccc5ccc2c3c54)c([2H])c1[2H] ; [2H]c1c([2H])c2c([2H])c([2H])c3c(-c4ccc5c(c4)c4cc6c(cc4n5-c4ccccc4)oc4ccccc46)c([2H])c([2H])c4c([2H])c([2H])c(c1-c1ccccc1)c2c43]

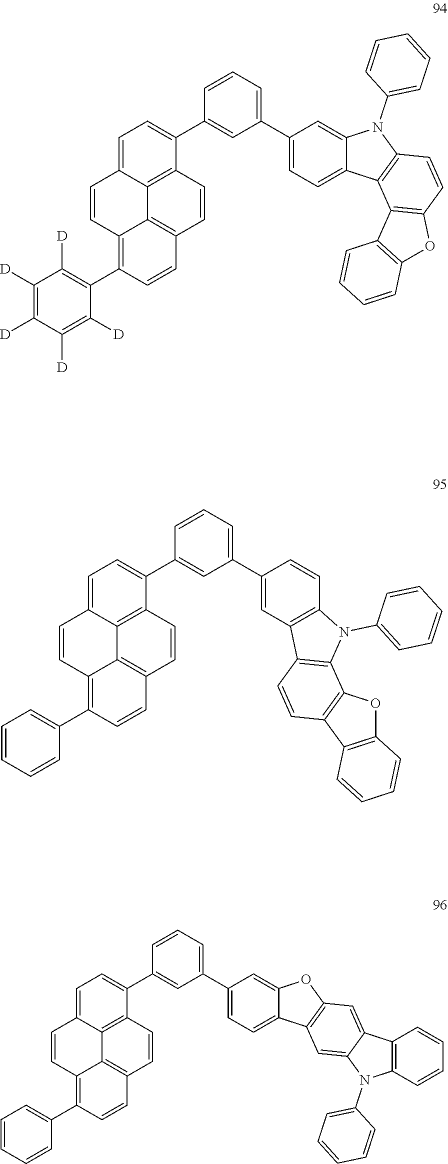 [SMILES: [2H]c1c([2H])c([2H])c(-c2ccc3ccc4c(-c5cccc(-c6ccc7c8c9c(ccc8n(-c8ccccc8)c7c6)oc6ccccc69)c5)ccc5ccc2c3c54)c([2H])c1[2H] ; c1ccc(-c2ccc3ccc4c(-c5cccc(-c6ccc7c(c6)c6ccc8c9ccccc9oc8c6n7-c6ccccc6)c5)ccc5ccc2c3c54)cc1 ; c1ccc(-c2ccc3ccc4c(-c5cccc(-c6ccc7c(c6)oc6cc8c9ccccc9n(-c9ccccc9)c8cc67)c5)ccc5ccc2c3c54)cc1]

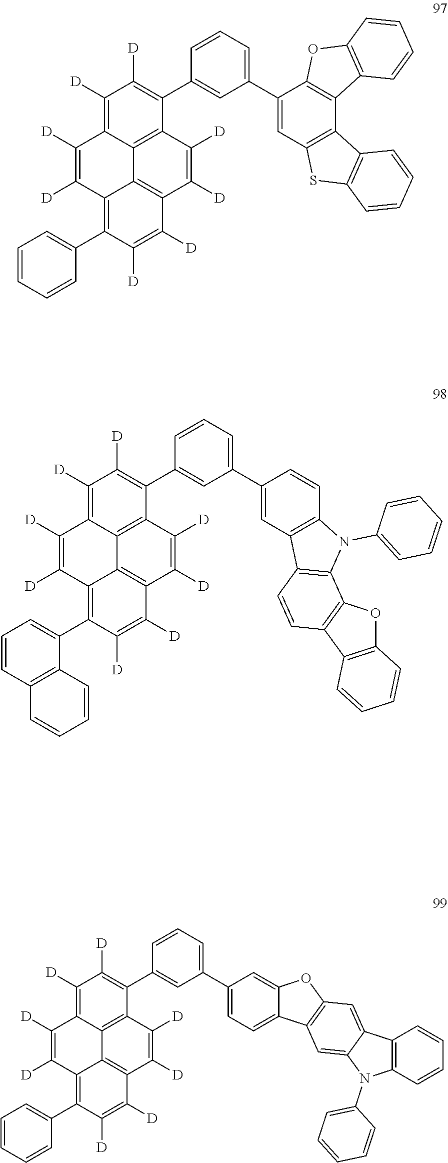 [SMILES: [2H]c1c([2H])c2c([2H])c([2H])c3c(-c4cccc(-c5cc6sc7ccccc7c6c6c5oc5ccccc56)c4)c([2H])c([2H])c4c([2H])c([2H])c(c1-c1ccccc1)c2c43 ; [2H]c1c([2H])c2c([2H])c([2H])c3c(-c4cccc5ccccc45)c([2H])c([2H])c4c([2H])c([2H])c(c1-c1cccc(-c5ccc6c(c5)c5ccc7c8ccccc8oc7c5n6-c5ccccc5)c1)c2c43 ; [2H]c1c([2H])c2c([2H])c([2H])c3c(-c4cccc(-c5ccc6c(c5)oc5cc7c8ccccc8n(-c8ccccc8)c7cc56)c4)c([2H])c([2H])c4c([2H])c([2H])c(c1-c1ccccc1)c2c43]

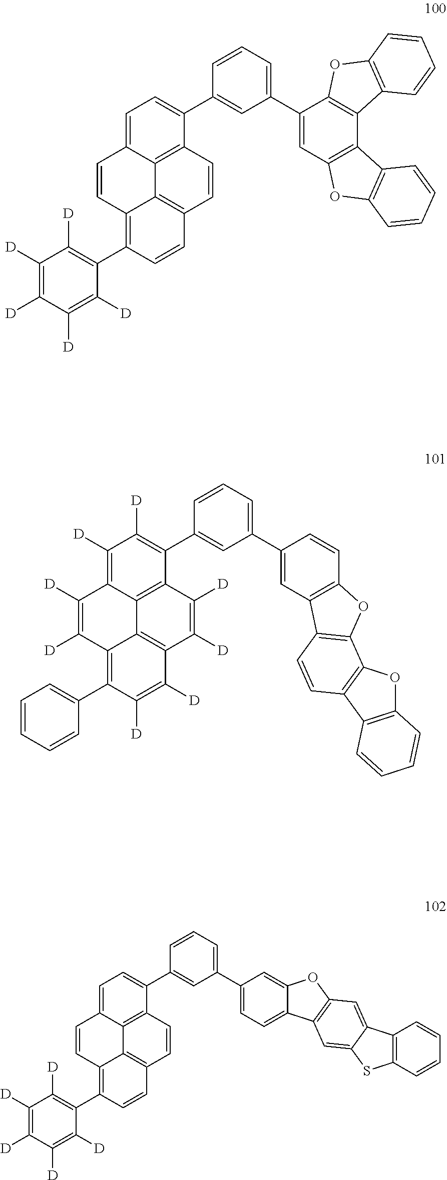 [SMILES: [2H]c1c([2H])c([2H])c(-c2ccc3ccc4c(-c5cccc(-c6cc7oc8ccccc8c7c7c6oc6ccccc67)c5)ccc5ccc2c3c54)c([2H])c1[2H] ; [2H]c1c([2H])c2c([2H])c([2H])c3c(-c4cccc(-c5ccc6oc7c(ccc8c9ccccc9oc87)c6c5)c4)c([2H])c([2H])c4c([2H])c([2H])c(c1-c1ccccc1)c2c43 ; [2H]c1c([2H])c([2H])c(-c2ccc3ccc4c(-c5cccc(-c6ccc7c(c6)oc6cc8c(cc67)sc6ccccc68)c5)ccc5ccc2c3c54)c([2H])c1[2H]]

However, these compounds are not intended to limit the scope of Formula 1.

A further aspect of the present invention is directed to an organic light emitting device including a first electrode, a second electrode opposite to the first electrode, and one or more organic layers interposed between the first and second electrodes wherein one of the organic layers, preferably a light emitting layer includes the pyrene derivative represented by Formula 1.

According to one embodiment of the present invention, the light emitting layer of the organic light emitting device may further include a dopant compound.

As used herein, the expression “(an organic layer) includes one or more organic compounds” can be interpreted that (the organic layer) includes one of the organic compounds belonging to the scope of the present invention or two or more different compounds belonging to the scope of the organic compounds.

The organic layers of the organic light emitting device according to the present invention may include a hole injecting layer, a hole transport layer, a functional layer having functions of both hole injection and hole transport, a light emitting layer, an electron transport layer, and/or an electron injecting layer.

According to a more preferred embodiment of the present invention, one of the organic layers interposed between the first and second electrodes may be a light emitting layer. The light emitting layer may be composed of a host and a dopant. The light emitting layer may include, as a host, at least one of the compounds that can be represented by Formula 1.

The dopant compound used in the light emitting layer may be selected from compounds represented by Formulas D1 to D10:

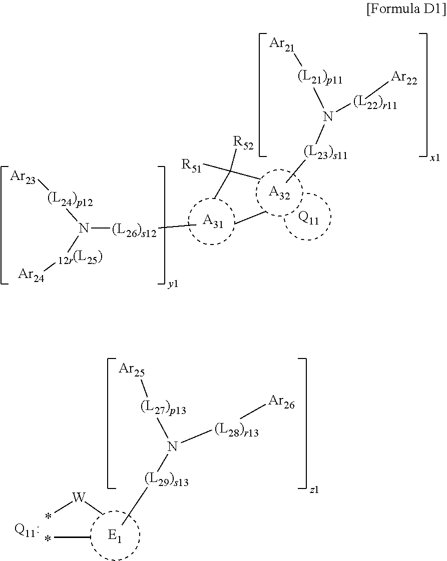

wherein A₃₁, A₃₂, and E₁ are each independently a substituted or unsubstituted C₆-C₅₀ aromatic hydrocarbon ring or a substituted or unsubstituted C₂-C₄₀ aromatic heterocyclic ring, with the proviso that two adjacent carbon atoms in the aromatic ring A₃₁ and two adjacent carbon atoms in the aromatic ring A₃₂ optionally form a 5-membered ring with the carbon atom to which R₅₁ and R₅₂ are attached, to form a fused ring, L₂₁ to L₂₉ are the same as or different from each other and are each independently selected from a single bond, substituted or unsubstituted C₁-C₆₀ alkylene, substituted or unsubstituted C₂-C₆₀ alkenylene, substituted or unsubstituted C₂-C₆₀ alkynylene, substituted or unsubstituted C₃-C₆₀ cycloalkylene, substituted or unsubstituted C₂-C₆₀ heterocycloalkylene, substituted or unsubstituted C₆-C₆₀ arylene, and substituted or unsubstituted C₂-C₆₀ heteroarylene, W is selected from N—R₅₃, CR₅₄R₅₅, SiR₅₆R₅₇, GeR₅₈R₅₉, O, S, and Se, R₅₁ to R₅₉ and Ar₂₁ to Ar₂₆ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₂-C₃₀ alkenyl, substituted or unsubstituted C₂-C₂₀ alkynyl, substituted or unsubstituted C₃-C₃₀ cycloalkyl, substituted or unsubstituted C₅-C₃₀ cycloalkenyl, substituted or unsubstituted C₂-C₅₀ heteroaryl, substituted or unsubstituted C₂-C₃₀ heterocycloalkyl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₅₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, substituted or unsubstituted C₁-C₃₀ alkylgermanium, substituted or unsubstituted C₁-C₃₀ arylgermanium, cyano, nitro, and halogen, with the proviso that R₅₁ and R₅₂ are optionally linked to each other to form an alicyclic or aromatic monocyclic or polycyclic ring in which carbon atoms are optionally substituted with one or more heteroatoms selected from the group consisting of N, O, P, Si, S, Ge, Se, and Te, p11 to p13, r11 to r13, and s11 to s13 are each independently an integer from 1 to 3, provided that when p11 is 2 or greater, the linkers L₂₁ are the same as or different from each other, when p12 is 2 or greater, the linkers L₂₄ are the same as or different from each other, when p13 is 2 or greater, the linkers L₂₇ are the same as or different from each other, when r11 is 2 or greater, the linkers L₂₂ are the same as or different from each other, when r12 is 2 or greater, the linkers L₂₅ are the same as or different from each other, when r13 is 2 or greater, the linkers L₂₈ are the same as or different from each other, when s11 is 2 or greater, the linkers L₂₃ are the same as or different from each other, when s12 is 2 or greater, the linkers L₂₆ are the same as or different from each other, and when s13 is 2 or greater, the linkers L₂₉ are the same as or different from each other, x1 is 1, and y1 and z1 are each independently an integer of 0 or 1, with the proviso that Ar₂₁ and Ar₂₂ are optionally linked to each other to form a ring, Ar₂₃ and Ar₂₄ are optionally linked to each other to form a ring, and Ar₂₅ and Ar₂₆ are optionally linked to each other to form a ring, and with the proviso that two adjacent carbon atoms in the ring A₃₂ are optionally bonded to Q₁₁ at the asterisks (*) to form a fused ring;

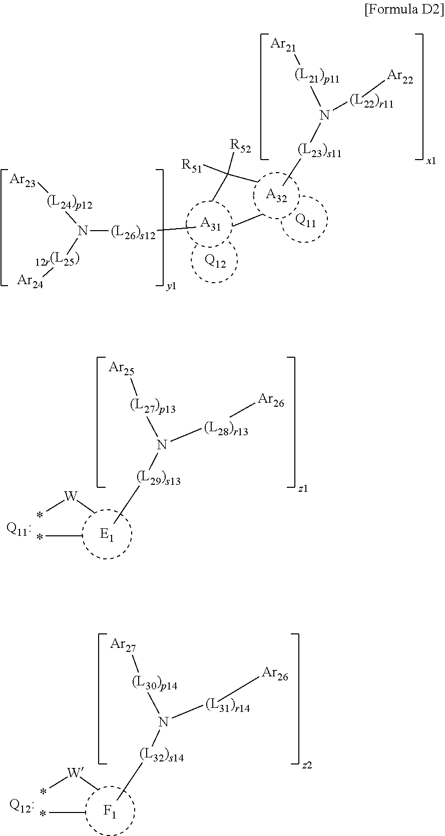

wherein A₃₁, A₃₂, E1, and F₁ are as defined for A₃₁, A₃₂, and E₁ in Formula D1, L₂₁ to L₃₂ are as defined for L₂₁ to L₂₉ in Formula D1, W and W′ are the same as or different from each other and are each independently as defined for W in Formula D1, R₅₁ to R₅₉ and Ar₂₁ to Ar₂₈ are as defined for R₅₁ to R₅₉ and Ar₂₁ to Ar₂₆ in Formula D1, p11 to p14, r11 to r14, and s11 to s14 are as defined for p11 to p13, r11 to r13, and s11 to s13 in Formula D1, provided that when p14 is 2 or greater, the linkers L₃₀ are the same as or different from each other, when r14 is 2 or greater, the linkers L₃₁ are the same as or different from each other, and when s14 is 2 or greater, the linkers L₃₂ are the same as or different from each other, x1 is as defined in Formula D1, and y1, z1, and z2 are as defined for y1 and z1 in Formula D1, with the proviso that Ar₂₁ and Ar₂₂ are optionally linked to each other to form a ring, Ar₂₃ and Ar₂₄ are optionally linked to each other to form a ring, Ar₂₅ and Ar₂₆ are optionally linked to each other to form a ring, and Ar₂₇ and Ar₂₈ are optionally linked to each other to form a ring, and with the proviso that two adjacent carbon atoms in the ring A₃₁ are optionally bonded to Q₁₂ at the asterisks (*) to form a fused ring and two adjacent carbon atoms in the ring A₃₂ are optionally bonded to Q₁₁ at the asterisks (*) to form a fused ring;

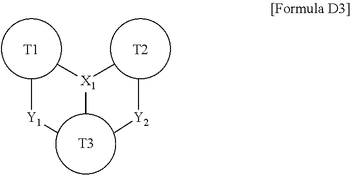

wherein X₁ is selected from B, P, and P═O, T1 to T3 are the same as or different from each other and are each independently a substituted or unsubstituted C₆-C₅₀ aromatic hydrocarbon ring or a substituted or unsubstituted C₂-C₄₀ aromatic heterocyclic ring, Y1 is selected from N—R₆₁, CR₆₂R₆₃, O, S, and SiR₆₄R₆₅, Y2 is selected from N—R₆₆, CR₆₇R₆₈, O, S, and SiR₆₉R₇₀, and R₆₁ to R₇₀ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₃-C₃₀ cycloalkyl, substituted or unsubstituted C₂-C₅₀ heteroaryl, substituted or unsubstituted C₁-C₃₀ alkoxy, substituted or unsubstituted C₆-C₅₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, cyano, and halogen, with the proviso that each of R₆₁ to R₇₀ is optionally bonded to one or more rings selected from T1 to T3 to form an alicyclic or aromatic monocyclic or polycyclic ring;

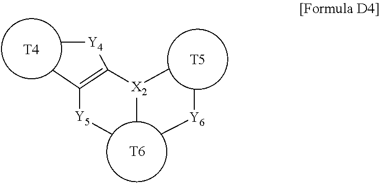

wherein X₂ is selected from B, P, and P═O, T4 to T6 are as defined for T1 to T3 in Formula D3, Y₄ is selected from N—R₆₁, CR₆₂R₆₃, O, S, and SiR₆₄R₆₅, Y5 is selected from N—R₆₆, CR₆₇R₆₈, O, S, and SiR₆₉R₇₀, Y₆ is selected from N—R₇₁, CR₇₂R₇₃, O, S, and SiR₇₄R₇₅, and R₆₁ to R₇₅ are as defined for R₆₁ to R₇₀ in Formula D3;

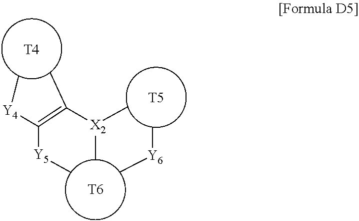

wherein X₂, T4 to T6, and Y₄ to Y₆ are as defined in Formula D4;

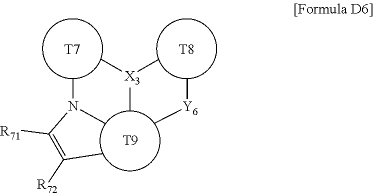

wherein X₃ is selected from B, P, and P═O, T7 to T9 are as defined for T1 to T3 in Formula D3, Y₆ is selected from N—R₆₁, CR₆₂R₆₃, O, S, and SiR₆₄R₆₅, and R₆₁ to R₆₅, R₇₁, and R₇₂ are as defined for R₆₁ to R₇₀ in Formula D3, with the proviso that R₇₁ and R₇₂ are optionally linked to each other to form an alicyclic or aromatic monocyclic or polycyclic ring or are optionally bonded to the ring T7 or T9 to form an alicyclic or aromatic monocyclic or polycyclic ring;

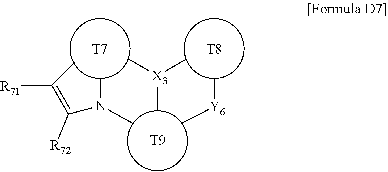

wherein X₃, T7 to T9, Y₆, R₇₁, and R₇₂ are as defined in Formula D6;

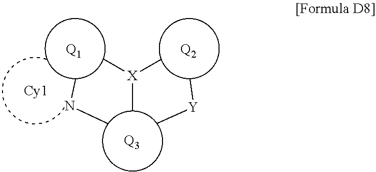

wherein X is selected from B, P, and P═O, Q₁ to Q₃ are as defined for T1 to T3 in Formula D3, Y is selected from N—R₃, CR₄R₅, O, S, and Se, R₃ to R₅ are as defined for R₆₁ to R₇₀ in Formula D3, with the proviso that each of R₃ to R₅ is optionally bonded to the ring Q₂ or Q₃ to form an alicyclic or aromatic monocyclic or polycyclic ring and each of R₄ and R₅ is optionally linked to each other to form an alicyclic or aromatic monocyclic or polycyclic ring, and Cy1 represents a ring formed by a substituted or unsubstituted C₁-C₁₀ alkylene group with the nitrogen (N) atom between the rings Q₁ and Q₃, the aromatic carbon atom in the ring Q₁ to which the nitrogen (N) atom is bonded, and the aromatic carbon atom in the ring Q₁ to which Cy1 is to be bonded;

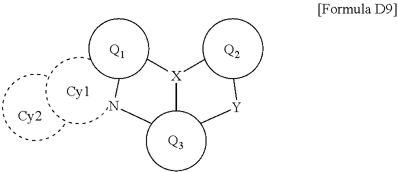

wherein X, Q₁ to Q₃, Y, and Cy1 are as defined in Formula D8 and Cy2 represents a saturated hydrocarbon ring attached to Cy1 and formed by a substituted or unsubstituted C₁-C₁₀ alkylene group with carbon atoms in Cy1; and

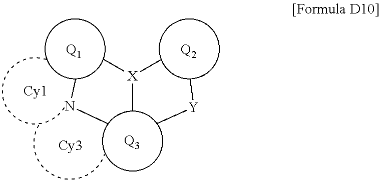

wherein X, Q₁ to Q₃, Y, and Cy1 are as defined in Formula D8 and Cy3 represents a ring formed by a substituted or unsubstituted C₁-C₁₀ alkylene group with the aromatic carbon atom in the ring Q₃ to which Cy3 is to be bonded, the nitrogen (N) atom between the rings Q₁ and Q₃, the aromatic carbon atom in the ring Q₃ to which the nitrogen (N) atom is bonded, and the carbon atom in Cy1 to which the nitrogen (N) atom is bonded.

As used herein, the term “substituted” in the definition of the substituents in Formulas D1 to D10 indicates substitution with one or more substituents selected from the group consisting of deuterium, cyano, halogen, hydroxyl, nitro, C₁-C₂₄ alkyl, C₃-C₂₄ cycloalkyl, C₁-C₂₄ haloalkyl, C₂-C₂₄ alkenyl, C₂-C₂₄ alkynyl, C₃-C₂₄ cycloalkyl, C₁-C₂₄ heteroalkyl, C₆-C₂₄ aryl, C₇-C₂₄ arylalkyl, C₇-C₂₄ alkylaryl, C₂-C₂₄ heteroaryl, C₂-C₂₄ heteroarylalkyl, C₁-C₂₄ alkoxy, C₁-C₂₄ alkylamino, C₁₂-C₂₄ diarylamino, C₂-C₂₄ diheteroarylamino, C₇-C₂₄ aryl(heteroaryl)amino, C₁-C₂₄ alkylsilyl, C₆-C₂₄ arylsilyl, C₆-C₂₄ aryloxy, and C₆-C₂₄ arylthionyl, more preferably deuterium, cyano, halogen, hydroxyl, nitro, C₁-C₁₂ alkyl, C₁-C₁₂ haloalkyl, C₂-C₁₂ alkenyl, C₂-C₁₂ alkynyl, C₃-C₁₂ cycloalkyl, C₁-C₁₂ heteroalkyl, C₆-C₁₈ aryl, C₇-C₂₀ arylalkyl, C₇-C₂₀ alkylaryl, C₂-C₁₈ heteroaryl, C₂-C₁₈ heteroarylalkyl, C₁-C₁₂ alkoxy, C₁-C₁₂ alkylamino, C₁₂-C₁₈ diarylamino, C₂-C₁₈ diheteroarylamino, C₇-C₁₈ aryl(heteroaryl)amino, C₁-C₁₂ alkylsilyl, C₆-C₁₈ arylsilyl, C₆-C₁₈ aryloxy, and C₆-C₁₈ arylthionyl.

In the boron compounds represented by Formulas D3 to D10 as dopant compounds, the aromatic hydrocarbon rings or the aromatic heterocyclic rings T1 to T9 and Q₁ to Q₃ are optionally substituted with one or more substituents selected from deuterium, C₁-C₂₄ alkyl, C₆-C₂₄ aryl, C₁-C₂₄ alkylamino, and C₆-C₂₄ arylamino. Here, the alkyl groups in the C₁-C₂₄ alkylamino may be linked to each other and the aryl groups in the C₆-C₂₄ arylamino may be linked to each other. The substituents are more preferably C₁-C₁₂ alkyl, C₆-C₁₈ aryl, C₁-C₁₂ alkylamino, and C₆-C₁₈ arylamino. Here, the alkyl groups in the C₁-C₁₂ alkylamino may be linked to each other and the aryl groups in the C₆-C₁₈ alkylamino may be linked to each other.

The compounds represented by Formulas D1 and D2 can be used as dopant compounds in the light emitting layer of the organic light emitting device according to the present invention and specific examples thereof include the following compounds d1 and d239:

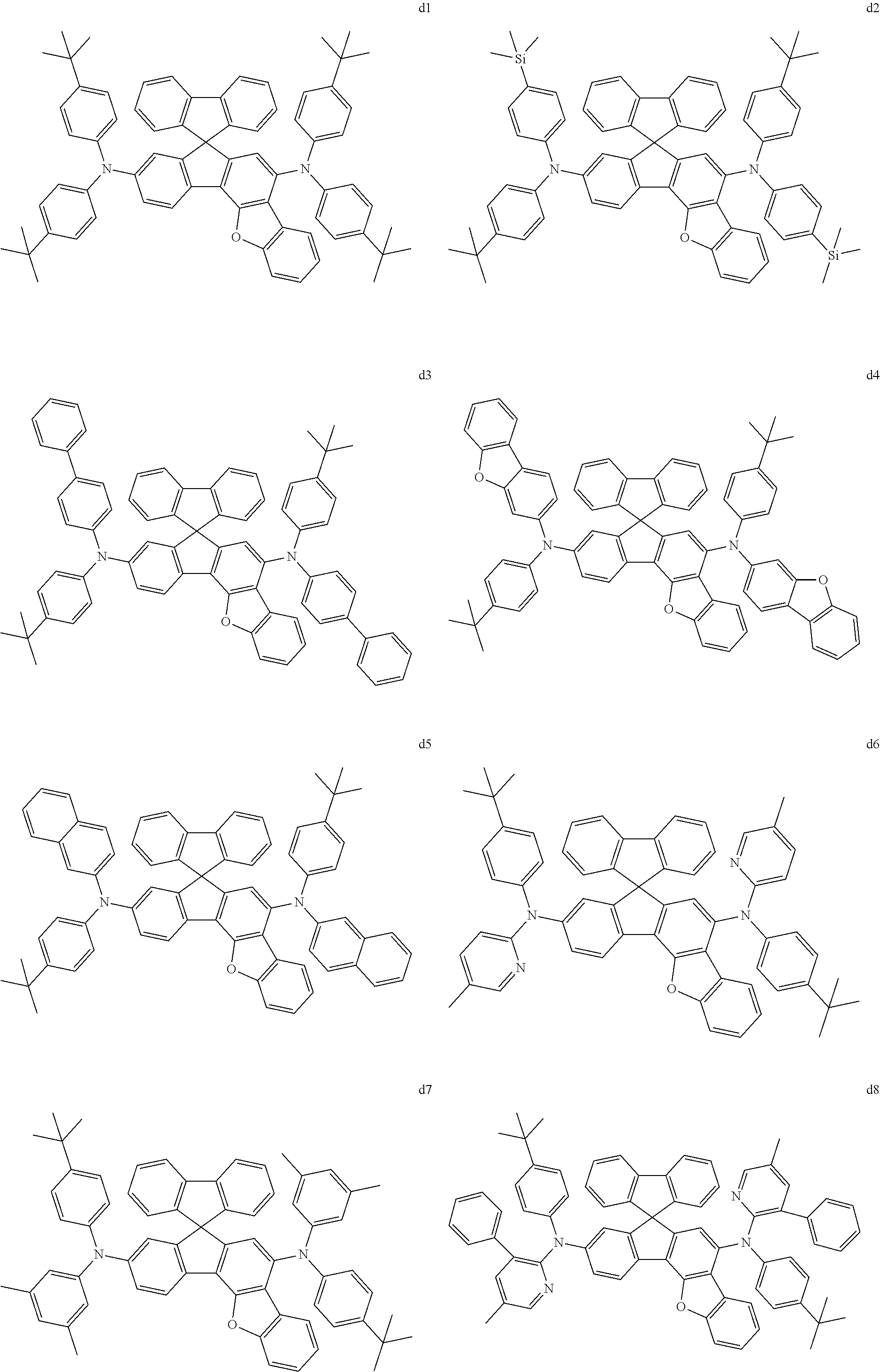

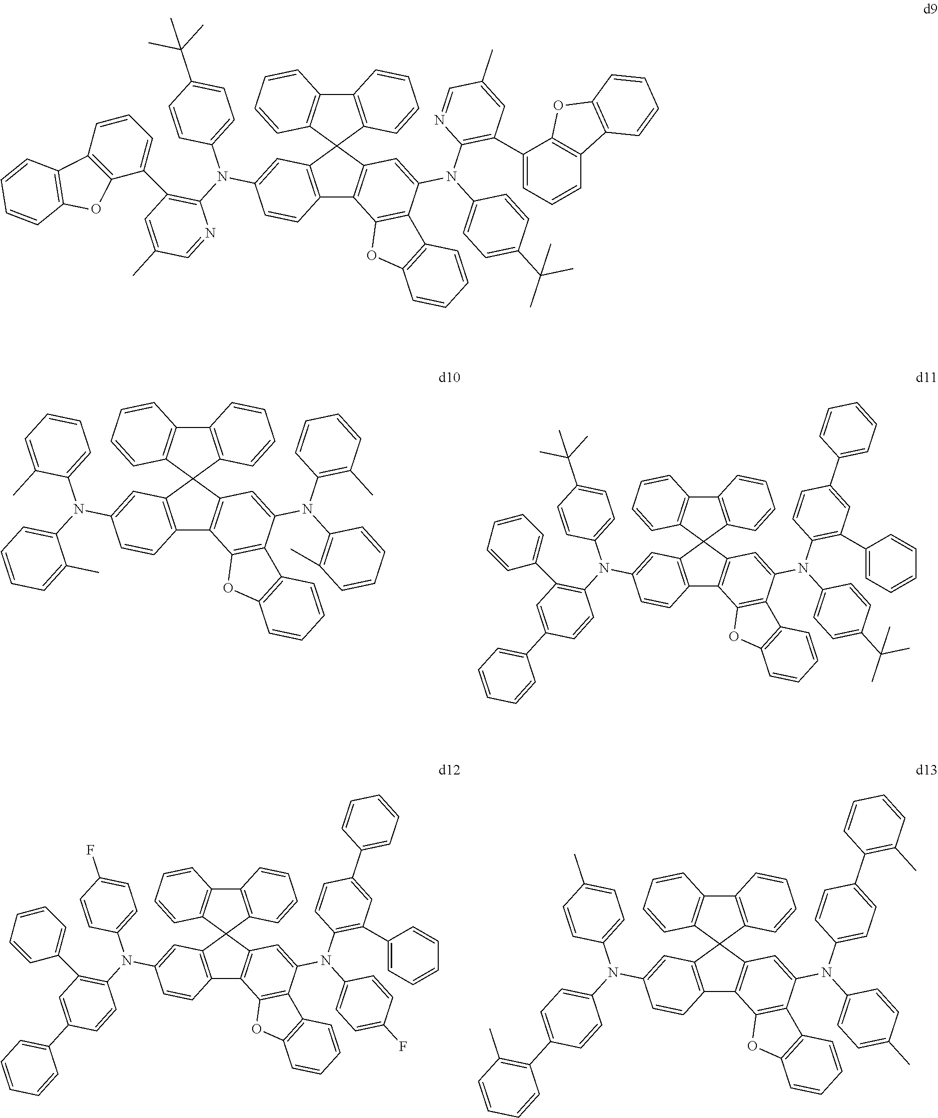

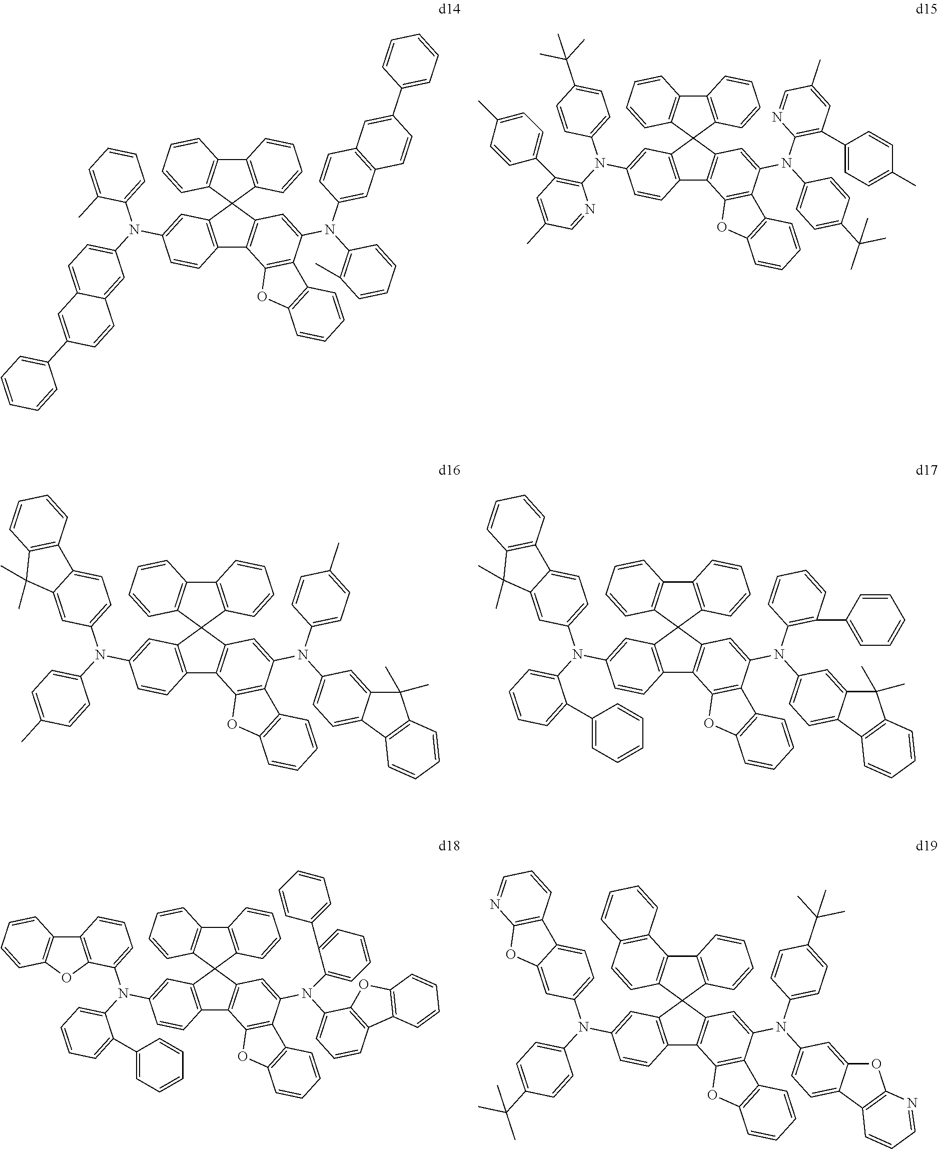

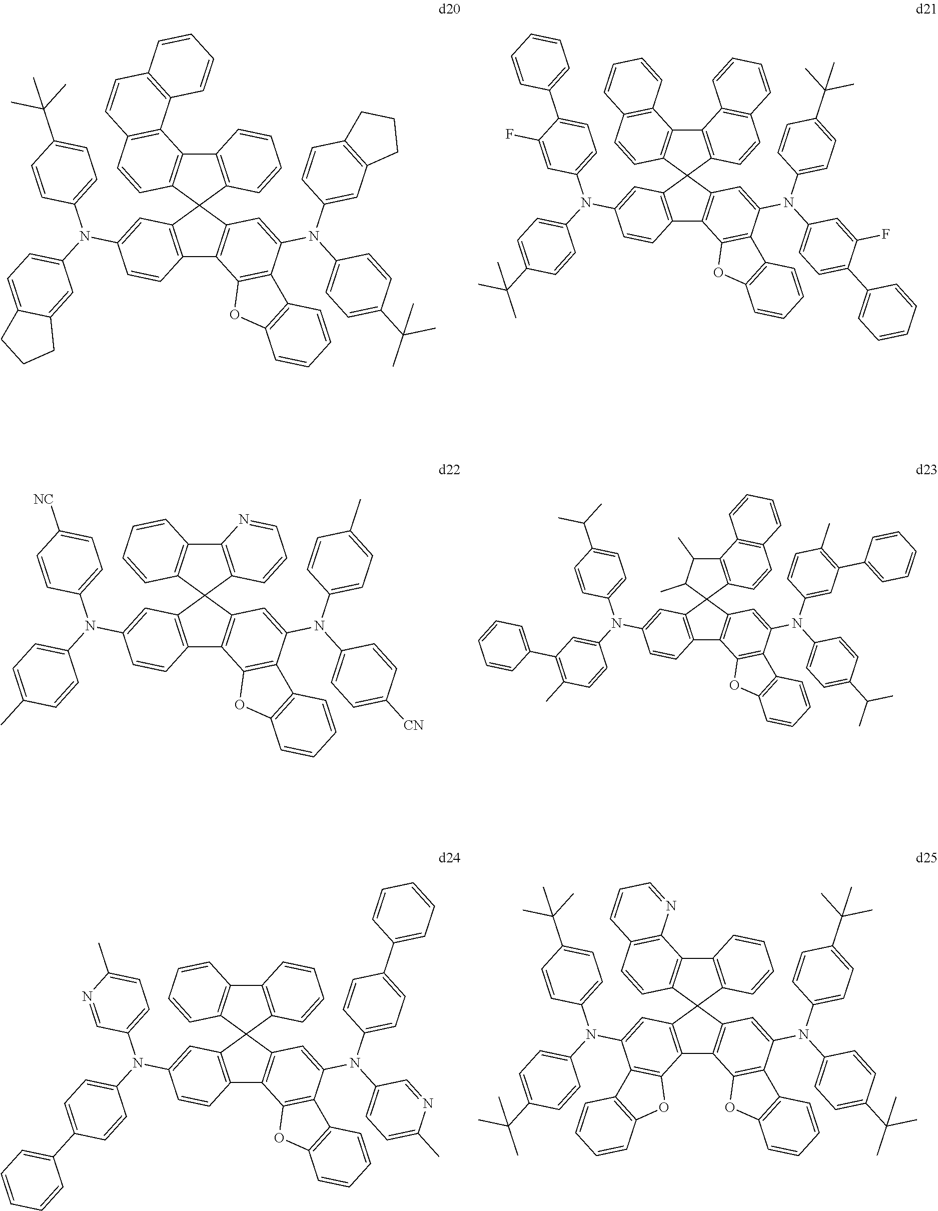

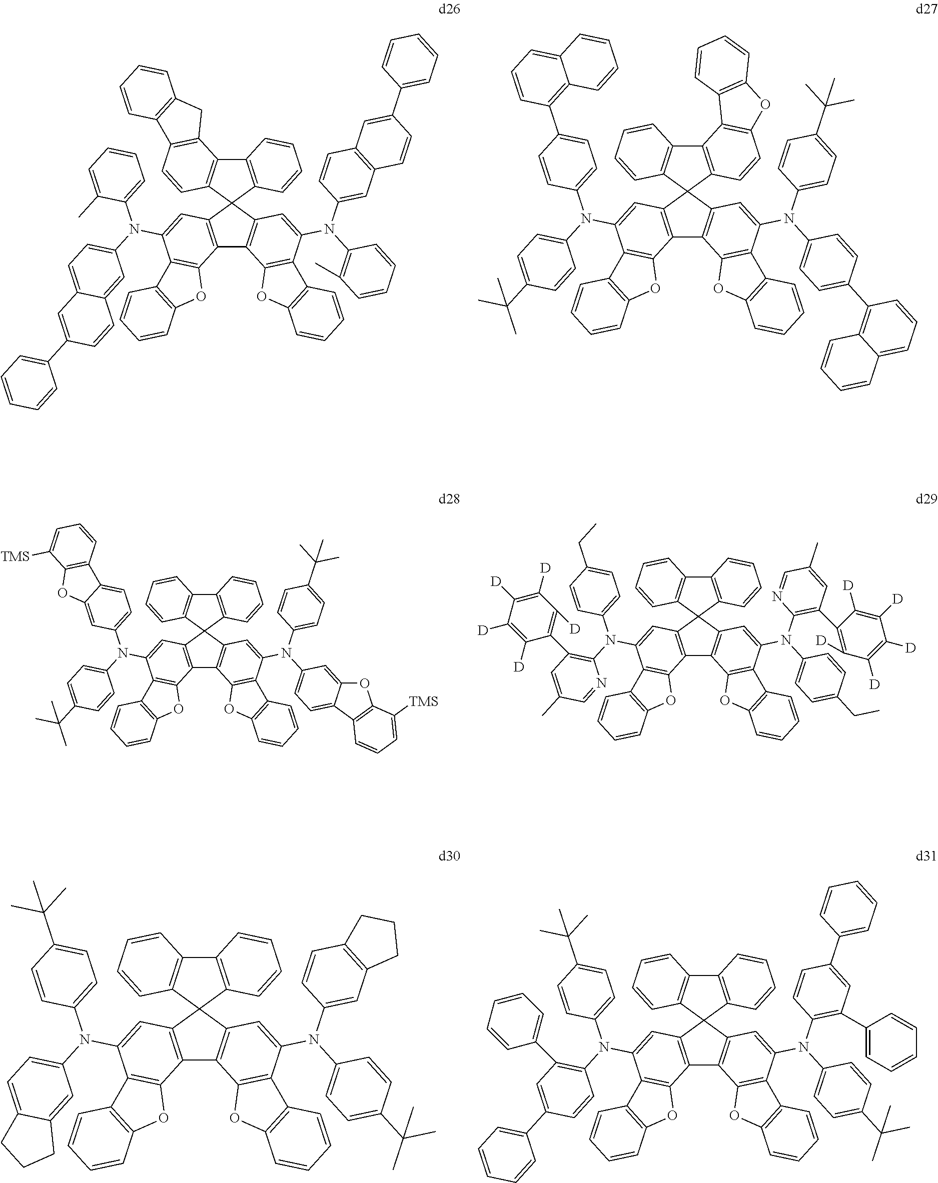

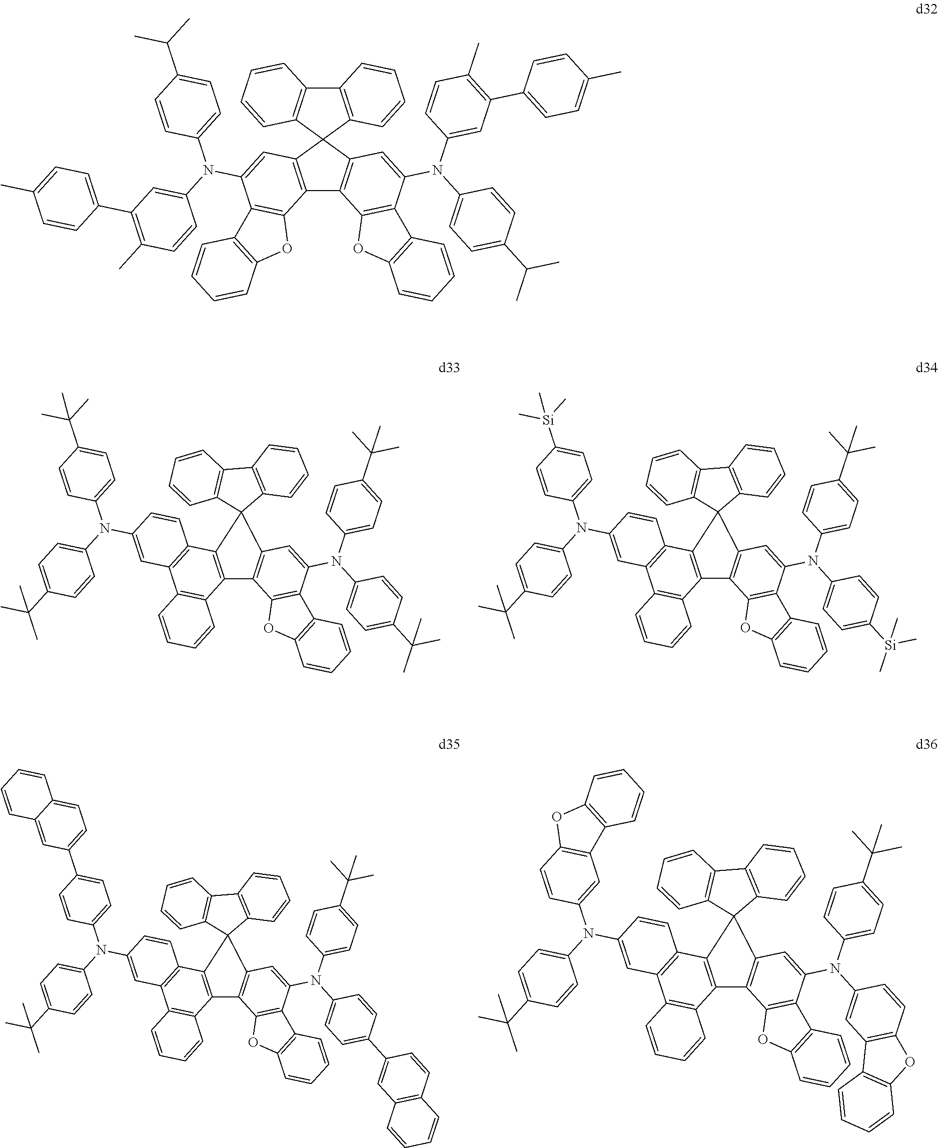

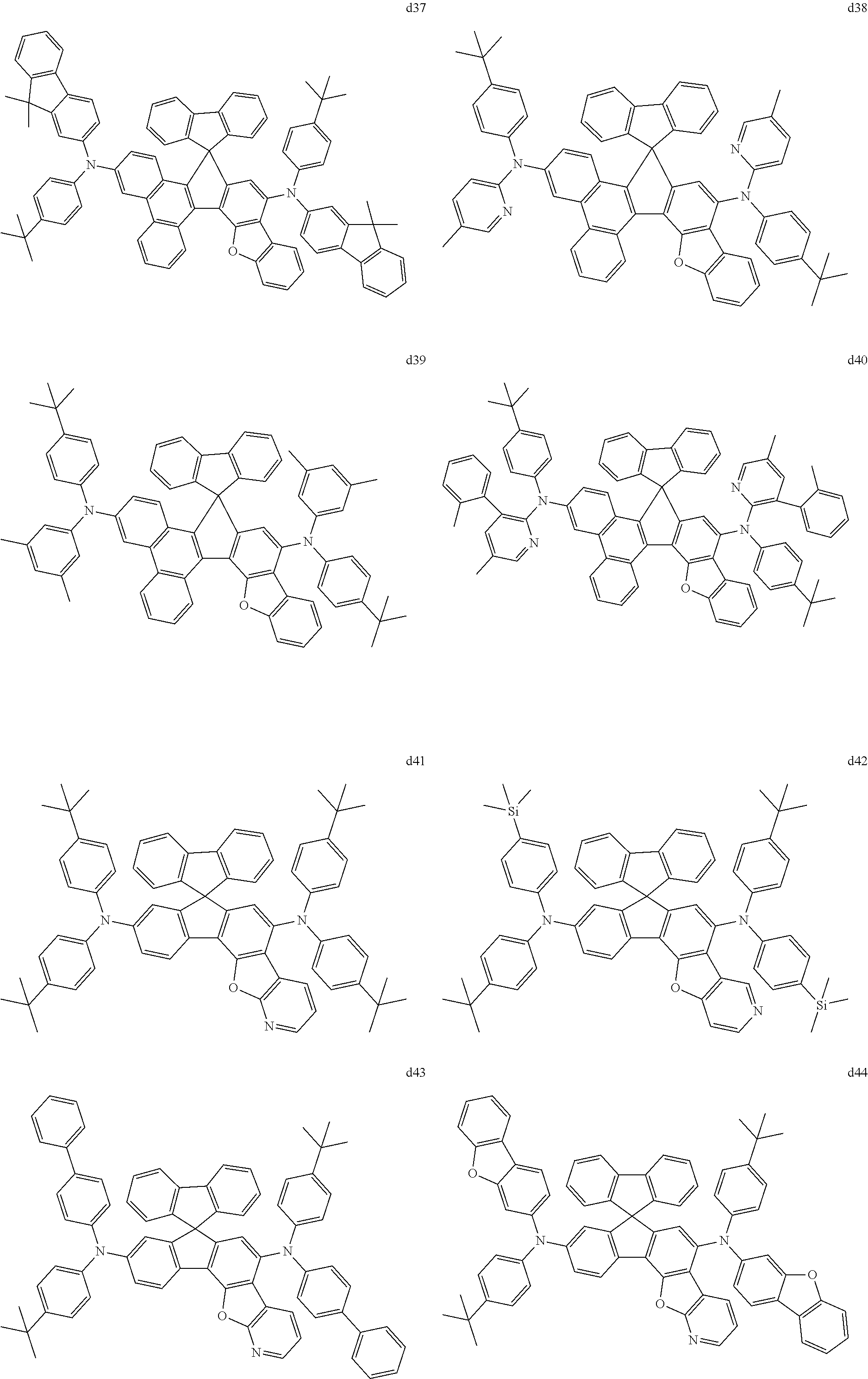

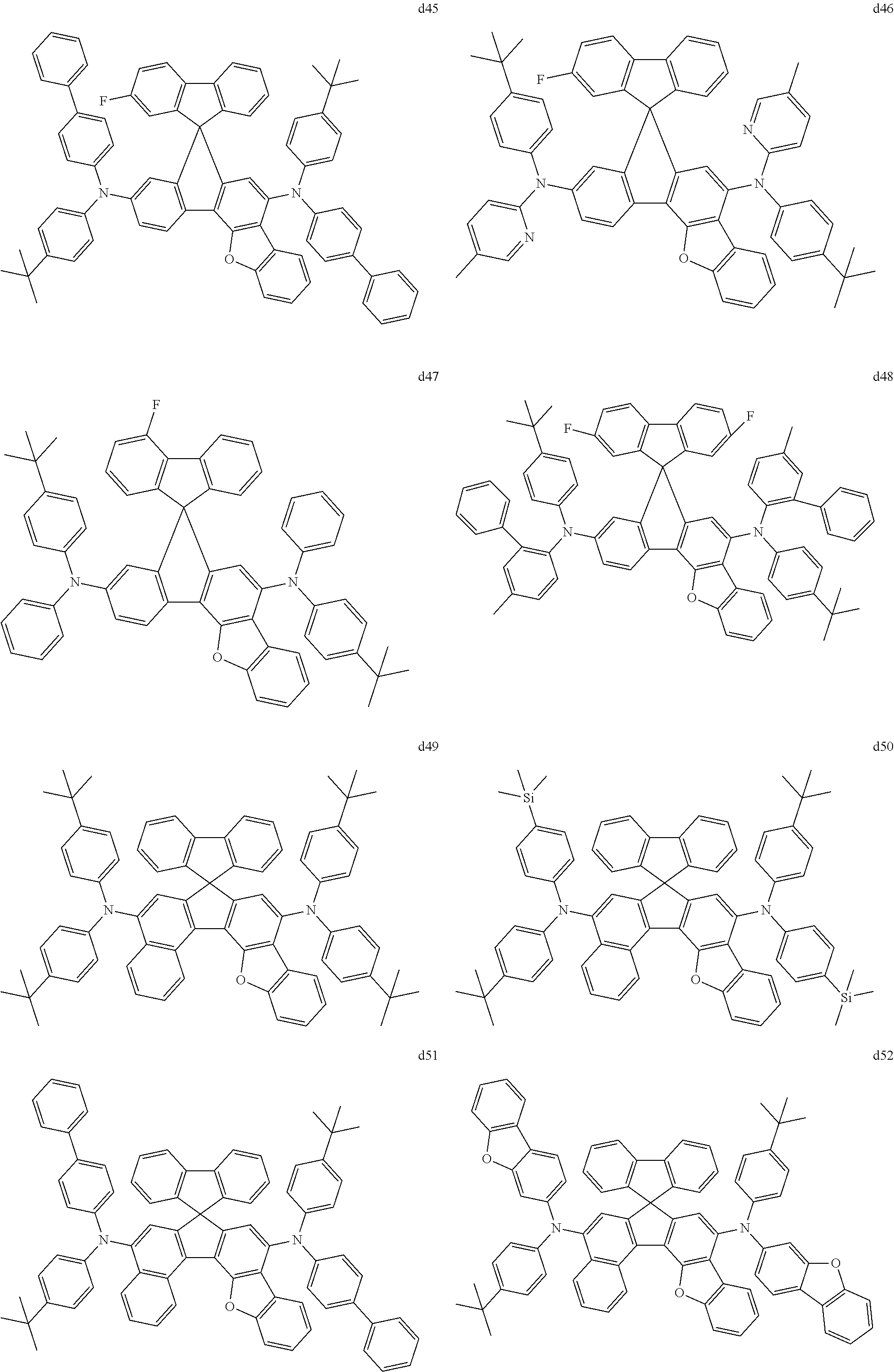

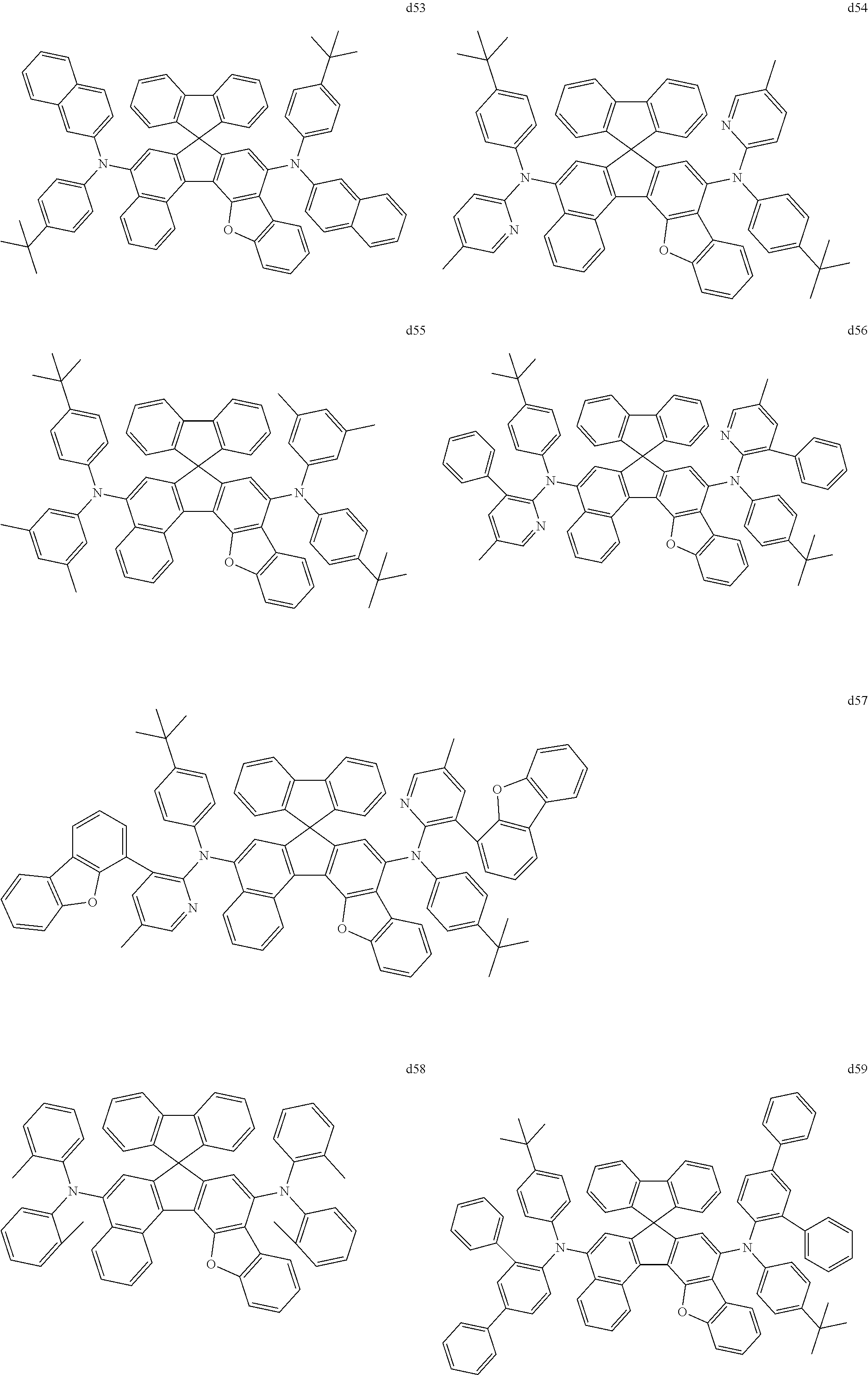

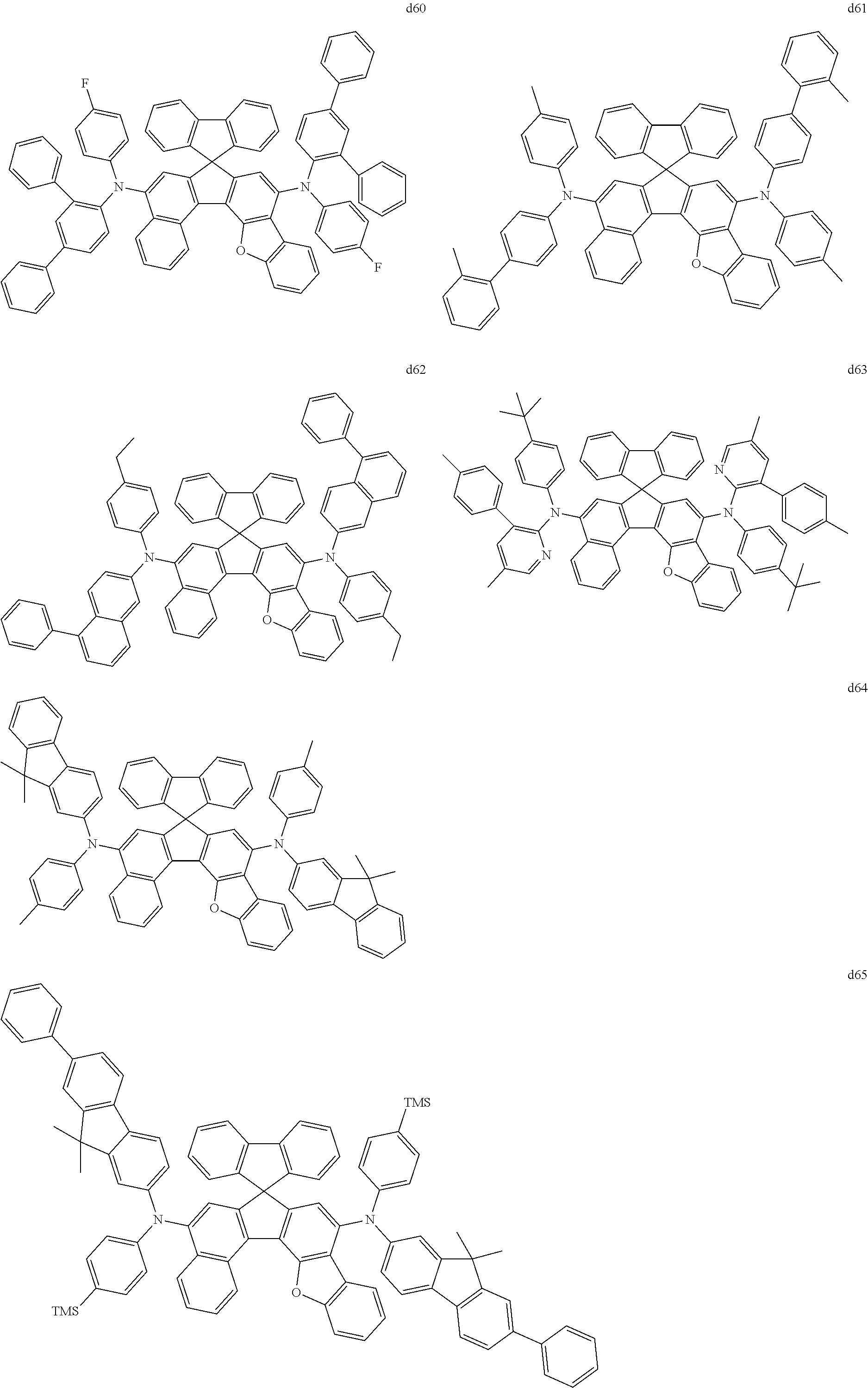

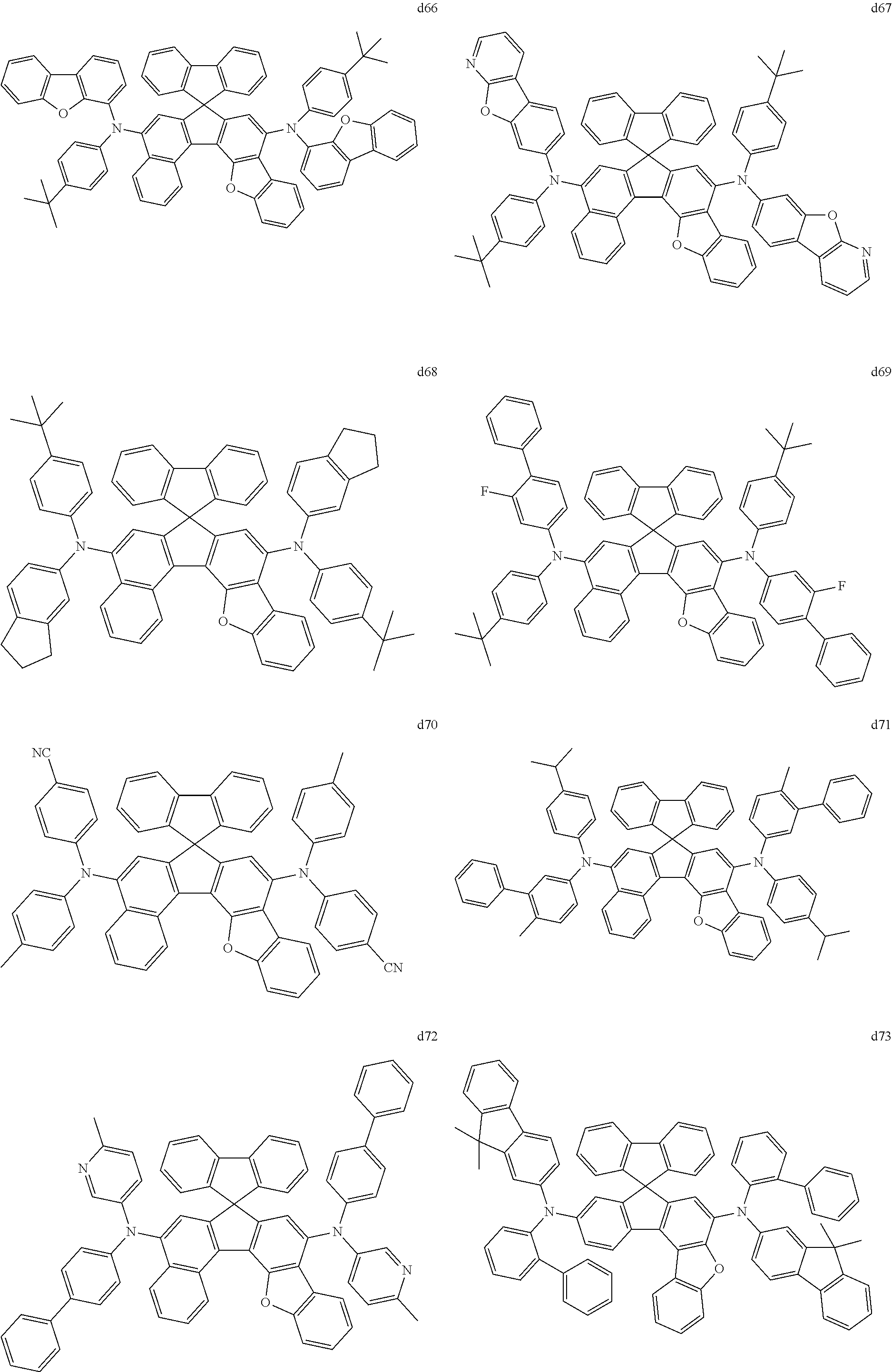

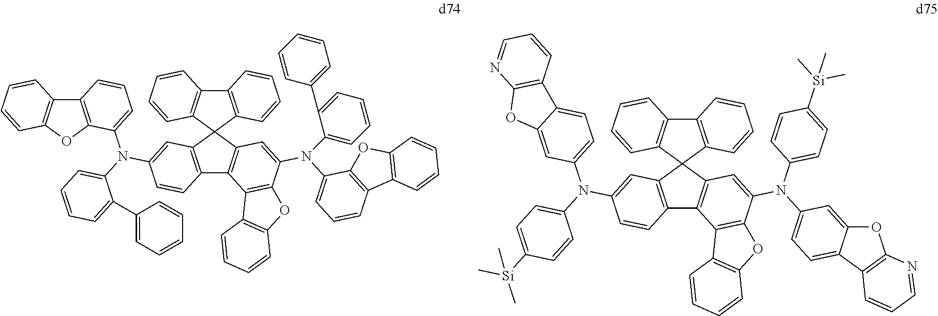 [SMILES: c1ccc(-c2ccccc2N(c2ccc3c(c2)C2(c4ccccc4-c4ccccc42)c2cc(N(c4ccccc4-c4ccccc4)c4cccc5c4oc4ccccc45)c4oc5ccccc5c4c2-3)c2cccc3c2oc2ccccc23)cc1 ; C[Si](C)(C)c1ccc(N(c2ccc3c(c2)C2(c4ccccc4-c4ccccc42)c2cc(N(c4ccc([Si](C)(C)C)cc4)c4ccc5c(c4)oc4ncccc45)c4oc5ccccc5c4c2-3)c2ccc3c(c2)oc2ncccc23)cc1]

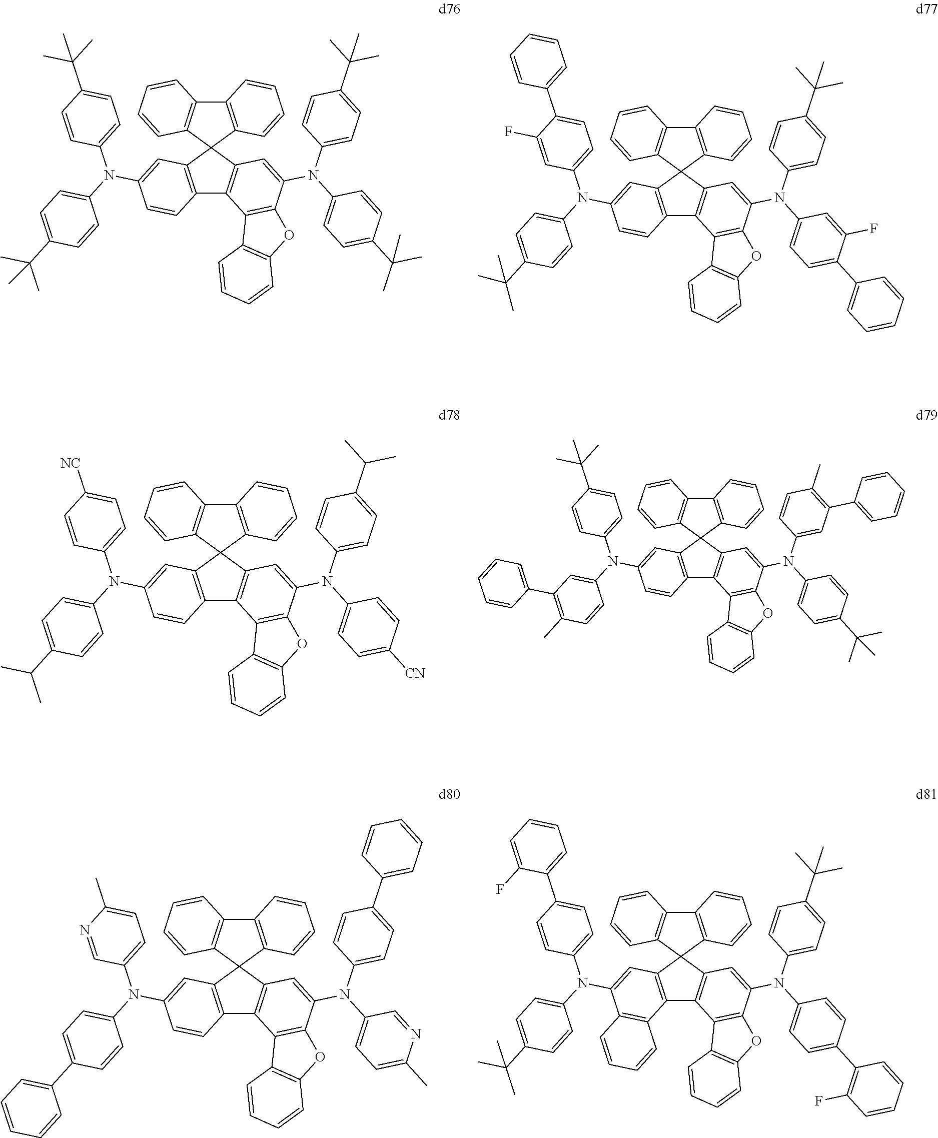

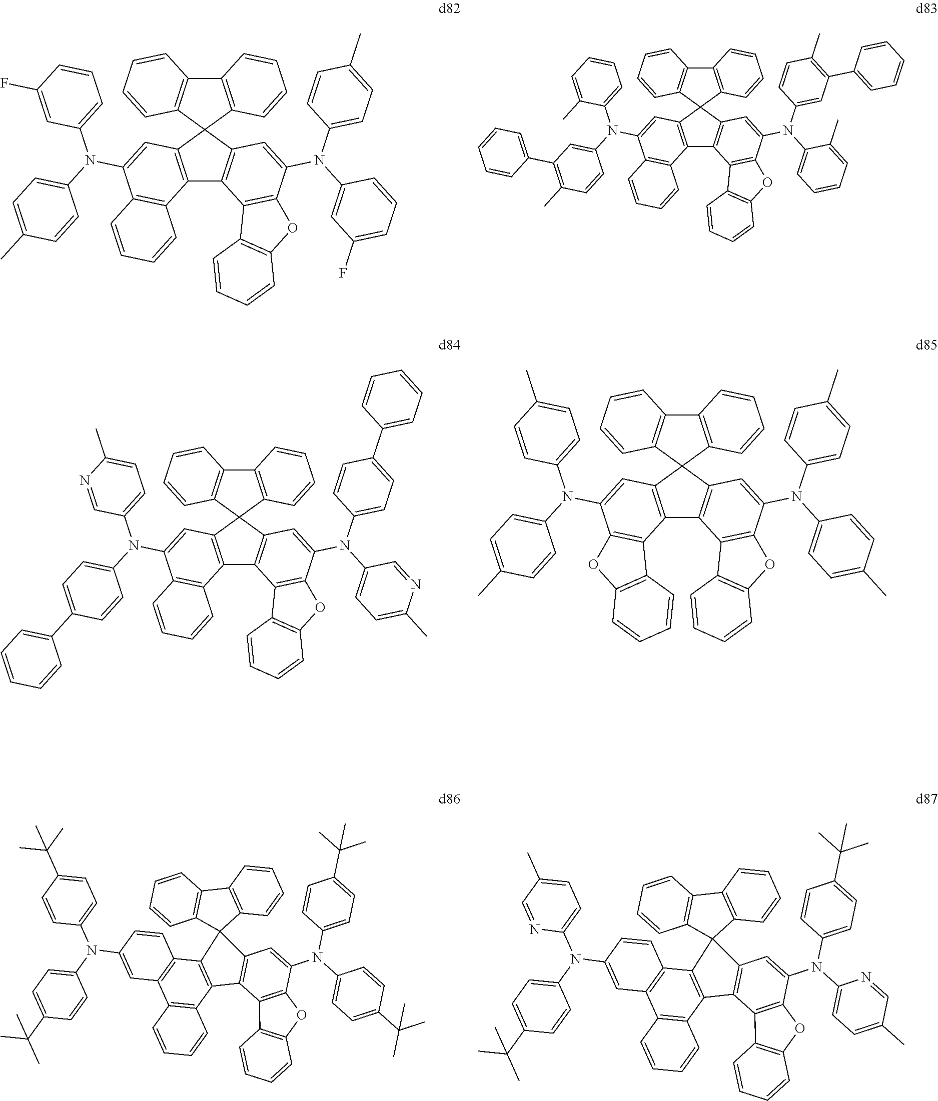

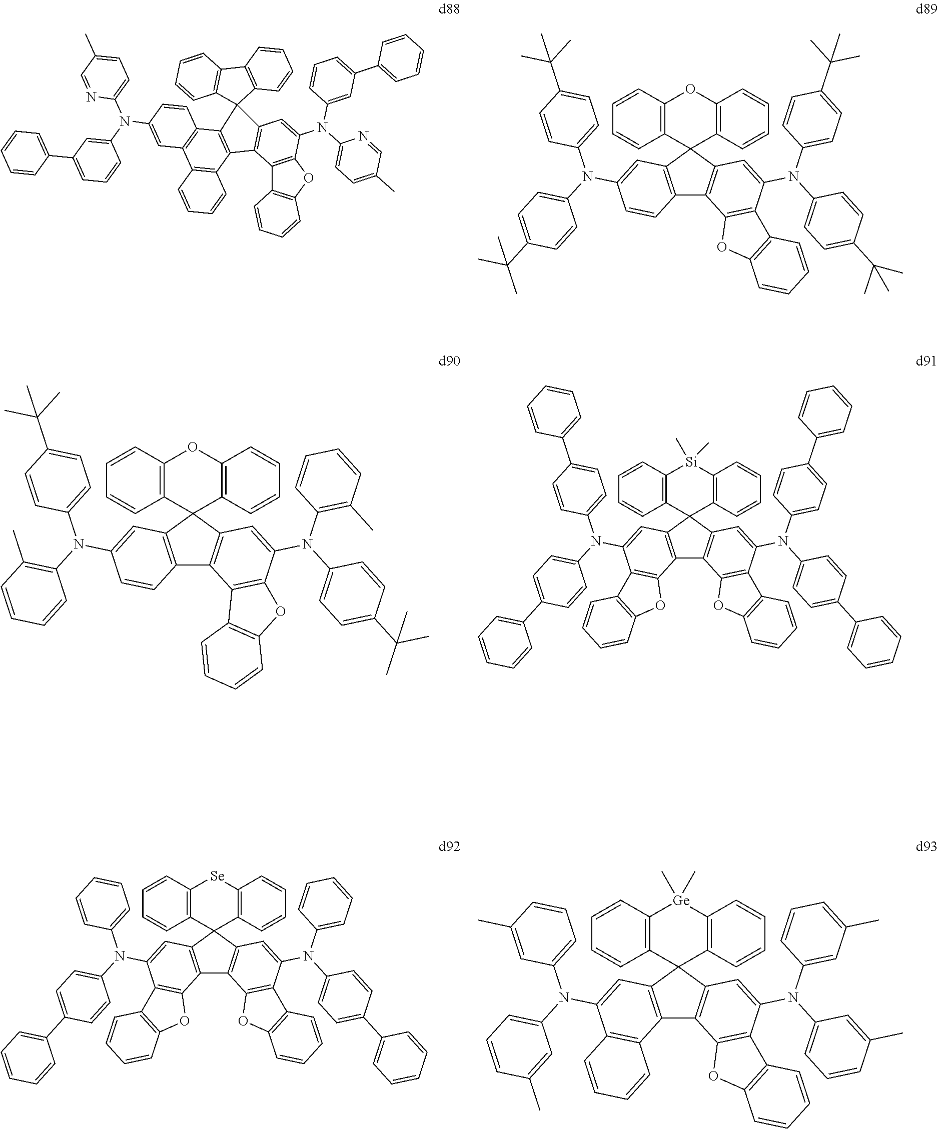

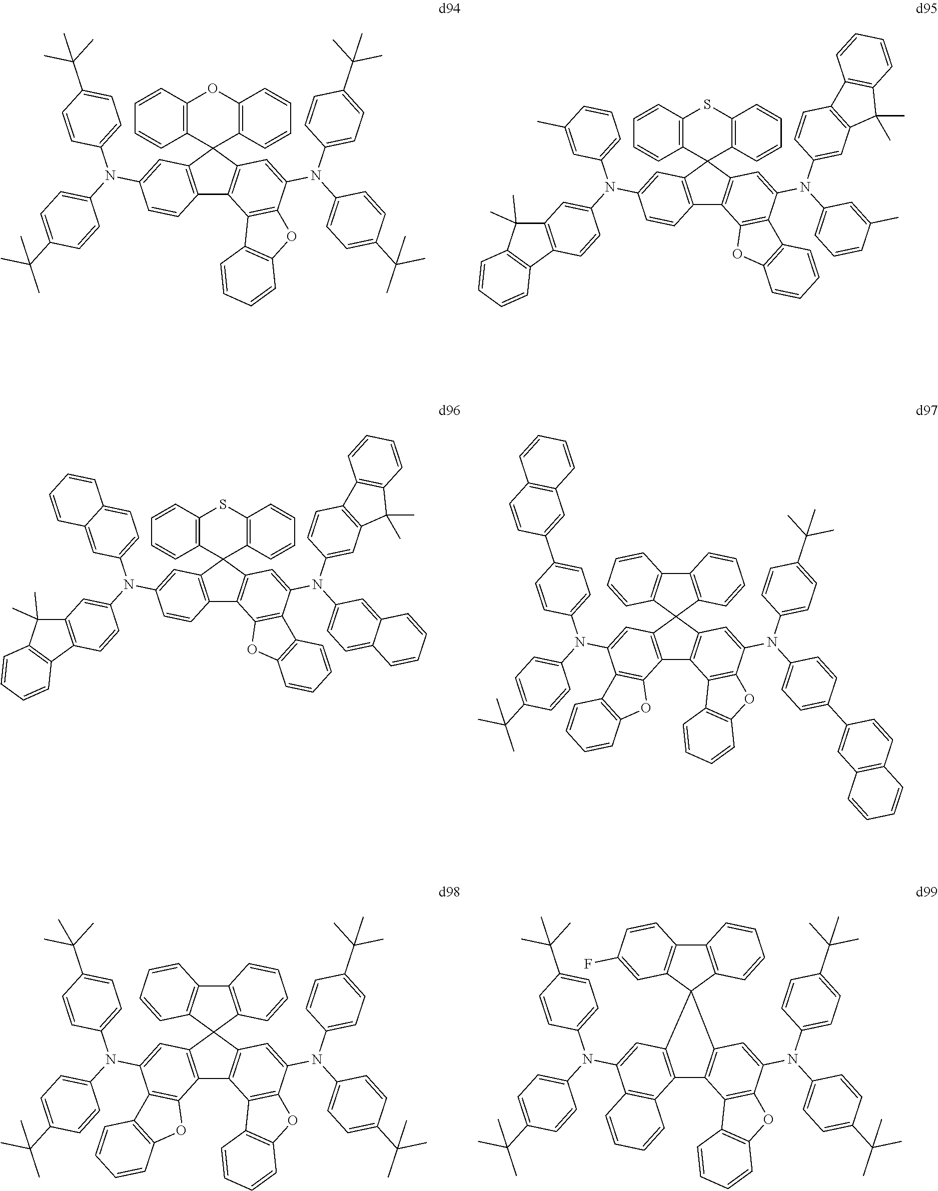

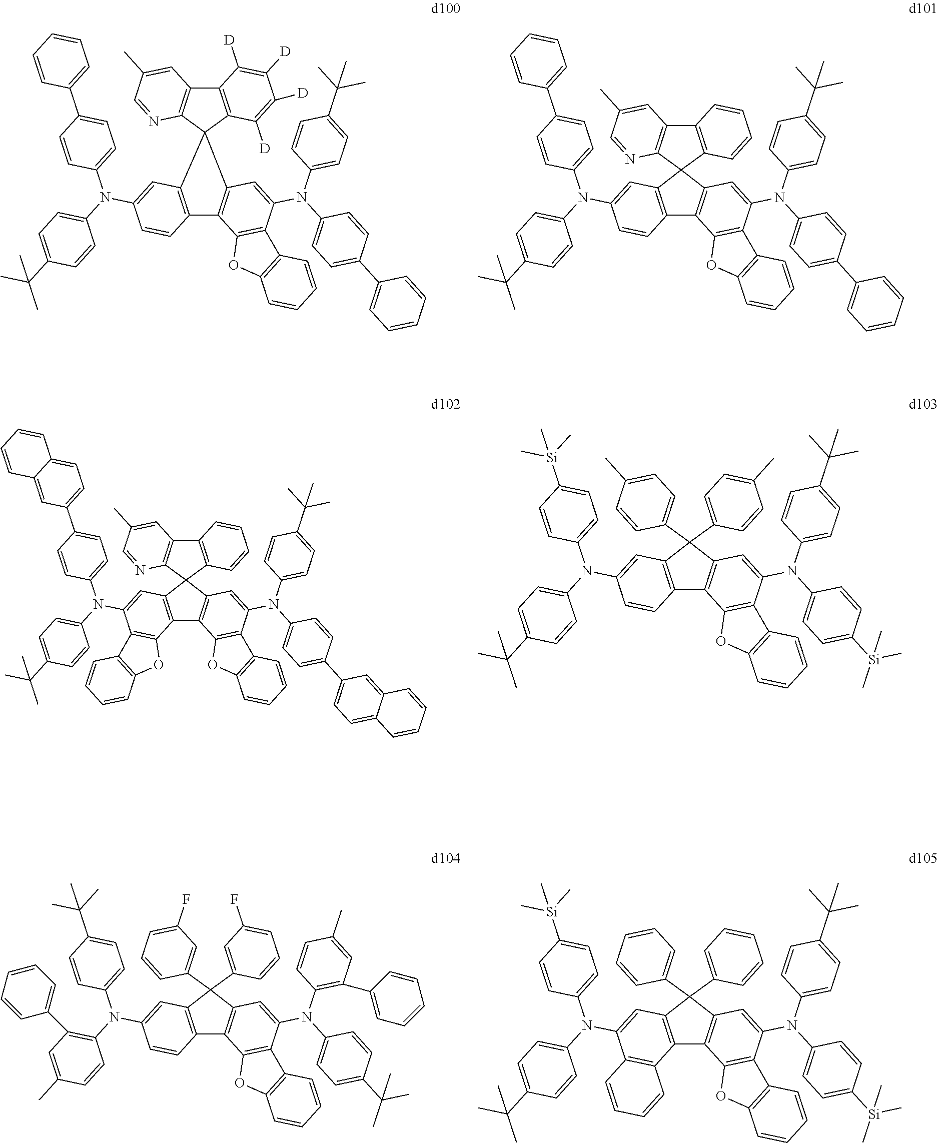

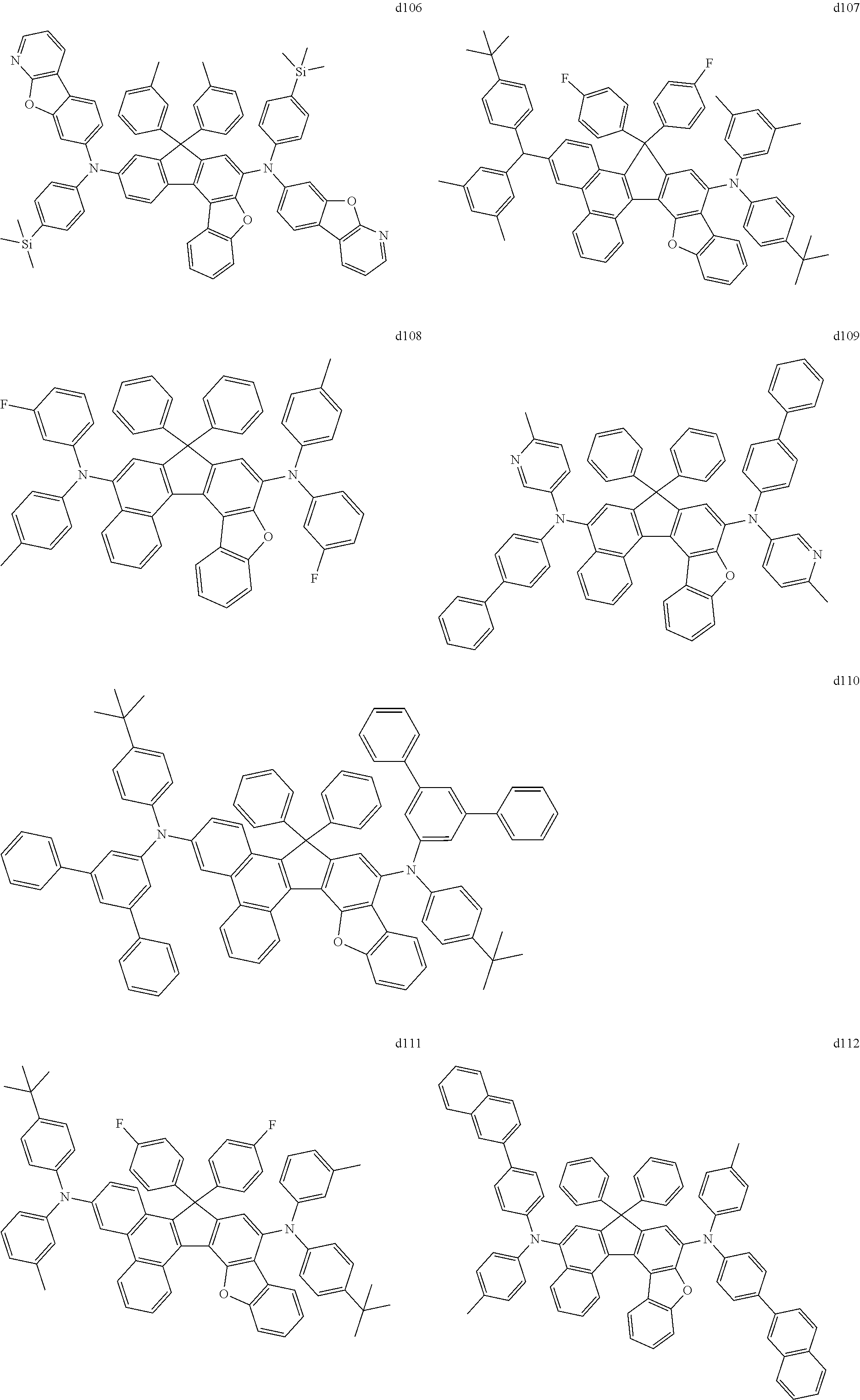

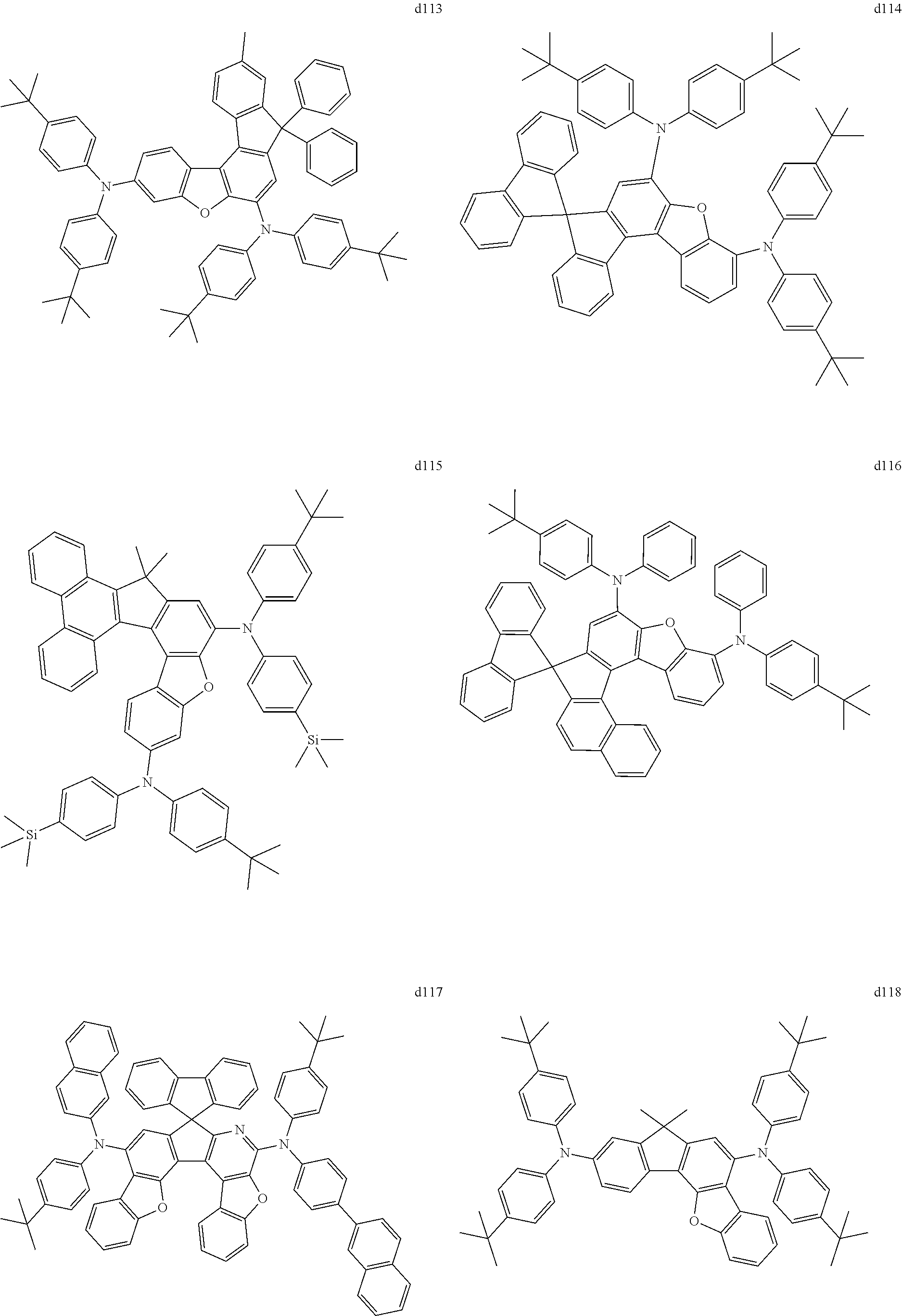

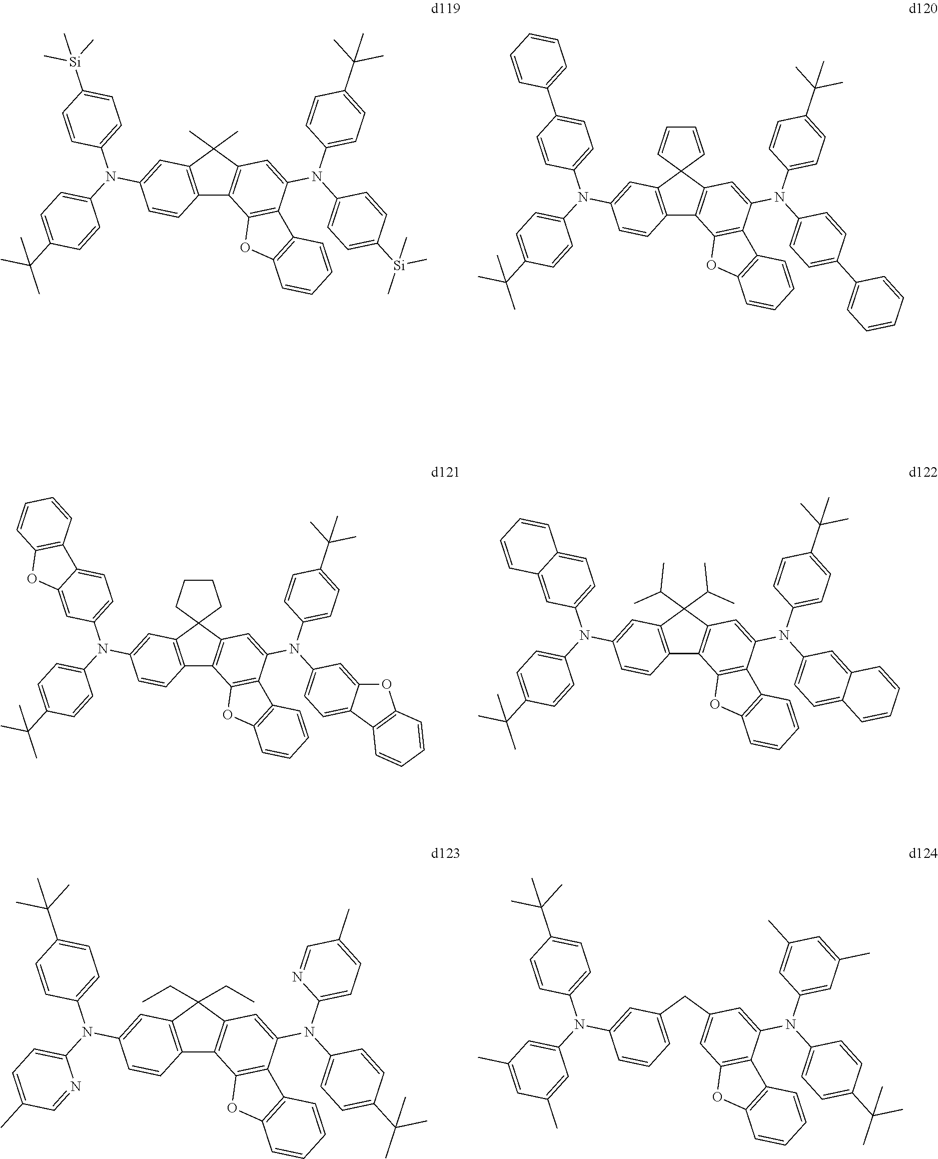

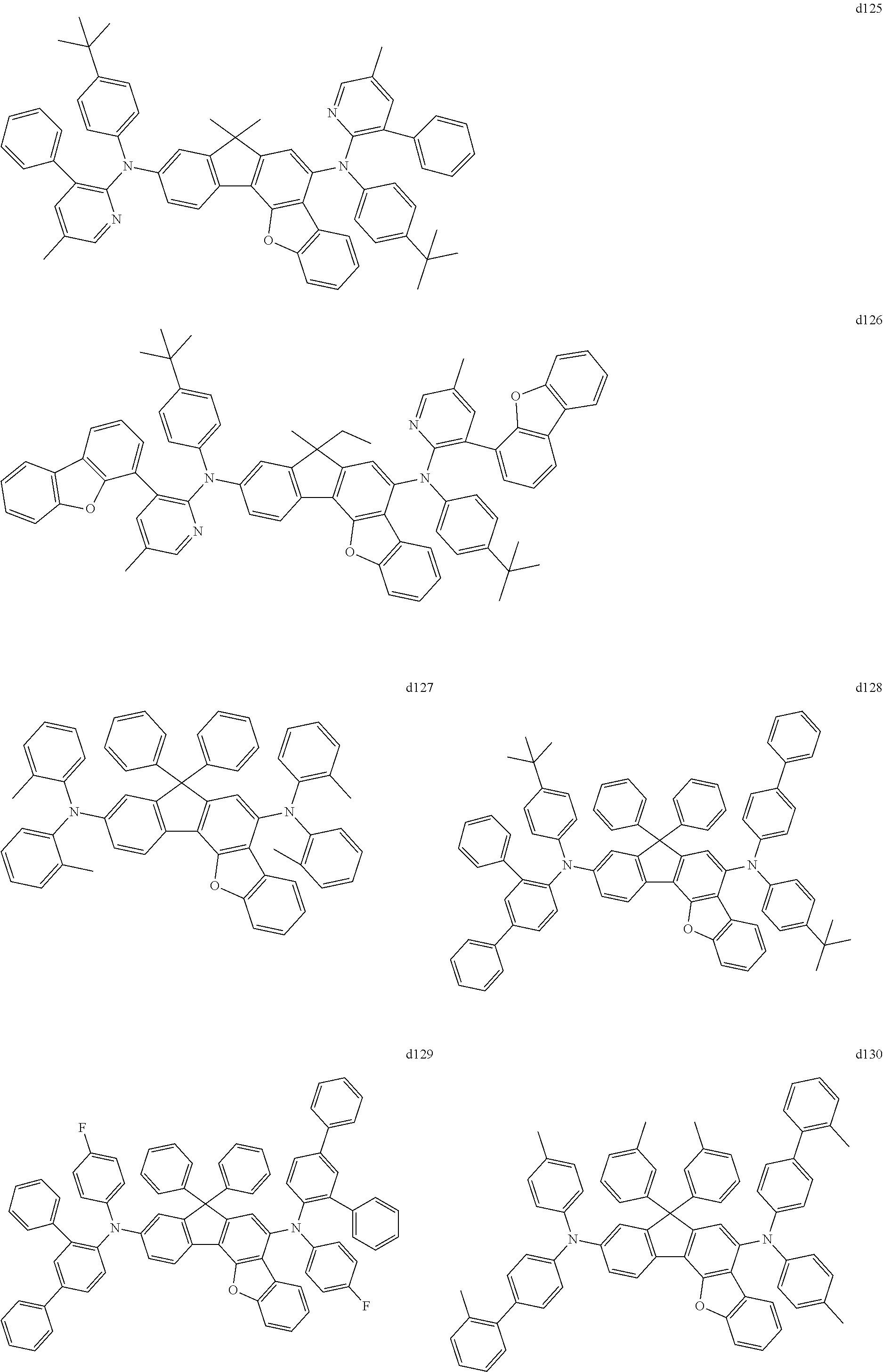

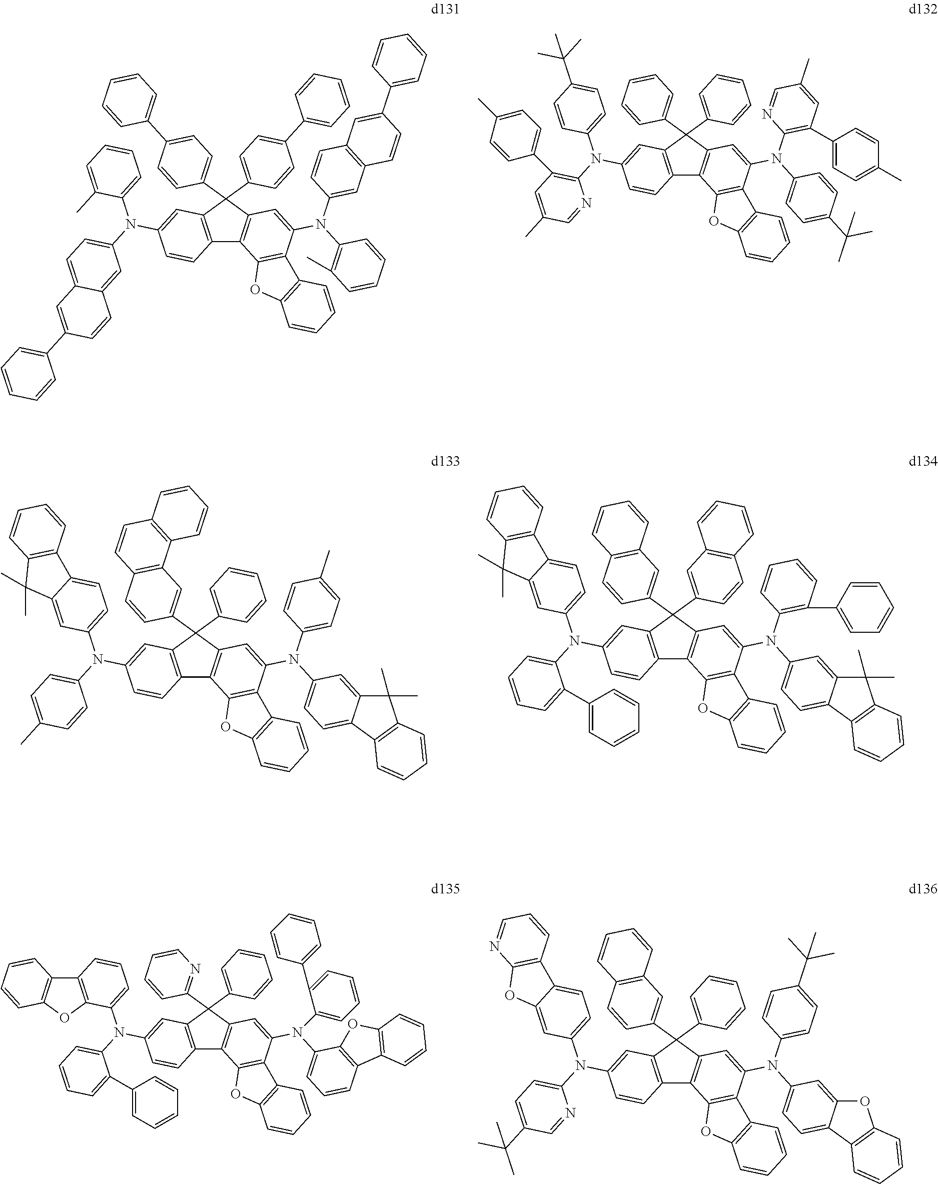

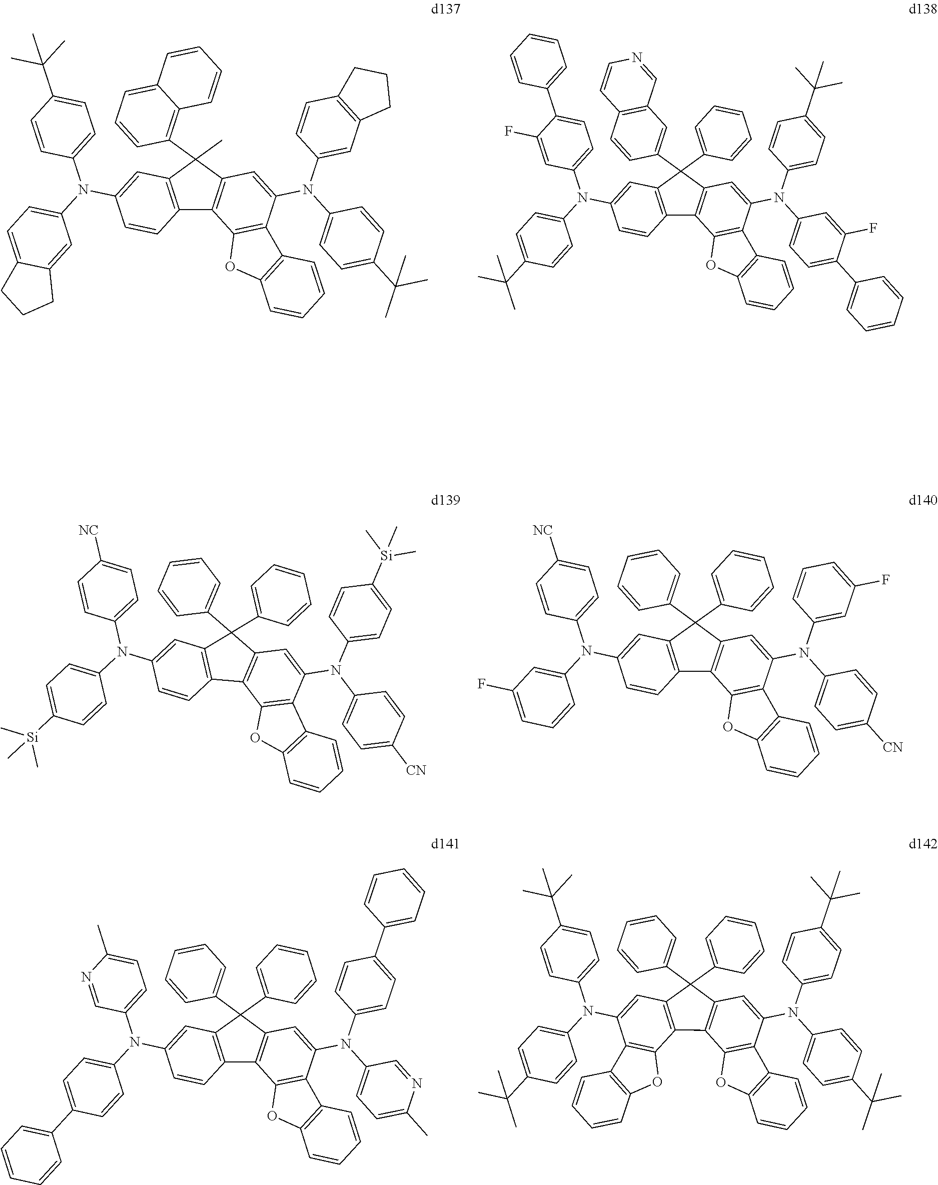

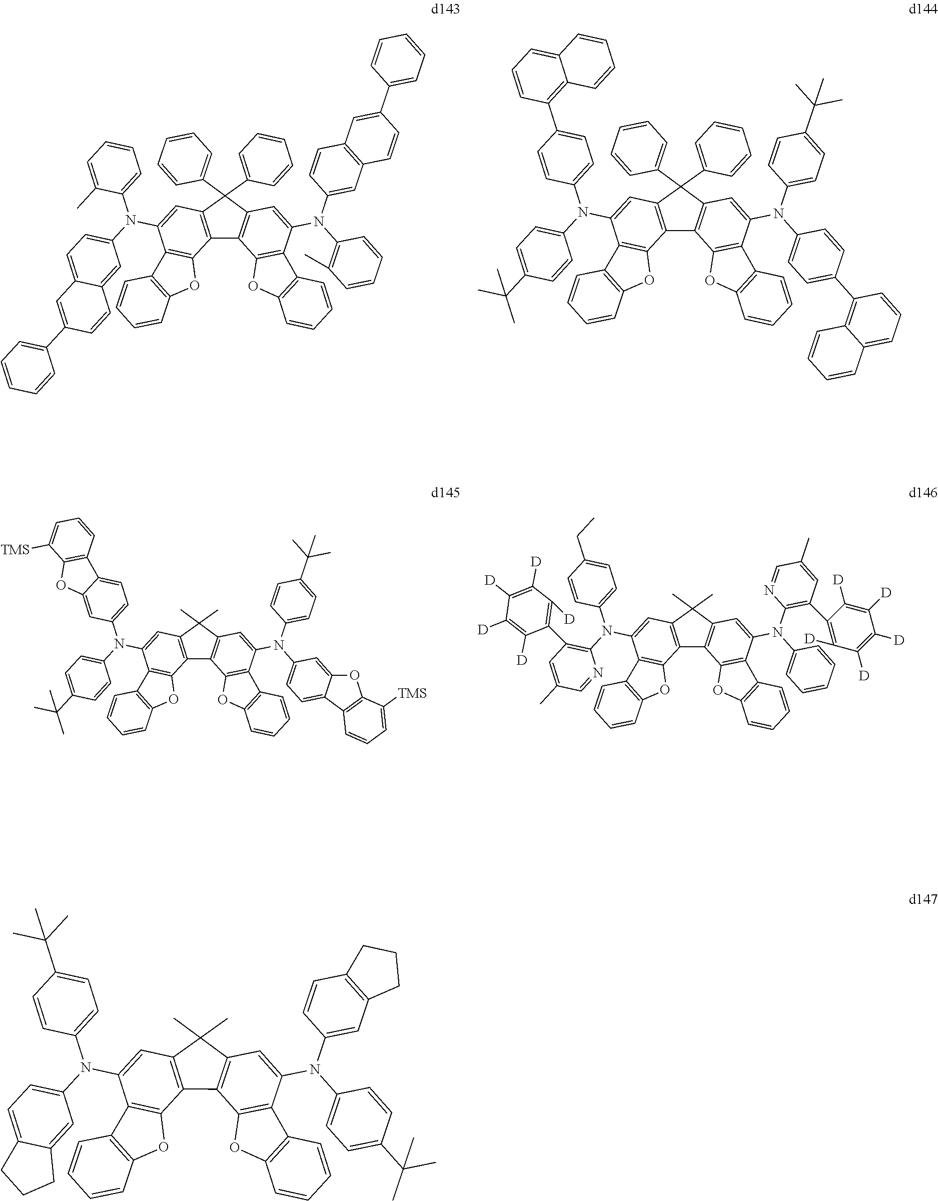

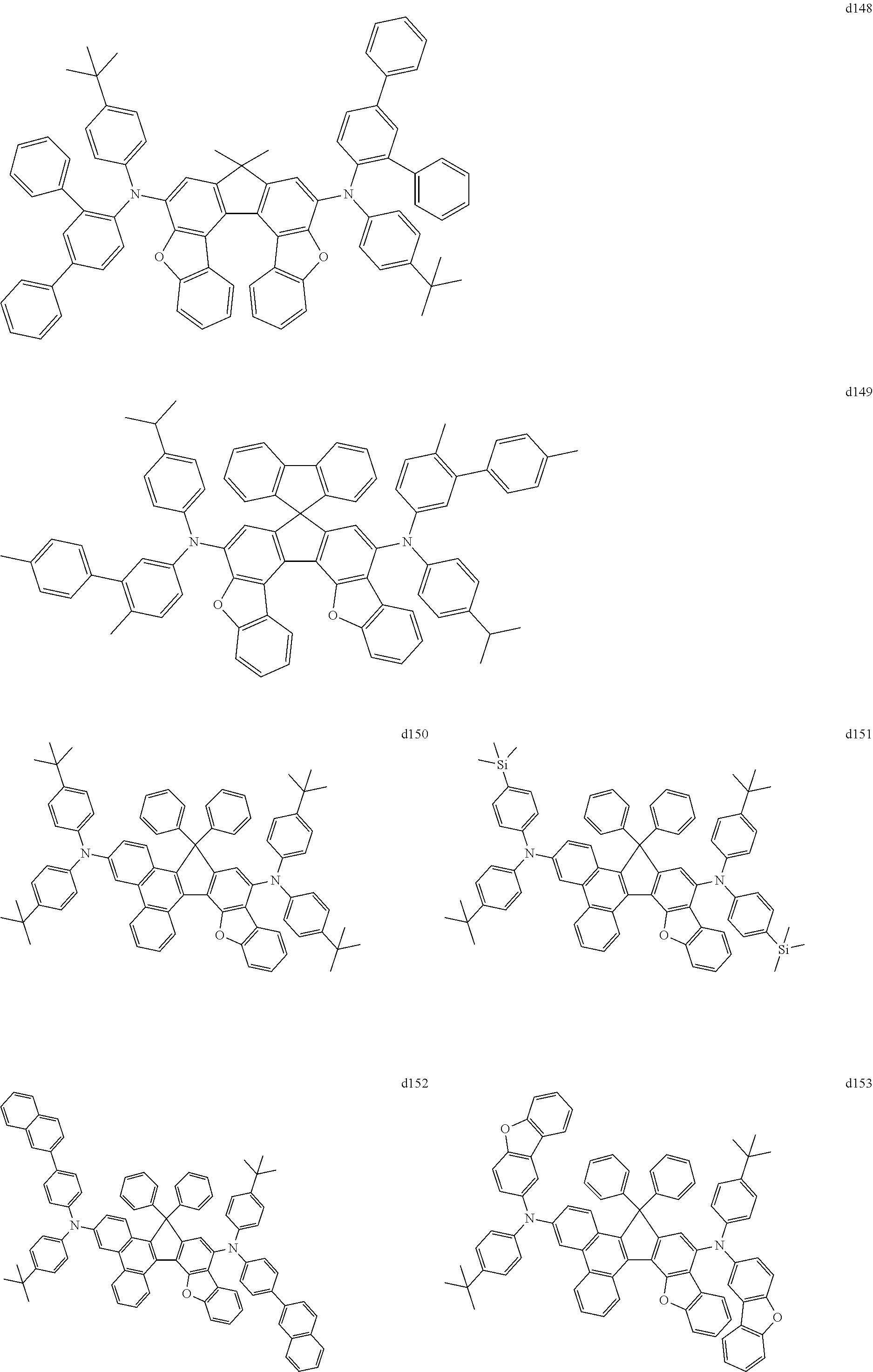

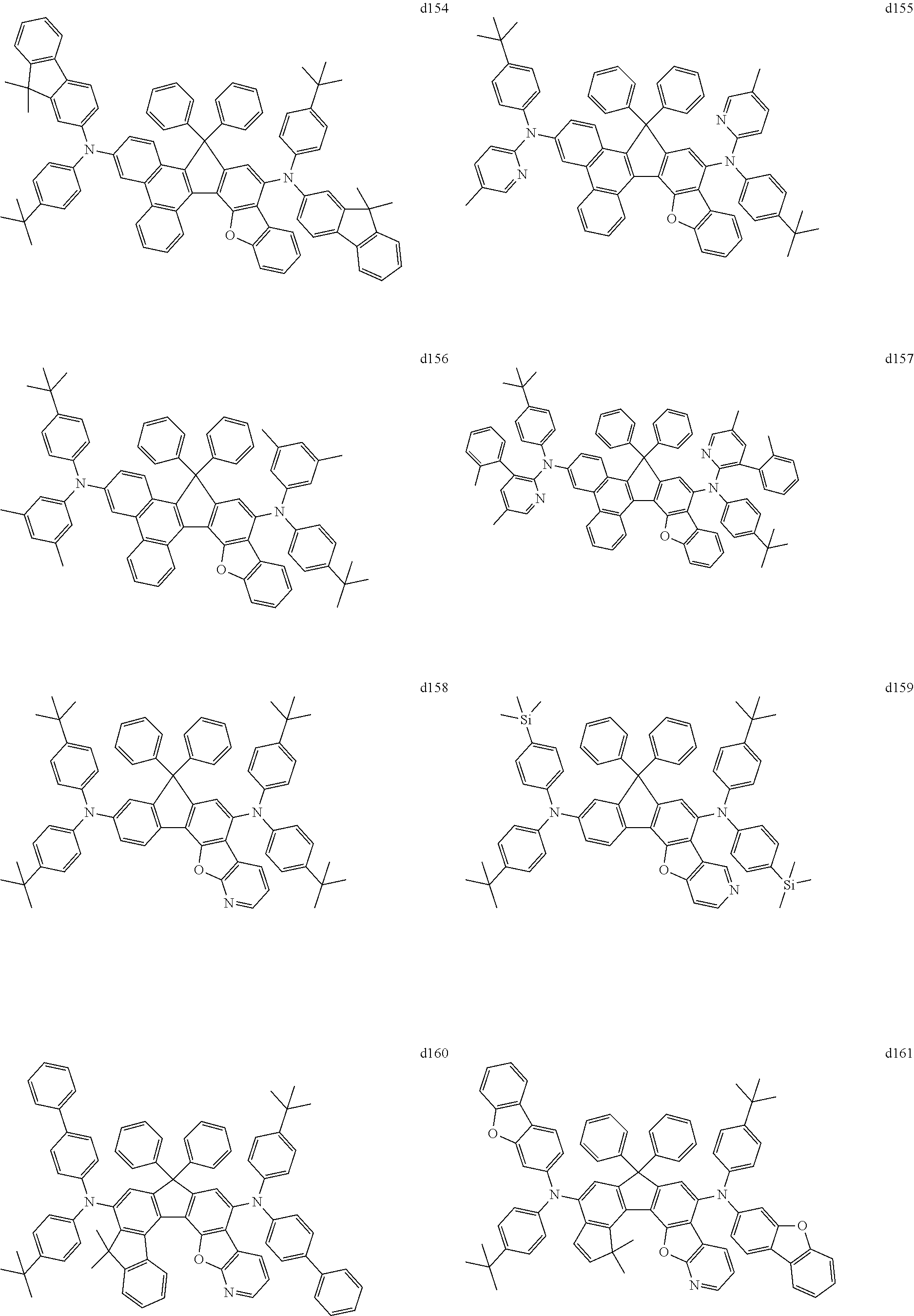

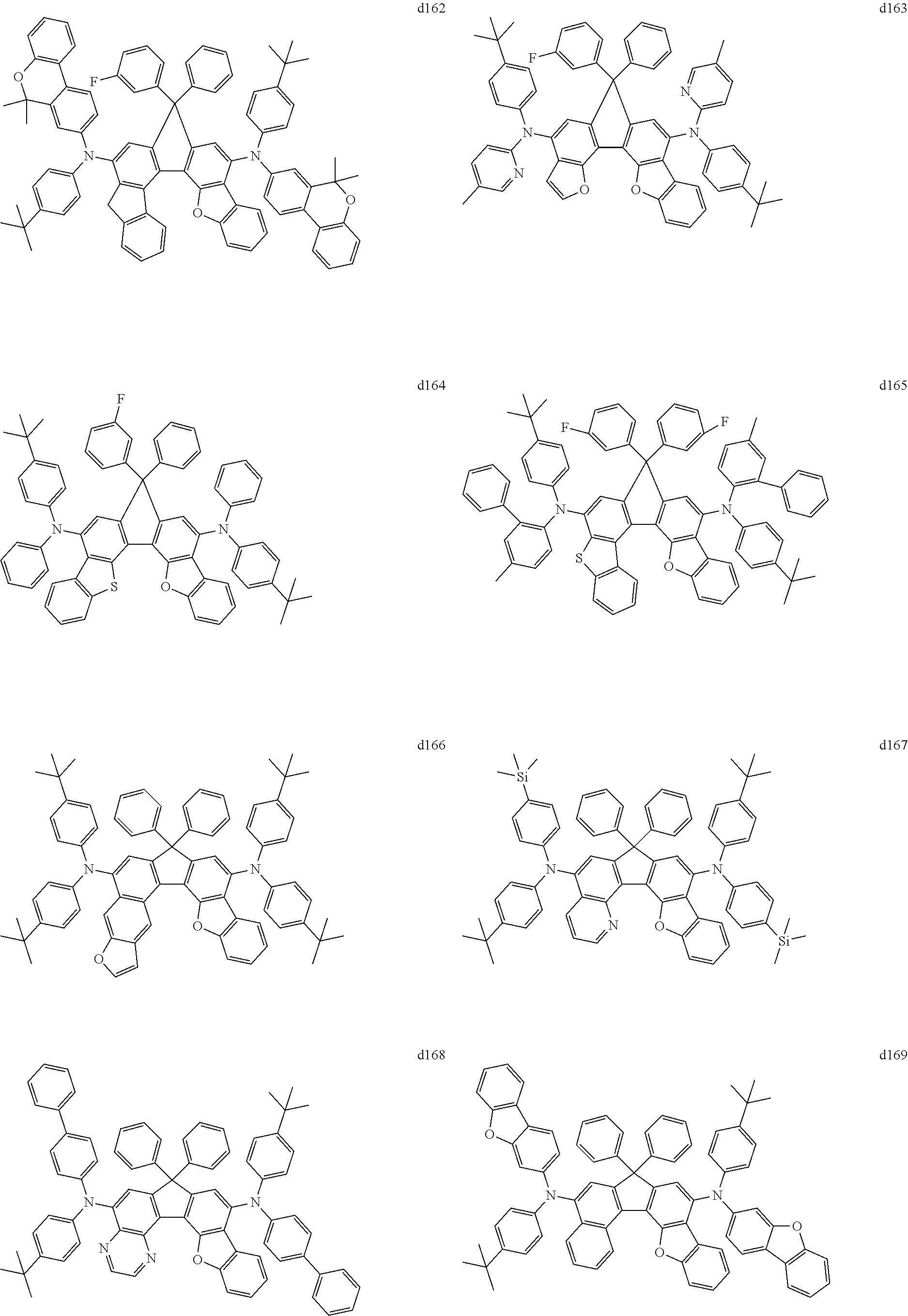

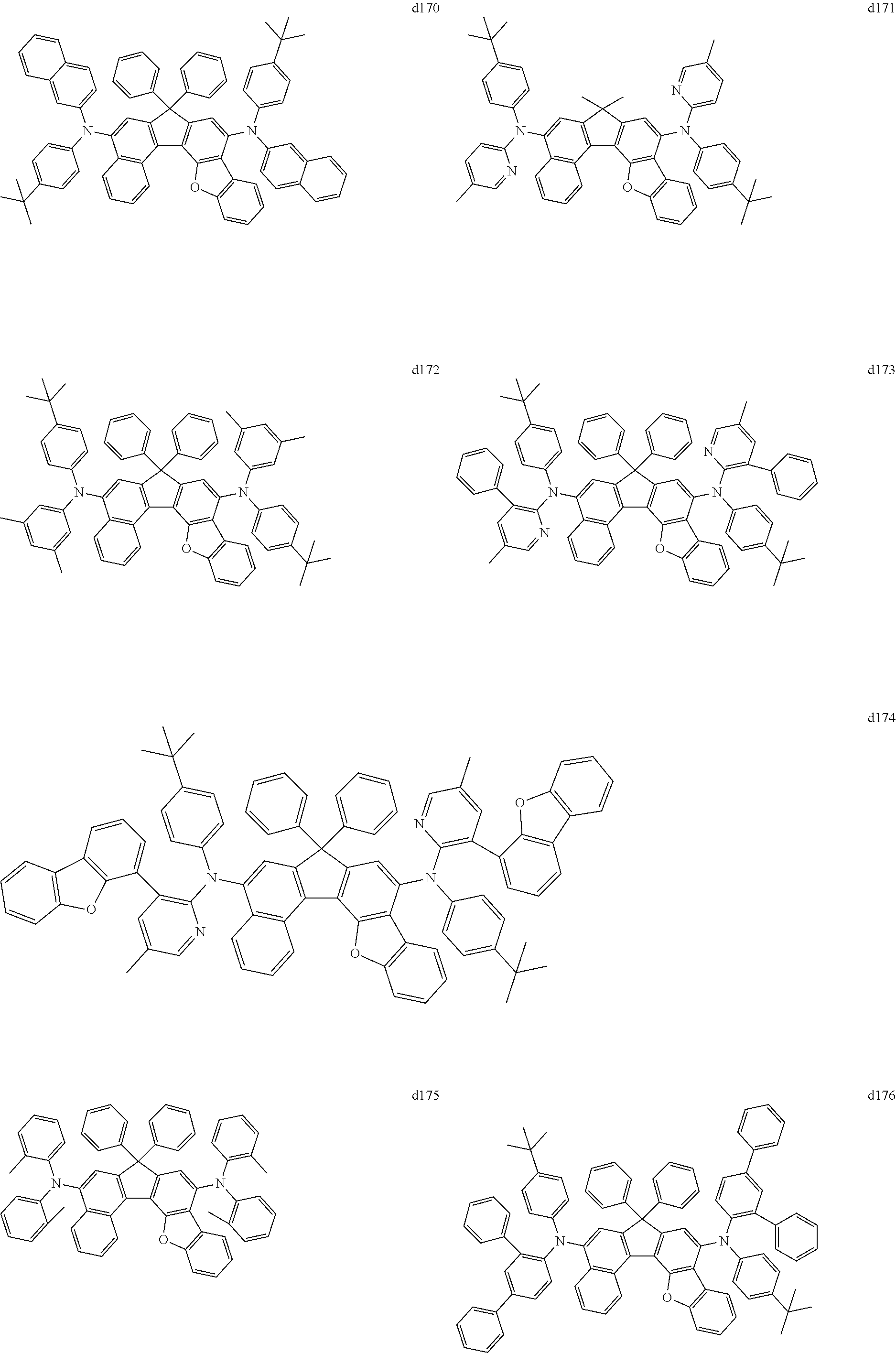

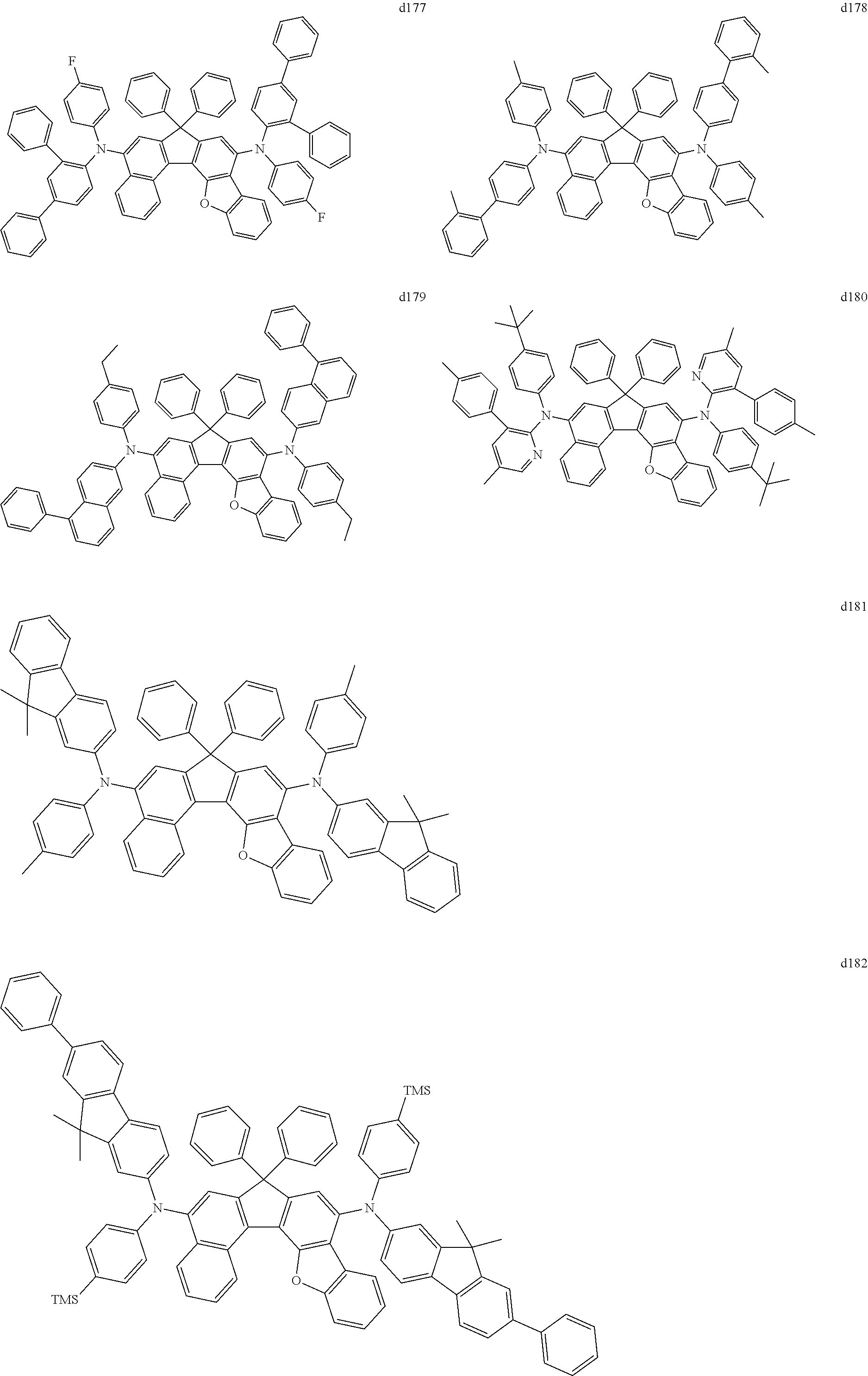

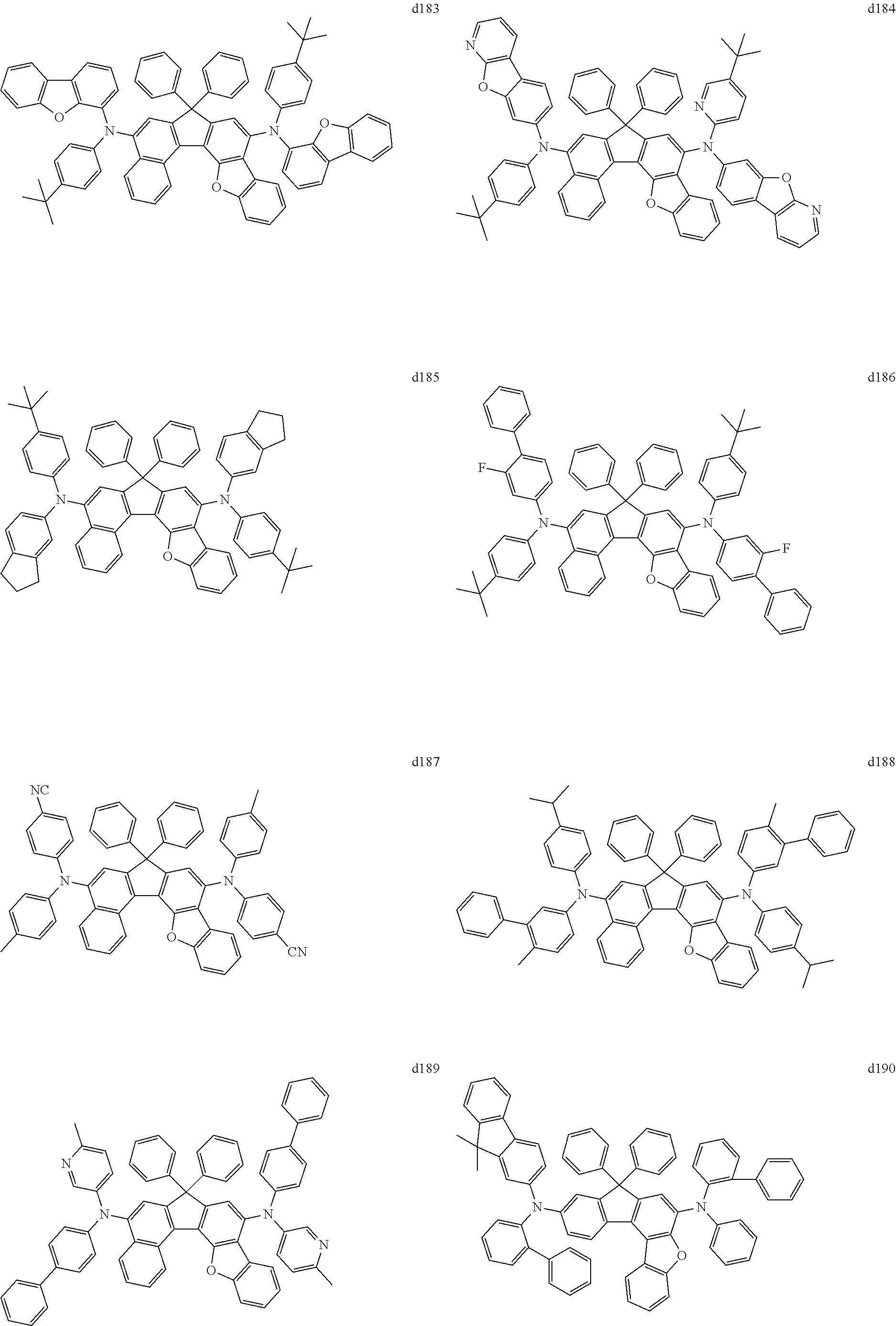

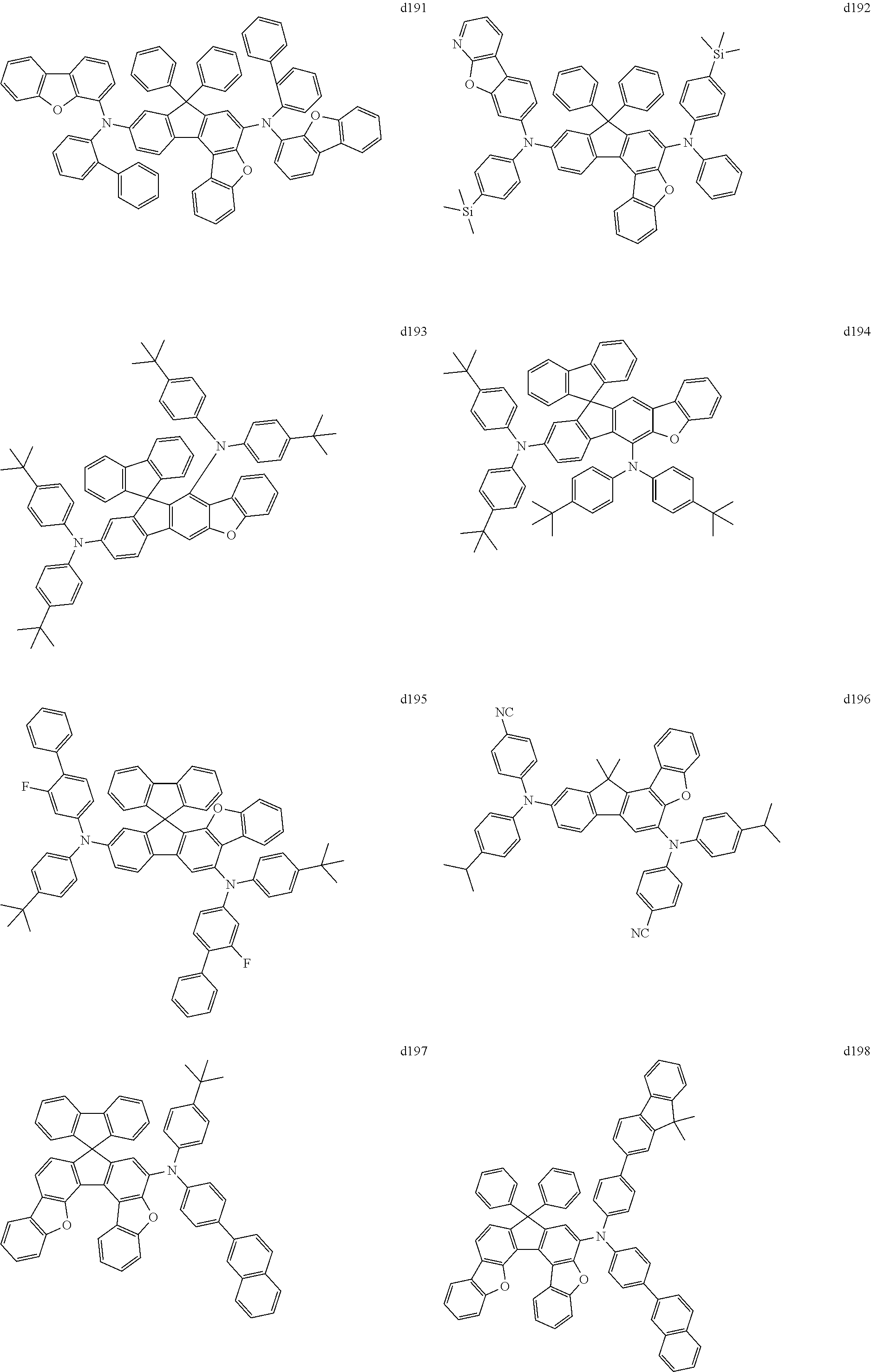

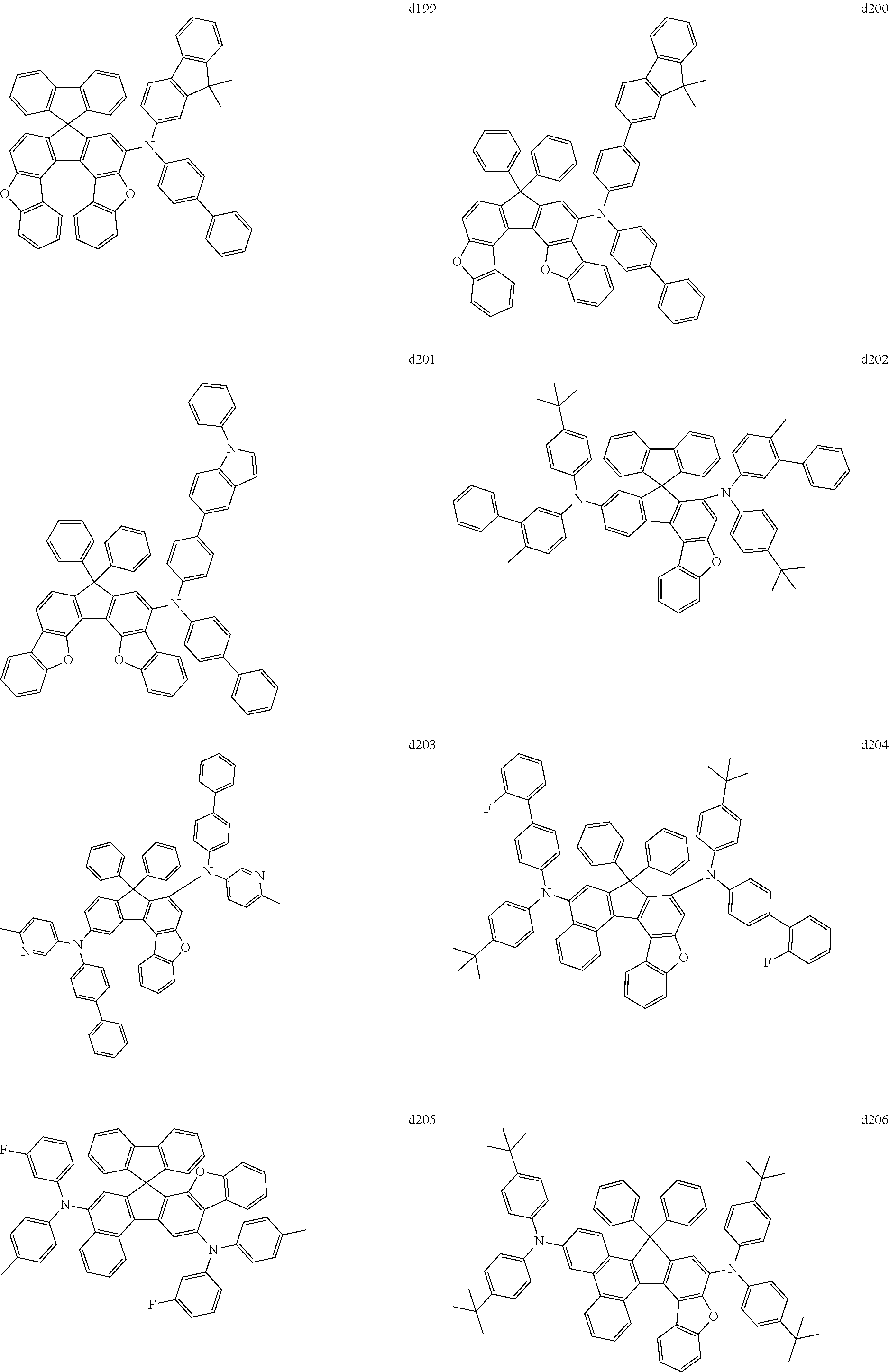

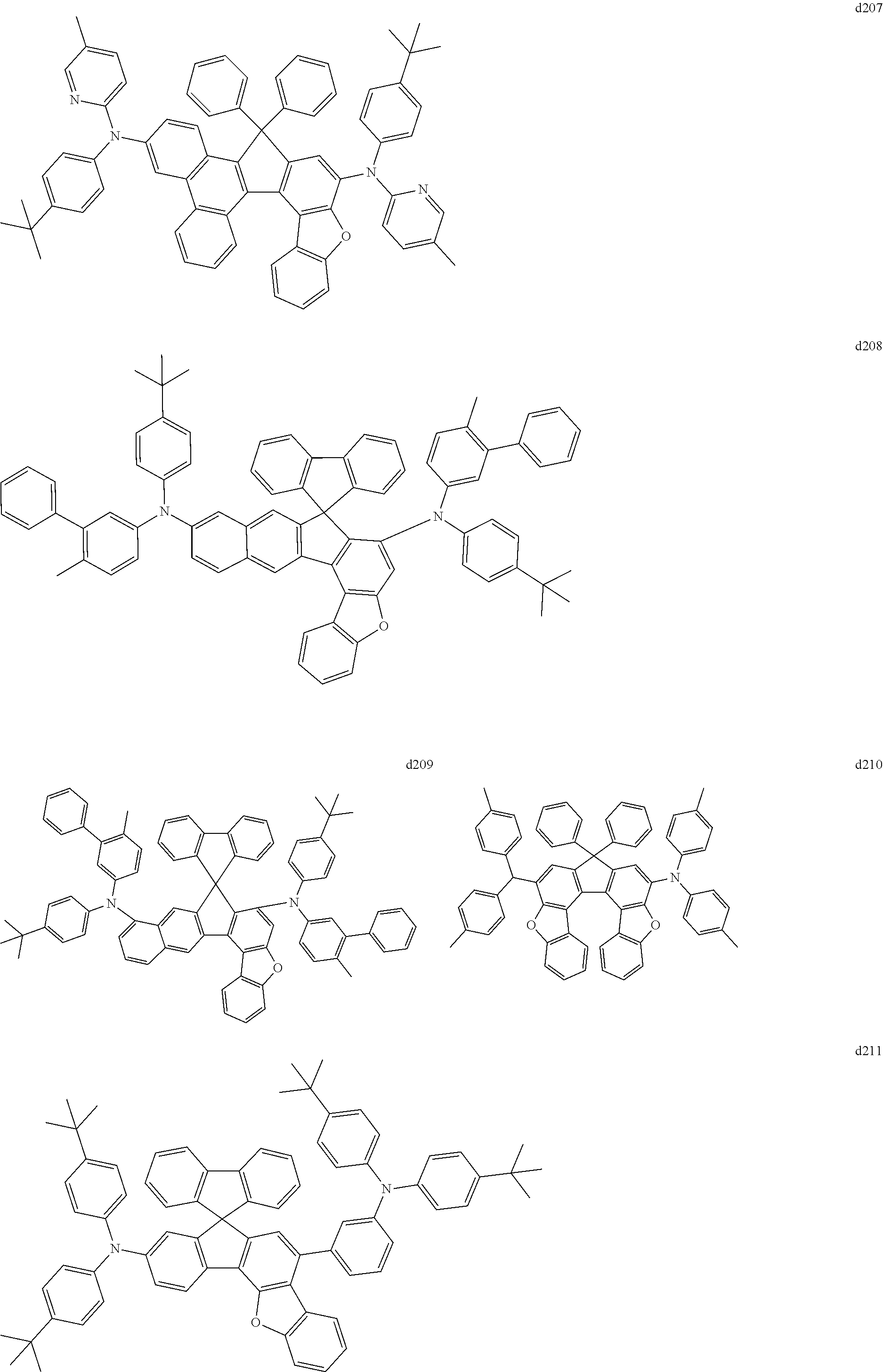

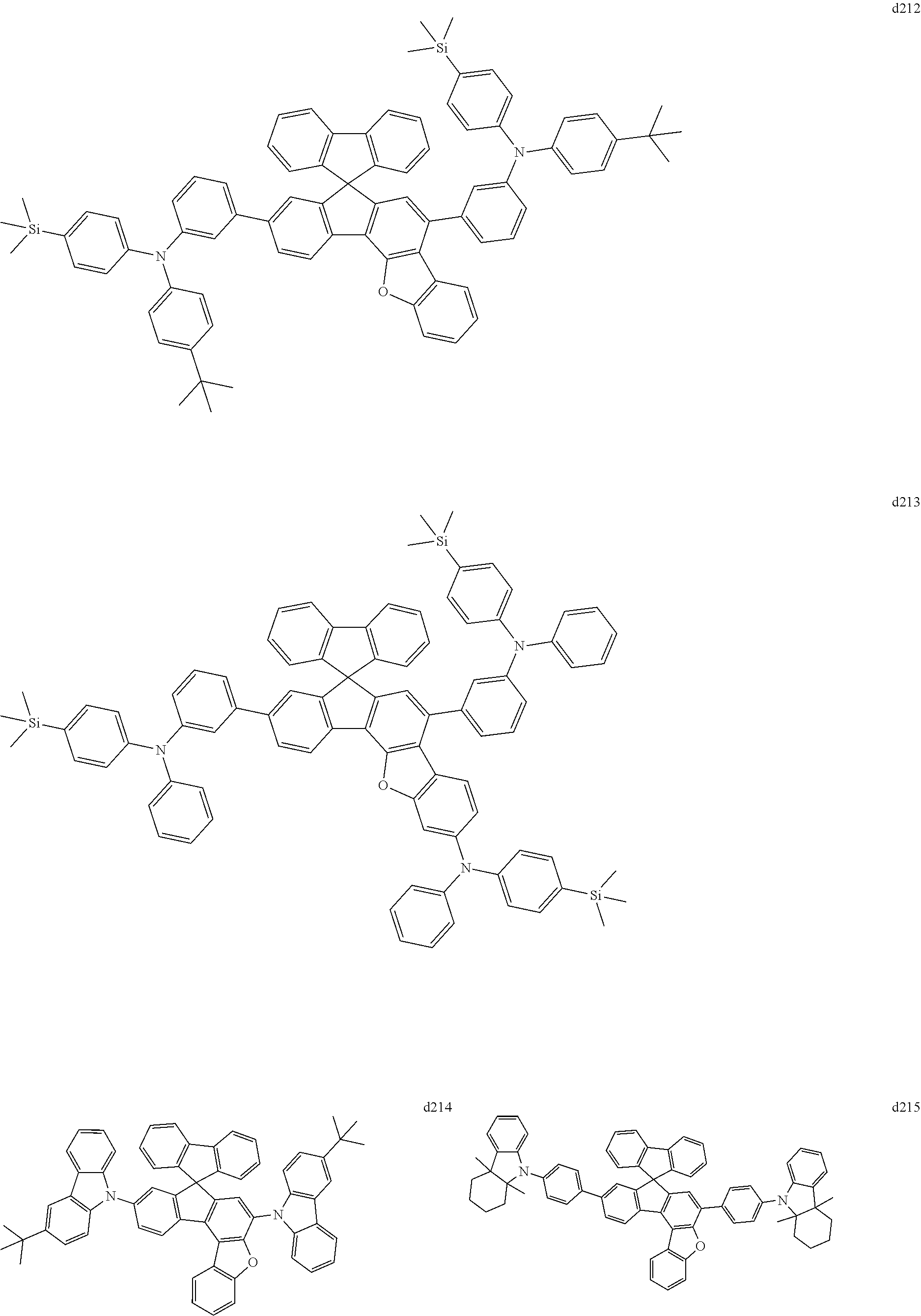

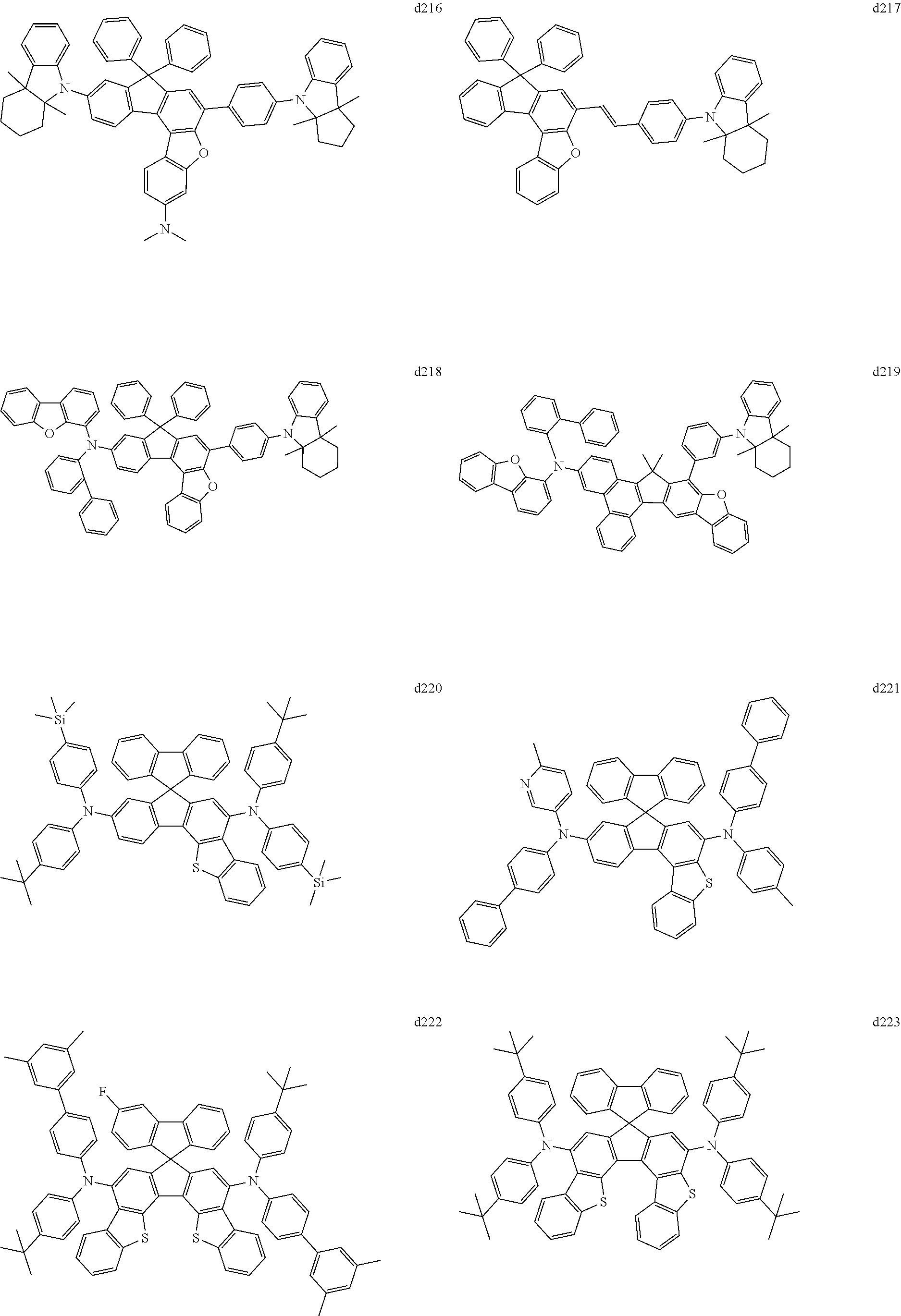

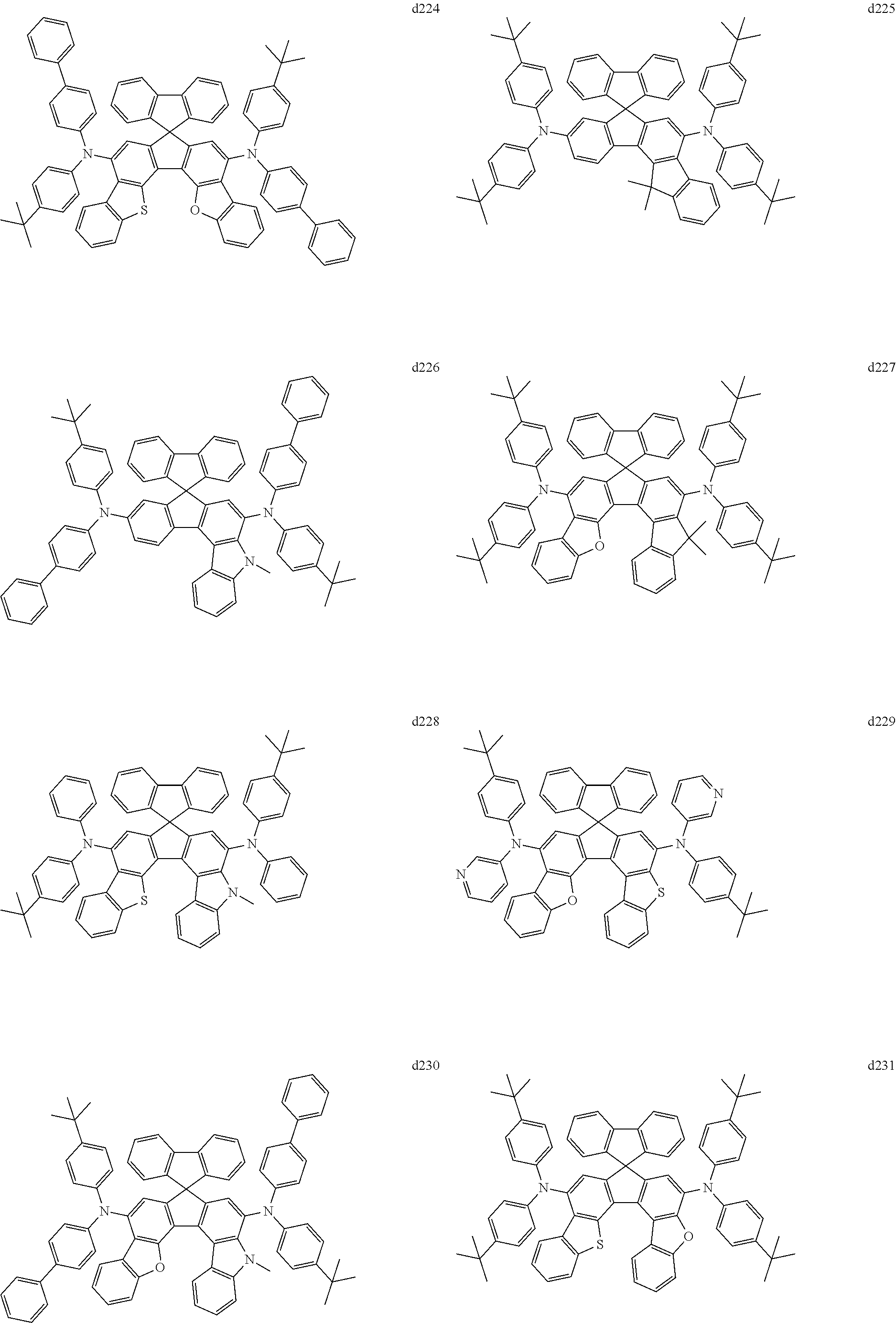

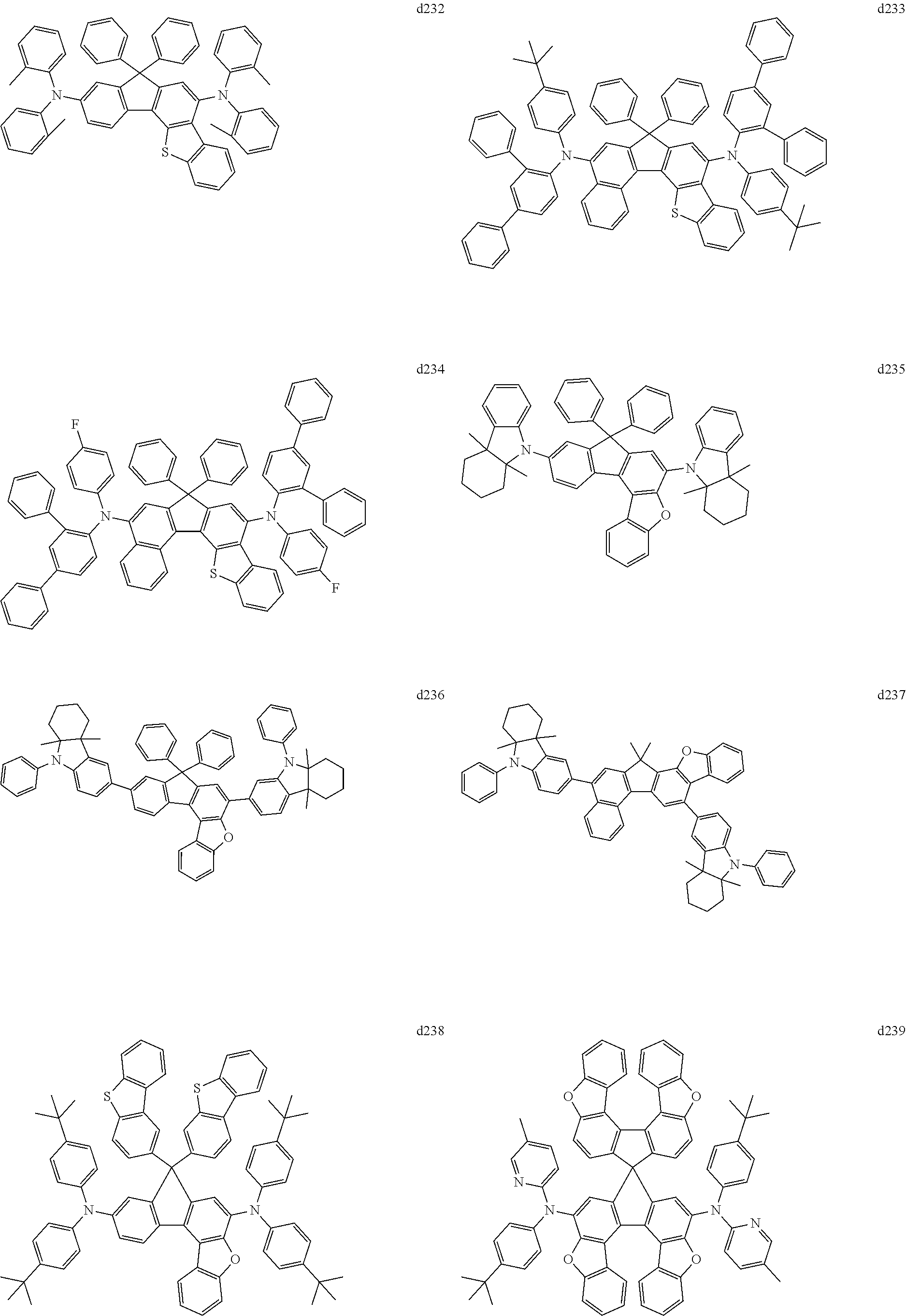

The compound represented by Formula D3 can be used as a dopant compound in the light emitting layer and may be selected from the following compounds D101 to D130:

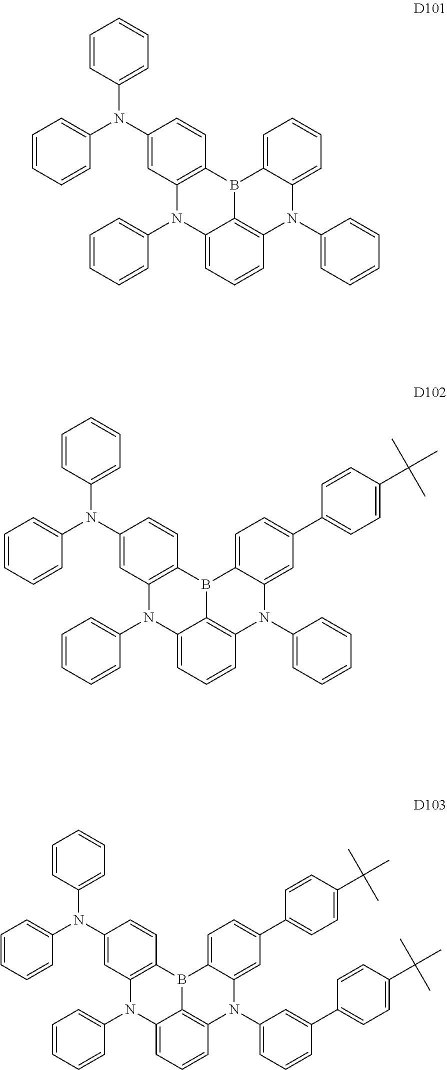 [SMILES: c1ccc(N(c2ccccc2)c2ccc3c(c2)N(c2ccccc2)c2cccc4c2B3c2ccccc2N4c2ccccc2)cc1 ; CC(C)(C)c1ccc(-c2ccc3c(c2)N(c2ccccc2)c2cccc4c2B3c2ccc(N(c3ccccc3)c3ccccc3)cc2N4c2ccccc2)cc1 ; CC(C)(C)c1ccc(-c2cccc(N3c4cc(-c5ccc(C(C)(C)C)cc5)ccc4B4c5ccc(N(c6ccccc6)c6ccccc6)cc5N(c5ccccc5)c5cccc3c54)c2)cc1]

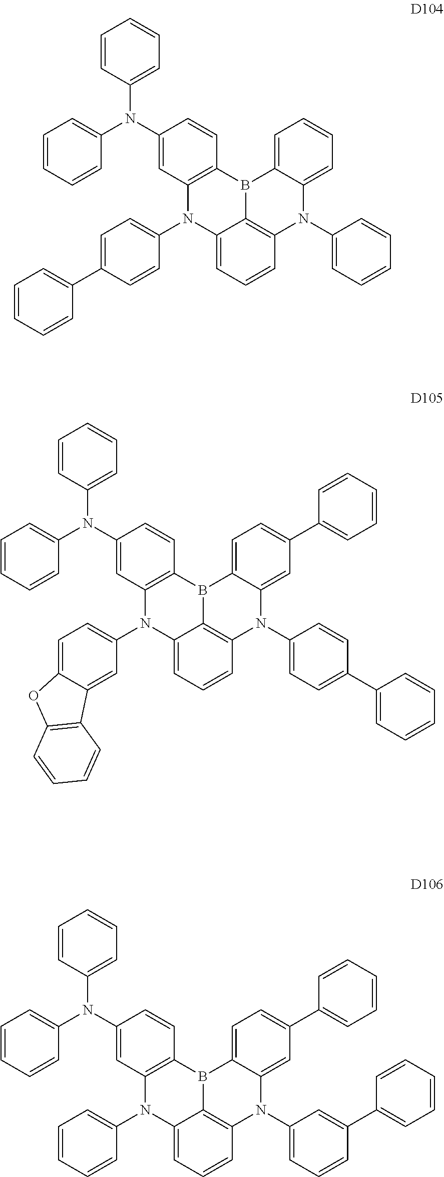 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(-c2ccc(N3c4cc(-c5ccccc5)ccc4B4c5ccc(N(c6ccccc6)c6ccccc6)cc5N(c5ccc6oc7ccccc7c6c5)c5cccc3c54)cc2)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

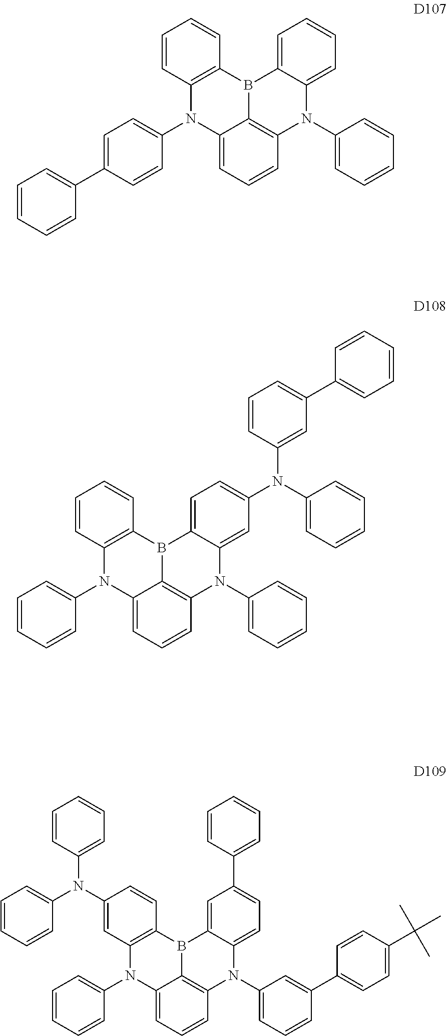 [SMILES: c1ccc(-c2ccc(N3c4ccccc4B4c5ccccc5N(c5ccccc5)c5cccc3c54)cc2)cc1 ; c1ccc(-c2cccc(N(c3ccccc3)c3ccc4c(c3)N(c3ccccc3)c3cccc5c3B4c3ccccc3N5c3ccccc3)c2)cc1 ; CC(C)(C)c1ccc(-c2cccc(N3c4ccc(-c5ccccc5)cc4B4c5ccc(N(c6ccccc6)c6ccccc6)cc5N(c5ccccc5)c5cccc3c54)c2)cc1]

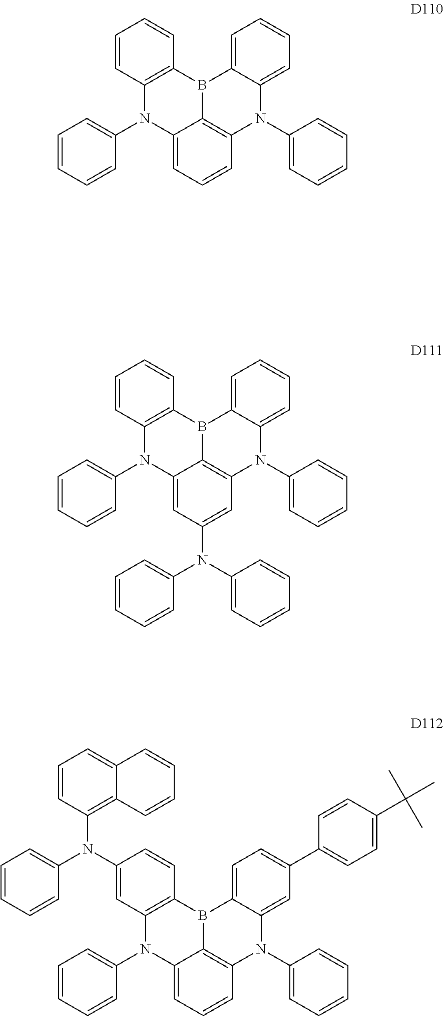 [SMILES: c1ccc(N2c3ccccc3B3c4ccccc4N(c4ccccc4)c4cccc2c43)cc1 ; c1ccc(N(c2ccccc2)c2cc3c4c(c2)N(c2ccccc2)c2ccccc2B4c2ccccc2N3c2ccccc2)cc1 ; CC(C)(C)c1ccc(-c2ccc3c(c2)N(c2ccccc2)c2cccc4c2B3c2ccc(N(c3ccccc3)c3cccc5ccccc35)cc2N4c2ccccc2)cc1]

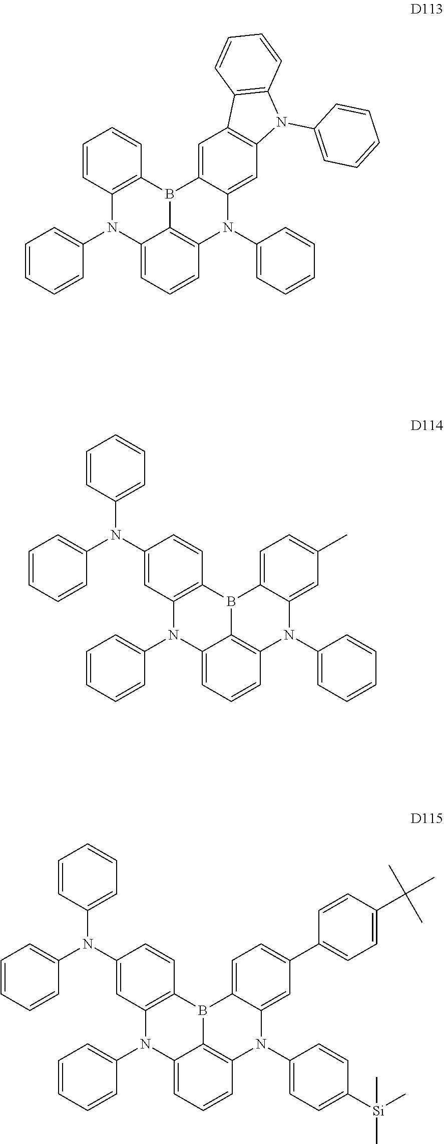 [SMILES: c1ccc(N2c3ccccc3B3c4cc5c6ccccc6n(-c6ccccc6)c5cc4N(c4ccccc4)c4cccc2c43)cc1 ; Cc1ccc2c(c1)N(c1ccccc1)c1cccc3c1B2c1ccc(N(c2ccccc2)c2ccccc2)cc1N3c1ccccc1 ; CC(C)(C)c1ccc(-c2ccc3c(c2)N(c2ccc([Si](C)(C)C)cc2)c2cccc4c2B3c2ccc(N(c3ccccc3)c3ccccc3)cc2N4c2ccccc2)cc1]

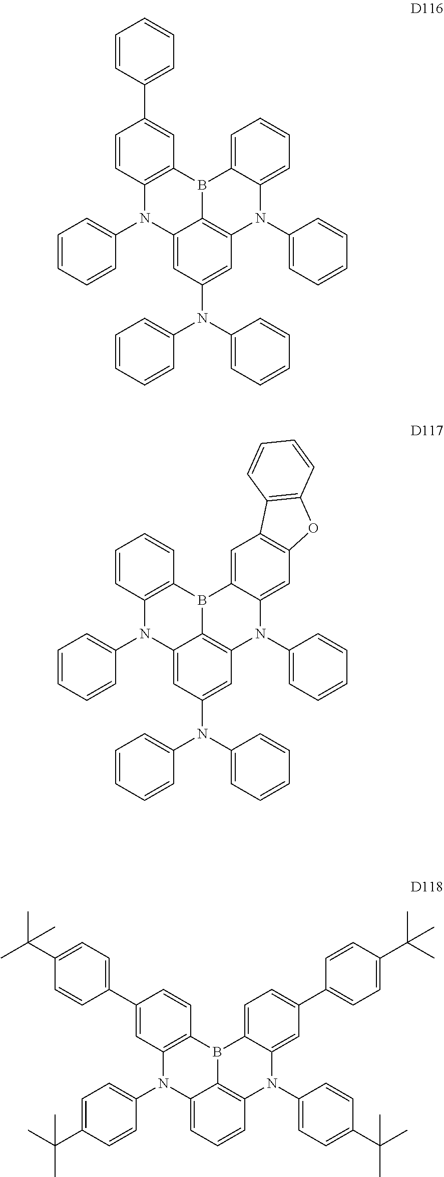 [SMILES: c1ccc(-c2ccc3c(c2)B2c4ccccc4N(c4ccccc4)c4cc(N(c5ccccc5)c5ccccc5)cc(c42)N3c2ccccc2)cc1 ; c1ccc(N(c2ccccc2)c2cc3c4c(c2)N(c2ccccc2)c2cc5oc6ccccc6c5cc2B4c2ccccc2N3c2ccccc2)cc1 ; CC(C)(C)c1ccc(-c2ccc3c(c2)N(c2ccc(C(C)(C)C)cc2)c2cccc4c2B3c2ccc(-c3ccc(C(C)(C)C)cc3)cc2N4c2ccc(C(C)(C)C)cc2)cc1]

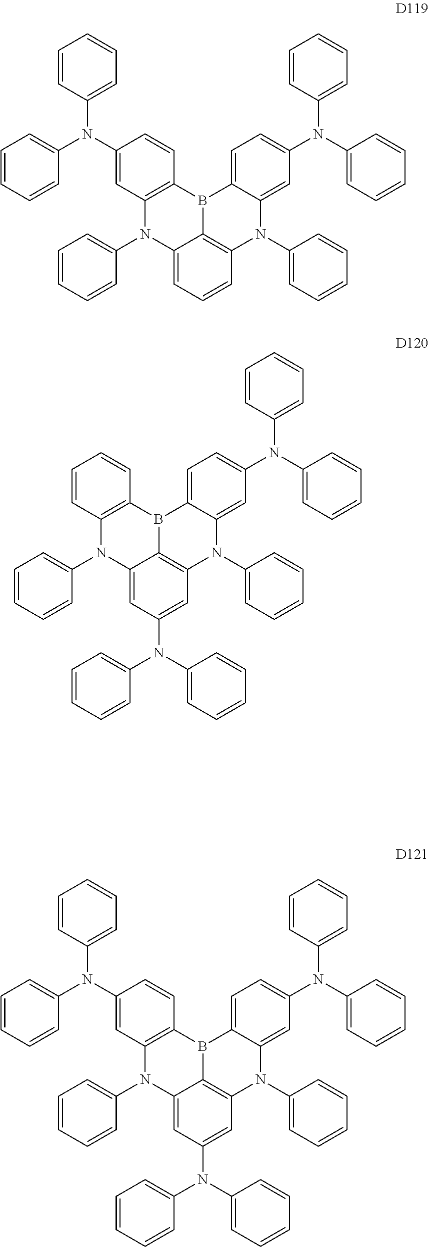 [SMILES: c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cc(N(c4ccccc4)c4ccccc4)cc4c2B3c2ccccc2N4c2ccccc2)cc1 ; c1ccc(N(c2ccccc2)c2ccc3c(c2)N(c2ccccc2)c2cc(N(c4ccccc4)c4ccccc4)cc4c2B3c2ccc(N(c3ccccc3)c3ccccc3)cc2N4c2ccccc2)cc1]

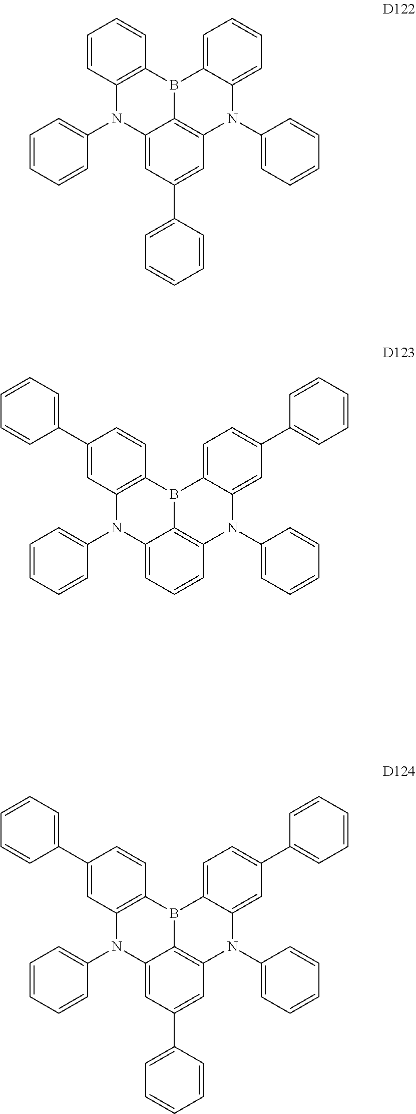 [SMILES: c1ccc(-c2cc3c4c(c2)N(c2ccccc2)c2ccccc2B4c2ccccc2N3c2ccccc2)cc1 ; c1ccc(-c2ccc3c(c2)N(c2ccccc2)c2cccc4c2B3c2ccc(-c3ccccc3)cc2N4c2ccccc2)cc1 ; c1ccc(-c2ccc3c(c2)N(c2ccccc2)c2cc(-c4ccccc4)cc4c2B3c2ccc(-c3ccccc3)cc2N4c2ccccc2)cc1]

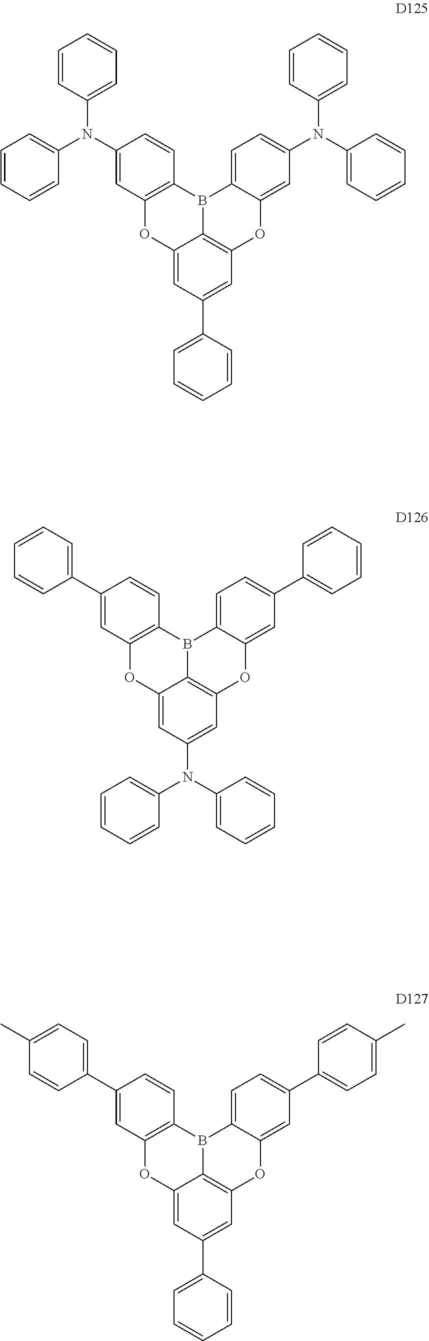

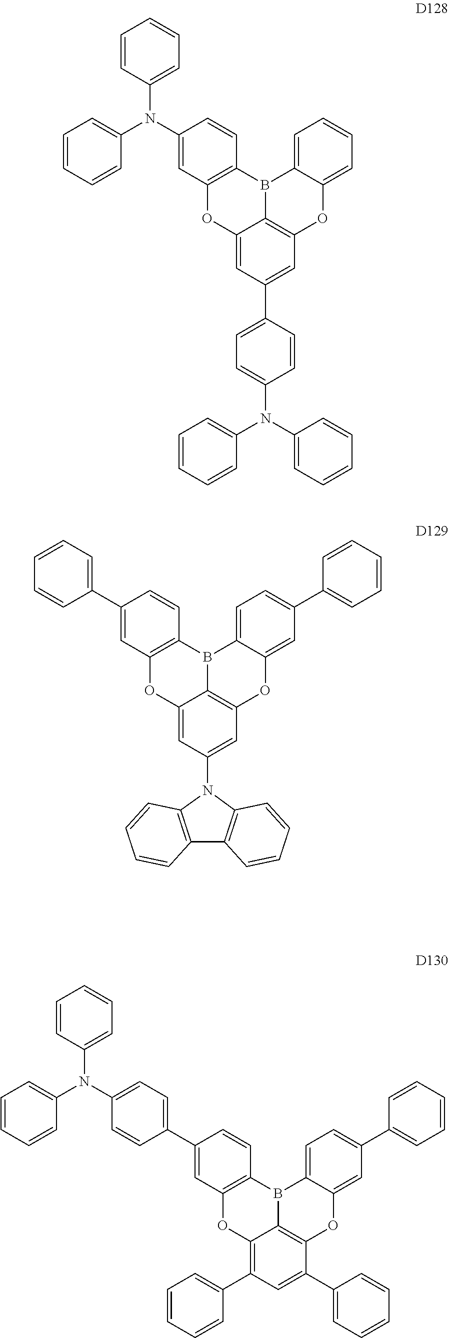 [SMILES: c1ccc(N(c2ccccc2)c2ccc(-c3cc4c5c(c3)Oc3cc(N(c6ccccc6)c6ccccc6)ccc3B5c3ccccc3O4)cc2)cc1 ; c1ccc(-c2ccc3c(c2)Oc2cc(-n4c5ccccc5c5ccccc54)cc4c2B3c2ccc(-c3ccccc3)cc2O4)cc1 ; c1ccc(-c2ccc3c(c2)Oc2c(-c4ccccc4)cc(-c4ccccc4)c4c2B3c2ccc(-c3ccc(N(c5ccccc5)c5ccccc5)cc3)cc2O4)cc1]

The compounds represented by Formulas D4, D5, and D8 to D10 can be used as dopant compounds in the light emitting layer and may be selected from the following compounds D201 to D476:

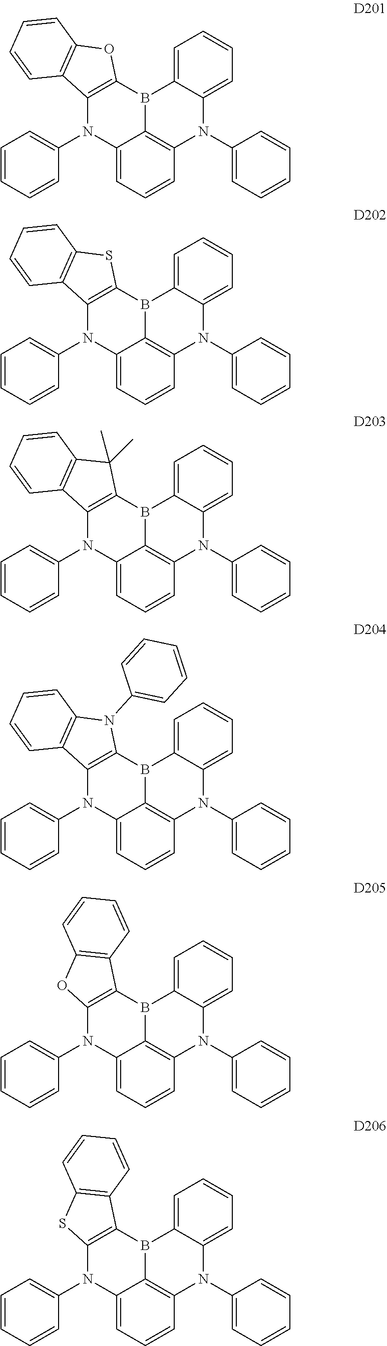

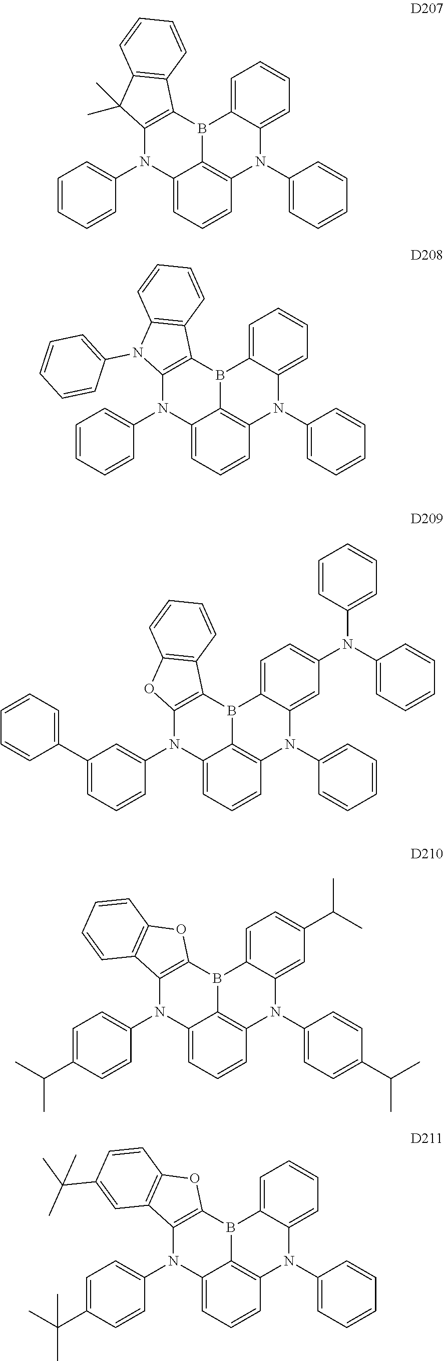

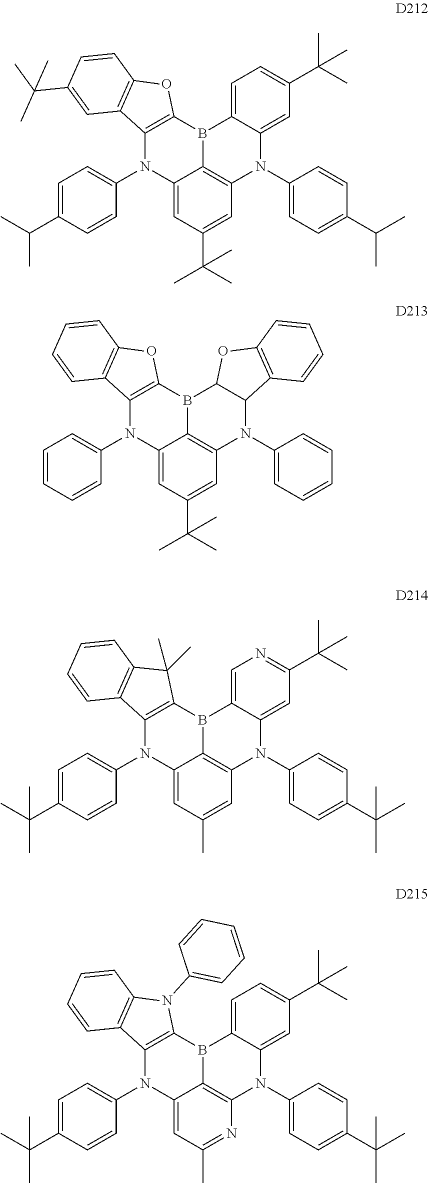

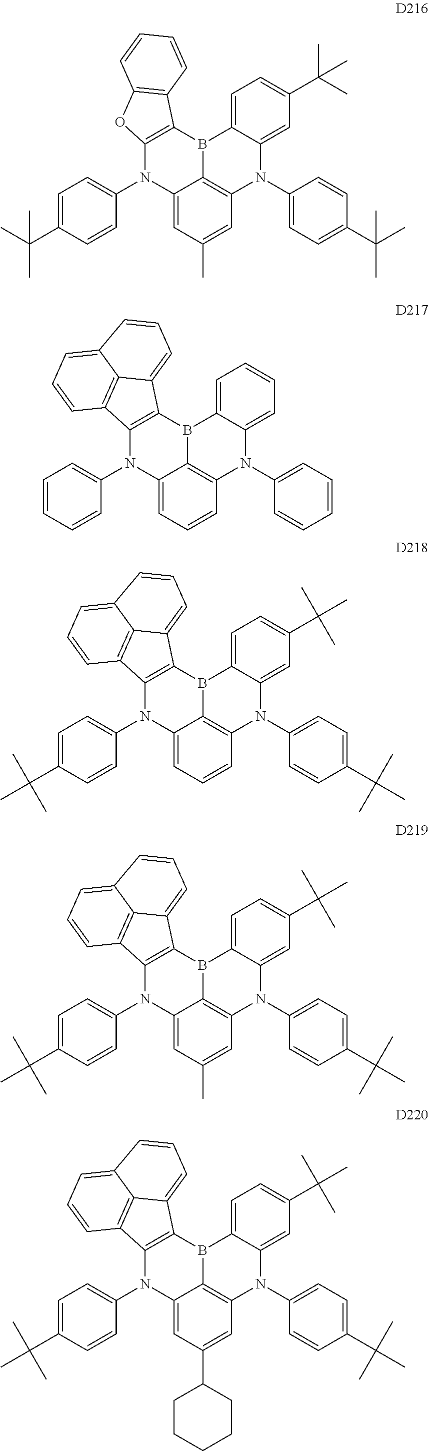

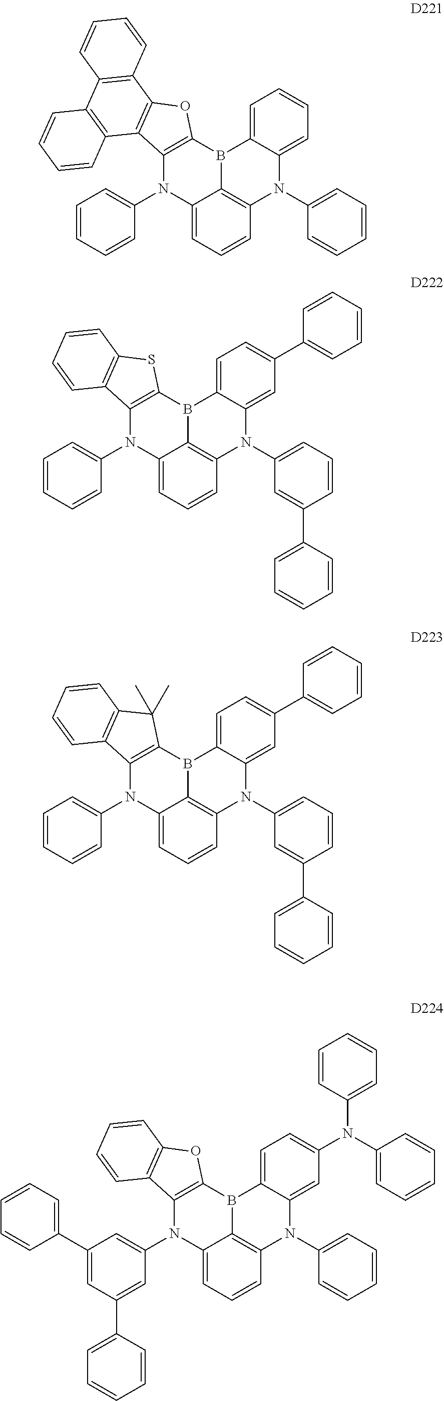

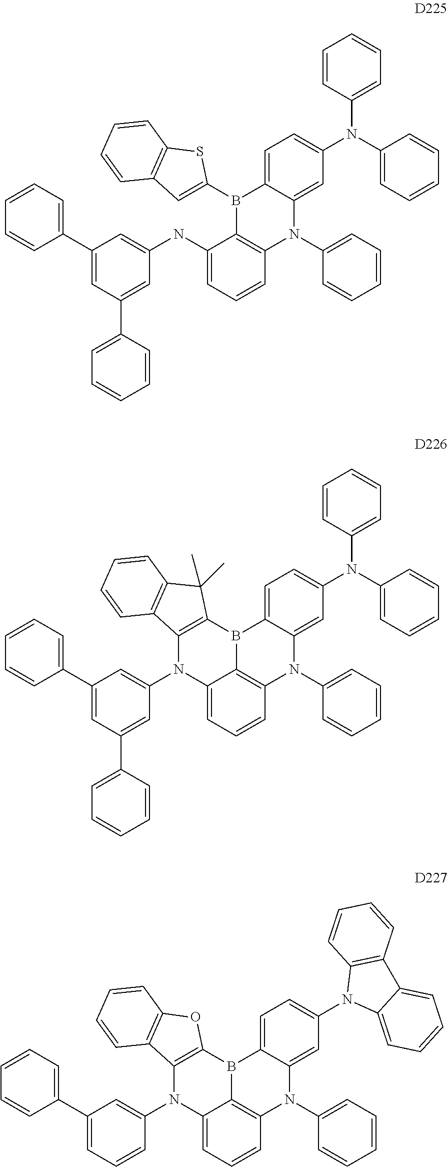 [SMILES: c1ccc(-c2cc(Nc3cccc4c3B(c3cc5ccccc5s3)c3ccc(N(c5ccccc5)c5ccccc5)cc3N4c3ccccc3)cc(-c3ccccc3)c2)cc1 ; CC1(C)C2=C(c3ccccc31)N(c1cc(-c3ccccc3)cc(-c3ccccc3)c1)c1cccc3c1B2c1ccc(N(c2ccccc2)c2ccccc2)cc1N3c1ccccc1 ; c1ccc(-c2cccc(N3c4cccc5c4B(c4ccc(-n6c7ccccc7c7ccccc76)cc4N5c4ccccc4)c4oc5ccccc5c43)c2)cc1]

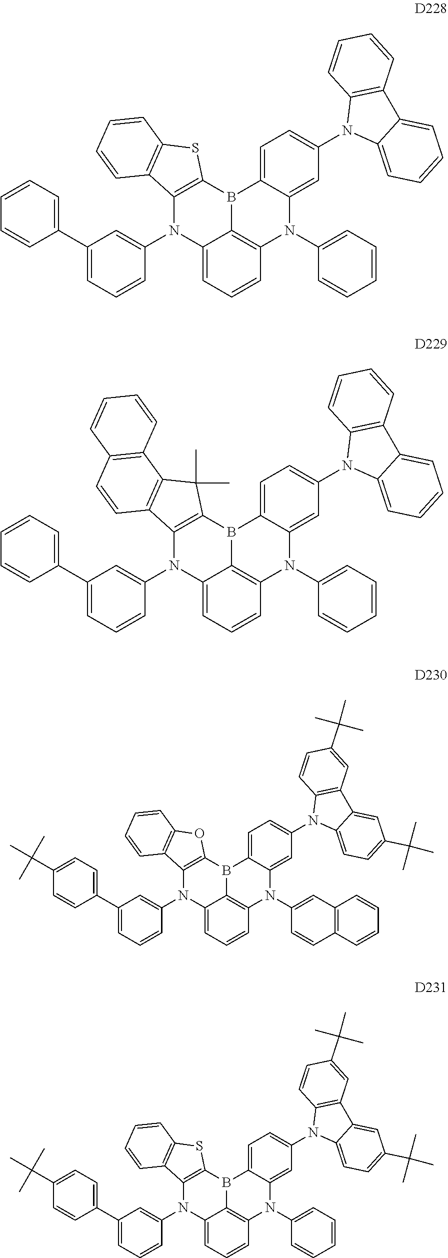

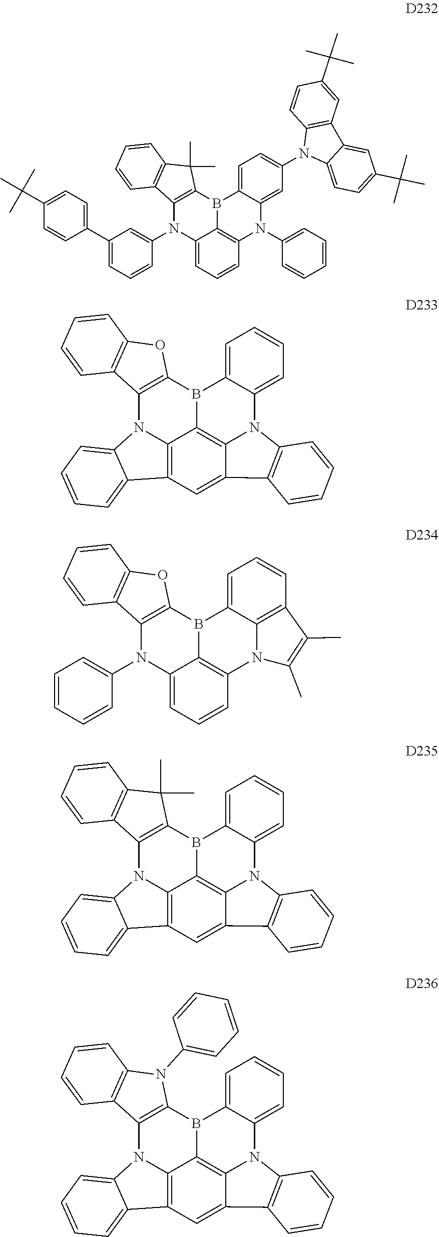

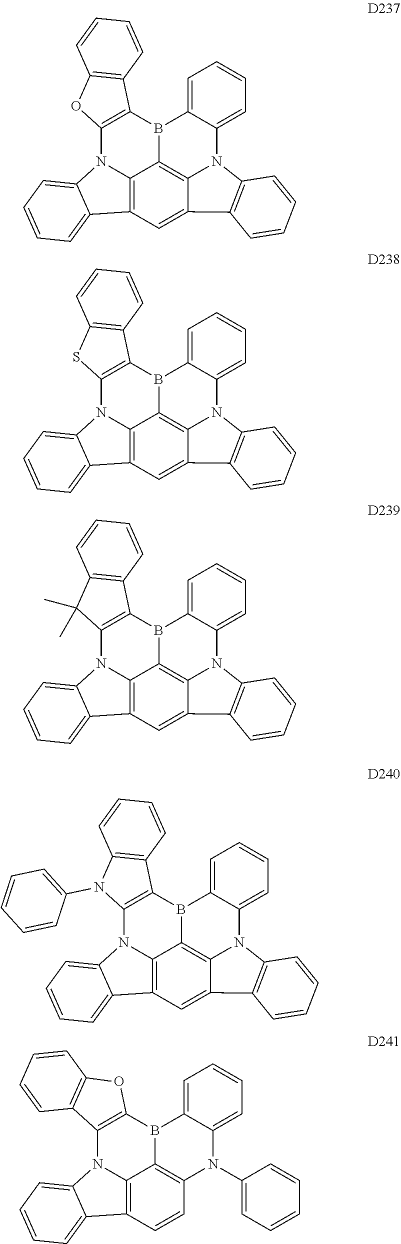

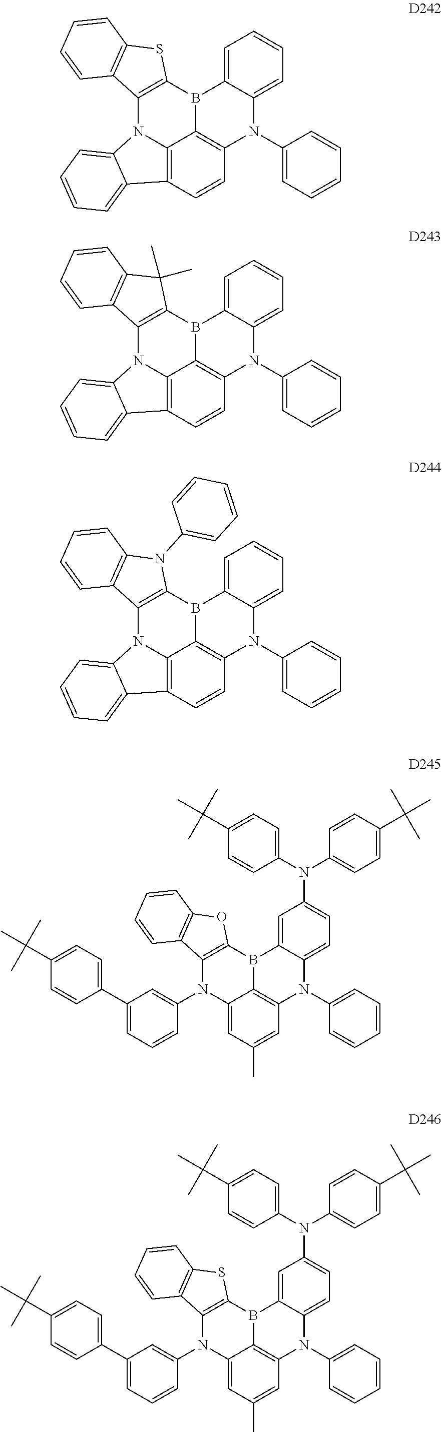

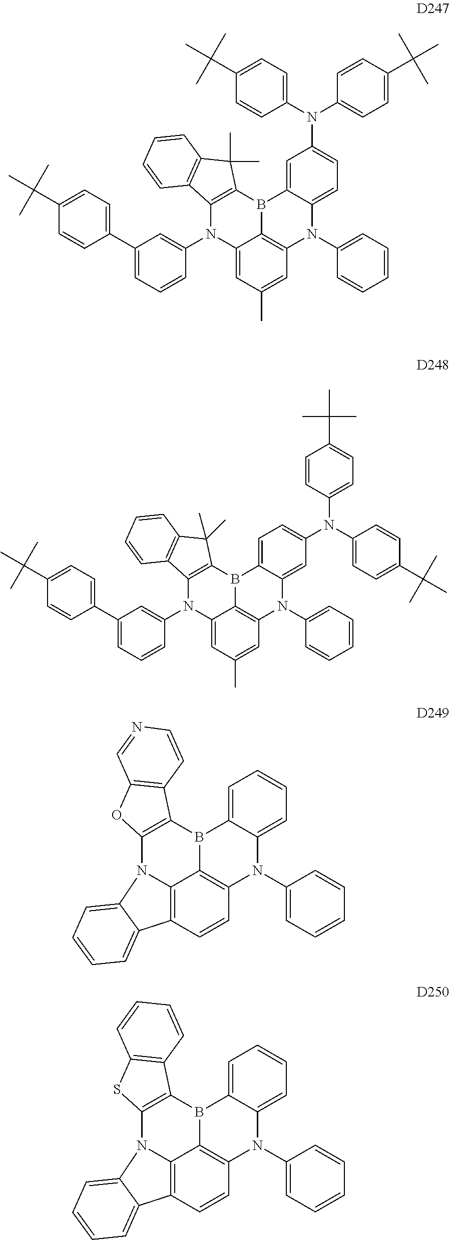

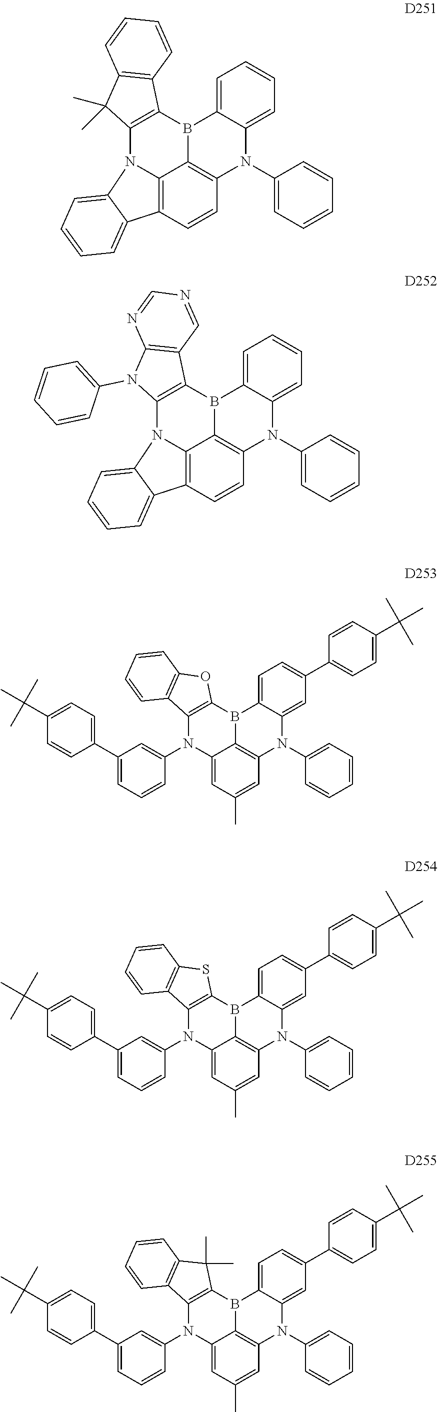

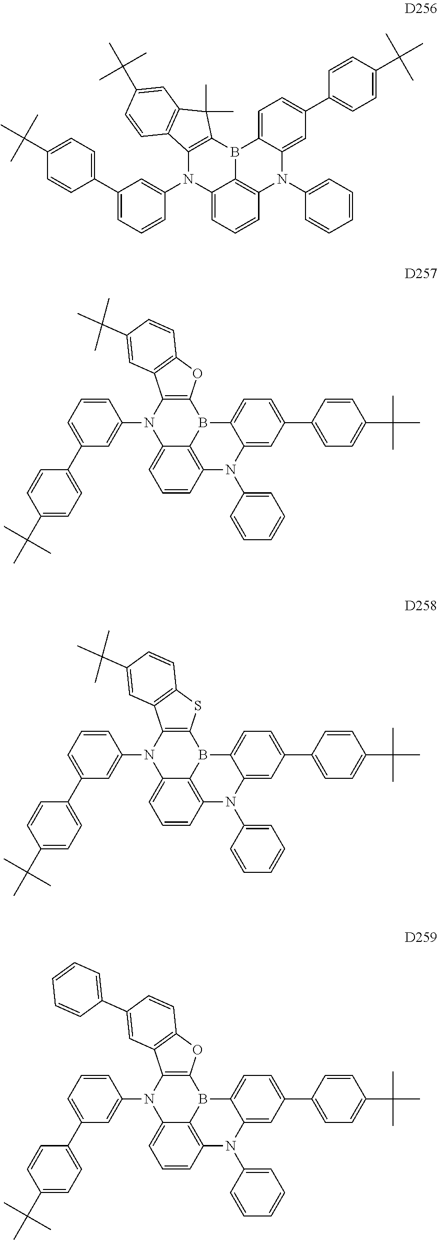

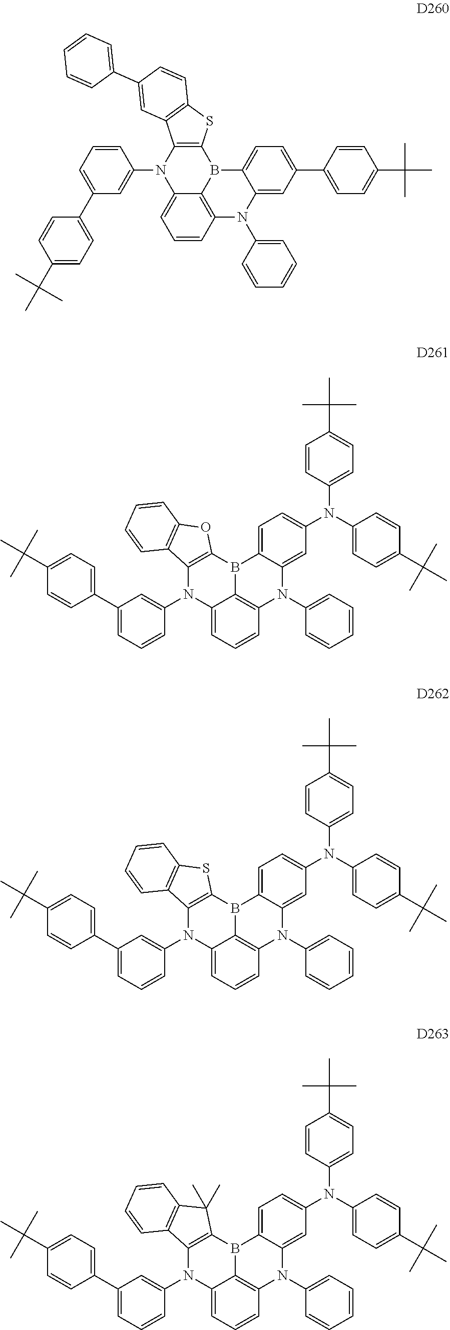

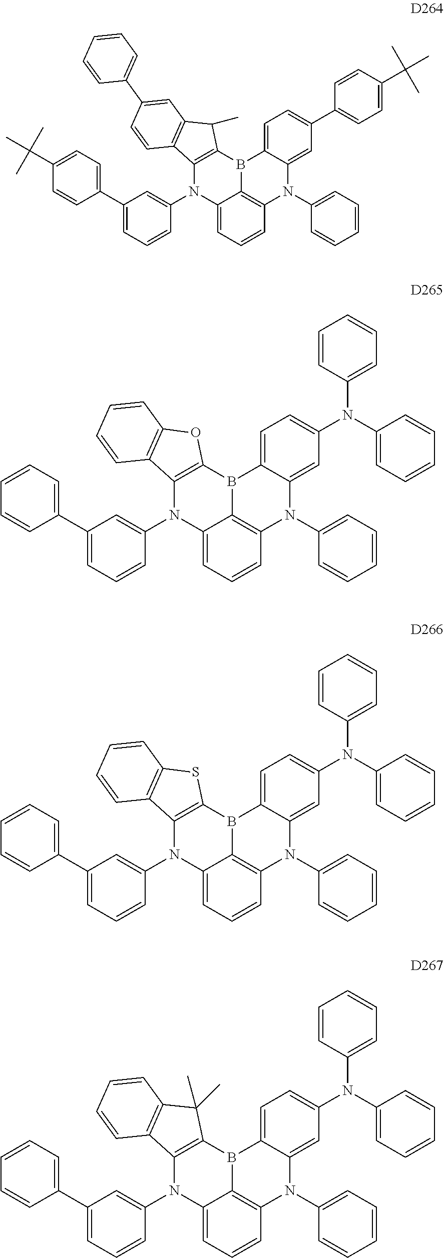 [SMILES: CC1C2=C(c3ccc(-c4ccccc4)cc31)N(c1cccc(-c3ccc(C(C)(C)C)cc3)c1)c1cccc3c1B2c1ccc(-c2ccc(C(C)(C)C)cc2)cc1N3c1ccccc1 ; c1ccc(-c2cccc(N3c4cccc5c4B(c4ccc(N(c6ccccc6)c6ccccc6)cc4N5c4ccccc4)c4oc5ccccc5c43)c2)cc1 ; c1ccc(-c2cccc(N3c4cccc5c4B(c4ccc(N(c6ccccc6)c6ccccc6)cc4N5c4ccccc4)c4sc5ccccc5c43)c2)cc1 ; CC1(C)C2=C(c3ccccc31)N(c1cccc(-c3ccccc3)c1)c1cccc3c1B2c1ccc(N(c2ccccc2)c2ccccc2)cc1N3c1ccccc1]

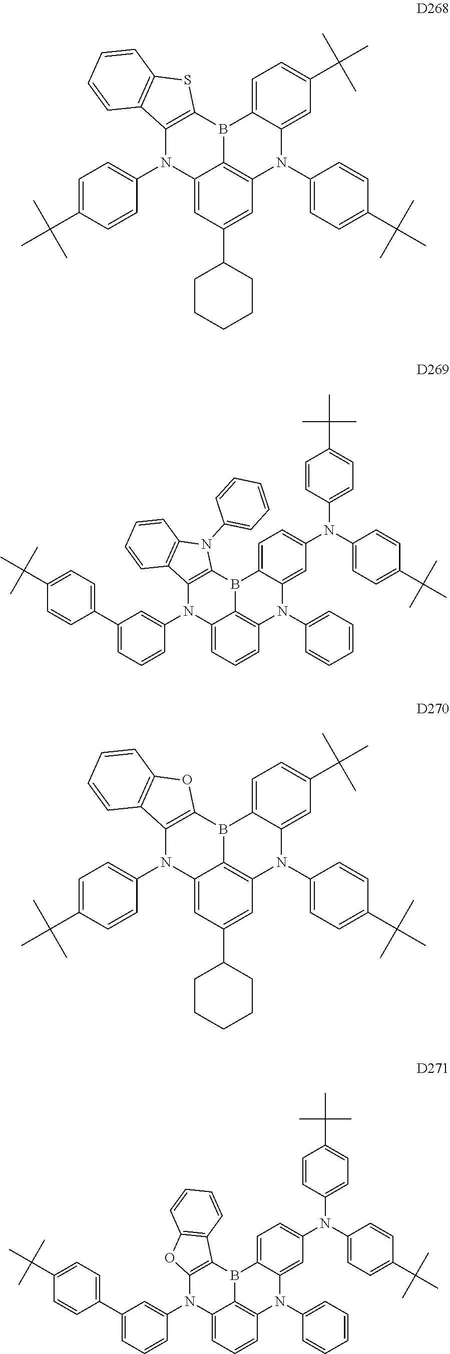

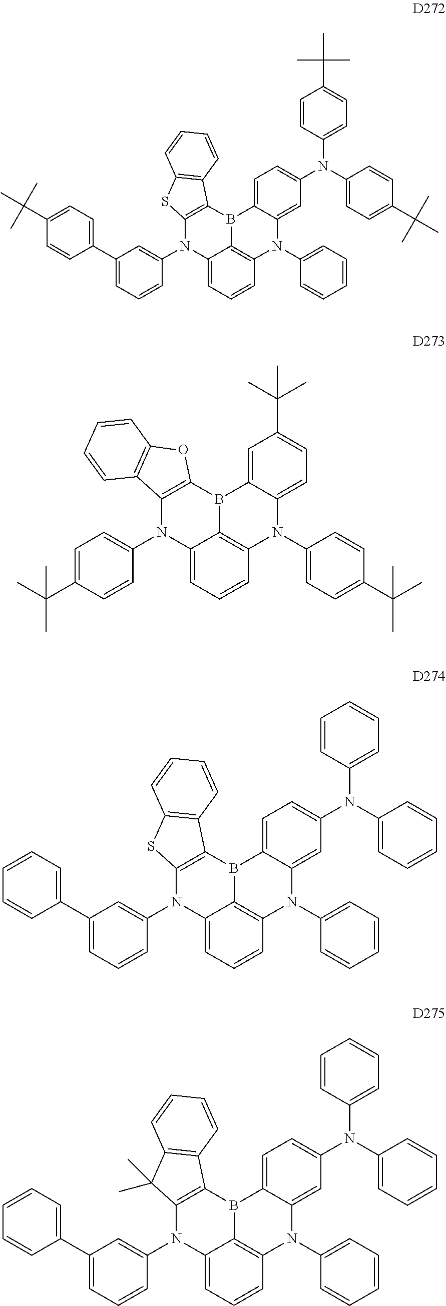

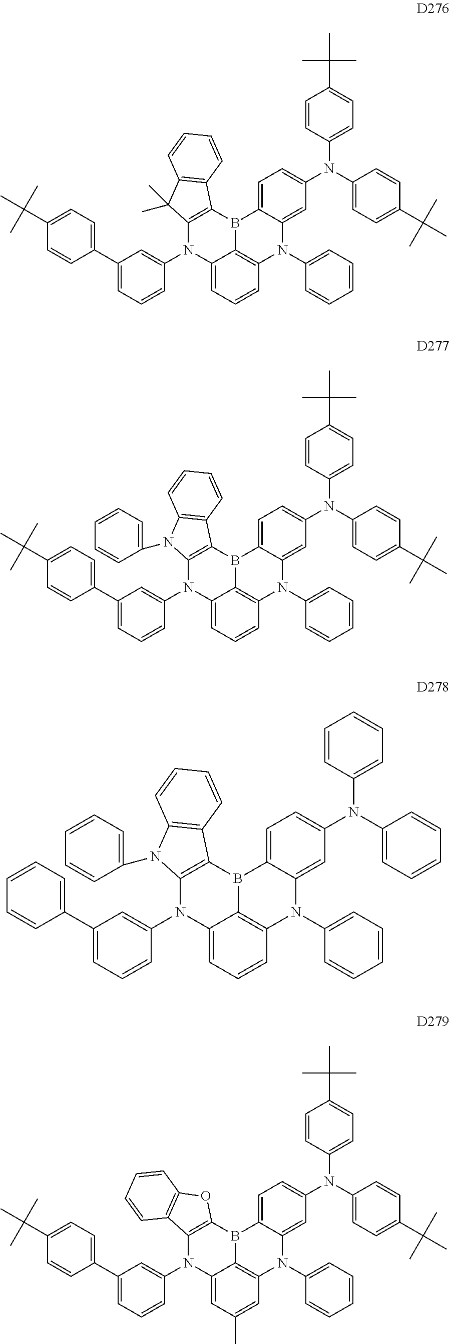

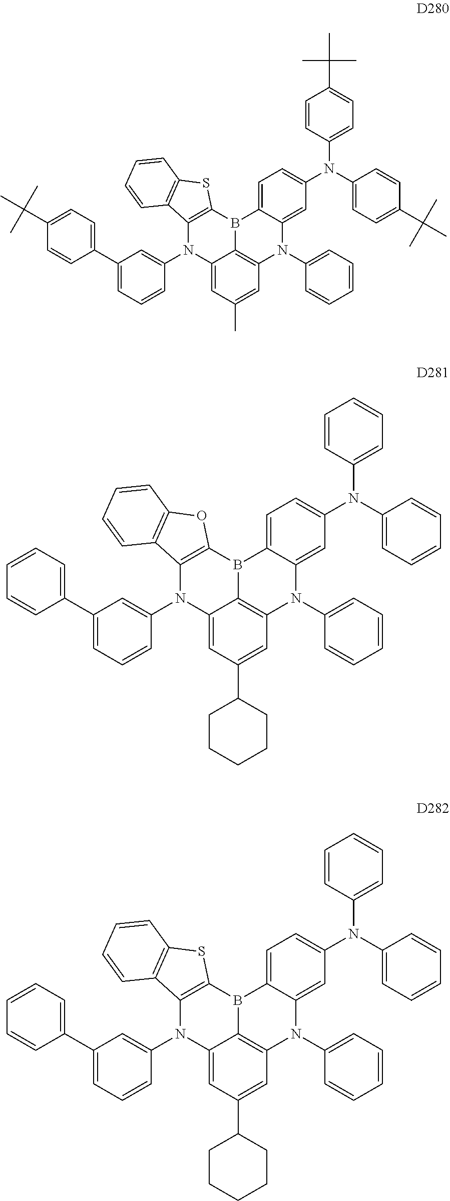 [SMILES: Cc1cc2c3c(c1)N(c1cccc(-c4ccc(C(C)(C)C)cc4)c1)c1c(sc4ccccc14)B3c1ccc(N(c3ccc(C(C)(C)C)cc3)c3ccc(C(C)(C)C)cc3)cc1N2c1ccccc1 ; c1ccc(-c2cccc(N3c4cc(C5CCCCC5)cc5c4B(c4ccc(N(c6ccccc6)c6ccccc6)cc4N5c4ccccc4)c4oc5ccccc5c43)c2)cc1 ; c1ccc(-c2cccc(N3c4cc(C5CCCCC5)cc5c4B(c4ccc(N(c6ccccc6)c6ccccc6)cc4N5c4ccccc4)c4sc5ccccc5c43)c2)cc1]

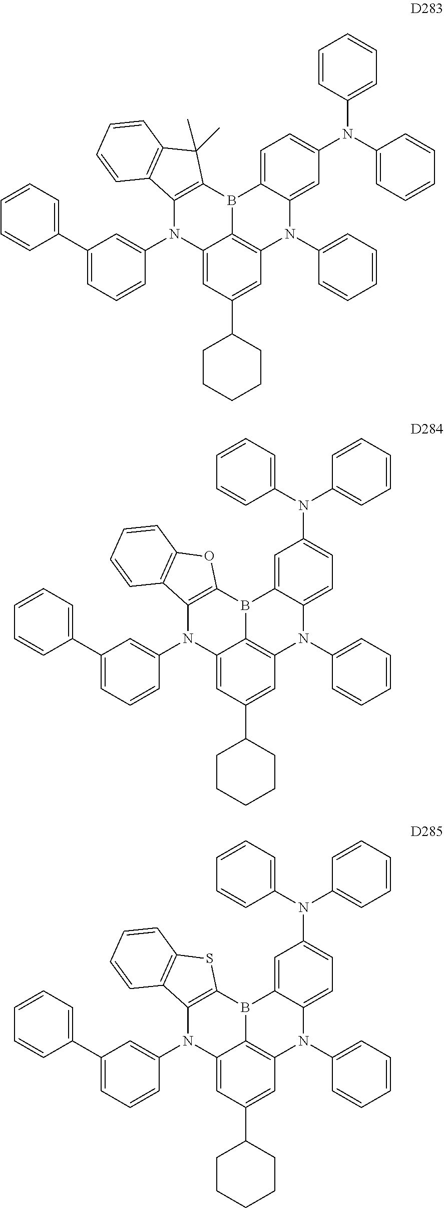

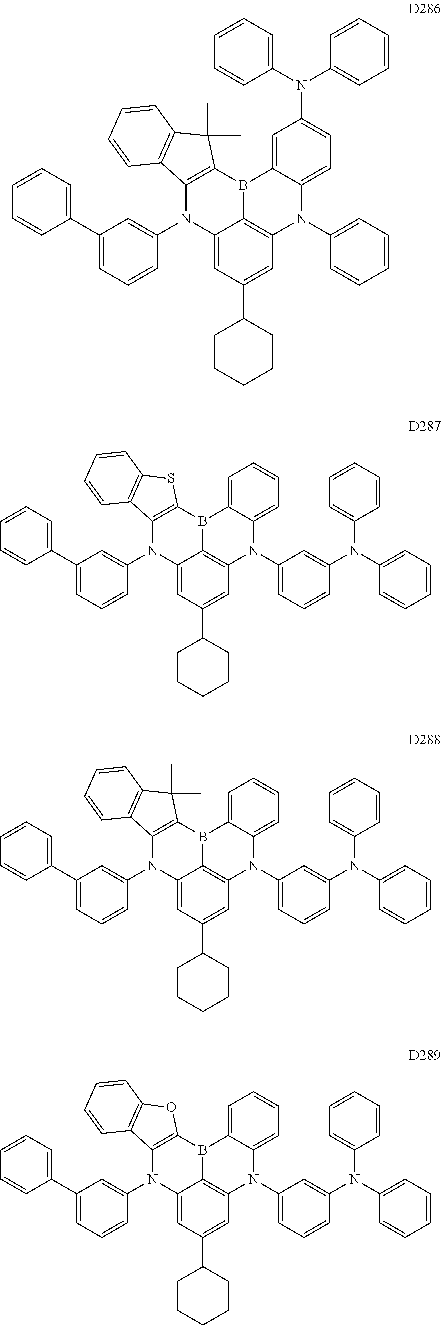 [SMILES: CC1(C)C2=C(c3ccccc31)N(c1cccc(-c3ccccc3)c1)c1cc(C3CCCCC3)cc3c1B2c1cc(N(c2ccccc2)c2ccccc2)ccc1N3c1ccccc1 ; c1ccc(-c2cccc(N3c4cc(C5CCCCC5)cc5c4B(c4ccccc4N5c4cccc(N(c5ccccc5)c5ccccc5)c4)c4sc5ccccc5c43)c2)cc1 ; CC1(C)C2=C(c3ccccc31)N(c1cccc(-c3ccccc3)c1)c1cc(C3CCCCC3)cc3c1B2c1ccccc1N3c1cccc(N(c2ccccc2)c2ccccc2)c1 ; c1ccc(-c2cccc(N3c4cc(C5CCCCC5)cc5c4B(c4ccccc4N5c4cccc(N(c5ccccc5)c5ccccc5)c4)c4oc5ccccc5c43)c2)cc1]

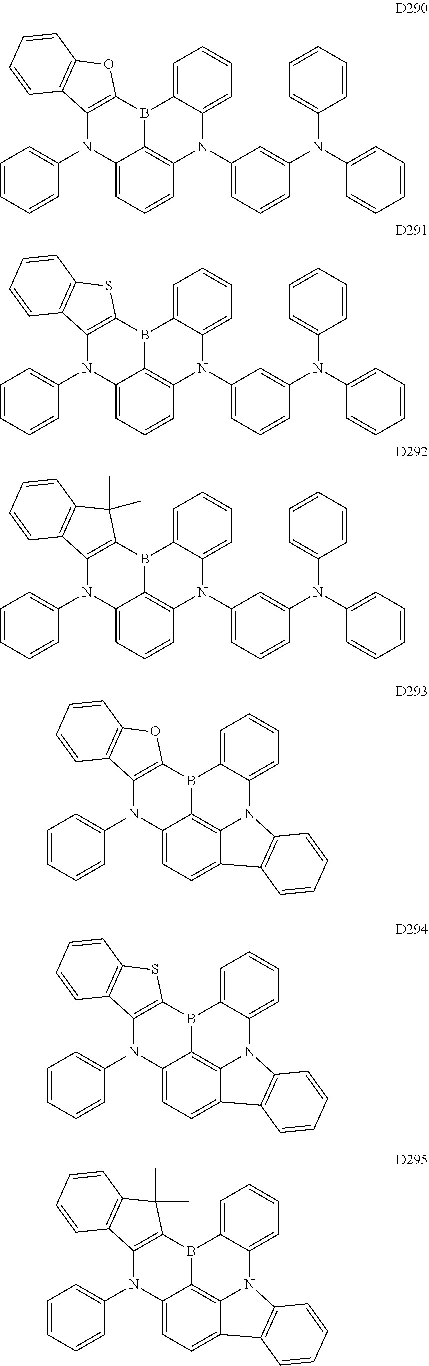

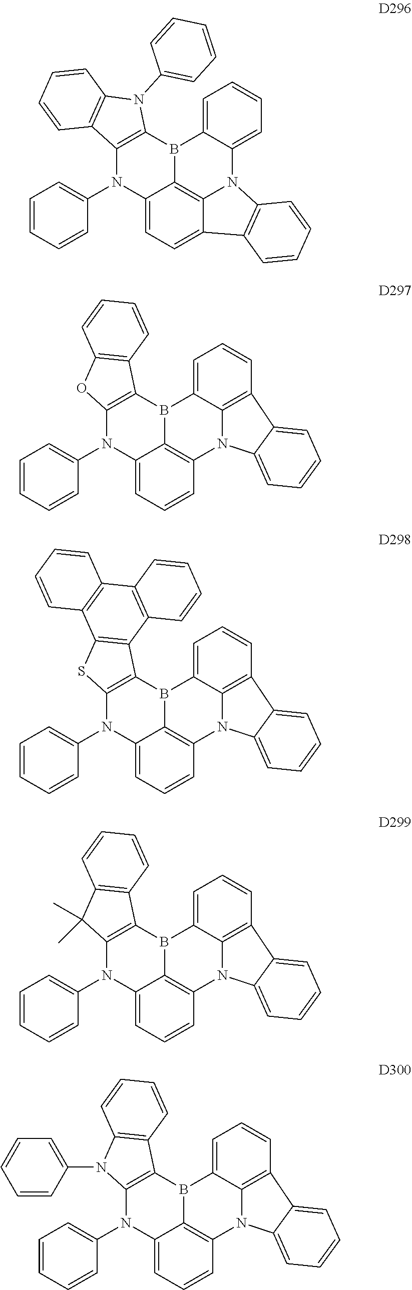

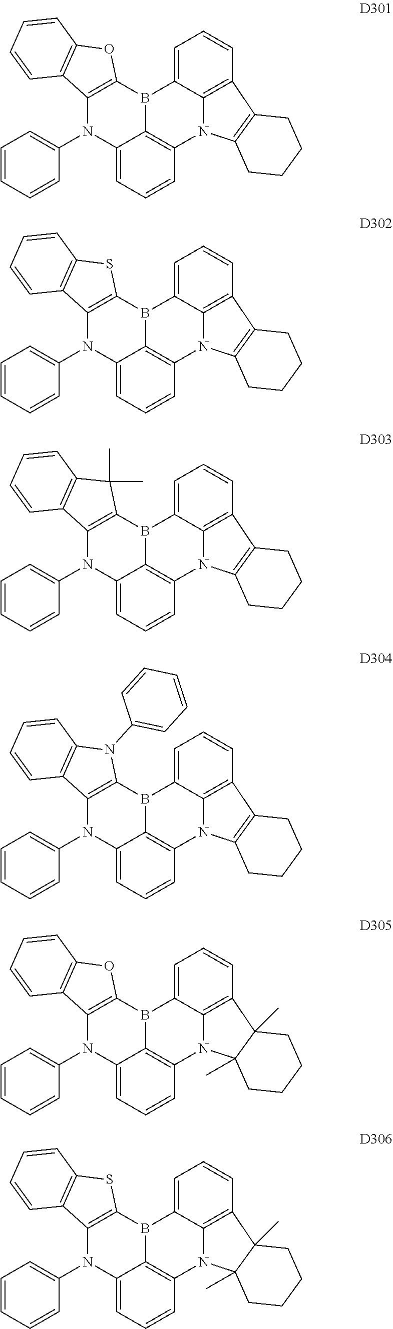

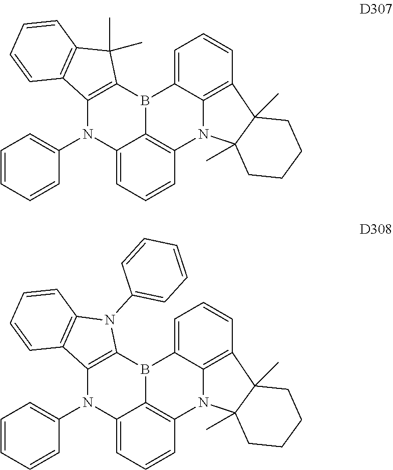

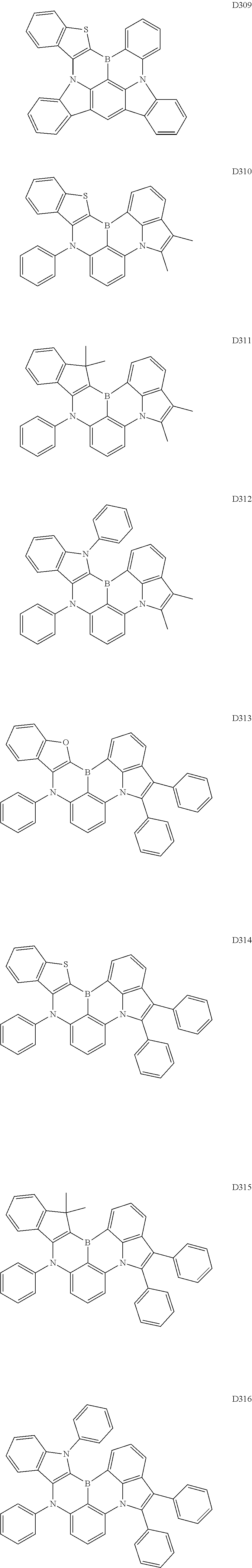

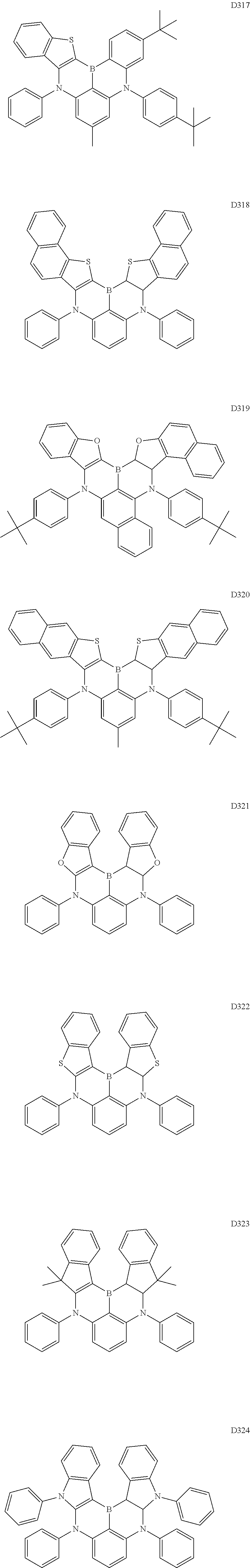

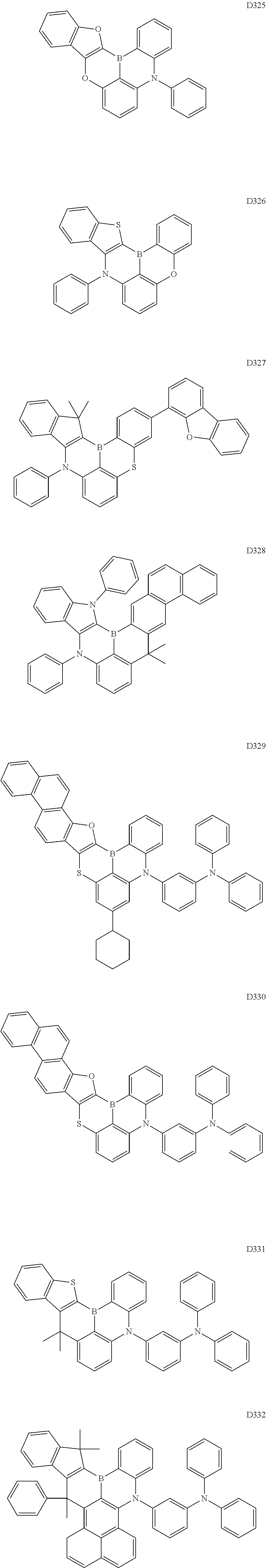

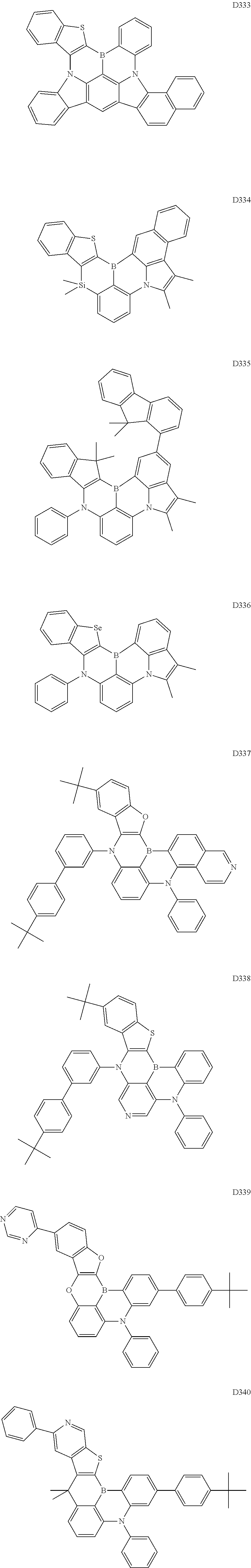

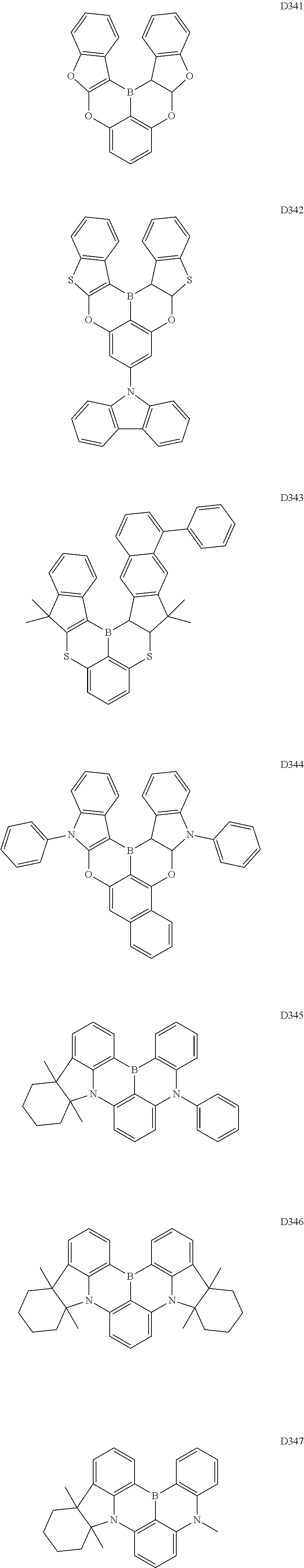

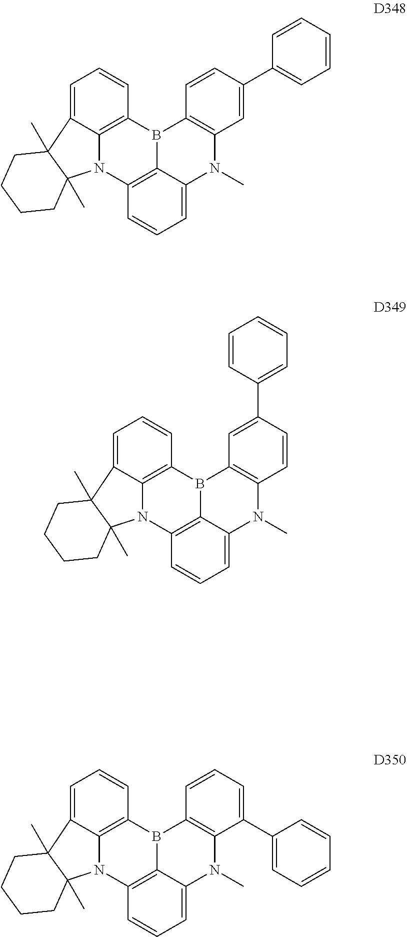 [SMILES: CN1c2cc(-c3ccccc3)ccc2B2c3cccc4c3N(c3cccc1c32)C1(C)CCCCC41C ; CN1c2ccc(-c3ccccc3)cc2B2c3cccc4c3N(c3cccc1c32)C1(C)CCCCC41C ; CN1c2cccc3c2B(c2cccc(-c4ccccc4)c21)c1cccc2c1N3C1(C)CCCCC21C]

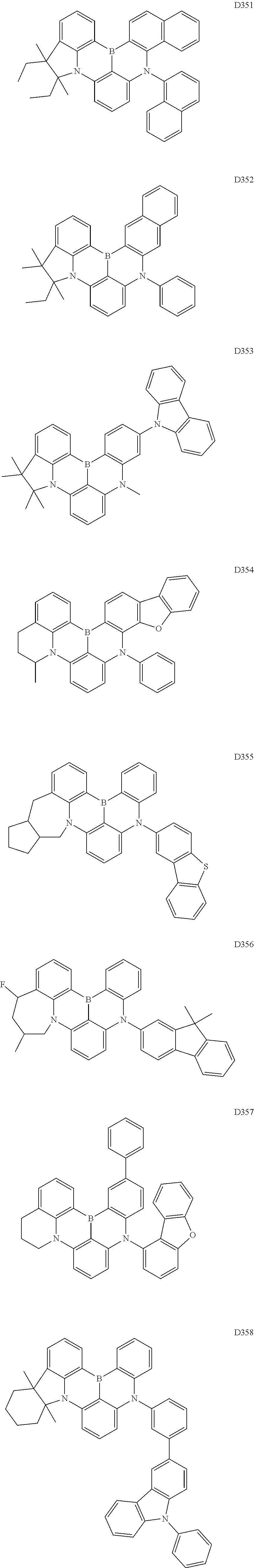

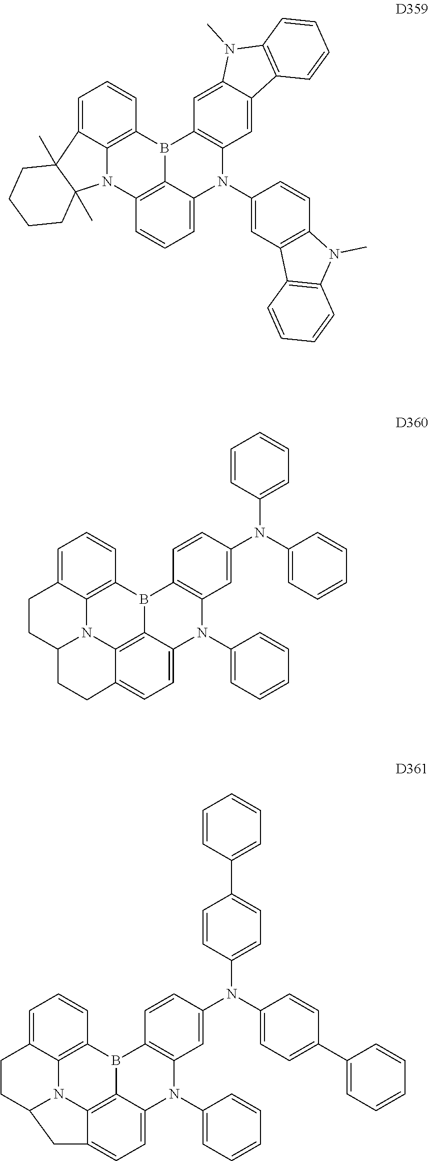 [SMILES: Cn1c2ccccc2c2cc(N3c4cc5c6ccccc6n(C)c5cc4B4c5cccc6c5N(c5cccc3c54)C3(C)CCCCC63C)ccc21 ; c1ccc(N(c2ccccc2)c2ccc3c(c2)N(c2ccccc2)c2ccc4c5c2B3c2cccc3c2N5C(CC3)CC4)cc1 ; c1ccc(-c2ccc(N(c3ccc(-c4ccccc4)cc3)c3ccc4c(c3)N(c3ccccc3)c3ccc5c6c3B4c3cccc4c3N6C(CC4)C5)cc2)cc1]

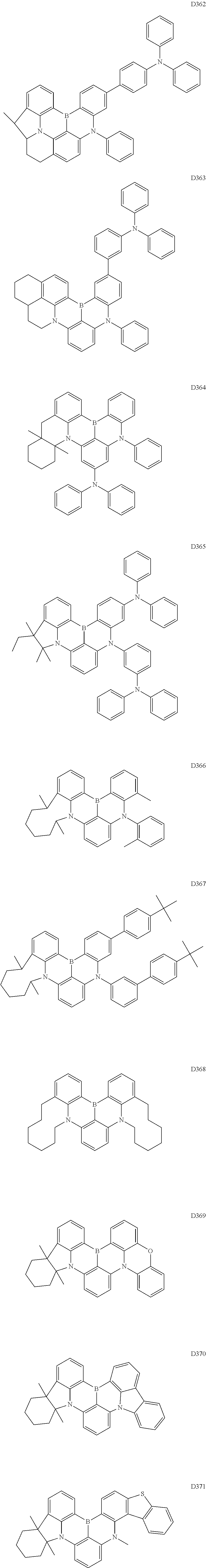

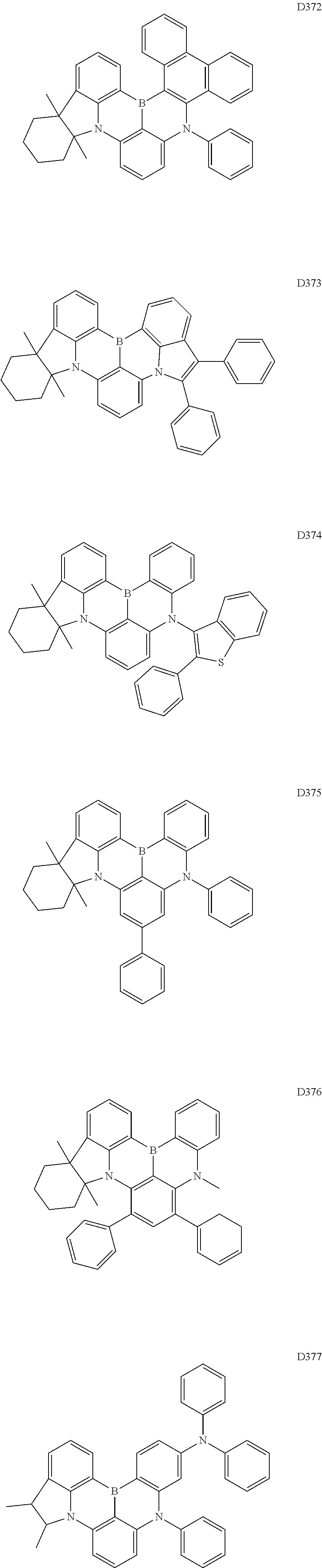 [SMILES: CC12CCCCC1(C)N1c3cccc4c3B(c3cccc2c31)c1c(c2ccccc2c2ccccc12)N4c1ccccc1 ; CC12CCCCC1(C)N1c3cccc4c3B(c3cccc2c31)c1cccc2c(-c3ccccc3)c(-c3ccccc3)n-4c12 ; CC12CCCCC1(C)N1c3cccc4c3B(c3ccccc3N4c3c(-c4ccccc4)sc4ccccc34)c3cccc2c31 ; CC12CCCCC1(C)N1c3cc(-c4ccccc4)cc4c3B(c3ccccc3N4c3ccccc3)c3cccc2c31 ; CN1c2ccccc2B2c3cccc4c3N(c3c(-c5ccccc5)cc(C5=CC=CCC5)c1c32)C1(C)CCCCC41C ; CC1c2cccc3c2N(c2cccc4c2B3c2ccc(N(c3ccccc3)c3ccccc3)cc2N4c2ccccc2)C1C]

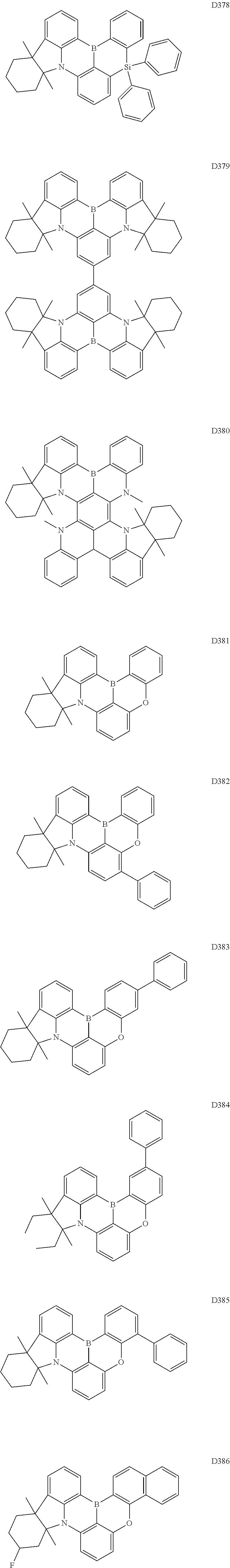

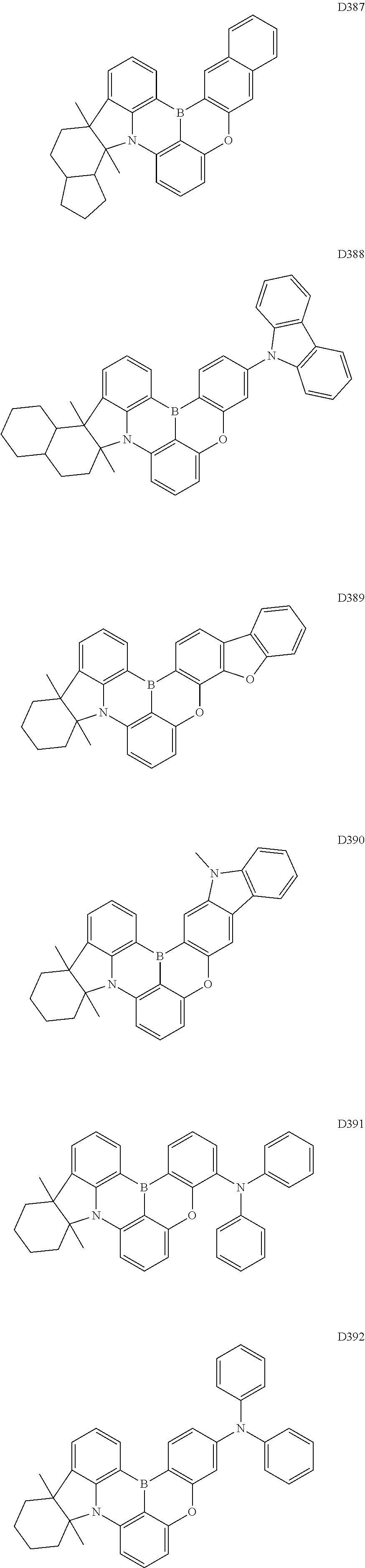 [SMILES: CC12CCC3CCCC3C1(C)N1c3cccc4c3B(c3cc5ccccc5cc3O4)c3cccc2c31 ; CC12c3cccc4c3N(c3cccc5c3B4c3ccc(-n4c6ccccc6c6ccccc64)cc3O5)C1(C)CCC1CCCCC12 ; CC12CCCCC1(C)N1c3cccc4c3B(c3cccc2c31)c1ccc2c(oc3ccccc32)c1O4 ; Cn1c2ccccc2c2cc3c(cc21)B1c2cccc4c2N(c2cccc(c21)O3)C1(C)CCCCC41C ; CC12CCCCC1(C)N1c3cccc4c3B(c3cccc(N(c5ccccc5)c5ccccc5)c3O4)c3cccc2c31 ; CC12CCCCC1(C)N1c3cccc4c3B(c3ccc(N(c5ccccc5)c5ccccc5)cc3O4)c3cccc2c31]

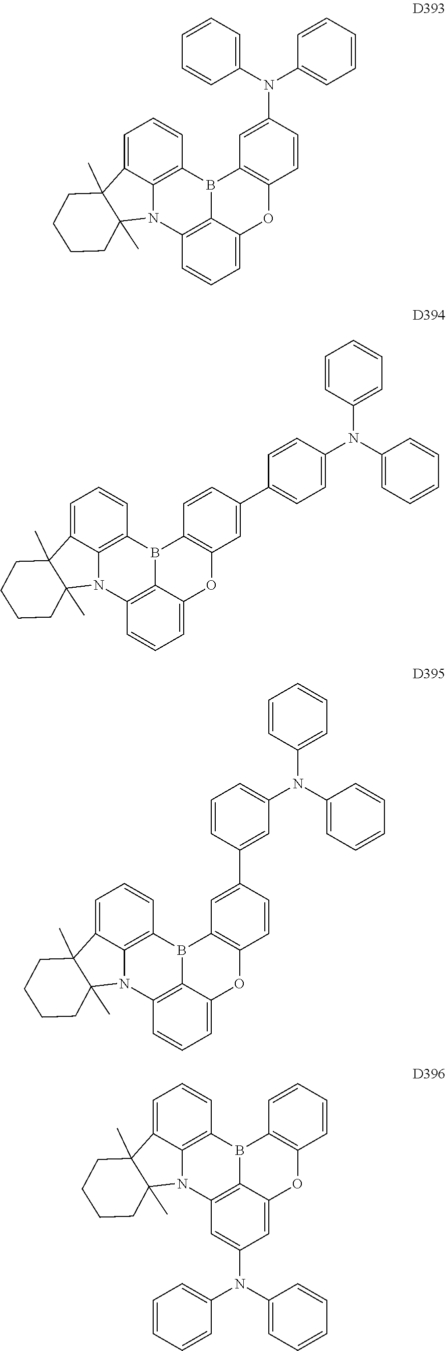

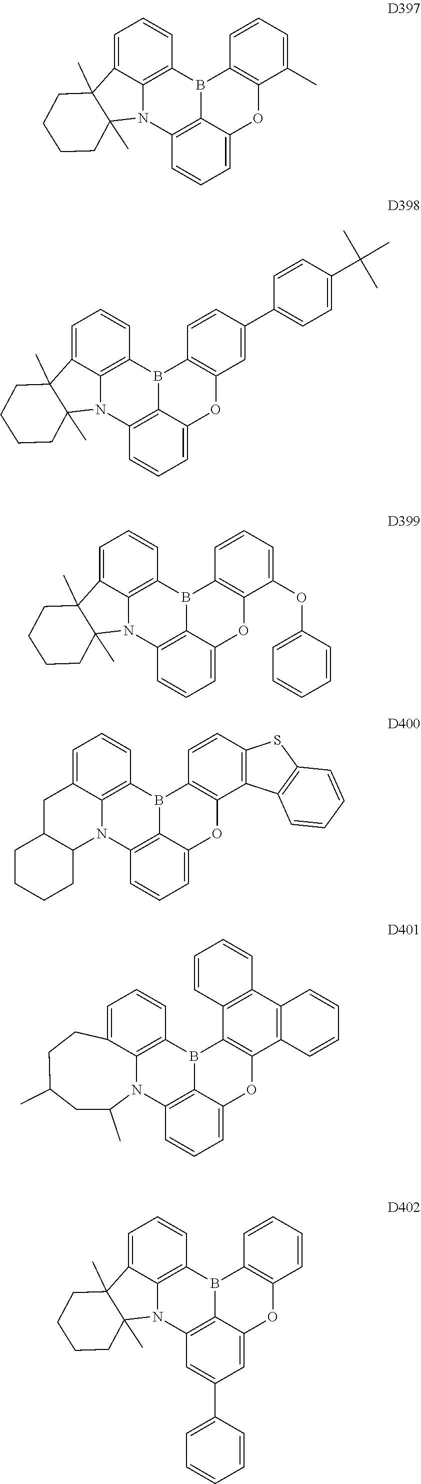

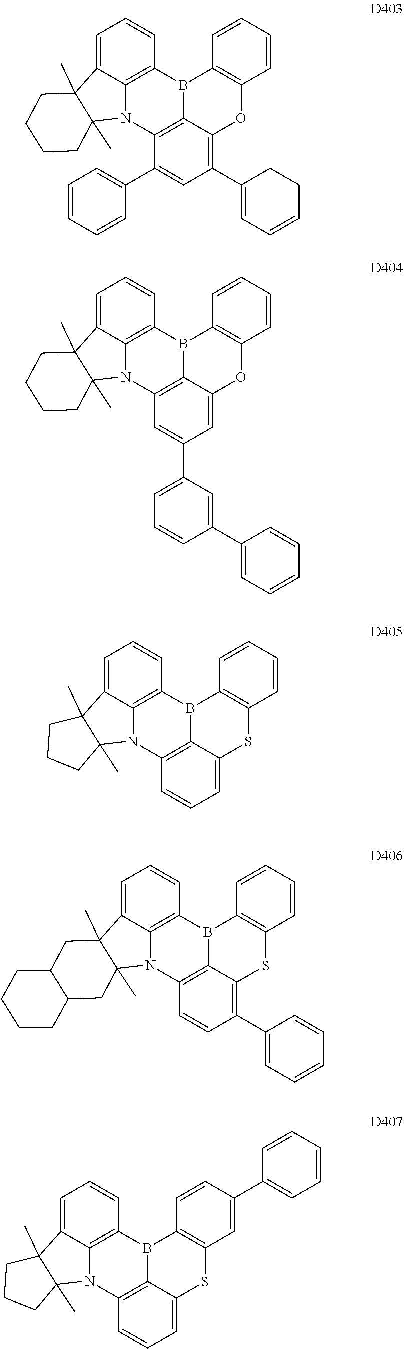 [SMILES: CC12CCCCC1(C)N1c3c(cccc32)B2c3ccccc3Oc3c(C4=CC=CCC4)cc(-c4ccccc4)c1c32 ; CC12CCCCC1(C)N1c3cc(-c4cccc(-c5ccccc5)c4)cc4c3B(c3ccccc3O4)c3cccc2c31 ; CC12CCCC1(C)N1c3cccc4c3B(c3ccccc3S4)c3cccc2c31 ; CC12CC3CCCCC3CC1(C)N1c3ccc(-c4ccccc4)c4c3B(c3ccccc3S4)c3cccc2c31 ; CC12CCCC1(C)N1c3cccc4c3B(c3ccc(-c5ccccc5)cc3S4)c3cccc2c31]

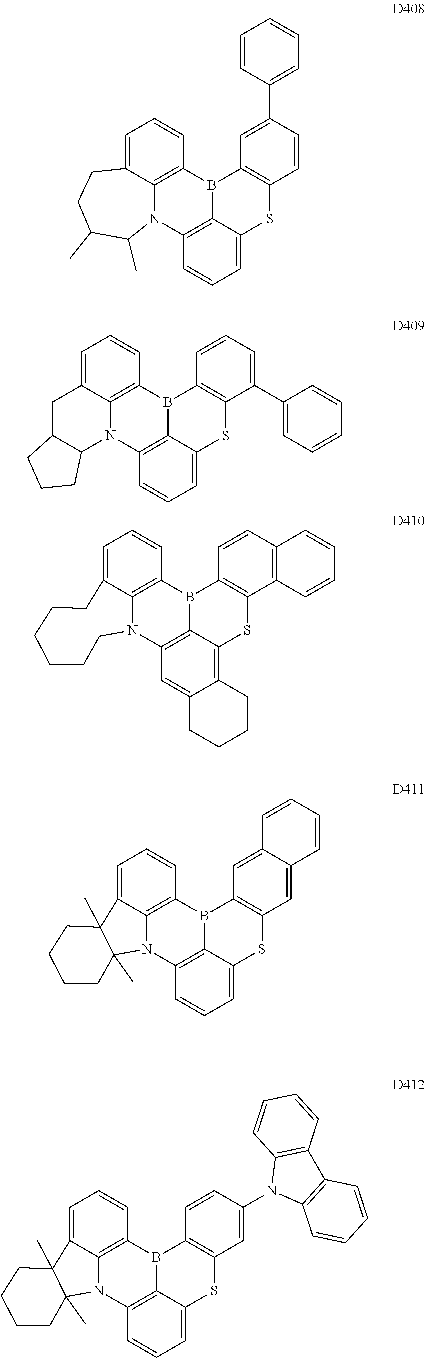

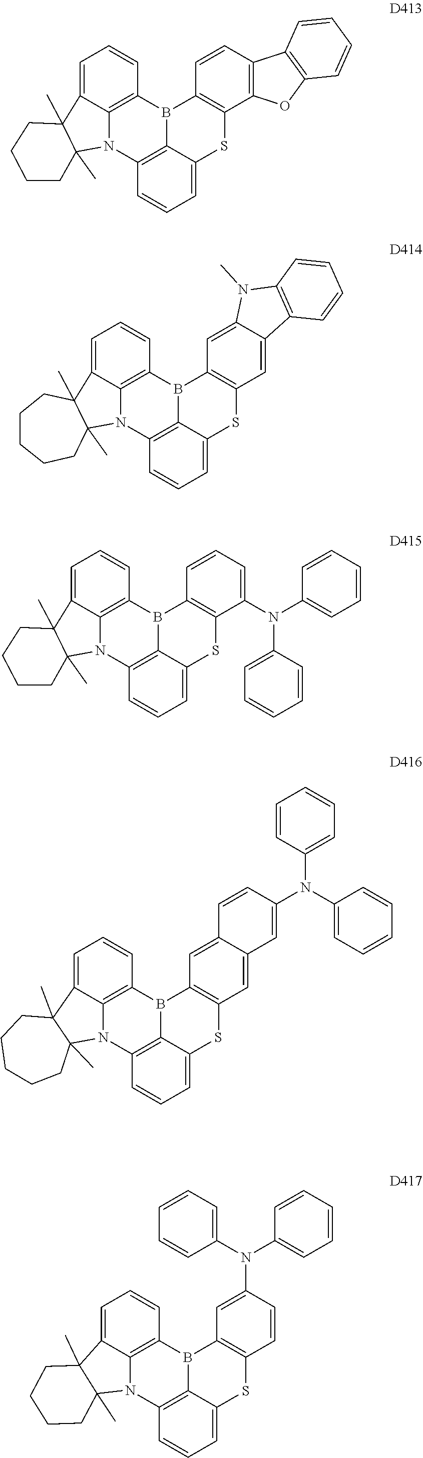 [SMILES: CC12CCCCC1(C)N1c3cccc4c3B(c3cccc2c31)c1ccc2c(oc3ccccc32)c1S4 ; Cn1c2ccccc2c2cc3c(cc21)B1c2cccc4c2N(c2cccc(c21)S3)C1(C)CCCCCC41C ; CC12CCCCC1(C)N1c3cccc4c3B(c3cccc(N(c5ccccc5)c5ccccc5)c3S4)c3cccc2c31 ; CC12CCCCCC1(C)N1c3cccc4c3B(c3cc5ccc(N(c6ccccc6)c6ccccc6)cc5cc3S4)c3cccc2c31 ; CC12CCCCC1(C)N1c3cccc4c3B(c3cc(N(c5ccccc5)c5ccccc5)ccc3S4)c3cccc2c31]

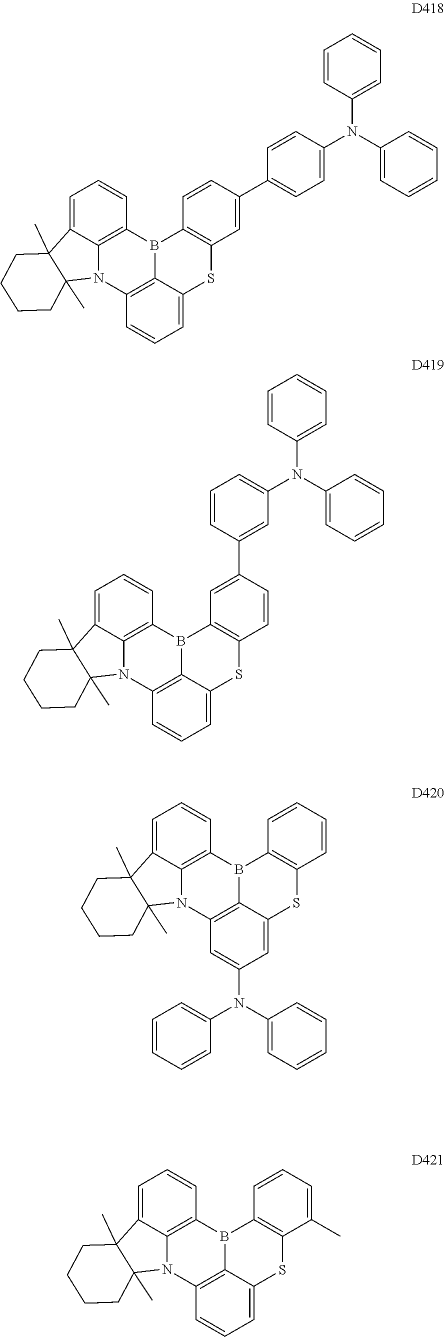 [SMILES: CC12CCCCC1(C)N1c3cccc4c3B(c3ccc(-c5ccc(N(c6ccccc6)c6ccccc6)cc5)cc3S4)c3cccc2c31 ; CC12CCCCC1(C)N1c3cccc4c3B(c3cc(-c5cccc(N(c6ccccc6)c6ccccc6)c5)ccc3S4)c3cccc2c31 ; CC12CCCCC1(C)N1c3cc(N(c4ccccc4)c4ccccc4)cc4c3B(c3ccccc3S4)c3cccc2c31 ; Cc1cccc2c1Sc1cccc3c1B2c1cccc2c1N3C1(C)CCCCC21C]

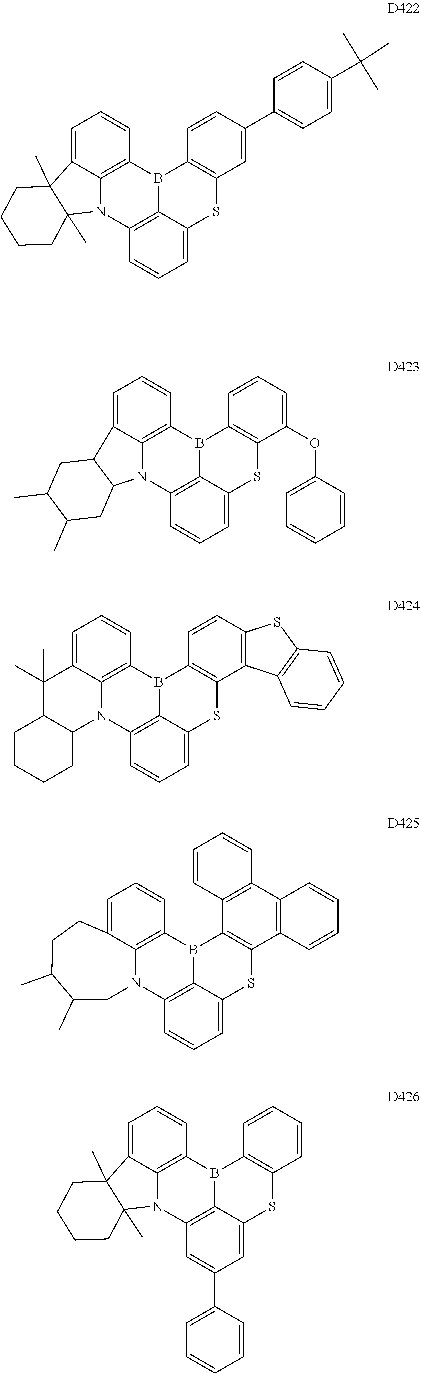

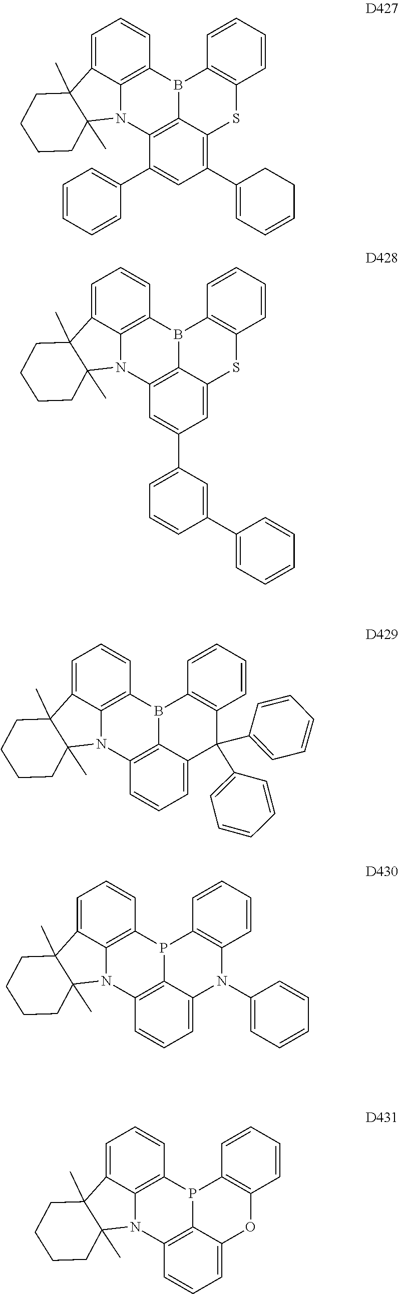

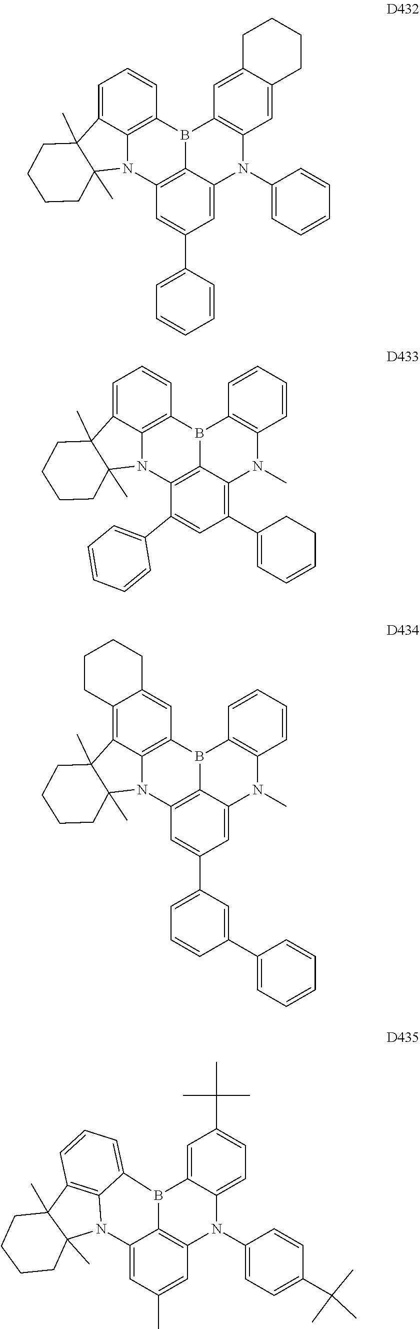 [SMILES: CC12CCCCC1(C)N1c3cc(-c4ccccc4)cc4c3B(c3cc5c(cc3N4c3ccccc3)CCCC5)c3cccc2c31 ; CN1c2ccccc2B2c3cccc4c3N(c3c(-c5ccccc5)cc(C5=CC=CCC5)c1c32)C1(C)CCCCC41C ; CN1c2ccccc2B2c3cc4c(c5c3N(c3cc(-c6cccc(-c7ccccc7)c6)cc1c32)C1(C)CCCCC51C)CCCC4 ; Cc1cc2c3c(c1)N1c4c(cccc4C4(C)CCCCC14C)B3c1cc(C(C)(C)C)ccc1N2c1ccc(C(C)(C)C)cc1]

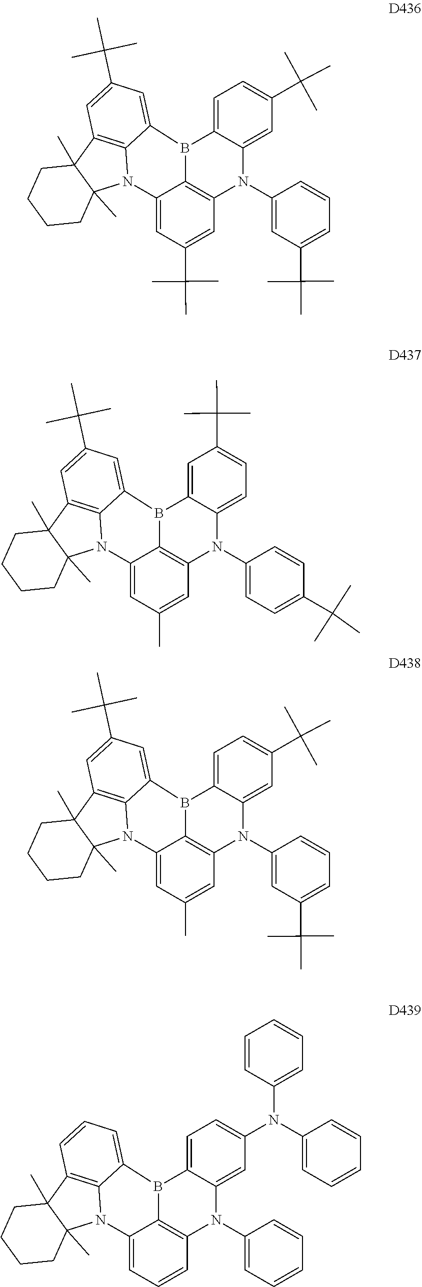 [SMILES: CC(C)(C)c1cccc(N2c3cc(C(C)(C)C)ccc3B3c4cc(C(C)(C)C)cc5c4N(c4cc(C(C)(C)C)cc2c43)C2(C)CCCCC52C)c1 ; Cc1cc2c3c(c1)N1c4c(cc(C(C)(C)C)cc4C4(C)CCCCC14C)B3c1cc(C(C)(C)C)ccc1N2c1ccc(C(C)(C)C)cc1 ; Cc1cc2c3c(c1)N1c4c(cc(C(C)(C)C)cc4C4(C)CCCCC14C)B3c1ccc(C(C)(C)C)cc1N2c1cccc(C(C)(C)C)c1 ; CC12CCCCC1(C)N1c3cccc4c3B(c3ccc(N(c5ccccc5)c5ccccc5)cc3N4c3ccccc3)c3cccc2c31]

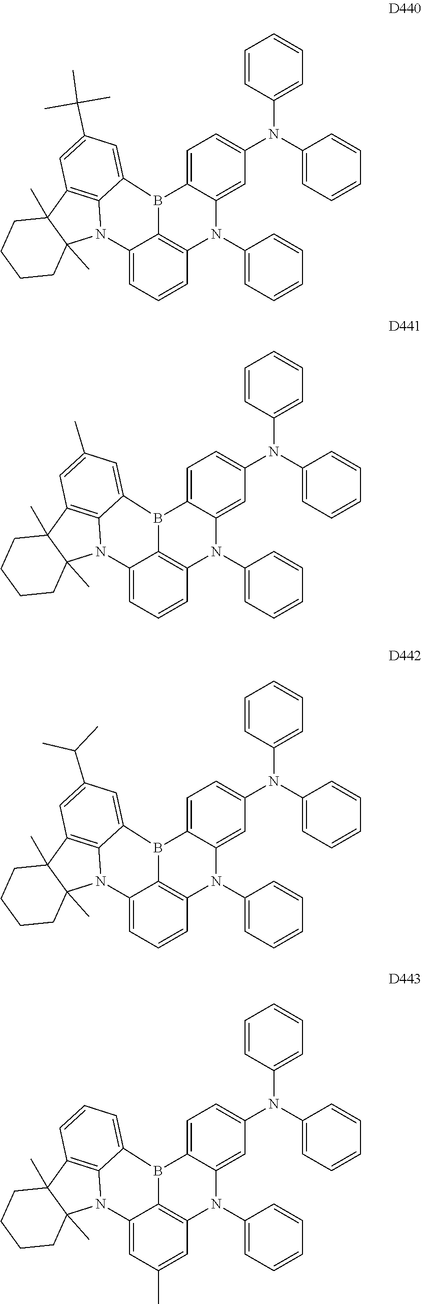 [SMILES: CC(C)(C)c1cc2c3c(c1)C1(C)CCCCC1(C)N3c1cccc3c1B2c1ccc(N(c2ccccc2)c2ccccc2)cc1N3c1ccccc1 ; Cc1cc2c3c(c1)C1(C)CCCCC1(C)N3c1cccc3c1B2c1ccc(N(c2ccccc2)c2ccccc2)cc1N3c1ccccc1 ; CC(C)c1cc2c3c(c1)C1(C)CCCCC1(C)N3c1cccc3c1B2c1ccc(N(c2ccccc2)c2ccccc2)cc1N3c1ccccc1 ; Cc1cc2c3c(c1)N1c4c(cccc4C4(C)CCCCC14C)B3c1ccc(N(c3ccccc3)c3ccccc3)cc1N2c1ccccc1]

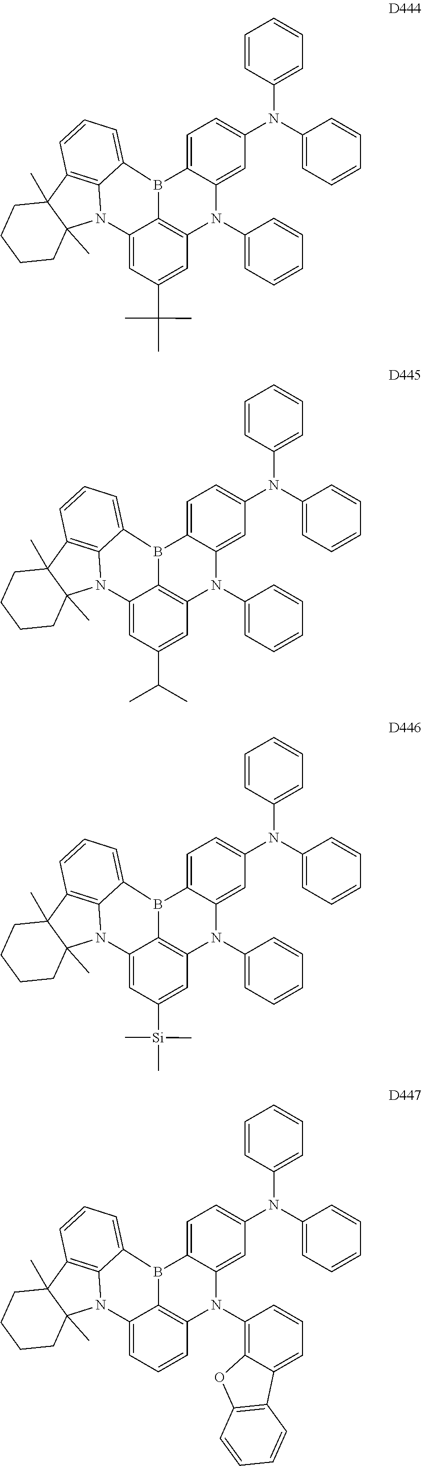 [SMILES: CC(C)(C)c1cc2c3c(c1)N1c4c(cccc4C4(C)CCCCC14C)B3c1ccc(N(c3ccccc3)c3ccccc3)cc1N2c1ccccc1 ; CC(C)c1cc2c3c(c1)N1c4c(cccc4C4(C)CCCCC14C)B3c1ccc(N(c3ccccc3)c3ccccc3)cc1N2c1ccccc1 ; CC12CCCCC1(C)N1c3cc([Si](C)(C)C)cc4c3B(c3ccc(N(c5ccccc5)c5ccccc5)cc3N4c3ccccc3)c3cccc2c31 ; CC12CCCCC1(C)N1c3cccc4c3B(c3ccc(N(c5ccccc5)c5ccccc5)cc3N4c3cccc4c3oc3ccccc34)c3cccc2c31]

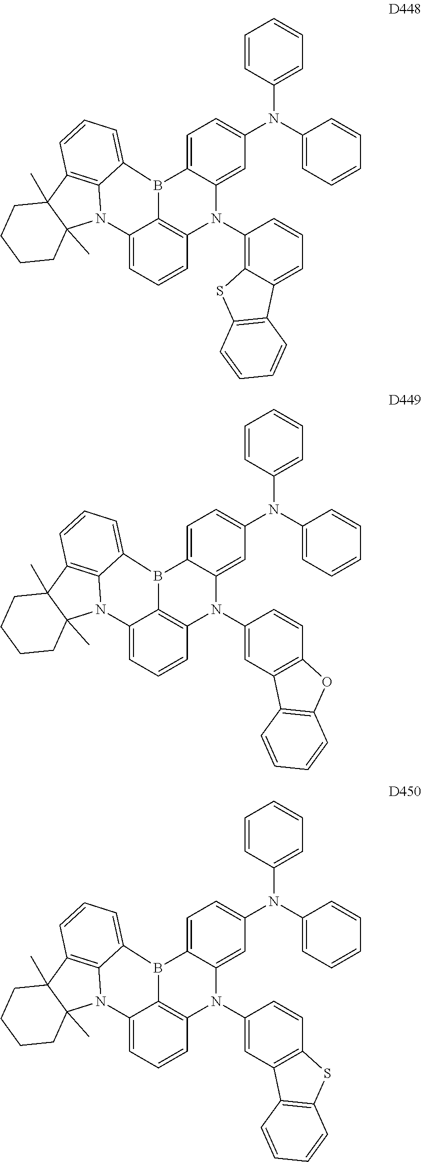

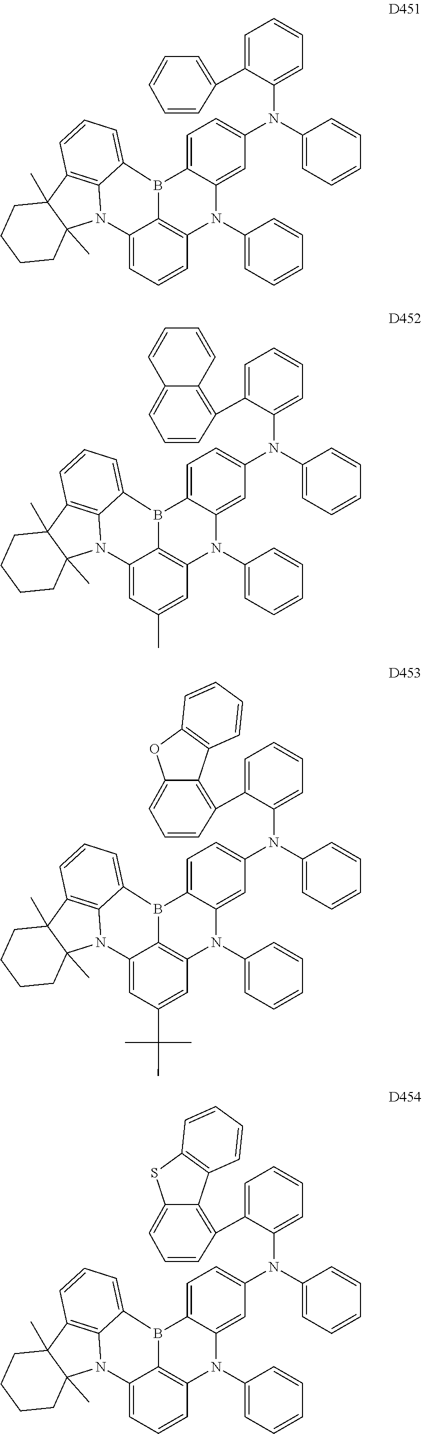

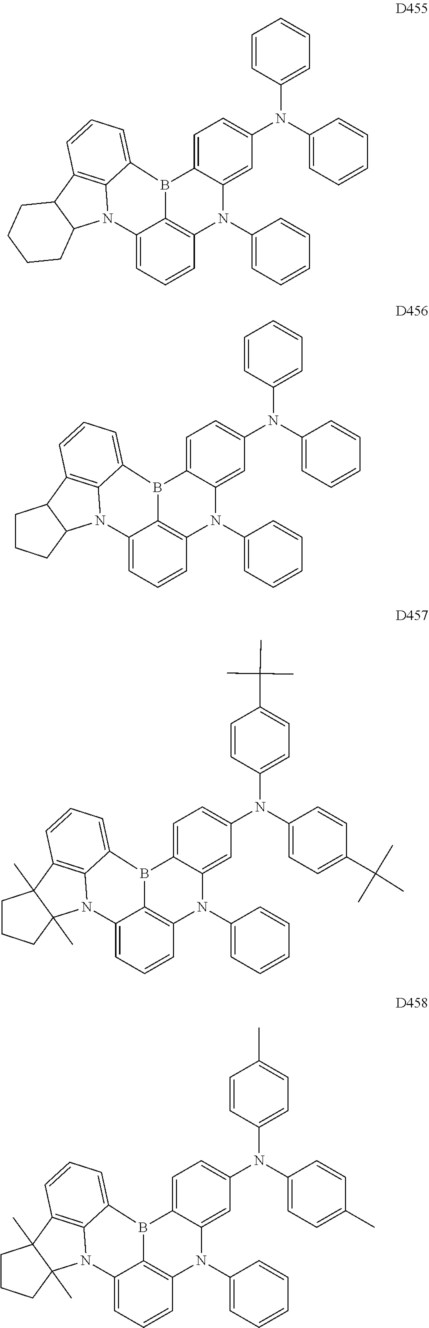

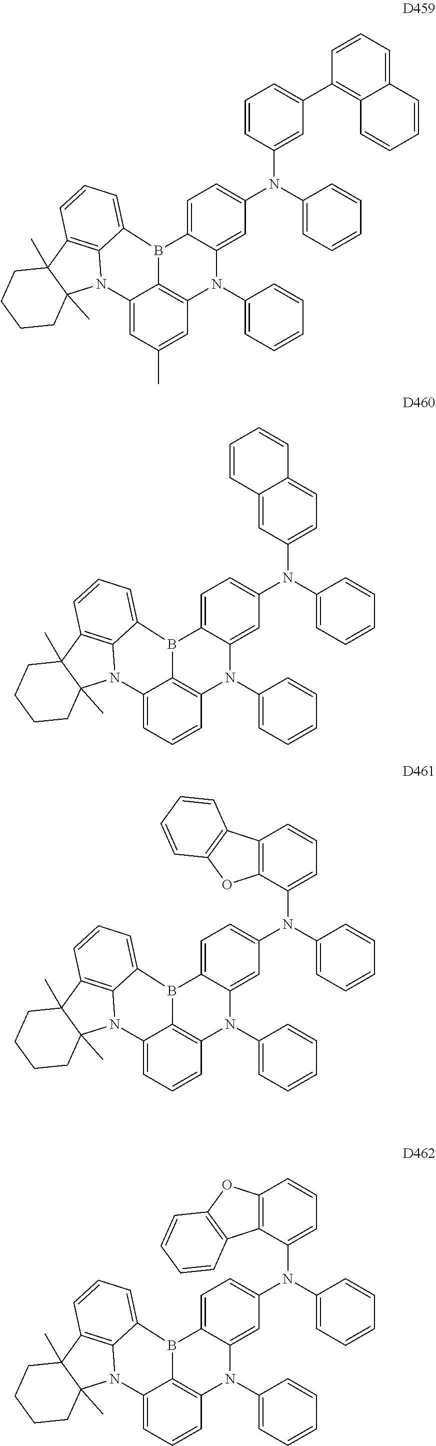 [SMILES: Cc1cc2c3c(c1)N1c4c(cccc4C4(C)CCCCC14C)B3c1ccc(N(c3ccccc3)c3cccc(-c4cccc5ccccc45)c3)cc1N2c1ccccc1 ; CC12CCCCC1(C)N1c3cccc4c3B(c3ccc(N(c5ccccc5)c5ccc6ccccc6c5)cc3N4c3ccccc3)c3cccc2c31 ; CC12CCCCC1(C)N1c3cccc4c3B(c3ccc(N(c5ccccc5)c5cccc6c5oc5ccccc56)cc3N4c3ccccc3)c3cccc2c31 ; CC12CCCCC1(C)N1c3cccc4c3B(c3ccc(N(c5ccccc5)c5cccc6oc7ccccc7c56)cc3N4c3ccccc3)c3cccc2c31]

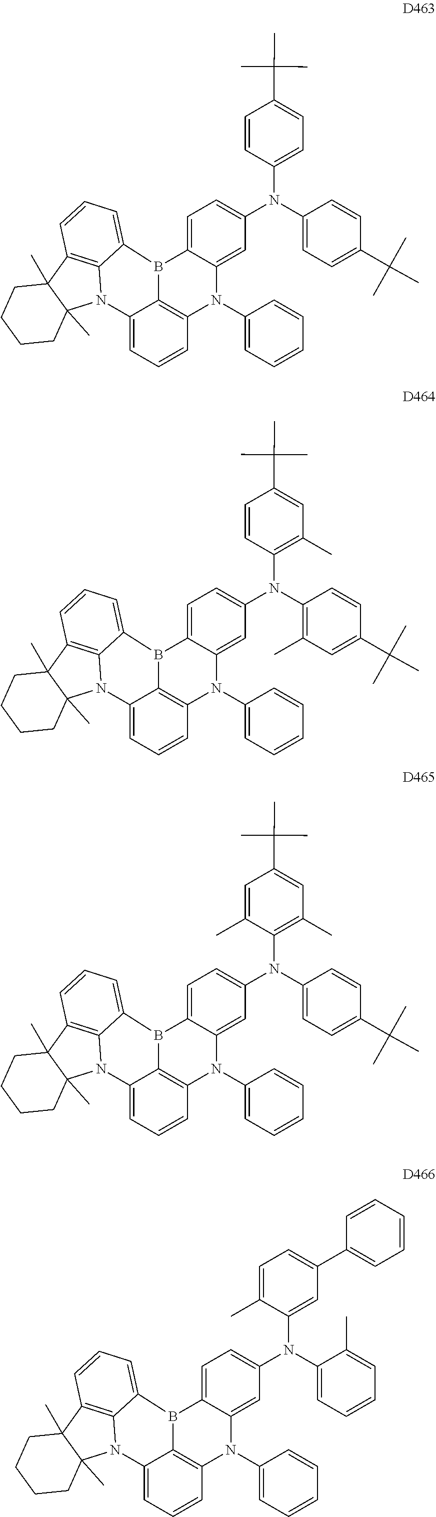 [SMILES: CC(C)(C)c1ccc(N(c2ccc(C(C)(C)C)cc2)c2ccc3c(c2)N(c2ccccc2)c2cccc4c2B3c2cccc3c2N4C2(C)CCCCC32C)cc1 ; Cc1cc(C(C)(C)C)ccc1N(c1ccc2c(c1)N(c1ccccc1)c1cccc3c1B2c1cccc2c1N3C1(C)CCCCC21C)c1ccc(C(C)(C)C)cc1C ; Cc1cc(C(C)(C)C)cc(C)c1N(c1ccc(C(C)(C)C)cc1)c1ccc2c(c1)N(c1ccccc1)c1cccc3c1B2c1cccc2c1N3C1(C)CCCCC21C ; Cc1ccccc1N(c1ccc2c(c1)N(c1ccccc1)c1cccc3c1B2c1cccc2c1N3C1(C)CCCCC21C)c1cc(-c2ccccc2)ccc1C]

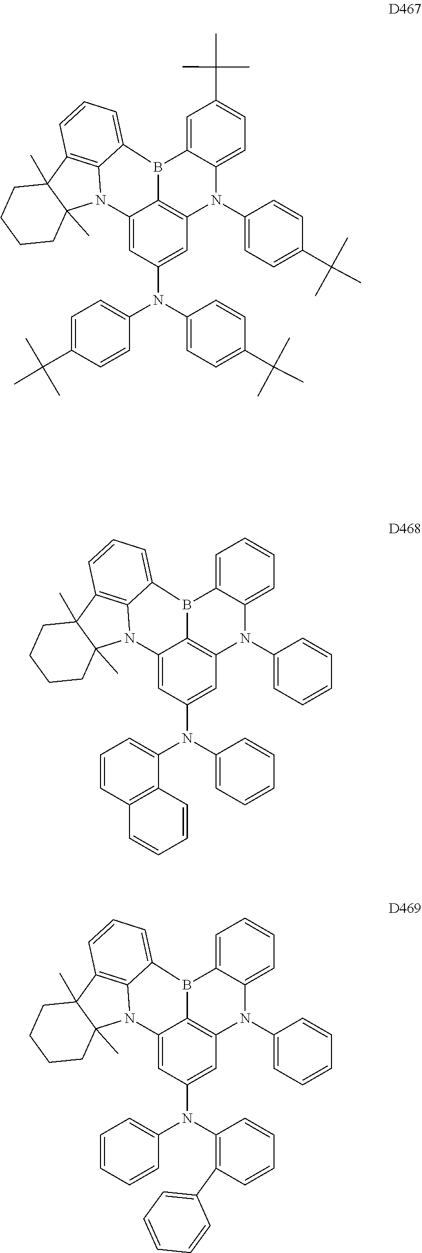 [SMILES: CC(C)(C)c1ccc(N(c2ccc(C(C)(C)C)cc2)c2cc3c4c(c2)N2c5c(cccc5C5(C)CCCCC25C)B4c2cc(C(C)(C)C)ccc2N3c2ccc(C(C)(C)C)cc2)cc1 ; CC12CCCCC1(C)N1c3cc(N(c4ccccc4)c4cccc5ccccc45)cc4c3B(c3ccccc3N4c3ccccc3)c3cccc2c31 ; CC12CCCCC1(C)N1c3cc(N(c4ccccc4)c4ccccc4-c4ccccc4)cc4c3B(c3ccccc3N4c3ccccc3)c3cccc2c31]

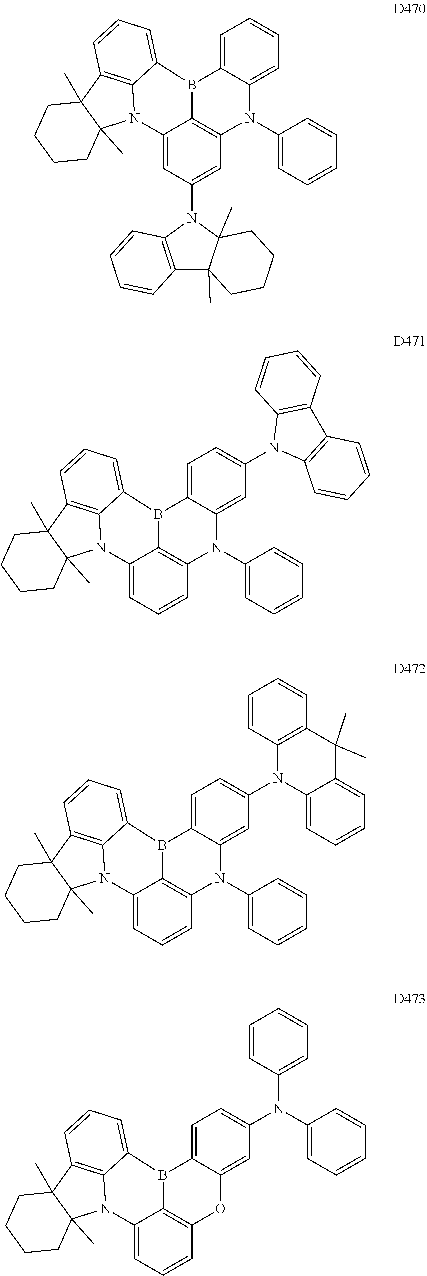 [SMILES: CC12CCCCC1(C)N(c1cc3c4c(c1)N1c5c(cccc5C5(C)CCCCC15C)B4c1ccccc1N3c1ccccc1)c1ccccc12 ; CC12CCCCC1(C)N1c3cccc4c3B(c3ccc(-n5c6ccccc6c6ccccc65)cc3N4c3ccccc3)c3cccc2c31 ; CC1(C)c2ccccc2N(c2ccc3c(c2)N(c2ccccc2)c2cccc4c2B3c2cccc3c2N4C2(C)CCCCC32C)c2ccccc21 ; CC12CCCCC1(C)N1c3cccc4c3B(c3ccc(N(c5ccccc5)c5ccccc5)cc3O4)c3cccc2c31]

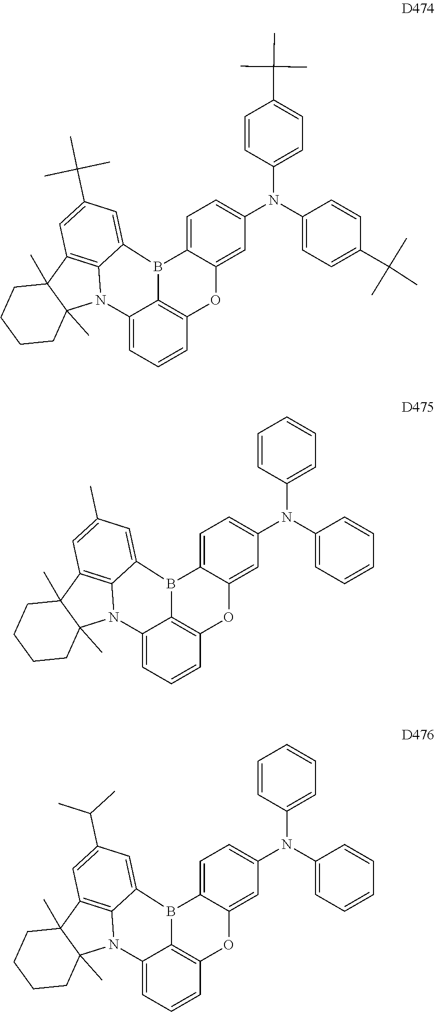 [SMILES: CC(C)(C)c1ccc(N(c2ccc(C(C)(C)C)cc2)c2ccc3c(c2)Oc2cccc4c2B3c2cc(C(C)(C)C)cc3c2N4C2(C)CCCCC32C)cc1 ; Cc1cc2c3c(c1)C1(C)CCCCC1(C)N3c1cccc3c1B2c1ccc(N(c2ccccc2)c2ccccc2)cc1O3 ; CC(C)c1cc2c3c(c1)C1(C)CCCCC1(C)N3c1cccc3c1B2c1ccc(N(c2ccccc2)c2ccccc2)cc1O3]

The compounds represented by Formulas D6 and D7 can be used as dopant compounds in the light emitting layer and may be selected from the following compounds D501 to D587:

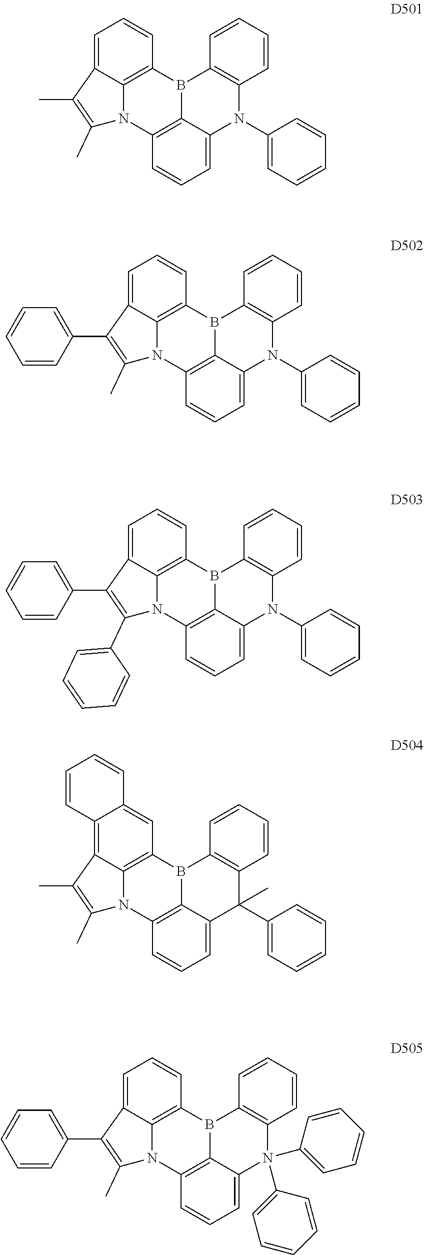

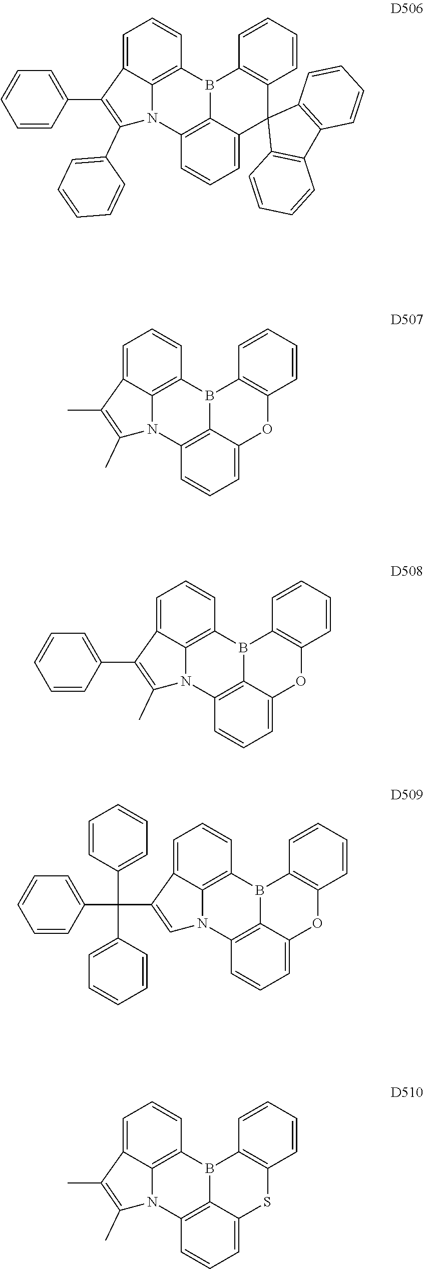 [SMILES: c1ccc(-c2c(-c3ccccc3)n3c4c(cccc24)B2c4ccccc4C4(c5ccccc5-c5ccccc54)c4cccc-3c42)cc1 ; Cc1c(C)n2c3c(cccc13)B1c3ccccc3Oc3cccc-2c31 ; Cc1c(-c2ccccc2)c2cccc3c2n1-c1cccc2c1B3c1ccccc1O2 ; c1ccc(C(c2ccccc2)(c2ccccc2)c2cn3c4c(cccc24)B2c4ccccc4Oc4cccc-3c42)cc1 ; Cc1c(C)n2c3c(cccc13)B1c3ccccc3Sc3cccc-2c31]

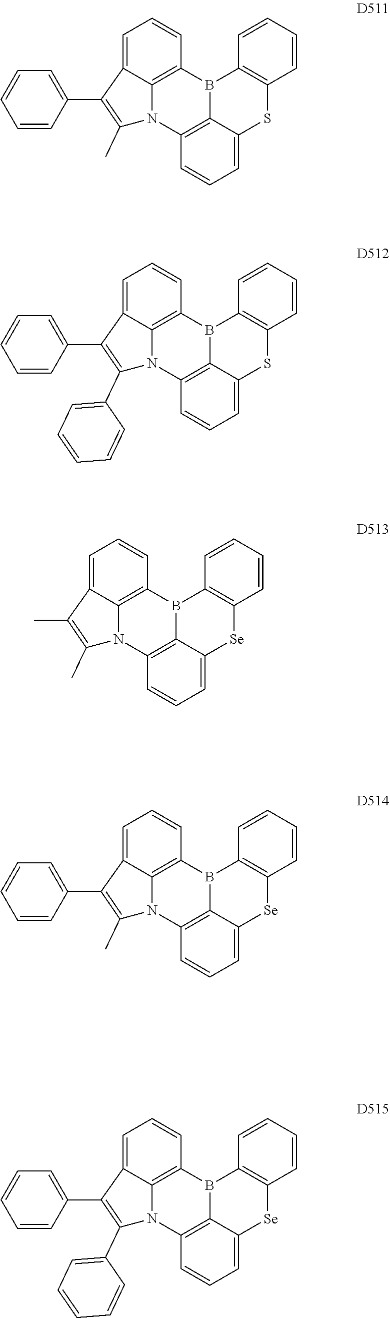 [SMILES: Cc1c(-c2ccccc2)c2cccc3c2n1-c1cccc2c1B3c1ccccc1S2 ; c1ccc(-c2c(-c3ccccc3)n3c4c(cccc24)B2c4ccccc4Sc4cccc-3c42)cc1 ; Cc1c(C)n2c3c(cccc13)B1c3ccccc3[Se]c3cccc-2c31 ; Cc1c(-c2ccccc2)c2cccc3c2n1-c1cccc2c1B3c1ccccc1[Se]2 ; c1ccc(-c2c(-c3ccccc3)n3c4c(cccc24)B2c4ccccc4[Se]c4cccc-3c42)cc1]

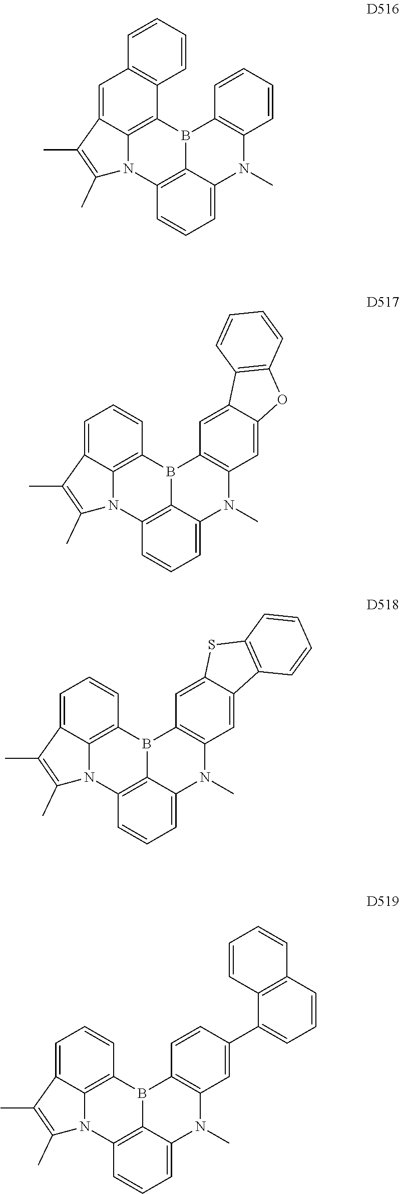

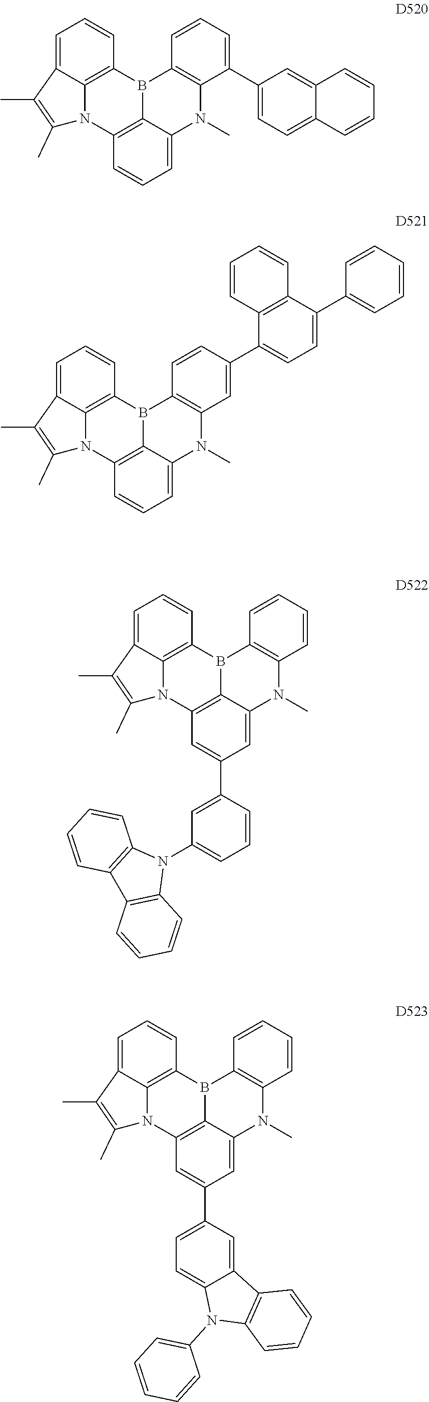 [SMILES: Cc1c(C)n2c3c(cccc13)B1c3cccc(-c4ccc5ccccc5c4)c3N(C)c3cccc-2c31 ; Cc1c(C)n2c3c(cccc13)B1c3ccc(-c4ccc(-c5ccccc5)c5ccccc45)cc3N(C)c3cccc-2c31 ; Cc1c(C)n2c3c(cccc13)B1c3ccccc3N(C)c3cc(-c4cccc(-n5c6ccccc6c6ccccc65)c4)cc-2c31 ; Cc1c(C)n2c3c(cccc13)B1c3ccccc3N(C)c3cc(-c4ccc5c(c4)c4ccccc4n5-c4ccccc4)cc-2c31]

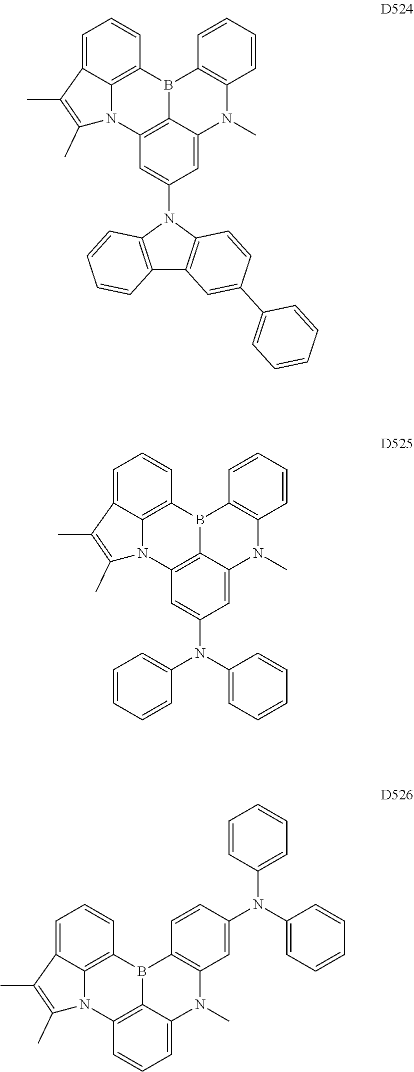 [SMILES: Cc1c(C)n2c3c(cccc13)B1c3ccccc3N(C)c3cc(-n4c5ccccc5c5cc(-c6ccccc6)ccc54)cc-2c31 ; Cc1c(C)n2c3c(cccc13)B1c3ccccc3N(C)c3cc(N(c4ccccc4)c4ccccc4)cc-2c31 ; Cc1c(C)n2c3c(cccc13)B1c3ccc(N(c4ccccc4)c4ccccc4)cc3N(C)c3cccc-2c31]

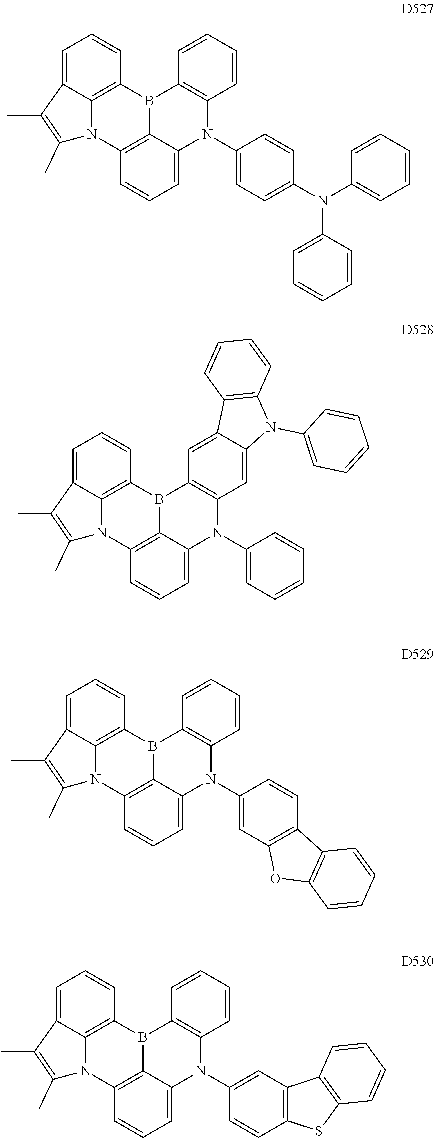 [SMILES: Cc1c(C)n2c3c(cccc13)B1c3ccccc3N(c3ccc(N(c4ccccc4)c4ccccc4)cc3)c3cccc-2c31 ; Cc1c(C)n2c3c(cccc13)B1c3cc4c5ccccc5n(-c5ccccc5)c4cc3N(c3ccccc3)c3cccc-2c31 ; Cc1c(C)n2c3c(cccc13)B1c3ccccc3N(c3ccc4c(c3)oc3ccccc34)c3cccc-2c31 ; Cc1c(C)n2c3c(cccc13)B1c3ccccc3N(c3ccc4sc5ccccc5c4c3)c3cccc-2c31]

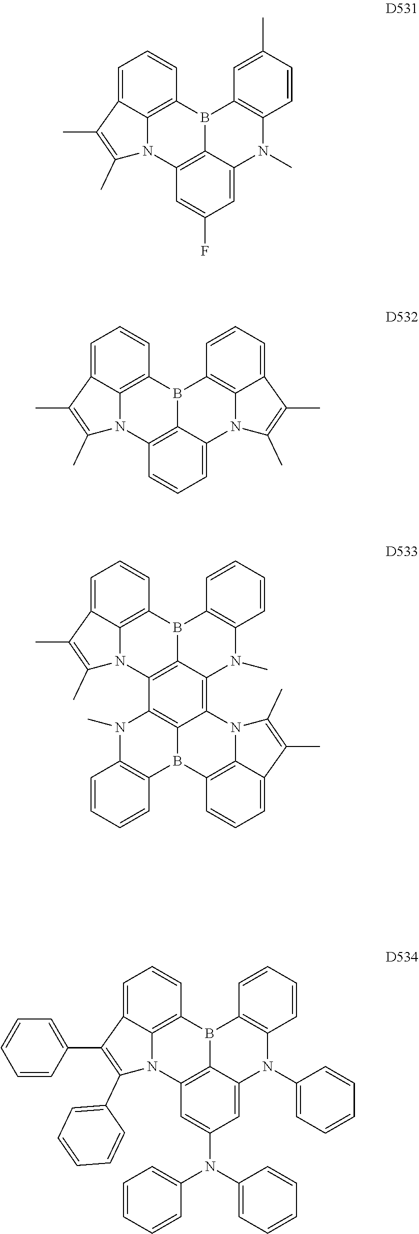

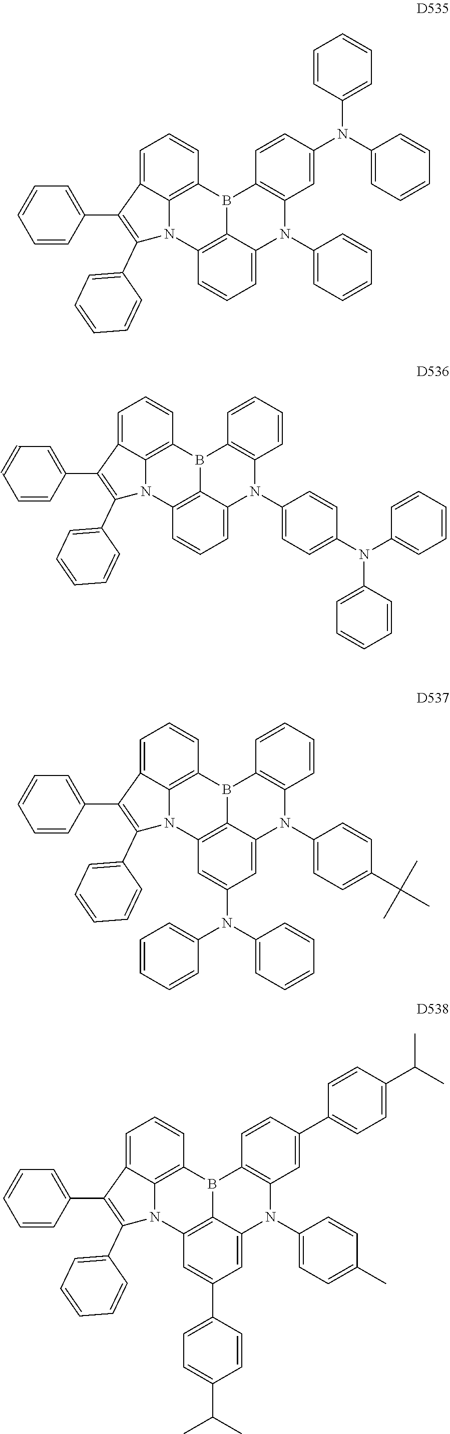 [SMILES: c1ccc(-c2c(-c3ccccc3)n3c4c(cccc24)B2c4ccc(N(c5ccccc5)c5ccccc5)cc4N(c4ccccc4)c4cccc-3c42)cc1 ; c1ccc(-c2c(-c3ccccc3)n3c4c(cccc24)B2c4ccccc4N(c4ccc(N(c5ccccc5)c5ccccc5)cc4)c4cccc-3c42)cc1 ; CC(C)(C)c1ccc(N2c3ccccc3B3c4c2cc(N(c2ccccc2)c2ccccc2)cc4-n2c(-c4ccccc4)c(-c4ccccc4)c4cccc3c42)cc1 ; Cc1ccc(N2c3cc(-c4ccc(C(C)C)cc4)ccc3B3c4c2cc(-c2ccc(C(C)C)cc2)cc4-n2c(-c4ccccc4)c(-c4ccccc4)c4cccc3c42)cc1]

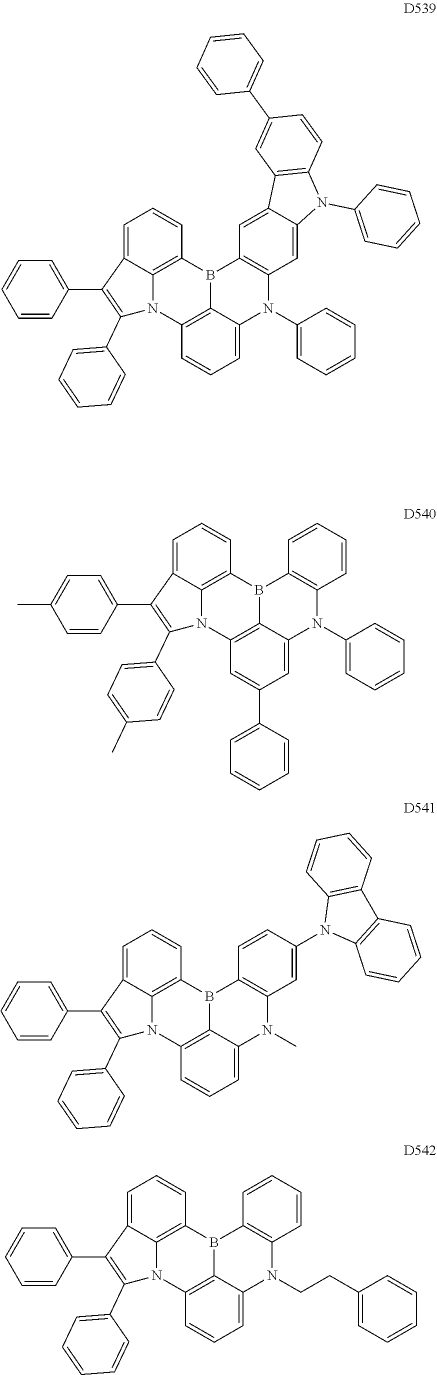 [SMILES: c1ccc(-c2ccc3c(c2)c2cc4c(cc2n3-c2ccccc2)N(c2ccccc2)c2cccc3c2B4c2cccc4c(-c5ccccc5)c(-c5ccccc5)n-3c24)cc1 ; Cc1ccc(-c2c(-c3ccc(C)cc3)n3c4c(cccc24)B2c4ccccc4N(c4ccccc4)c4cc(-c5ccccc5)cc-3c42)cc1 ; CN1c2cc(-n3c4ccccc4c4ccccc43)ccc2B2c3c1cccc3-n1c(-c3ccccc3)c(-c3ccccc3)c3cccc2c31 ; c1ccc(CCN2c3ccccc3B3c4c2cccc4-n2c(-c4ccccc4)c(-c4ccccc4)c4cccc3c42)cc1]

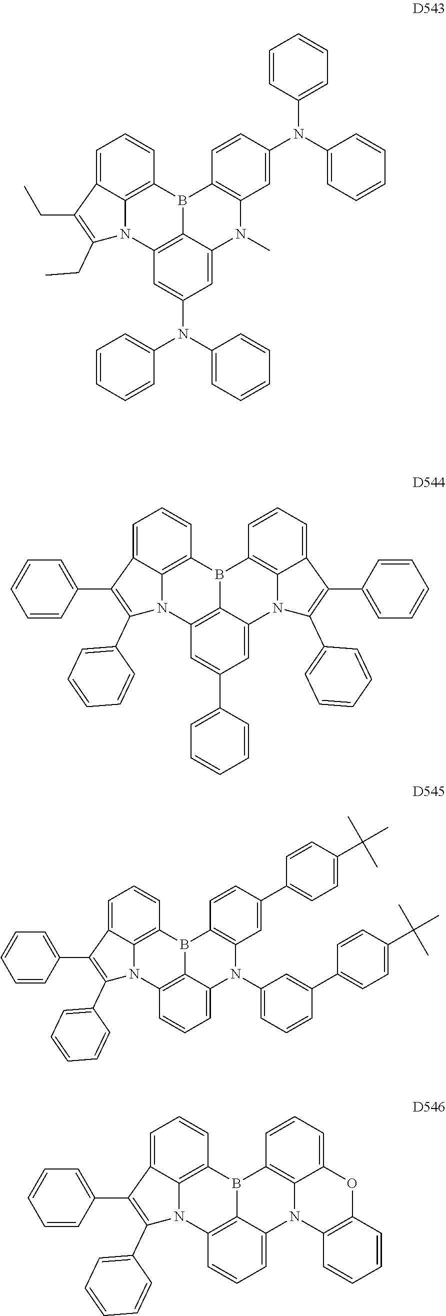 [SMILES: CCc1c(CC)n2c3c(cccc13)B1c3ccc(N(c4ccccc4)c4ccccc4)cc3N(C)c3cc(N(c4ccccc4)c4ccccc4)cc-2c31 ; c1ccc(-c2cc3c4c(c2)-n2c(-c5ccccc5)c(-c5ccccc5)c5cccc(c52)B4c2cccc4c(-c5ccccc5)c(-c5ccccc5)n-3c24)cc1 ; CC(C)(C)c1ccc(-c2cccc(N3c4cc(-c5ccc(C(C)(C)C)cc5)ccc4B4c5c3cccc5-n3c(-c5ccccc5)c(-c5ccccc5)c5cccc4c53)c2)cc1 ; c1ccc(-c2c(-c3ccccc3)n3c4c(cccc24)B2c4cccc5c4N(c4ccccc4O5)c4cccc-3c42)cc1]

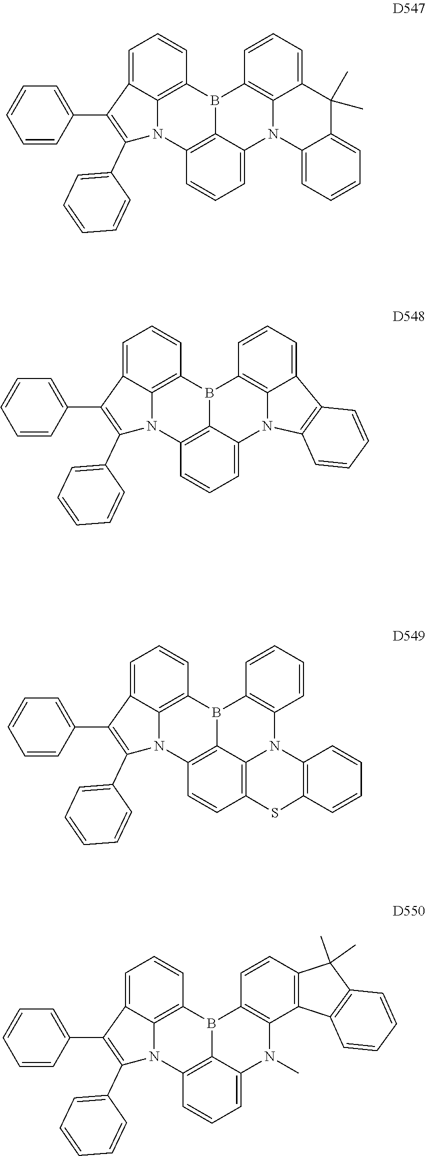 [SMILES: CC1(C)c2ccccc2N2c3cccc4c3B(c3cccc1c32)c1cccc2c(-c3ccccc3)c(-c3ccccc3)n-4c12 ; c1ccc(-c2c(-c3ccccc3)n3c4c(cccc24)B2c4c-3cccc4-n3c4ccccc4c4cccc2c43)cc1 ; c1ccc(-c2c(-c3ccccc3)n3c4c(cccc24)B2c4ccccc4N4c5ccccc5Sc5ccc-3c2c54)cc1 ; CN1c2cccc3c2B(c2ccc4c(c21)-c1ccccc1C4(C)C)c1cccc2c(-c4ccccc4)c(-c4ccccc4)n-3c12]

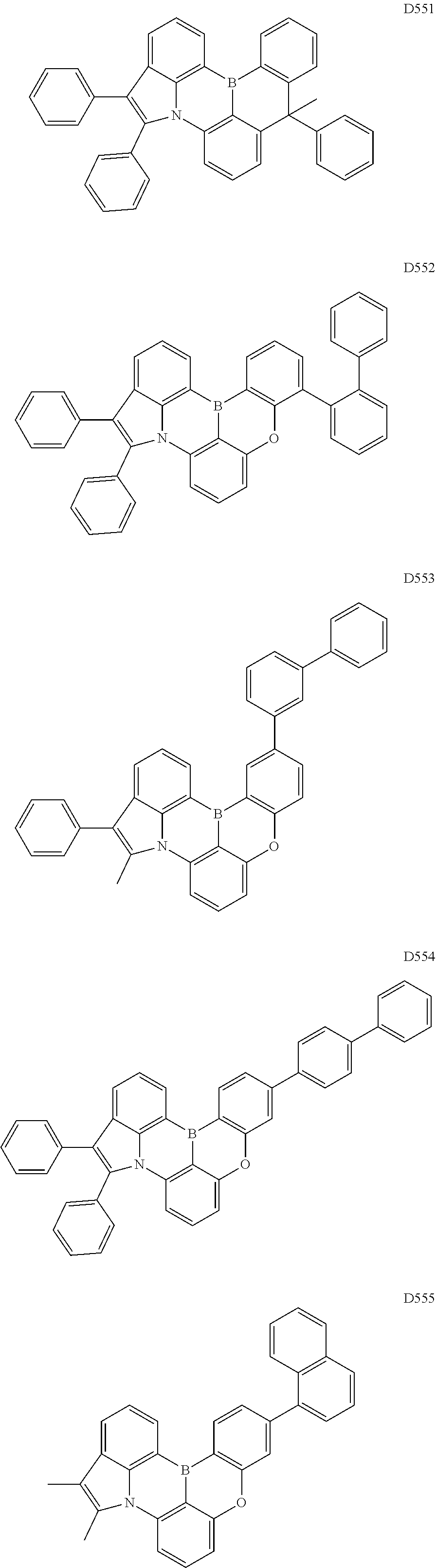 [SMILES: CC1(c2ccccc2)c2ccccc2B2c3c(cccc31)-n1c(-c3ccccc3)c(-c3ccccc3)c3cccc2c31 ; c1ccc(-c2ccccc2-c2cccc3c2Oc2cccc4c2B3c2cccc3c(-c5ccccc5)c(-c5ccccc5)n-4c23)cc1 ; Cc1c(-c2ccccc2)c2cccc3c2n1-c1cccc2c1B3c1cc(-c3cccc(-c4ccccc4)c3)ccc1O2 ; c1ccc(-c2ccc(-c3ccc4c(c3)Oc3cccc5c3B4c3cccc4c(-c6ccccc6)c(-c6ccccc6)n-5c34)cc2)cc1 ; Cc1c(C)n2c3c(cccc13)B1c3ccc(-c4cccc5ccccc45)cc3Oc3cccc-2c31]

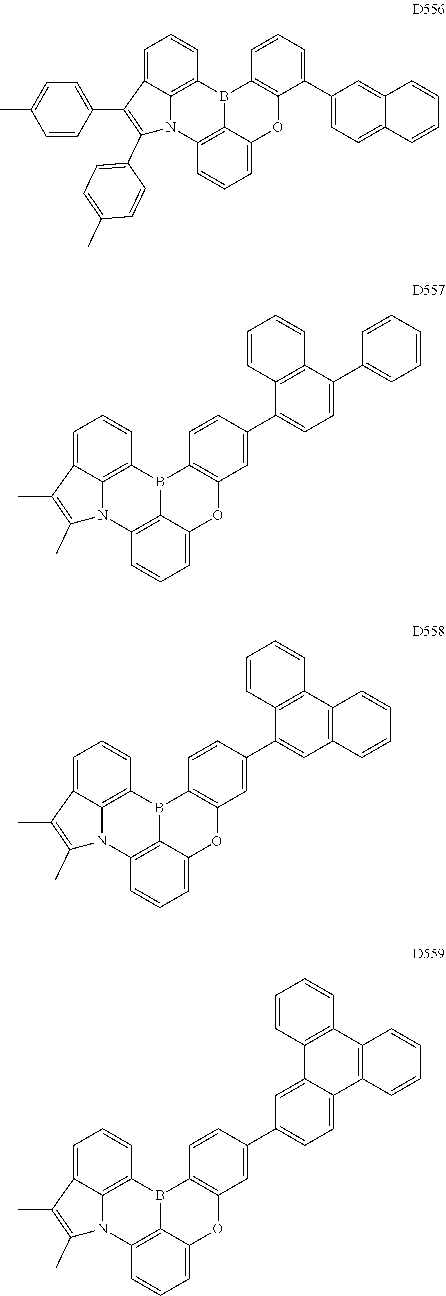 [SMILES: Cc1ccc(-c2c(-c3ccc(C)cc3)n3c4c(cccc24)B2c4cccc(-c5ccc6ccccc6c5)c4Oc4cccc-3c42)cc1 ; Cc1c(C)n2c3c(cccc13)B1c3ccc(-c4ccc(-c5ccccc5)c5ccccc45)cc3Oc3cccc-2c31 ; Cc1c(C)n2c3c(cccc13)B1c3ccc(-c4cc5ccccc5c5ccccc45)cc3Oc3cccc-2c31 ; Cc1c(C)n2c3c(cccc13)B1c3ccc(-c4ccc5c6ccccc6c6ccccc6c5c4)cc3Oc3cccc-2c31]

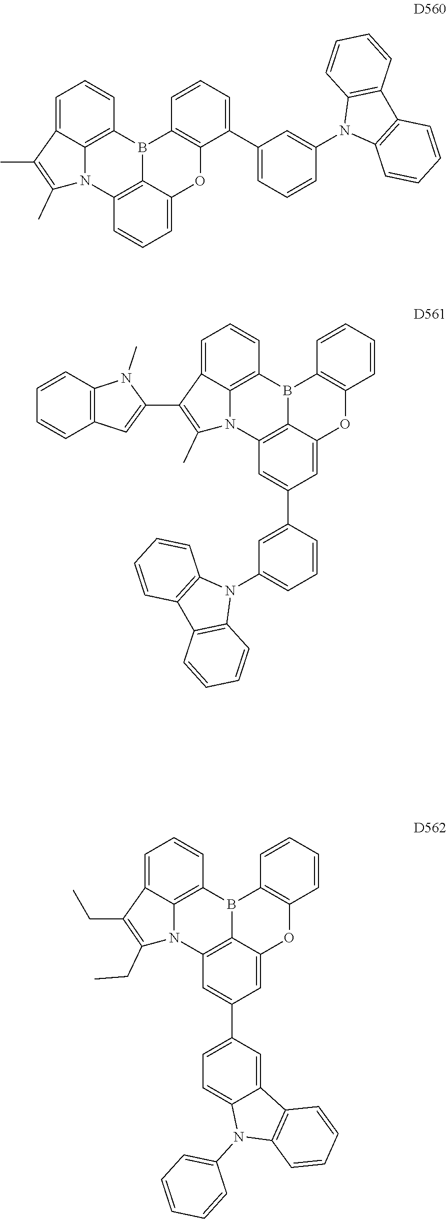 [SMILES: Cc1c(C)n2c3c(cccc13)B1c3cccc(-c4cccc(-n5c6ccccc6c6ccccc65)c4)c3Oc3cccc-2c31 ; Cc1c(-c2cc3ccccc3n2C)c2cccc3c2n1-c1cc(-c2cccc(-n4c5ccccc5c5ccccc54)c2)cc2c1B3c1ccccc1O2 ; CCc1c(CC)n2c3c(cccc13)B1c3ccccc3Oc3cc(-c4ccc5c(c4)c4ccccc4n5-c4ccccc4)cc-2c31]

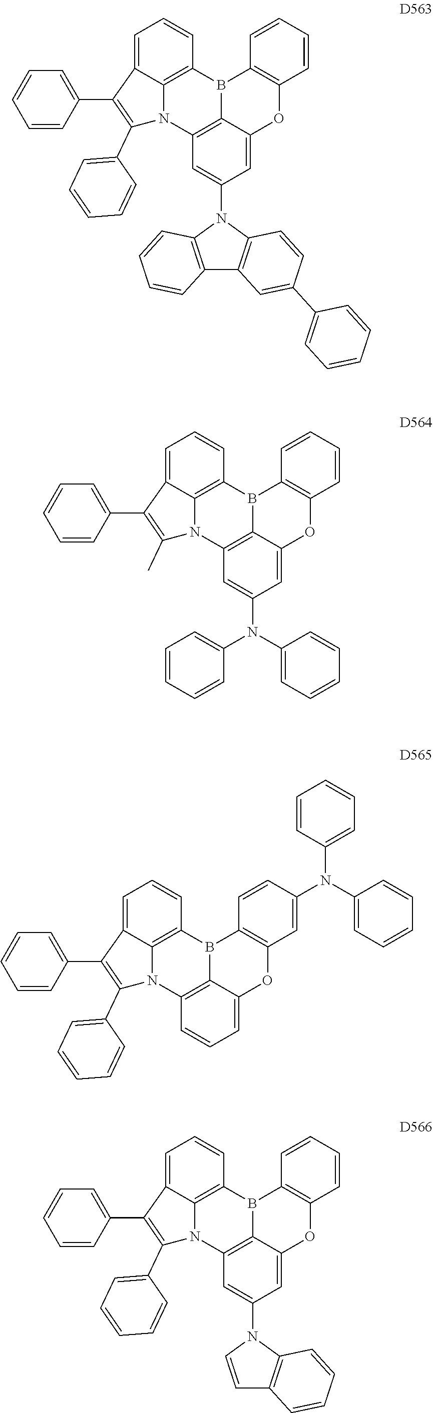 [SMILES: c1ccc(-c2ccc3c(c2)c2ccccc2n3-c2cc3c4c(c2)-n2c(-c5ccccc5)c(-c5ccccc5)c5cccc(c52)B4c2ccccc2O3)cc1 ; Cc1c(-c2ccccc2)c2cccc3c2n1-c1cc(N(c2ccccc2)c2ccccc2)cc2c1B3c1ccccc1O2 ; c1ccc(-c2c(-c3ccccc3)n3c4c(cccc24)B2c4ccc(N(c5ccccc5)c5ccccc5)cc4Oc4cccc-3c42)cc1 ; c1ccc(-c2c(-c3ccccc3)n3c4c(cccc24)B2c4ccccc4Oc4cc(-n5ccc6ccccc65)cc-3c42)cc1]

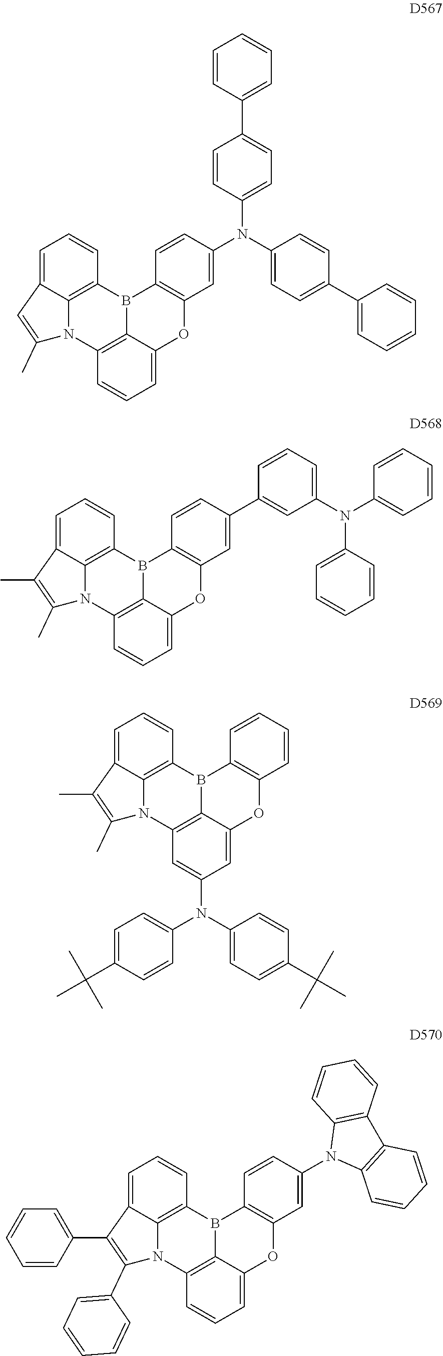 [SMILES: Cc1cc2cccc3c2n1-c1cccc2c1B3c1ccc(N(c3ccc(-c4ccccc4)cc3)c3ccc(-c4ccccc4)cc3)cc1O2 ; Cc1c(C)n2c3c(cccc13)B1c3ccc(-c4cccc(N(c5ccccc5)c5ccccc5)c4)cc3Oc3cccc-2c31 ; Cc1c(C)n2c3c(cccc13)B1c3ccccc3Oc3cc(N(c4ccc(C(C)(C)C)cc4)c4ccc(C(C)(C)C)cc4)cc-2c31 ; c1ccc(-c2c(-c3ccccc3)n3c4c(cccc24)B2c4ccc(-n5c6ccccc6c6ccccc65)cc4Oc4cccc-3c42)cc1]

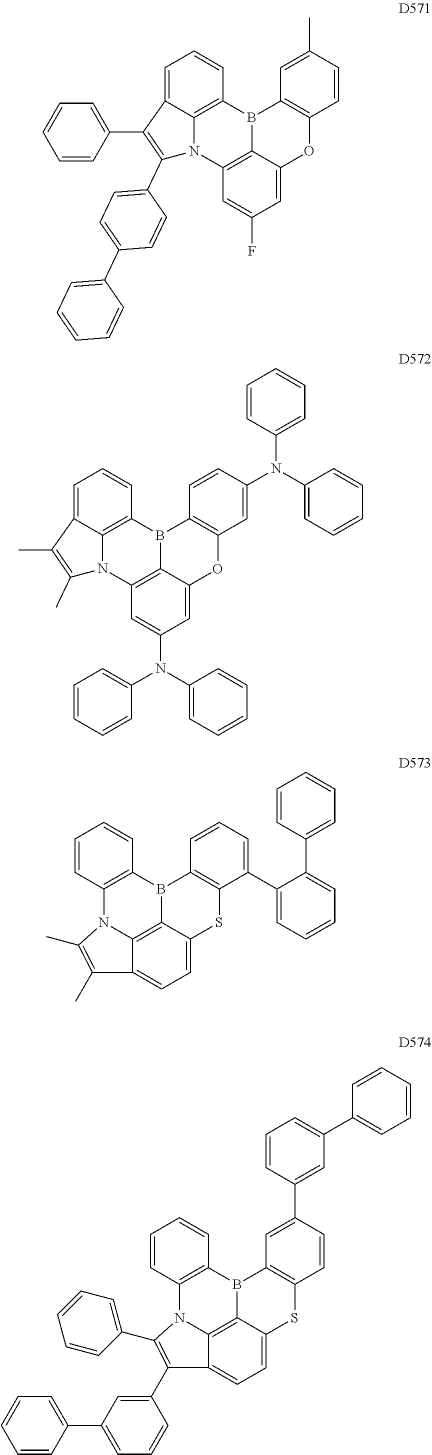 [SMILES: Cc1ccc2c(c1)B1c3c(cc(F)cc3-n3c(-c4ccc(-c5ccccc5)cc4)c(-c4ccccc4)c4cccc1c43)O2 ; Cc1c(C)n2c3c(cccc13)B1c3ccc(N(c4ccccc4)c4ccccc4)cc3Oc3cc(N(c4ccccc4)c4ccccc4)cc-2c31 ; Cc1c(C)n2c3c4c(ccc13)Sc1c(cccc1-c1ccccc1-c1ccccc1)B4c1ccccc1-2 ; c1ccc(-c2cccc(-c3ccc4c(c3)B3c5ccccc5-n5c(-c6ccccc6)c(-c6cccc(-c7ccccc7)c6)c6ccc(c3c65)S4)c2)cc1]

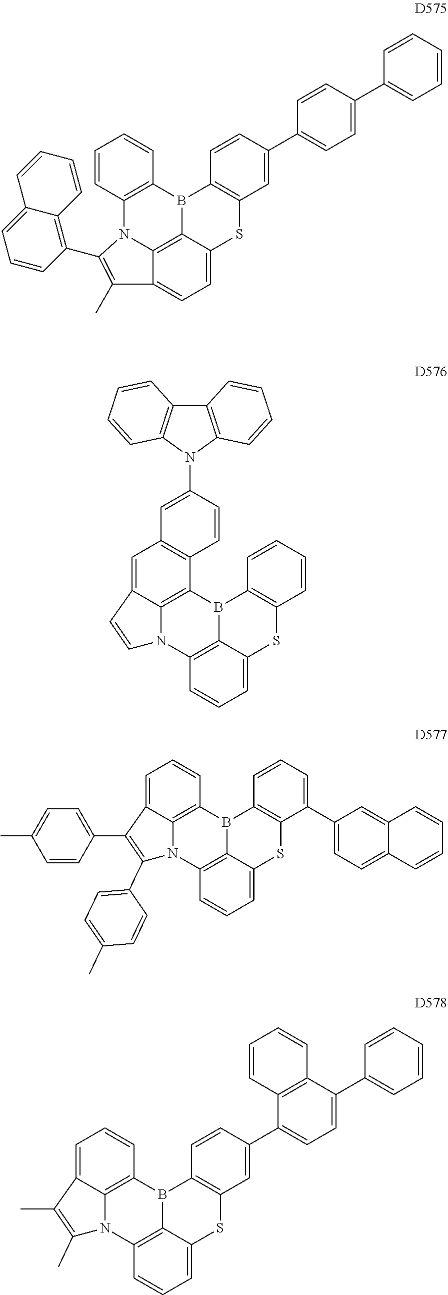 [SMILES: Cc1c(-c2cccc3ccccc23)n2c3c4c(ccc13)Sc1cc(-c3ccc(-c5ccccc5)cc3)ccc1B4c1ccccc1-2 ; c1ccc2c(c1)Sc1cccc3c1B2c1c2ccc(-n4c5ccccc5c5ccccc54)cc2cc2ccn-3c12 ; Cc1ccc(-c2c(-c3ccc(C)cc3)n3c4c(cccc24)B2c4cccc(-c5ccc6ccccc6c5)c4Sc4cccc-3c42)cc1 ; Cc1c(C)n2c3c(cccc13)B1c3ccc(-c4ccc(-c5ccccc5)c5ccccc45)cc3Sc3cccc-2c31]

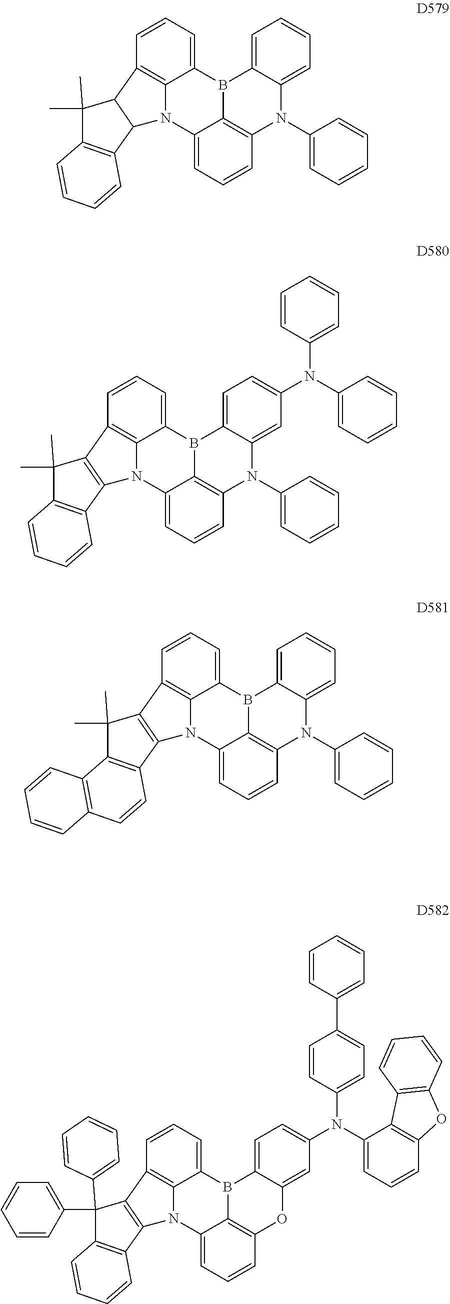 [SMILES: CC1(C)c2ccccc2C2C1c1cccc3c1N2c1cccc2c1B3c1ccccc1N2c1ccccc1 ; CC1(C)c2ccccc2-c2c1c1cccc3c1n2-c1cccc2c1B3c1ccc(N(c3ccccc3)c3ccccc3)cc1N2c1ccccc1 ; CC1(C)c2c(ccc3ccccc23)-c2c1c1cccc3c1n2-c1cccc2c1B3c1ccccc1N2c1ccccc1 ; c1ccc(-c2ccc(N(c3ccc4c(c3)Oc3cccc5c3B4c3cccc4c6c(n-5c34)-c3ccccc3C6(c3ccccc3)c3ccccc3)c3cccc4oc5ccccc5c34)cc2)cc1]

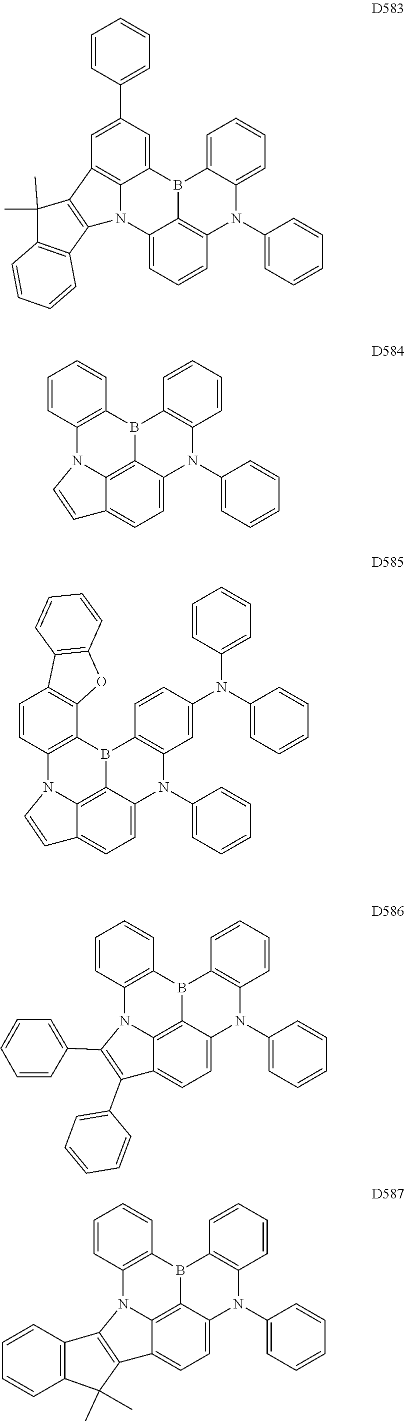

The content of the dopant in the light emitting layer is typically in the range of about 0.01 to about 20 parts by weight, based on about 100 parts by weight of the host but is not limited to this range.

The light emitting layer may further include one or more dopants other than the dopants represented by Formulas D1 to D10 and one or more hosts other than the host represented by Formula 1.

The organic layers of the organic light emitting device according to the present invention may form a monolayer structure. Alternatively, the organic layers may be stacked together to form a multilayer structure. For example, the organic layers may have a structure including a hole injecting layer, a hole transport layer, a hole blocking layer, a light emitting layer, an electron blocking layer, an electron transport layer, and an electron injecting layer but are not limited to this structure. The number of the organic layers is not limited and may be increased or decreased. Preferred structures of the organic layers of the organic light emitting device according to the present invention will be explained in more detail in the Examples section that follows.

According to one embodiment of the present invention, the organic electroluminescent device may include a substrate, a first electrode (anode), one or more organic layers, a second electrode (cathode), and a capping layer formed under the first electrode (bottom emission type) or on the second electrode (top emission type).

When the organic electroluminescent device is of a top emission type, light from the light emitting layer is emitted to the cathode and passes through the capping layer (CPL) formed using the compound of the present invention having a relatively high refractive index. The wavelength of the light is amplified in the capping layer, resulting in an increase in luminous efficiency. Also when the organic electroluminescent device is of a bottom emission type, the compound of the present invention can be employed in the capping layer to improve the luminous efficiency of the organic electroluminescent device based on the same principle.

A more detailed description will be given concerning exemplary embodiments of the organic light emitting device according to the present invention.

The organic light emitting device of the present invention includes an anode, a hole transport layer, a light emitting layer, an electron transport layer, and a cathode. The organic light emitting device of the present invention may optionally further include a hole injecting layer between the anode and the hole transport layer and an electron injecting layer between the electron transport layer and the cathode. If necessary, the organic light emitting device of the present invention may further include one or two intermediate layers such as a hole blocking layer or an electron blocking layer. The organic light emitting device of the present invention may further include one or more organic layers such as a capping layer that have various functions depending on the desired characteristics of the device.

A specific structure of the organic light emitting device according to one embodiment of the present invention, a method for fabricating the device, and materials for the organic layers are as follows.

First, an anode material is coated on a substrate to form an anode. The substrate may be any of those used in general organic light emitting devices. The substrate is preferably an organic substrate or a transparent plastic substrate that is excellent in transparency, surface smoothness, ease of handling, and waterproofness. A highly transparent and conductive metal oxide such as indium tin oxide (ITO), indium zinc oxide (IZO), tin oxide (SnO₂) or zinc oxide (ZnO) is used as the anode material.

A hole injecting material is coated on the anode by vacuum thermal evaporation or spin coating to form a hole injecting layer. Then, a hole transport material is coated on the hole injecting layer by vacuum thermal evaporation or spin coating to form a hole transport layer.

The hole injecting material is not specially limited so long as it is usually used in the art. Specific examples of such materials include 4,4′,4″-tris(2-naphthylphenyl-phenylamino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis(4-(phenyl-m-tolylamino)phenyl)biphenyl-4,4′-diamine (DNTPD).

The hole transport material is not specially limited so long as it is commonly used in the art. Examples of such materials include N,N′-bis(3-methylphenyl)-N,N′-diphenyl-(1,1-biphenyl)-4,4′-diamine (TPD) and N,N′-di(naphthalen-1-yl)-N,N′-diphenylbenzidine (α-NPD).

Subsequently, a hole auxiliary layer and a light emitting layer are sequentially laminated on the hole transport layer. A hole blocking layer may be optionally formed on the light emitting layer by vacuum thermal evaporation or spin coating. The hole blocking layer is formed as a thin film and blocks holes from entering a cathode through the organic light emitting layer. This role of the hole blocking layer prevents the lifetime and efficiency of the device from deteriorating. A material having a very low highest occupied molecular orbital (HOMO) energy level is used for the hole blocking layer. The hole blocking material is not particularly limited so long as it can transport electrons and has a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, NTAZ, BeBq₂, OXD-7, and Liq.

An electron transport layer is deposited on the hole blocking layer by vacuum thermal evaporation or spin coating, and an electron injecting layer is formed thereon. A cathode metal is deposited on the electron injecting layer by vacuum thermal evaporation to form a cathode, completing the fabrication of the organic light emitting device.

For example, lithium (Li), magnesium (Mg), aluminum (AI), aluminum-lithium (Al—Li), calcium (Ca), magnesium-indium (Mg—In) or magnesium-silver (Mg—Ag) may be used as the metal for the formation of the cathode. The organic light emitting device may be of top emission type. In this case, a transmissive material such as ITO or IZO may be used to form the cathode.

A material for the electron transport layer functions to stably transport electrons injected from the cathode. The electron transport material may be any of those known in the art and examples thereof include, but are not limited to, quinoline derivatives, particularly tris(8-quinolinolate)aluminum (Alq3), TAZ, Balq, beryllium bis(benzoquinolin-10-olate) (Bebq2), and oxadiazole derivatives such as PBD, BMD, and BND.

Each of the organic layers can be formed by a monomolecular deposition or solution process. According to the monomolecular deposition process, the material for each layer is evaporated into a thin film under heat and vacuum or reduced pressure. According to the solution process, the material for each layer is mixed with a suitable solvent and the mixture is then formed into a thin film by a suitable method such as ink-jet printing, roll-to-roll coating, screen printing, spray coating, dip coating or spin coating.

The organic light emitting device of the present invention can be used in a display or lighting system selected from flat panel displays, flexible displays, monochromatic flat panel lighting systems, white flat panel lighting systems, flexible monochromatic lighting systems, and flexible white lighting systems.

### MODE FOR CARRYING OUT THE INVENTION

The present invention will be more specifically explained with reference to the following examples. However, it will be obvious to those skilled in the art that these examples are in no way intended to limit the scope of the invention.

### Synthesis Example 1: Synthesis of 4

### Synthesis Example 1-1: Synthesis of 1-a

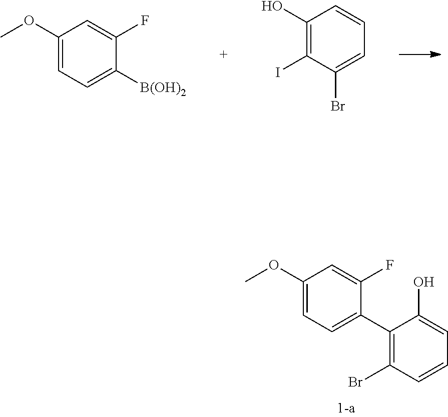 [SMILES: COc1ccc(B(O)O)c(F)c1 ; Oc1cccc(Br)c1I ; COc1ccc(-c2c(O)cccc2Br)c(F)c1]

50 g (0.294 mol) of 2-fluoro-4-methoxyphenylboronic acid, 105.5 g (0.353 mol) of 3-bromo-2-iodophenol, 6.8 g (0.006 mol) of tetrakis(triphenylphosphine)palladium, 69.1 g (0.500 mol) of potassium carbonate, 350 mL of toluene, 85 mL of ethanol, and 250 mL of water were placed in a round-bottom flask. The mixture was refluxed for 8 h. After completion of the reaction, the reaction solution was cooled to room temperature, extracted with ethyl acetate and water, and concentrated under reduced pressure. Column chromatography of the concentrate afforded 68 g (yield 77.8%) of 1-a.

### Synthesis Example 1-2. Synthesis of 1-b

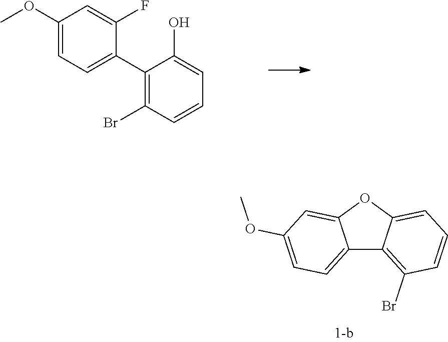 [SMILES: COc1ccc(-c2c(O)cccc2Br)c(F)c1 ; COc1ccc2c(c1)oc1cccc(Br)c12]

68 g (0.229 mol) of 1-a, 79.1 g (0.572 mol) of potassium carbonate, and 340 mL of 1-methyl-2-pyrrolidine were placed in a round-bottom flask under a nitrogen atmosphere. The mixture was refluxed for 12 h. After completion of the reaction, the reaction solution was cooled to room temperature and 200 mL of a 2 N aqueous hydrochloric acid solution was slowly added thereto. The mixture was sufficiently stirred and extracted with ethyl acetate and water. The organic layer was concentrated and purified by column chromatography to afford 50 g (yield 78.8%) of 1-b.

### Synthesis Example 1-3. Synthesis of 1-c

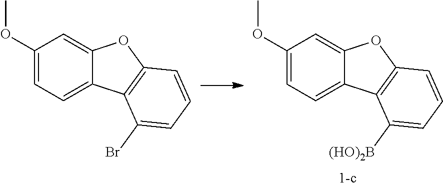 [SMILES: COc1ccc2c(c1)oc1cccc(Br)c12 ; COc1ccc2c(c1)oc1cccc(B(O)O)c12]

50 g (0.180 mol) of 1-b and 400 mL of tetrahydrofuran were placed in a 1000 mL round-bottom flask under a nitrogen atmosphere. The mixture was stirred. The reaction solution was cooled to −78° C. and 135.3 mL (0.217 mol) of 1.6 M n-butyllithium was slowly added dropwise thereto. After the dropwise addition was completed, the resulting mixture was stirred while maintaining the same temperature for 2 h. 2 h later, 26.2 g (0.253 mol) of trimethyl borate was slowly added dropwise. The reaction solution was heated to room temperature and stirred until the reaction was completed. After completion of the reaction, a 2 N aqueous hydrochloric acid solution was slowly added dropwise, followed by stirring for 1 h. 1 h later, extraction was performed with ethyl acetate and water. The organic layer was washed twice with water, made anhydrous, concentrated under reduced pressure, and crystallized from heptane to afford 32 g (yield 73.5%) of 1-c.

### Synthesis Example 1-4. Synthesis of 1-d

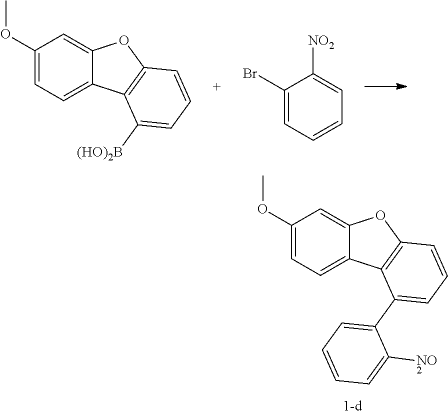 [SMILES: COc1ccc2c(c1)oc1cccc(B(O)O)c12 ; O=[N+]([O-])c1ccccc1Br ; COc1ccc2c(c1)oc1cccc(-c3ccccc3[N+](=O)[O-])c12]

23.4 g (0.116 mol) of 2-bromonitrobenzene, 32.2 g (0.133 mol) of 1-c, 2.7 g (0.002 mol) of tetrakis(triphenylphosphine)palladium, 27.3 g (0.198 mol) of potassium carbonate, 165 mL of toluene, 40 mL of ethanol, and 100 mL of water were placed in a 500 mL round-bottom flask under a nitrogen atmosphere. The mixture was refluxed for 8 h. After completion of the reaction, the reaction solution was cooled to room temperature, extracted with ethyl acetate and water, and concentrated under reduced pressure. Column chromatography of the concentrate afforded 26.3 g (yield 71.1%) of 1-d.

### Synthesis Example 1-5. Synthesis of 1-e

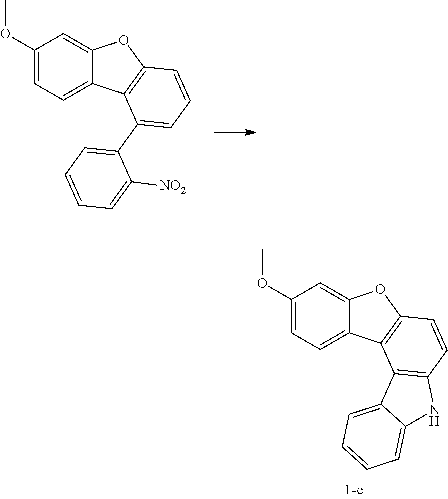 [SMILES: COc1ccc2c(c1)oc1cccc(-c3ccccc3[N+](=O)[O-])c12 ; COc1ccc2c(c1)oc1ccc3[nH]c4ccccc4c3c12]

24.6 g (0.077 mol) of 1-d, 62.2 g (0.231 mol) of triphenylphosphine, and 246 mL of 1,2-dichlorobenzene were placed in a 500 mL round-bottom flask. The mixture was refluxed for 18 h. After completion of the reaction, the reaction solution was cooled to room temperature and extracted with dichloromethane and water. The organic layer was concentrated under reduced pressure. Column chromatography of the concentrate afforded 14 g (yield 63.2%) of 1-e.

### Synthesis Example 1-6. Synthesis of 1-f

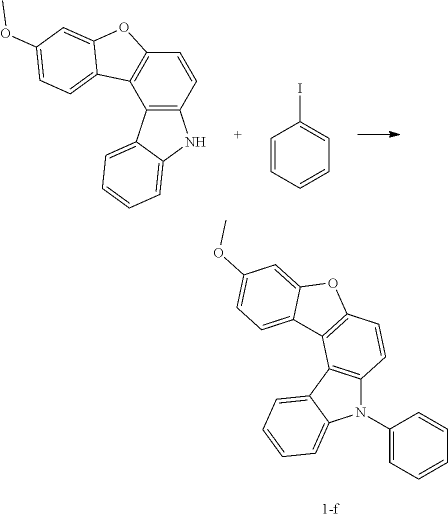 [SMILES: COc1ccc2c(c1)oc1ccc3[nH]c4ccccc4c3c12 ; Ic1ccccc1 ; COc1ccc2c(c1)oc1ccc3c(c4ccccc4n3-c3ccccc3)c12]

14.1 g (0.049 mol) of 1-e, 14.9 g (0.073 mol) of iodobenzene, 0.5 g (0.002 mol) of copper iodide, 31 g (0.146 mol) of potassium phosphate, 16.7 g (0.146 mol) of 1,2-cyclohexanediamine (cis:trans=1:1), and 140 mL of 1,4-dioxane were placed in a 500 mL round-bottom flask. The mixture was refluxed for 6 h. After completion of the reaction, the reaction solution was cooled to room temperature and filtered through Celite. The filtrate was concentrated under reduced pressure and purified by column chromatography to afford 15.1 g (yield 84.7%) of 1-f.

### Synthesis Example 1-7. Synthesis of 1-q

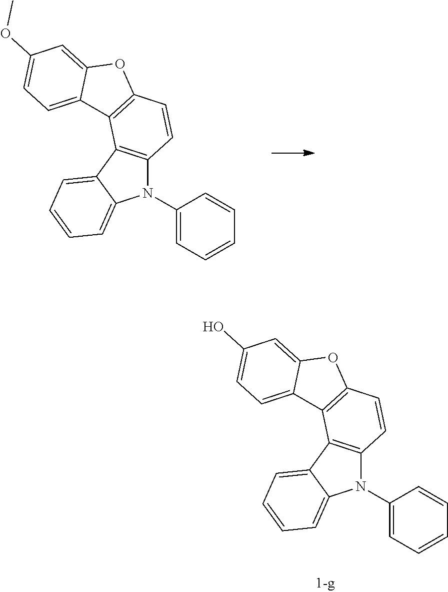 [SMILES: COc1ccc2c(c1)oc1ccc3c(c4ccccc4n3-c3ccccc3)c12 ; Oc1ccc2c(c1)oc1ccc3c(c4ccccc4n3-c3ccccc3)c12]

14.9 g (0.041 mol) of 1-f and 100 mL of dichloroethane were placed in a 300 mL round-bottom flask under a nitrogen atmosphere. The mixture was stirred. The reaction solution was cooled to 0° C. and 20.7 g (0.083 mol) of boron tribromide was slowly added dropwise thereto. The reaction solution was heated to room temperature and stirred until the reaction was completed. After completion of the reaction, the reaction solution was slowly poured into 200 mL of water. The resulting mixture was stirred for 1 h. 1 hour later, the reaction mixture was extracted with dichloromethane and water and the organic layer was filtered through Celite. The filtrate was made anhydrous and concentrated under reduced pressure to afford 14.2 g (yield 99.2%) of 1-g.

### Synthesis Example 1-8. Synthesis of 1-h

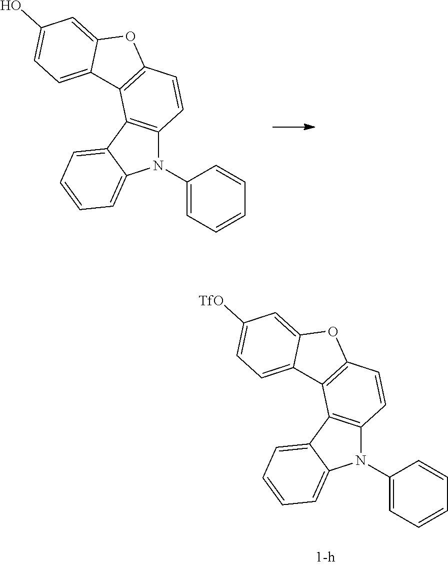 [SMILES: Oc1ccc2c(c1)oc1ccc3c(c4ccccc4n3-c3ccccc3)c12 ; O=S(=O)(Oc1ccc2c(c1)oc1ccc3c(c4ccccc4n3-c3ccccc3)c12)C(F)(F)F]

14.3 g (0.041 mol) of 1-g, 4.2 g (0.053 mol) of pyridine, and 140 mL of dichloromethane were placed in a 500 mL round-bottom flask under a nitrogen atmosphere. The mixture was stirred. The reaction solution was cooled to 0° C. and 13.9 g (0.049 mol) of trifluoromethanesulfonic anhydride was slowly added dropwise thereto. After the dropwise addition was completed, the reaction solution was heated to room temperature and stirred until the reaction was completed. After completion of the reaction, the reaction solution was poured into 300 mL of water, followed by stirring for 1 h. 1 hour later, the resulting mixture was extracted with dichloromethane and water. The organic layer was concentrated under reduced pressure and purified by column chromatography to afford 15.3 g (yield 77.6%) of 1-h.

### Synthesis Example 1-9. Synthesis of 1-i

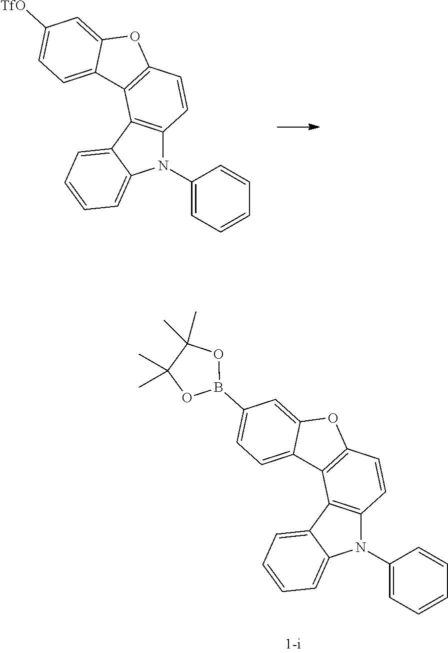 [SMILES: O=S(=O)(Oc1ccc2c(c1)oc1ccc3c(c4ccccc4n3-c3ccccc3)c12)C(F)(F)F ; CC1(C)OB(c2ccc3c(c2)oc2ccc4c(c5ccccc5n4-c4ccccc4)c23)OC1(C)C]

15.4 g (0.032 mol) of 1-h, 10.5 g (0.041 mol) of bis(pinacolato)diboron, 0.5 g (0.001 mol) of bis(diphenylphosphino)ferrocene dichloropalladium, 7.8 g (0.079 mol) of potassium acetate, and 150 mL of 1,4-dioxane were placed in a 300 mL round-bottom flask under a nitrogen atmosphere. The mixture was refluxed for 7 h. After completion of the reaction, the reaction solution was cooled to room temperature and filtered through Celite. The filtrate was concentrated and purified by column chromatography to afford 11.1 g (yield 75.4%) of 1-i.

### Synthesis Example 1-10. Synthesis of 1-i

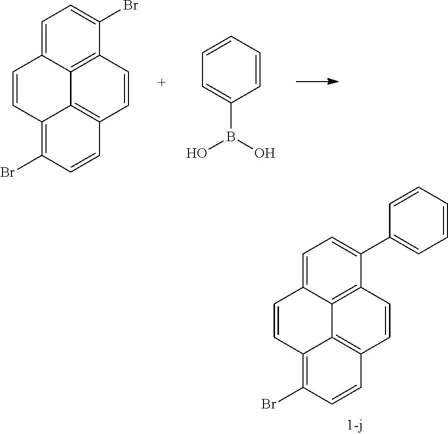 [SMILES: Brc1ccc2ccc3c(Br)ccc4ccc1c2c43 ; OB(O)c1ccccc1 ; Brc1ccc2ccc3c(-c4ccccc4)ccc4ccc1c2c43]

100 g (0.278 mol) of 1,6-dibromopyrene, 33.9 g (0.278 mol) of phenylboronic acid, 6.4 g (0.006 mol) of tetrakis(triphenylphosphine)palladium (Pd(PPh₃)₄), 88.3 g (0.833 mol) of sodium carbonate, 1400 mL of toluene, and 420 mL of water were placed in a 3000 mL round-bottom flask that had been purged with nitrogen. The mixture was refluxed for 9 h. After completion of the reaction, the reaction mixture was cooled to room temperature. The resulting solid was collected by filtration and discarded. The filtrate was extracted with ethyl acetate and water. The organic layer was made anhydrous and concentrated under reduced pressure. Column chromatography of the concentrate afforded 46 g (yield 45.7%) of 1-j.

### Synthesis Example 1-11. Synthesis of 4

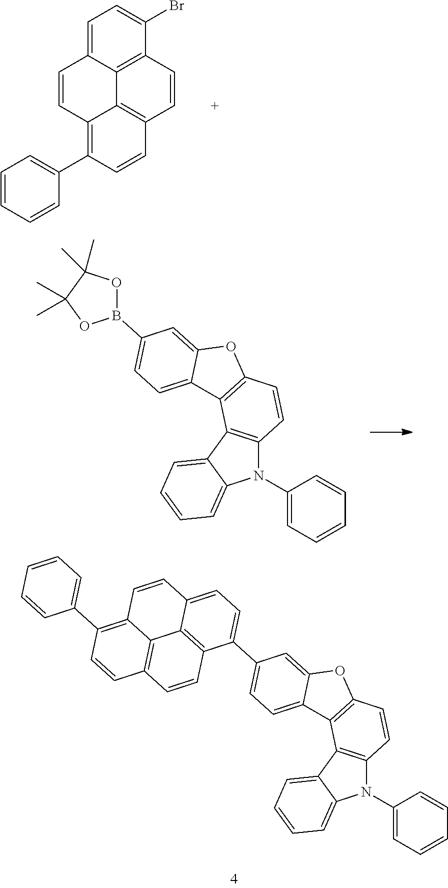

8 g (0.022 mol) of 1-j, 11 g (0.025 mol) of 1-i, 0.5 g (0.0004 mol) of tetrakis(triphenylphosphine)palladium, 5.3 g (0.038 mol) of potassium carbonate, 56 mL of toluene, 14 mL of ethanol, and 19 mL of water were placed in a 300 mL round-bottom flask under a nitrogen atmosphere. The mixture was refluxed for 6 h. After completion of the reaction, the reaction mixture was cooled to room temperature and extracted with ethyl acetate and water. The organic layer was made anhydrous and concentrated. Column chromatography of the concentrate gave 7.2 g (yield 52.7%) of 4.

MS (MALDI-TOF): m/z 609.21 [M]⁺

### Synthesis Example 2. Synthesis of 24

### Synthesis Example 2-1. Synthesis of 2-a

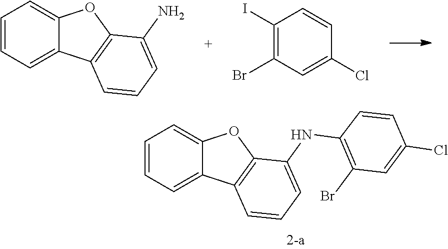

25 g (0.136 mol) of 4-aminodibenzofuran, 45.5 g (0.143 mol) of 2-bromo-4-chloro-1-iodobenzene, 0.6 g (0.003 mol) of palladium acetate, 1.7 g (0.003 mol) of 2,2′-bisdiphenylphosphino-1,1′-binaphthyl, 26.2 g (0.273 mol) of sodium tert-butoxide, and 250 mL of toluene were placed in a 500 mL round-bottom flask. The mixture was refluxed for 6 h. After completion of the reaction, the reaction solution was cooled to room temperature and extracted with toluene and water. The organic layer was concentrated. Column chromatography of the concentrate afforded 37 g (yield 72.8%) of 2-a.

### Synthesis Example 2-2. Synthesis of 2-b

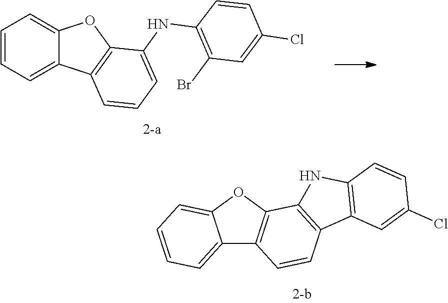 [SMILES: Clc1ccc(Nc2cccc3c2oc2ccccc23)c(Br)c1 ; Clc1ccc2[nH]c3c(ccc4c5ccccc5oc43)c2c1]

37 g (0.099 mol) of 2-a, 2.3 g (0.002 mol) of tetrakis(triphenylphosphine)palladium, 19.5 g (0.199 mol) of potassium acetate, and 300 mL of dimethylformamide were placed in a 500 mL round-bottom flask. The mixture was refluxed for 12 h. After completion of the reaction, the reaction solution was cooled to room temperature and filtered through Celite. The filtrate was concentrated under reduced pressure and purified by column chromatography to afford 20 g (yield 69%) of 2-b.

### Synthesis Example 2-3. Synthesis of 2-c

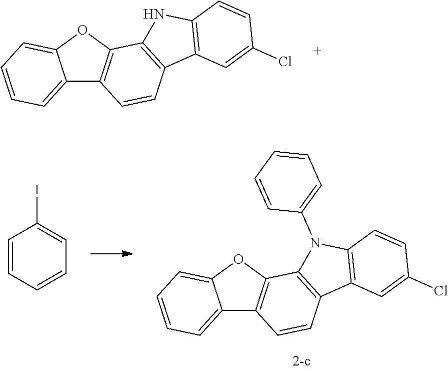 [SMILES: Clc1ccc2[nH]c3c(ccc4c5ccccc5oc43)c2c1 ; Ic1ccccc1 ; Clc1ccc2c(c1)c1ccc3c4ccccc4oc3c1n2-c1ccccc1]

2-c (yield 73.5%) was synthesized in the same manner as in Synthesis Example 1-6, except that 2-b was used instead of 1-e.

### Synthesis Example 2-4. Synthesis of 2-d

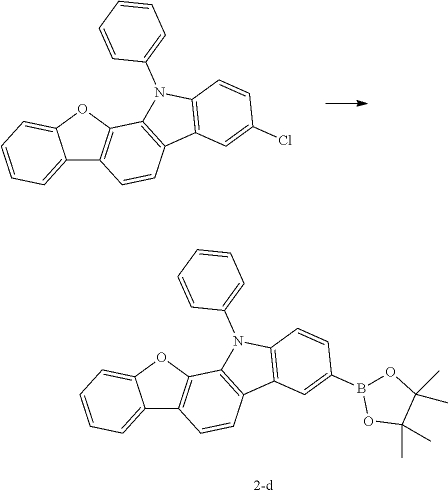 [SMILES: Clc1ccc2c(c1)c1ccc3c4ccccc4oc3c1n2-c1ccccc1 ; CC1(C)OB(c2ccc3c(c2)c2ccc4c5ccccc5oc4c2n3-c2ccccc2)OC1(C)C]

16 g (0.043 mol) of 2-c, 14.4 g (0.057 mol) of bis(pinacolato)diboron, 0.5 g (0.002 mol) of palladium acetate, 23.1 g (0.109 mol) of potassium phosphate, 2.3 g (0.006 mol) of S-Phos, and 160 mL of 1,4-dioxane were placed in a 300 mL round-bottom flask. The mixture was refluxed for 12 h. After completion of the reaction, the reaction solution was cooled to room temperature and filtered through Celite. The filtrate was concentrated and purified by column chromatography to afford 14 g (yield 70.1%) of 2-d.

### Synthesis Example 2-5. Synthesis of 24

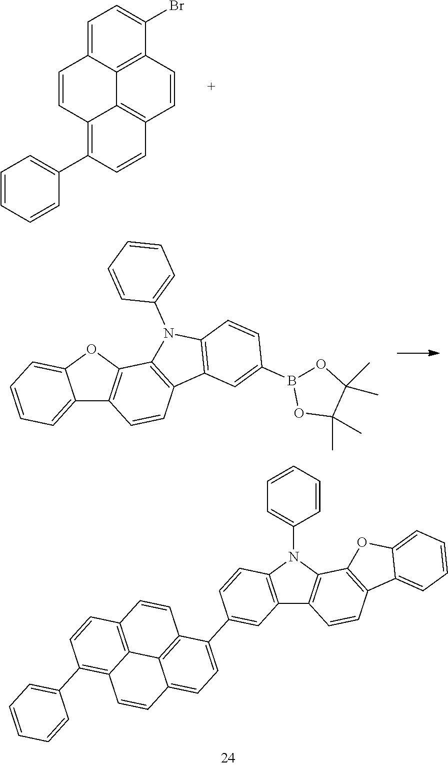

24 (yield 75.2%) was synthesized in the same manner as in Synthesis Example 1-11, except that 2-d was used instead of 1-i.

MS (MALDI-TOF): m/z 609.21 [M]⁺

### Synthesis Example 3. Synthesis of 57

### Synthesis Example 3-1. Synthesis of 3-a

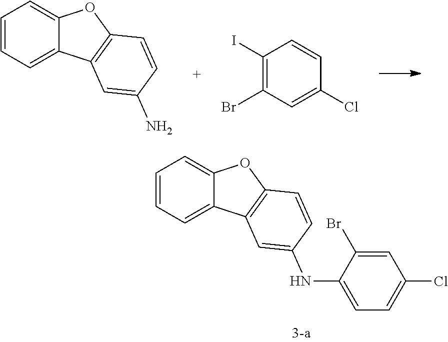

3-a (yield 74.6%) was synthesized in the same manner as in Synthesis Example 2-1, except that 2-aminodibenzofuran was used instead of 4-aminodibenzofuran.

### Synthesis Example 3-2. Synthesis of 3-b

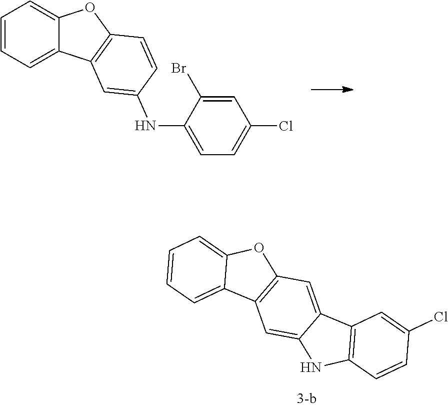 [SMILES: Clc1ccc(Nc2ccc3oc4ccccc4c3c2)c(Br)c1 ; Clc1ccc2[nH]c3cc4c(cc3c2c1)oc1ccccc14]

3-b (yield 71%) was synthesized in the same manner as in Synthesis Example 2-2, except that 3-a was used instead of 2-a.

### Synthesis Example 3-3. Synthesis of 3-c

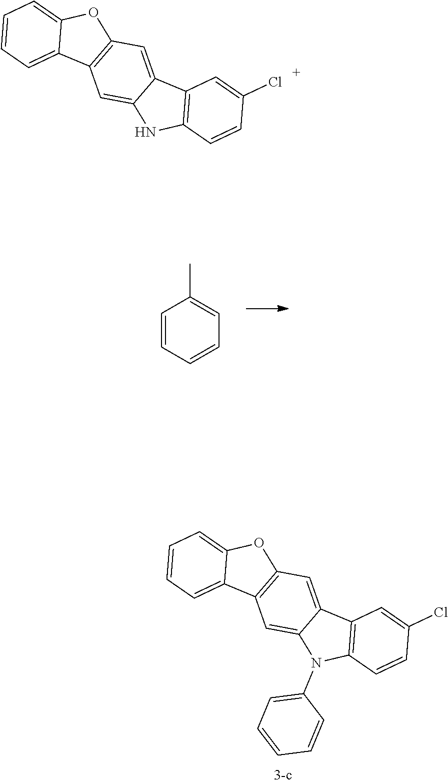

3-c (yield 63.5%) was synthesized in the same manner as in Synthesis Example 1-6, except that 3-b was used instead of 1-e.

### Synthesis Example 3-4. Synthesis of 3-d

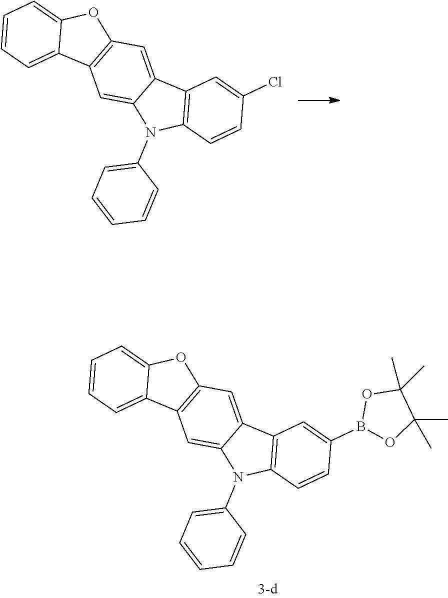 [SMILES: Clc1ccc2c(c1)c1cc3oc4ccccc4c3cc1n2-c1ccccc1 ; CC1(C)OB(c2ccc3c(c2)c2cc4oc5ccccc5c4cc2n3-c2ccccc2)OC1(C)C]

3-d (yield 72.4%) was synthesized in the same manner as in Synthesis Example 2-4, except that 3-c was used instead of 2-c.

### Synthesis Example 3-5. Synthesis of 57

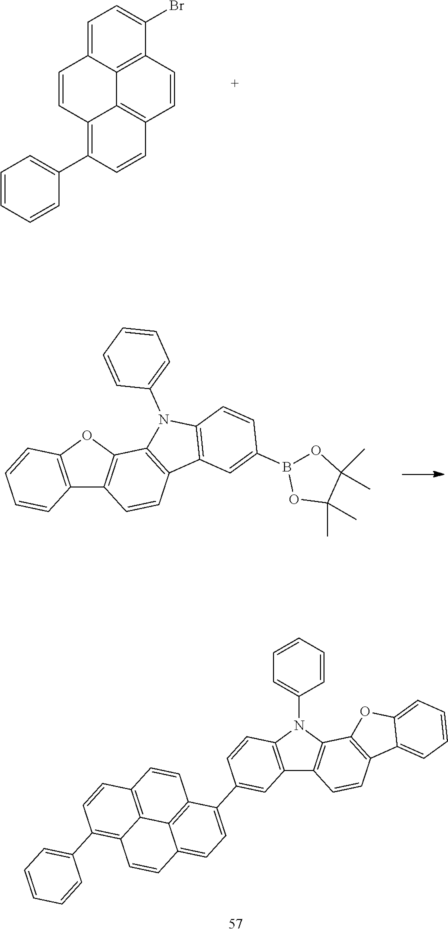 [SMILES: Brc1ccc2ccc3c(-c4ccccc4)ccc4ccc1c2c43 ; CC1(C)OB(c2ccc3c(c2)c2ccc4c5ccccc5oc4c2n3-c2ccccc2)OC1(C)C ; c1ccc(-c2ccc3ccc4c(-c5ccc6c(c5)c5ccc7c8ccccc8oc7c5n6-c5ccccc5)ccc5ccc2c3c54)cc1]

57 (yield 62%) was synthesized in the same manner as in Synthesis Example 1-11, except that 3-d was used instead of 1-i.

MS (MALDI-TOF): m/z 609.21 [M]⁺

### Synthesis Example 4. Synthesis of 73

### Synthesis Example 4-1. Synthesis of 4-a

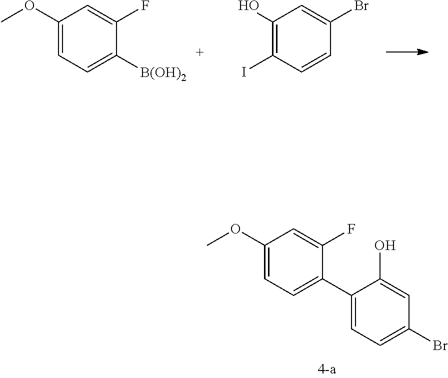 [SMILES: COc1ccc(B(O)O)c(F)c1 ; Oc1cc(Br)ccc1I ; COc1ccc(-c2ccc(Br)cc2O)c(F)c1]

4-a (yield 75%) was synthesized in the same manner as in Synthesis Example 1-1, except that 2-bromo-6-iodophenol was used instead of 3-bromo-2-iodophenol.

### Synthesis Example 4-2. Synthesis of 4-b

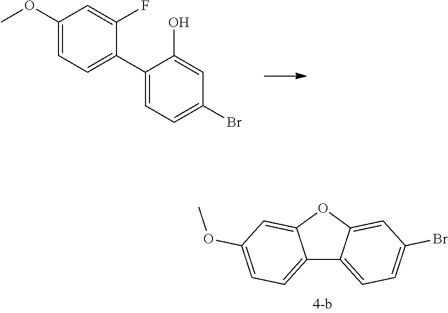 [SMILES: COc1ccc(-c2ccc(Br)cc2O)c(F)c1 ; COc1ccc2c(c1)oc1cc(Br)ccc12]

4-b (yield 76.5%) was synthesized in the same manner as in Synthesis Example 1-2, except that 4-a was used instead of 1-a.

### Synthesis Example 4-3. Synthesis of 4-c

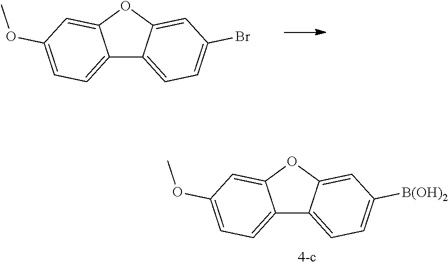 [SMILES: COc1ccc2c(c1)oc1cc(Br)ccc12 ; COc1ccc2c(c1)oc1cc(B(O)O)ccc12]

4-c (yield 72%) was synthesized in the same manner as in Synthesis Example 1-3, except that 4-b was used instead of 1-b.

### Synthesis Example 4-4. Synthesis of 4-d

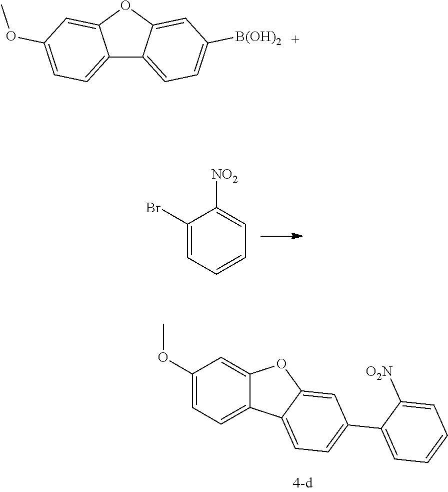 [SMILES: COc1ccc2c(c1)oc1cc(B(O)O)ccc12 ; O=[N+]([O-])c1ccccc1Br ; COc1ccc2c(c1)oc1cc(-c3ccccc3[N+](=O)[O-])ccc12]

4-d (yield 70%) was synthesized in the same manner as in Synthesis Example 1-4, except that 4-c was used instead of 1-c.

### Synthesis Example 4-5. Synthesis of 4-e

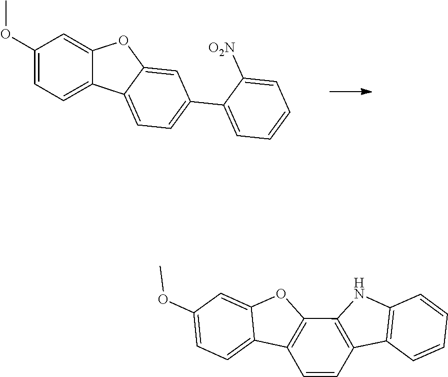 [SMILES: COc1ccc2c(c1)oc1cc(-c3ccccc3[N+](=O)[O-])ccc12 ; COc1ccc2c(c1)oc1c2ccc2c3ccccc3[nH]c21]

4-e (yield 64%) was synthesized in the same manner as in Synthesis Example 1-5, except that 4-d was used instead of 1-d.

### Synthesis Example 4-6. Synthesis of 4-f

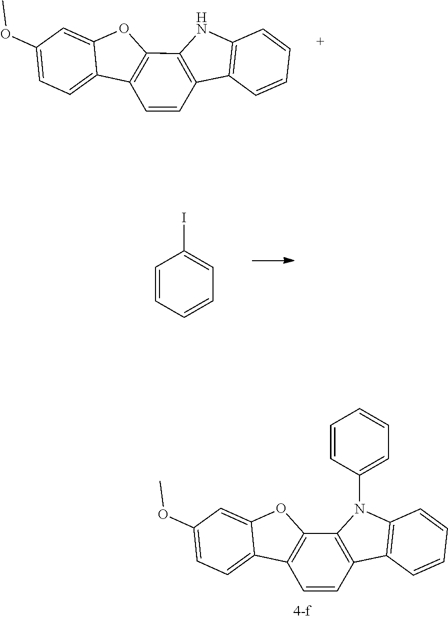 [SMILES: COc1ccc2c(c1)oc1c2ccc2c3ccccc3[nH]c21 ; Ic1ccccc1 ; COc1ccc2c(c1)oc1c2ccc2c3ccccc3n(-c3ccccc3)c21]

4-f (yield 83%) was synthesized in the same manner as in Synthesis Example 1-6, except that 4-e was used instead of 1-e.

### Synthesis Example 4-7. Synthesis of 4-q

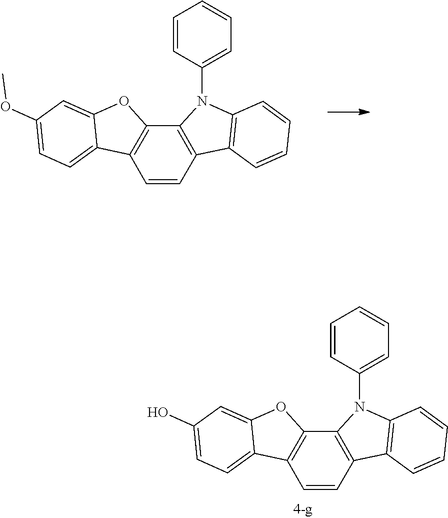 [SMILES: COc1ccc2c(c1)oc1c2ccc2c3ccccc3n(-c3ccccc3)c21 ; Oc1ccc2c(c1)oc1c2ccc2c3ccccc3n(-c3ccccc3)c21]

4-g (yield 98.5%) was synthesized in the same manner as in Synthesis Example 1-7, except that 4-f was used instead of 1-f.

### Synthesis Example 4-8. Synthesis of 4-h

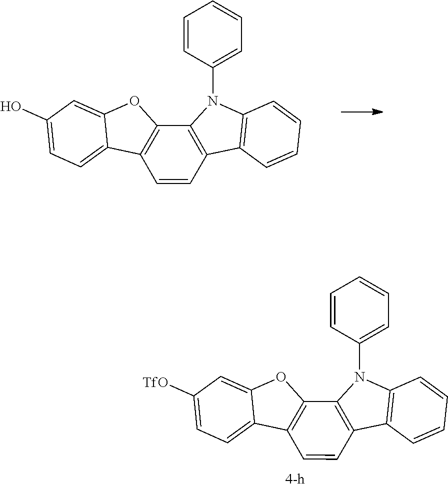 [SMILES: Oc1ccc2c(c1)oc1c2ccc2c3ccccc3n(-c3ccccc3)c21 ; O=S(=O)(Oc1ccc2c(c1)oc1c2ccc2c3ccccc3n(-c3ccccc3)c21)C(F)(F)F]

4-h (yield 78.2%) was synthesized in the same manner as in Synthesis Example 1-8, except that 4-g was used instead of 1-g.

### Synthesis Example 4-9. Synthesis of 4-i

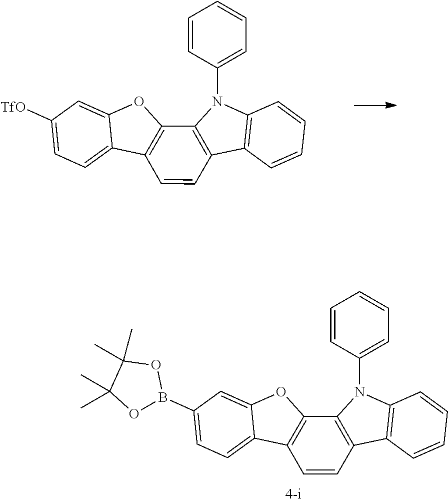 [SMILES: O=S(=O)(Oc1ccc2c(c1)oc1c2ccc2c3ccccc3n(-c3ccccc3)c21)C(F)(F)F ; CC1(C)OB(c2ccc3c(c2)oc2c3ccc3c4ccccc4n(-c4ccccc4)c32)OC1(C)C]

4-i (yield 76.4%) was synthesized in the same manner as in Synthesis Example 1-9, except that 4-h was used instead of 1-h.

### Synthesis Example 4-10. Synthesis of 4-i

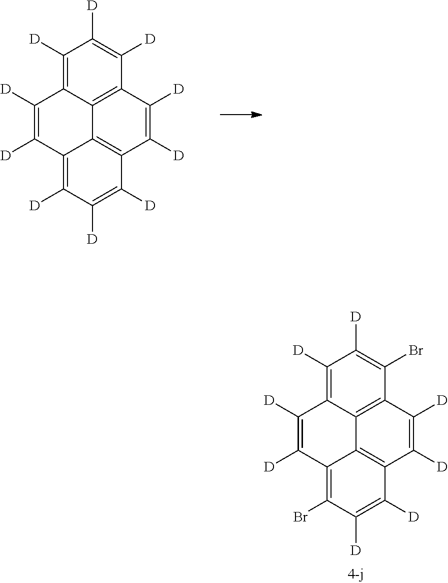 [SMILES: [2H]c1c([2H])c2c([2H])c([2H])c3c([2H])c([2H])c([2H])c4c([2H])c([2H])c(c1[2H])c2c34 ; [2H]c1c([2H])c2c([2H])c([2H])c3c(Br)c([2H])c([2H])c4c([2H])c([2H])c(c1Br)c2c43]

40 g (0.188 mol) of pyrene (D10) and 800 mL of dichloromethane were placed in a 2000 mL reactor under a nitrogen atmosphere. The mixture was stirred. The internal temperature of the reactor was lowered to ≤0° C. and a mixed solution of 58.7 g (0.367 mol) of bromine in 200 mL of dichloromethane was slowly added dropwise. After completion of the dropwise addition, the temperature was raised to room temperature, followed by stirring for 3 h. After completion of the reaction, the reaction solution was added with an aqueous sodium thiosulfate solution. The resulting mixture was stirred for 1 h, followed by filtration. The solid was purified with 1,2-dichlorobenzene to afford 31 g (yield 44.7%) of 4-j.

### Synthesis Example 4-11. Synthesis of 4-k

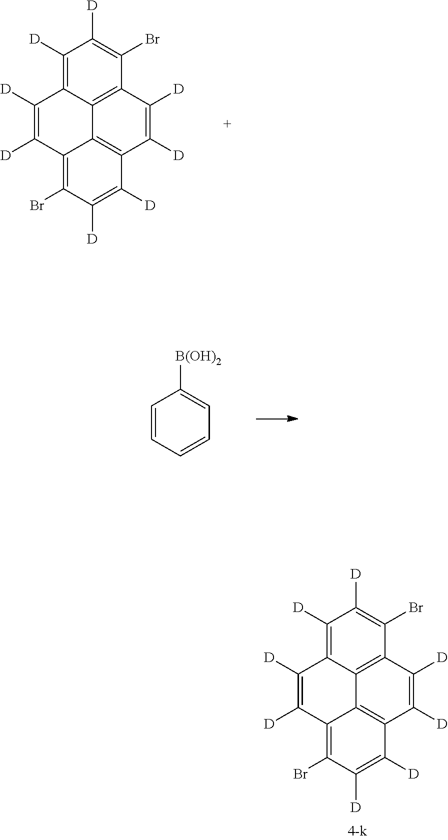 [SMILES: [2H]c1c([2H])c2c([2H])c([2H])c3c(Br)c([2H])c([2H])c4c([2H])c([2H])c(c1Br)c2c43 ; OB(O)c1ccccc1 ; [2H]c1c([2H])c2c([2H])c([2H])c3c(Br)c([2H])c([2H])c4c([2H])c([2H])c(c1Br)c2c43]

4-k (yield 68.4%) was synthesized in the same manner as in Synthesis Example 1-10, except that 4-j was used instead of 1,6-dibromopyrene.

### Synthesis Example 4-12. Synthesis of 4-1

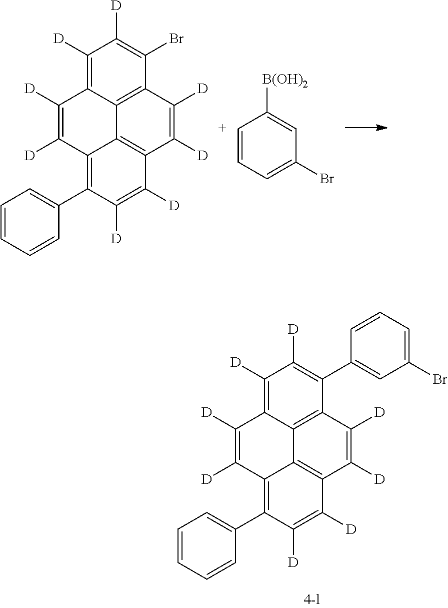 [SMILES: [2H]c1c([2H])c2c([2H])c([2H])c3c(-c4ccccc4)c([2H])c([2H])c4c([2H])c([2H])c(c1Br)c2c43 ; OB(O)C1=CC(Br)=CC=[C+]1 ; [2H]c1c([2H])c2c([2H])c([2H])c3c(-c4cccc(Br)c4)c([2H])c([2H])c4c([2H])c([2H])c(c1-c1ccccc1)c2c43]

4-1 (yield 70%) was synthesized in the same manner as in Synthesis Example 1-10, except that 4-k and 3-bromophenylboronic acid were used instead of 1,6-dibromopyrene and phenylboronic acid, respectively.

### Synthesis Example 4-13. Synthesis of 73

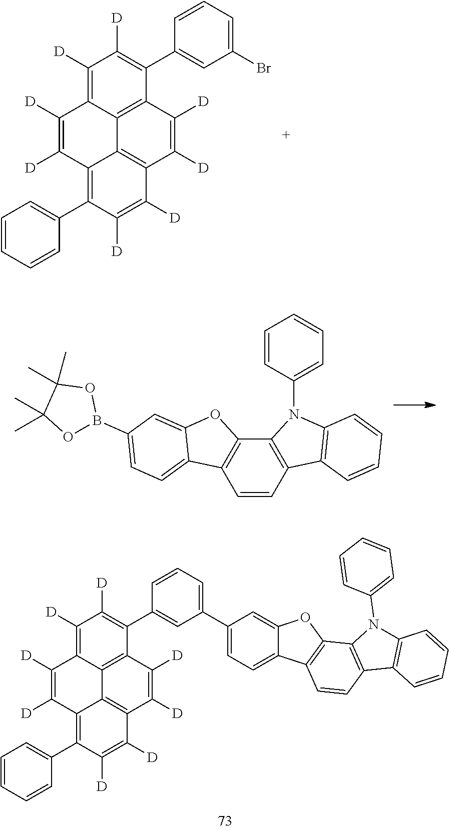 [SMILES: [2H]c1c([2H])c2c([2H])c([2H])c3c(-c4cccc(Br)c4)c([2H])c([2H])c4c([2H])c([2H])c(c1-c1ccccc1)c2c43 ; CC1(C)OB(c2ccc3c(c2)oc2c3ccc3c4ccccc4n(-c4ccccc4)c32)OC1(C)C ; [2H]c1c([2H])c2c([2H])c([2H])c3c(-c4cccc(-c5ccc6c(c5)oc5c6ccc6c7ccccc7n(-c7ccccc7)c65)c4)c([2H])c([2H])c4c([2H])c([2H])c(c1-c1ccccc1)c2c43]

73 (yield 68%) was synthesized in the same manner as in Synthesis Example 1-11, except that 4-1 and 4-i were used instead of 1-j and 1-i, respectively. MS (MALDI-TOF): m/z 609.21 [M]⁺

### Synthesis Example 5. Synthesis of 79

### Synthesis Example 5-1. Synthesis of 5-a

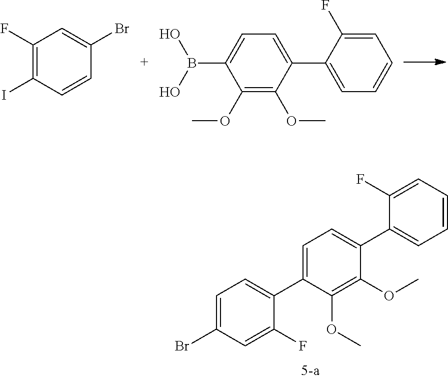 [SMILES: Fc1cc(Br)ccc1I ; COc1c(B(O)O)ccc(-c2ccccc2F)c1OC ; COc1c(-c2ccccc2F)ccc(-c2ccc(Br)cc2F)c1OC]

5-a (yield 76.4%) was synthesized in the same manner as in Synthesis Example 1-10, except that 4-bromo-2-fluoro-1-iodobenzene and B-(2′-fluoro-2,3-dimethoxy(1,1′-biphenyl)-4-yl)boronic acid were used instead of 1,6-dibromopyrene and phenylboronic acid, respectively.

### Synthesis Example 5-2. Synthesis of 5-b

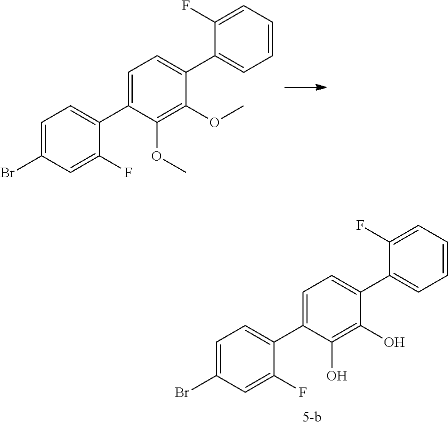 [SMILES: COc1c(-c2ccccc2F)ccc(-c2ccc(Br)cc2F)c1OC ; Oc1c(-c2ccccc2F)ccc(-c2ccc(Br)cc2F)c1O]

5-b (yield 88%) was synthesized in the same manner as in Synthesis Example 1-7, except that 5-a was used instead of 1-f.

### Synthesis Example 5-3. Synthesis of 5-c

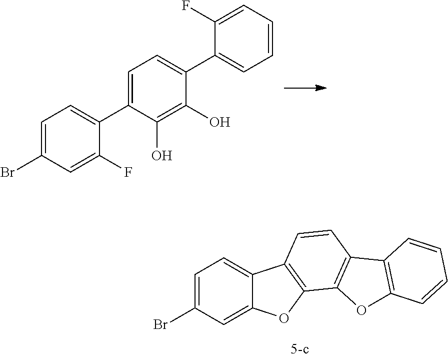 [SMILES: Oc1c(-c2ccccc2F)ccc(-c2ccc(Br)cc2F)c1O ; Brc1ccc2c(c1)oc1c2ccc2c3ccccc3oc21]

22.3 g (59 mmol) of 5-b, 13 g (94.5 mmol) of potassium carbonate, and 200 mL of 1-methyl-pyrrolidinone were placed in a round-bottom flask under a nitrogen atmosphere. The mixture was stirred at 150° C. for 12 h. After completion of the reaction, the reaction mixture was allowed to stand for layer separation. The organic layer was concentrated under reduced pressure and purified by column chromatography to afford 14.3 g (yield 72%) of 5-c.

### Synthesis Example 5-4. Synthesis of 5-d

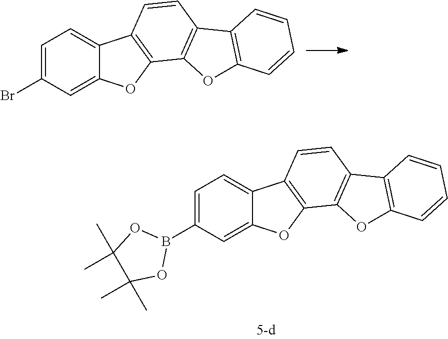 [SMILES: Brc1ccc2c(c1)oc1c2ccc2c3ccccc3oc21 ; CC1(C)OB(c2ccc3c(c2)oc2c3ccc3c4ccccc4oc32)OC1(C)C]

13.8 g (41 mmol) of 5-c, 12.4 g (49 mmol) of bis(pinacolato)diborane, 2 g (2.4 mmol) of tris(dibenzylideneacetone)palladium, 11.6 g (122 mmol) of potassium acetate, 2.7 g (9.8 mmol) of tricyclohexylphosphine, and 150 mL of N-dimethylformamide were placed in a round-bottom flask under a nitrogen atmosphere. The mixture was refluxed. After completion of the reaction, the reaction mixture was allowed to stand for layer separation. The organic layer was concentrated under reduced pressure and purified by column chromatography to afford 10.2 g (65%) of 5-d.

### Synthesis Example 5-5. Synthesis of 5-e

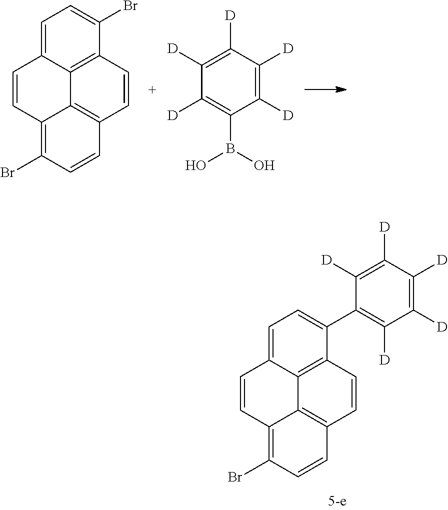

5-e (yield 48%) was synthesized in the same manner as in Synthesis Example 1-10, except that phenyl(D5)boronic acid was used instead of phenylboronic acid.

### Synthesis Example 5-6. Synthesis of 79

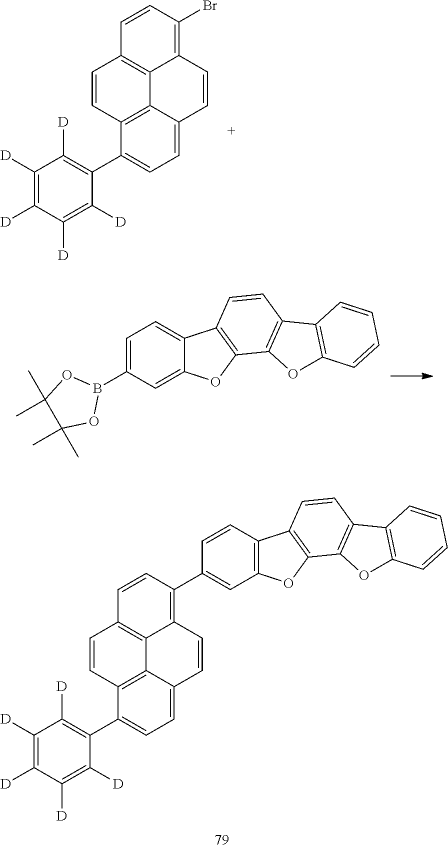 [SMILES: [2H]c1c([2H])c([2H])c(-c2ccc3ccc4c(Br)ccc5ccc2c3c54)c([2H])c1[2H] ; CC1(C)OB(c2ccc3c(c2)oc2c3ccc3c4ccccc4oc32)OC1(C)C ; [2H]c1c([2H])c([2H])c(-c2ccc3ccc4c(-c5ccc6c(c5)oc5c6ccc6c7ccccc7oc65)ccc5ccc2c3c54)c([2H])c1[2H]]

79 (yield 62%) was synthesized in the same manner as in Synthesis Example 1-11, except that 5-e and 5-d were used instead of 1-j and 1-i, respectively. MS (MALDI-TOF): m/z 609.21 [M]⁺

### Synthesis Example 6. Synthesis of 93

### Synthesis Example 6-1. Synthesis of 6-a

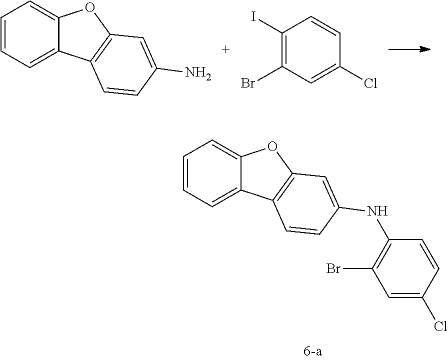 [SMILES: Nc1ccc2c(c1)oc1ccccc12 ; Clc1ccc(I)c(Br)c1 ; Clc1ccc(Nc2ccc3c(c2)oc2ccccc23)c(Br)c1]

6-a (yield 74%) was synthesized in the same manner as in Synthesis Example 2-1, except that 3-aminodibenzofuran was used instead of 4-aminodibenzofuran.

### Synthesis Example 6-2. Synthesis of 6-b

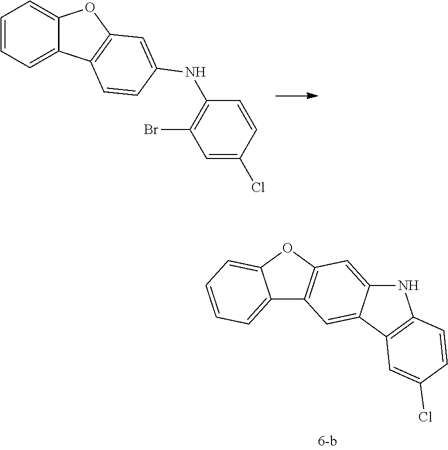 [SMILES: Clc1ccc(Nc2ccc3c(c2)oc2ccccc23)c(Br)c1 ; Clc1ccc2[nH]c3cc4oc5ccccc5c4cc3c2c1]

6-b (yield 68%) was synthesized in the same manner as in Synthesis Example 2-2, except that 6-a was used instead of 2-a.

### Synthesis Example 6-3. Synthesis of 6-c

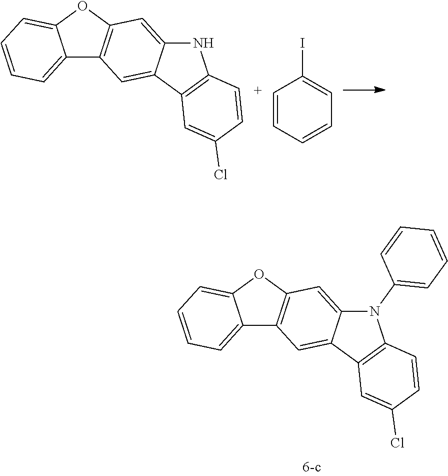 [SMILES: Clc1ccc2[nH]c3cc4oc5ccccc5c4cc3c2c1 ; IC1=CC=CC=[C+]1 ; Clc1ccc2c(c1)c1cc3c(cc1n2-c1ccccc1)oc1ccccc13]

6-c (yield 65%) was synthesized in the same manner as in Synthesis Example 1-6, except that 6-b was used instead of 1-e.

### Synthesis Example 6-4. Synthesis of 6-d

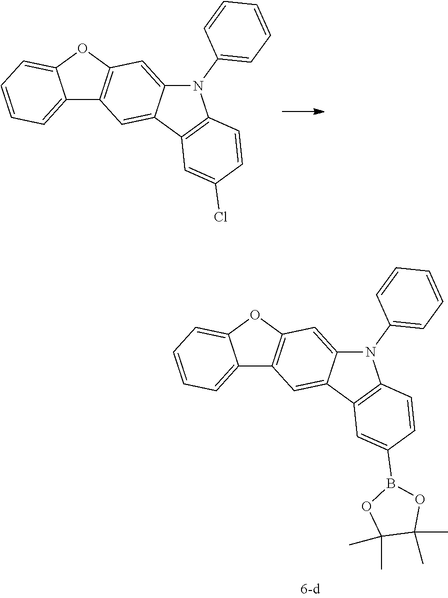 [SMILES: Clc1ccc2c(c1)c1cc3c(cc1n2-c1ccccc1)oc1ccccc13 ; CC1(C)OB(c2ccc3c(c2)c2cc4c(cc2n3-c2ccccc2)oc2ccccc24)OC1(C)C]

6-d (yield 73%) was synthesized in the same manner as in Synthesis Example 2-4, except that 6-c was used instead of 2-c.

### Synthesis Example 6-5. Synthesis of 93

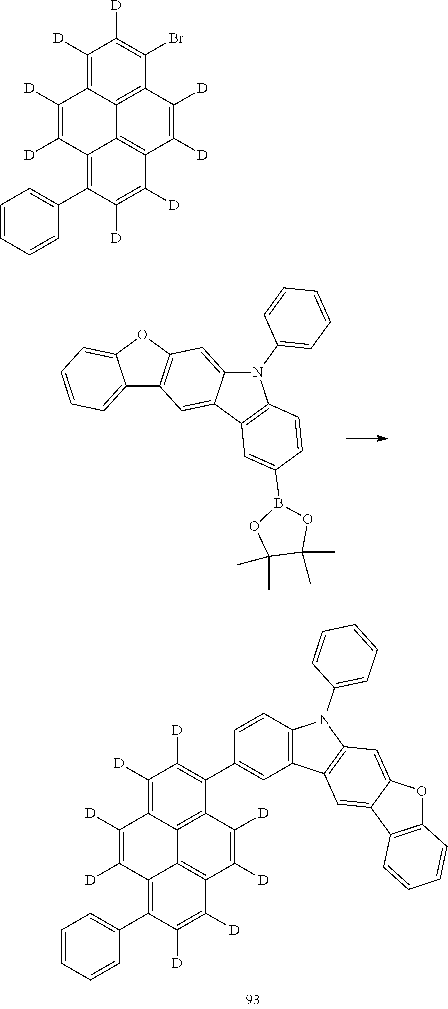 [SMILES: [2H]c1c([2H])c2c([2H])c([2H])c3c(-c4ccccc4)c([2H])c([2H])c4c([2H])c([2H])c(c1Br)c2c43 ; CC1(C)OB(c2ccc3c(c2)c2cc4c(cc2n3-c2ccccc2)oc2ccccc24)OC1(C)C ; [2H]c1c([2H])c2c([2H])c([2H])c3c(-c4ccc5c(c4)c4cc6c(cc4n5-c4ccccc4)oc4ccccc46)c([2H])c([2H])c4c([2H])c([2H])c(c1-c1ccccc1)c2c43]

93 (yield 76%) was synthesized in the same manner as in Synthesis Example 1-11, except that 4-k and 6-d were used instead of 1-j and 1-i, respectively.

MS (MALDI-TOF): m/z 617.26 [M]⁺

### Examples 1-5: Fabrication of Organic Light Emitting Devices

ITO glass was patterned to have a light emitting area of 2 mm×2 mm, followed by cleaning. After the cleaned ITO glass was mounted in a vacuum chamber, the base pressure was adjusted to 1×10⁻⁷ torr. 2-TNATA (400 Å) and HT (200 Å) were sequentially formed into layers on the ITO. A mixture of the inventive host compound shown in Table 1 and BD as a dopant compound (3 wt %) was formed into a 250 Å thick light emitting layer. Thereafter, the compound represented by Formula E-1 was formed into a 300 Å thick electron transport layer on the light emitting layer. Liq was formed into a 10 Å thick electron injecting layer on the electron transport layer. Al was formed into a 1000 Å thick cathode on the electron injecting layer, completing the fabrication of an organic light emitting device. The luminescent properties of the organic light emitting device were measured at 10 mA/cm².

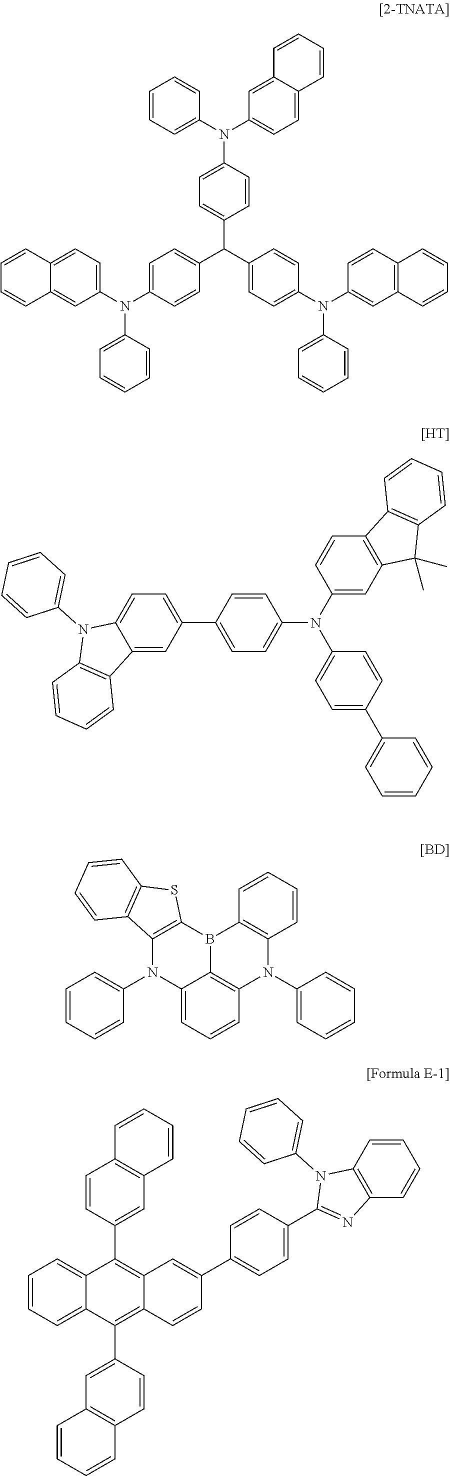 [SMILES: c1ccc(N(c2ccc(C(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N2c3ccccc3B3c4sc5ccccc5c4N(c4ccccc4)c4cccc2c43)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Comparative Examples 1-2

Organic light emitting devices were fabricated in the same manner as in Examples 1-5, except that BH1 or BH2 was used instead of the inventive host compound. The luminescent properties of the organic light emitting devices were measured at 10 mA/cm². The structures of BH1 and BH2 are as follow:

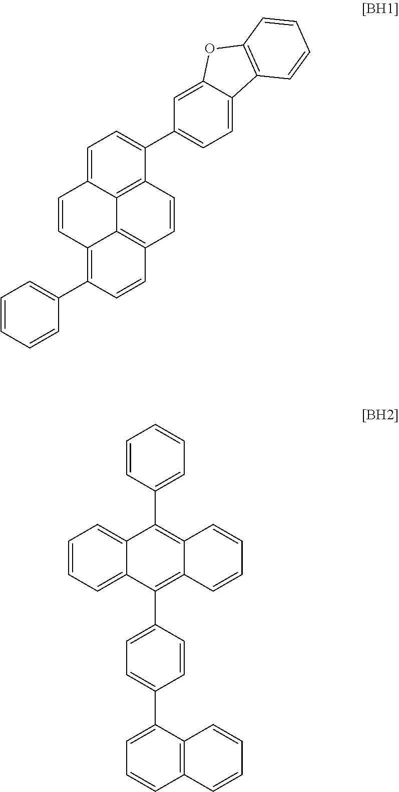

TABLE 1

| Example No. | Host | V | EQE | T97 |
|---|---|---|---|---|
| Example 1 | 4 | 3.7 | 12.2 | 200 |
| Example 2 | 24 | 3.6 | 12.4 | 210 |
| Example 3 | 57 | 3.6 | 11.9 | 240 |
| Example 4 | 73 | 3.7 | 12 | 220 |
| Example 5 | 79 | 3.6 | 11.5 | 210 |
| Example 6 | 93 | 3.7 | 11.4 | 235 |
| Comparative Example 1 | BH1 | 4.1 | 9.2 | 150 |
| Comparative Example 2 | BH2 | 3.9 | 9.7 | 135 |

As can be seen from the results in Table 1, the organic light emitting devices of Examples 1-5, each of which employed the inventive host compound for the light emitting layer, were driven at low voltages and showed high external quantum efficiencies and significantly improved life characteristics compared to the organic light emitting device of Comparative Example 1 employing the compound whose structural features are contrasted with those of the inventive compound and the organic light emitting device of Comparative Example 2 employing the anthracene derivative widely used in the art. These results concluded that the use of the inventive compounds makes the organic light emitting devices highly efficient.

### INDUSTRIAL APPLICABILITY

The organic light emitting device of the present invention includes a light emitting layer employing the pyrene derivative with a specific structure as a host. The use of the host ensures excellent luminescent properties and high efficiency of the device. Due to these advantages, the organic light emitting device of the present invention can find useful industrial applications in not only lighting systems but also a variety of displays, including flat panel displays, flexible displays, and wearable displays.

What is claimed is:

1. A compound represented by Formula I: 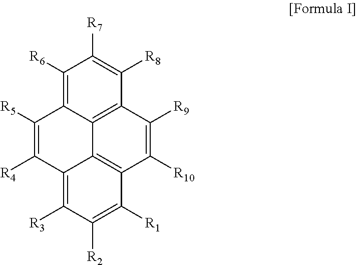
wherein R₁ to R₁₀ are the same as or different from each other and are each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₁-C₃₀ alkylamine, substituted or unsubstituted C₆-C₅₀ arylamine, substituted or unsubstituted C₁-C₃₀ alkylsilyl, substituted or unsubstituted C₆-C₅₀ arylsilyl, substituted or unsubstituted C₁-C₃₀ alkoxy, and substituted or unsubstituted C₆-C₅₀ aryloxy, with the proviso that at least one of R₁ to R₁₀ is linked to one of Structural Formulas 1 to 4: 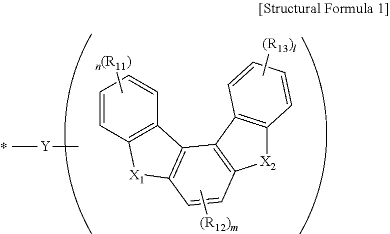
wherein X₁ and X₂ are each independently S, O or NR₁₄, R₁₄ is selected from hydrogen, deuterium, substituted or unsubstituted C₁-C₃₀ alkyl, substituted or unsubstituted C₃-C₃₀ cycloalkyl, substituted or unsubstituted C₆-C₅₀ aryl, and substituted or unsubstituted C₂-C₅₀ heteroaryl, R₁₁ to R₁₃ are the same as or different from each other and are each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₁-C₃₀ alkylamine, substituted or unsubstituted C₆-C₅₀ arylamine, substituted or unsubstituted C₁-C₃₀ alkylsilyl, substituted or unsubstituted C₆-C₅₀ arylsilyl, substituted or unsubstituted C₁-C₃₀ alkoxy, and substituted or unsubstituted C₆-C₅₀ aryloxy, the asterisk (*) represents a site where one of R₁ to R₁₀ is bonded, Y is a single bond or is selected from substituted or unsubstituted C₆-C₃₀ arylene and substituted or unsubstituted C₂-C₃₀ heteroarylene, with the proviso that one of R₁₁ to R₁₃ is linked to Y, each of l and n is an integer from 1 to 4, and m is an integer of 1 or 2, provided that when l is 2 or greater, the groups R₁₃ are the same as or different from each other, when m is 2 or greater, the groups R₁₂ are the same as or different from each other, and when n is 2 or greater, the groups R₁₁ are the same as or different from each other; 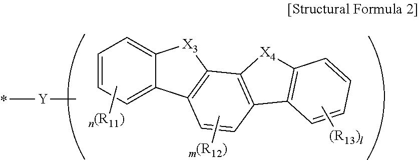
wherein X₃ to X₄ are as defined for X₁ and X₂ in Structural Formula 1 and R₁₁ to R₁₃, the asterisk (*), Y, l, n, and m are as defined in Structural Formula 1; 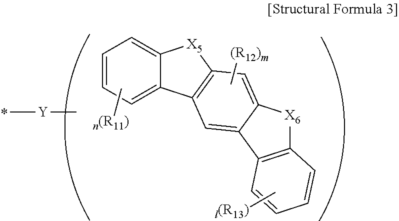
wherein X₅ to X₆ are as defined for X₁ and X₂ in Structural Formula 1 and R₁₁ to R₁₃, the asterisk (*), Y, l, n, and m are as defined in Structural Formula 1; and 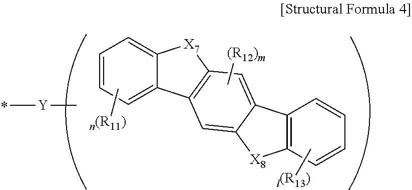
wherein X₇ to X₈ are as defined for X₁ and X₂ in Structural Formula 1 and R₁₁ to R₁₃, the asterisk (*), Y, l, n, and m are as defined in Structural Formula 1.

2. The compound according to claim 1, wherein at least one of R₁, R₃, R₆, and R₈ is linked to the structure represented by one of Structural Formulas 1 to 4.

3. The compound according to claim 1, wherein at least one of R₁ to R₁₀ is substituted or unsubstituted C₆-C₁₈ aryl.

4. The compound according to claim 1, wherein the compound represented by Formula I is selected from the following compounds 1 to 102:

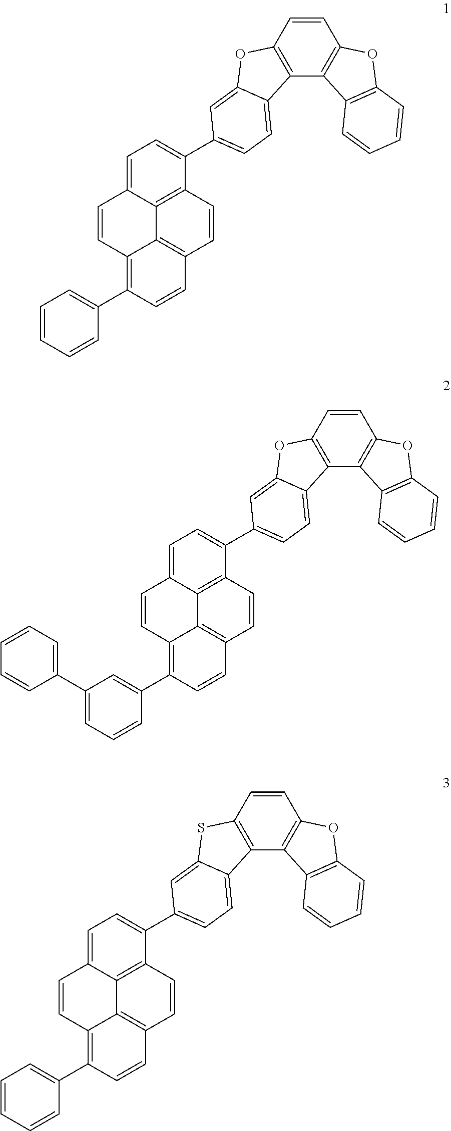 [SMILES: c1ccc(-c2ccc3ccc4c(-c5ccc6c(c5)oc5ccc7oc8ccccc8c7c56)ccc5ccc2c3c54)cc1 ; c1ccc(-c2cccc(-c3ccc4ccc5c(-c6ccc7c(c6)oc6ccc8oc9ccccc9c8c67)ccc6ccc3c4c65)c2)cc1 ; c1ccc(-c2ccc3ccc4c(-c5ccc6c(c5)sc5ccc7oc8ccccc8c7c56)ccc5ccc2c3c54)cc1]

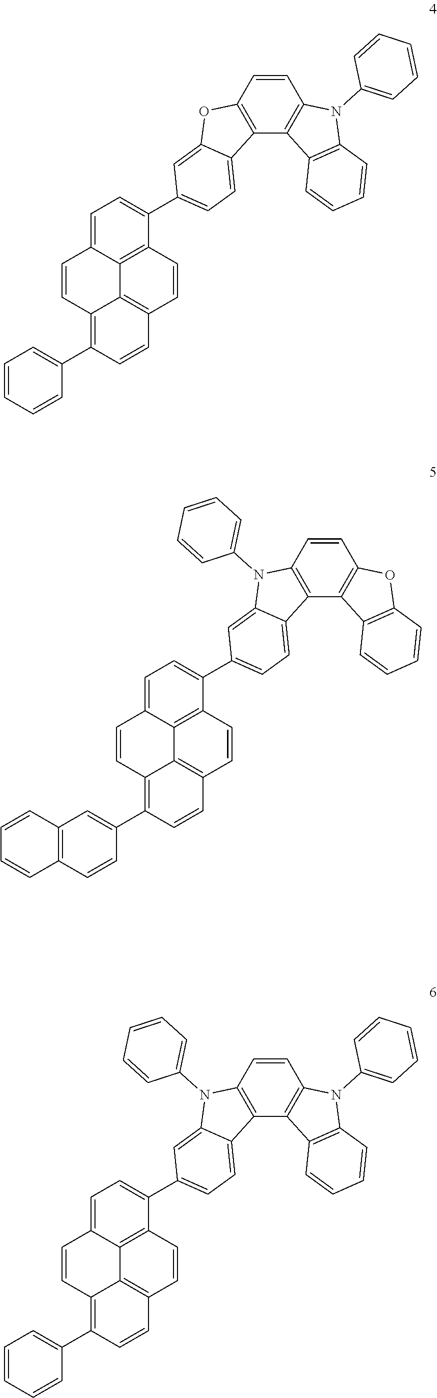 [SMILES: c1ccc(-c2ccc3ccc4c(-c5ccc6c(c5)oc5ccc7c(c8ccccc8n7-c7ccccc7)c56)ccc5ccc2c3c54)cc1 ; c1ccc(-n2c3cc(-c4ccc5ccc6c(-c7ccc8ccccc8c7)ccc7ccc4c5c76)ccc3c3c4c(ccc32)oc2ccccc24)cc1 ; c1ccc(-c2ccc3ccc4c(-c5ccc6c7c8c9ccccc9n(-c9ccccc9)c8ccc7n(-c7ccccc7)c6c5)ccc5ccc2c3c54)cc1]

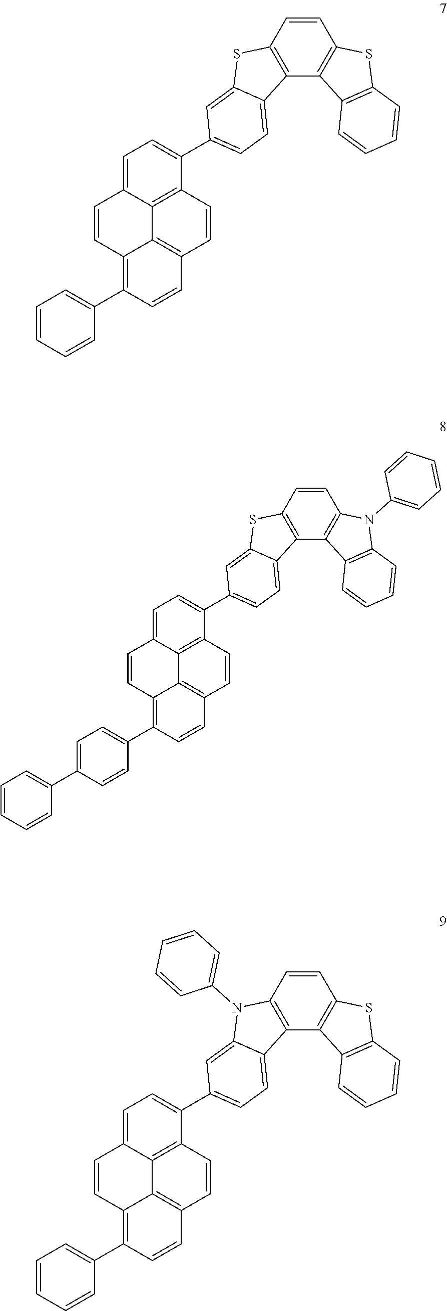 [SMILES: c1ccc(-c2ccc3ccc4c(-c5ccc6c(c5)sc5ccc7sc8ccccc8c7c56)ccc5ccc2c3c54)cc1 ; c1ccc(-c2ccc(-c3ccc4ccc5c(-c6ccc7c(c6)sc6ccc8c(c9ccccc9n8-c8ccccc8)c67)ccc6ccc3c4c65)cc2)cc1 ; c1ccc(-c2ccc3ccc4c(-c5ccc6c7c8c(ccc7n(-c7ccccc7)c6c5)sc5ccccc58)ccc5ccc2c3c54)cc1]

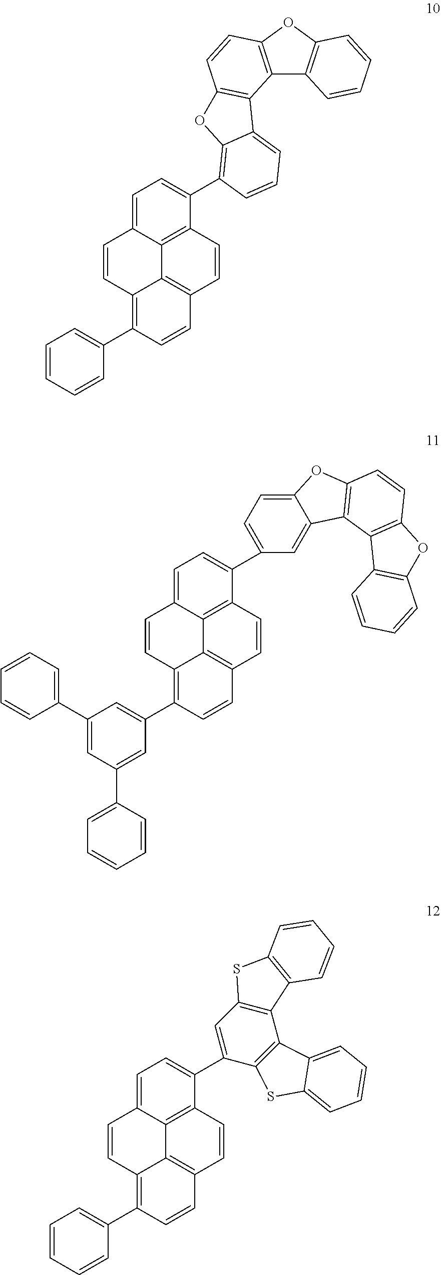 [SMILES: c1ccc(-c2ccc3ccc4c(-c5cccc6c5oc5ccc7oc8ccccc8c7c56)ccc5ccc2c3c54)cc1 ; c1ccc(-c2cc(-c3ccccc3)cc(-c3ccc4ccc5c(-c6ccc7oc8ccc9oc%10ccccc%10c9c8c7c6)ccc6ccc3c4c65)c2)cc1 ; c1ccc(-c2ccc3ccc4c(-c5cc6sc7ccccc7c6c6c5sc5ccccc56)ccc5ccc2c3c54)cc1]

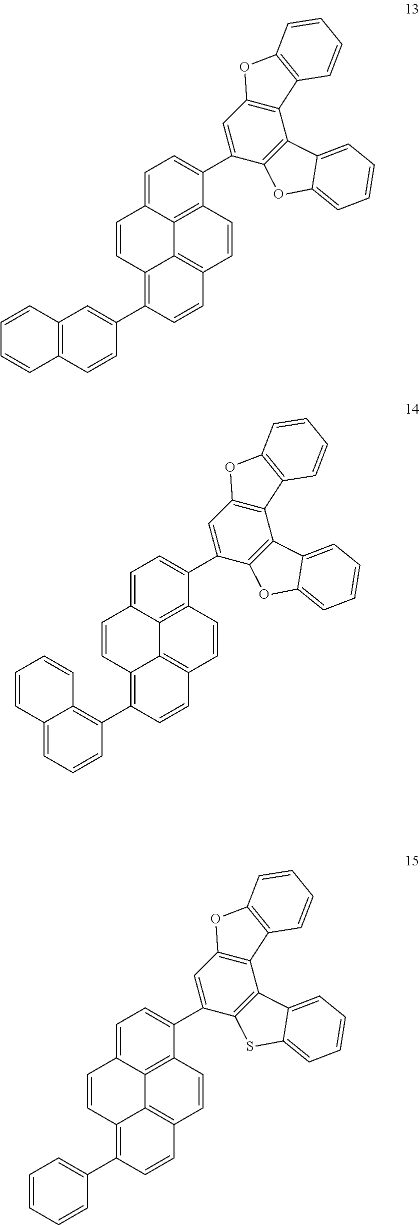 [SMILES: c1ccc2cc(-c3ccc4ccc5c(-c6cc7oc8ccccc8c7c7c6oc6ccccc67)ccc6ccc3c4c65)ccc2c1 ; c1ccc2c(-c3ccc4ccc5c(-c6cc7oc8ccccc8c7c7c6oc6ccccc67)ccc6ccc3c4c65)cccc2c1 ; c1ccc(-c2ccc3ccc4c(-c5cc6oc7ccccc7c6c6c5sc5ccccc56)ccc5ccc2c3c54)cc1]

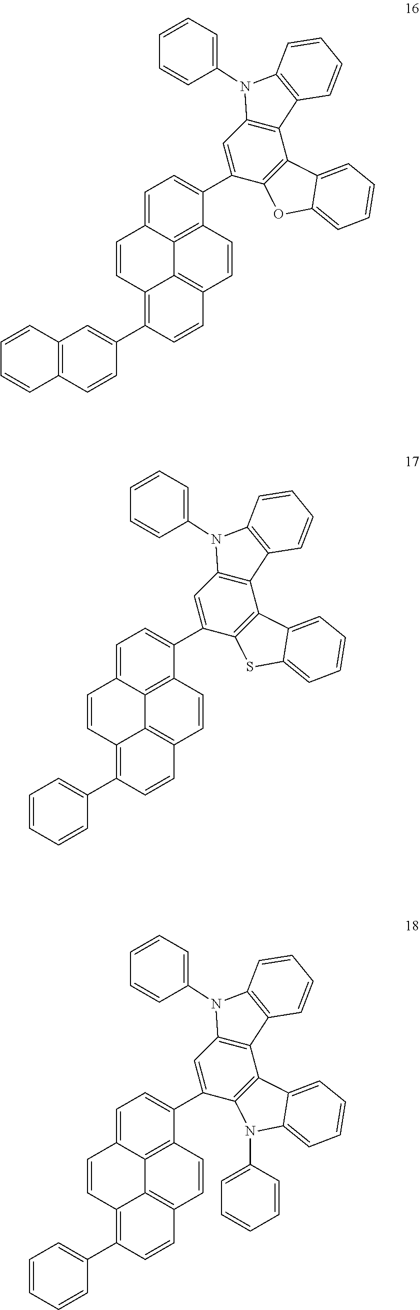 [SMILES: c1ccc(-n2c3ccccc3c3c4c(oc5ccccc54)c(-c4ccc5ccc6c(-c7ccc8ccccc8c7)ccc7ccc4c5c76)cc32)cc1 ; c1ccc(-c2ccc3ccc4c(-c5cc6c(c7ccccc7n6-c6ccccc6)c6c5sc5ccccc56)ccc5ccc2c3c54)cc1 ; c1ccc(-c2ccc3ccc4c(-c5cc6c(c7ccccc7n6-c6ccccc6)c6c7ccccc7n(-c7ccccc7)c56)ccc5ccc2c3c54)cc1]

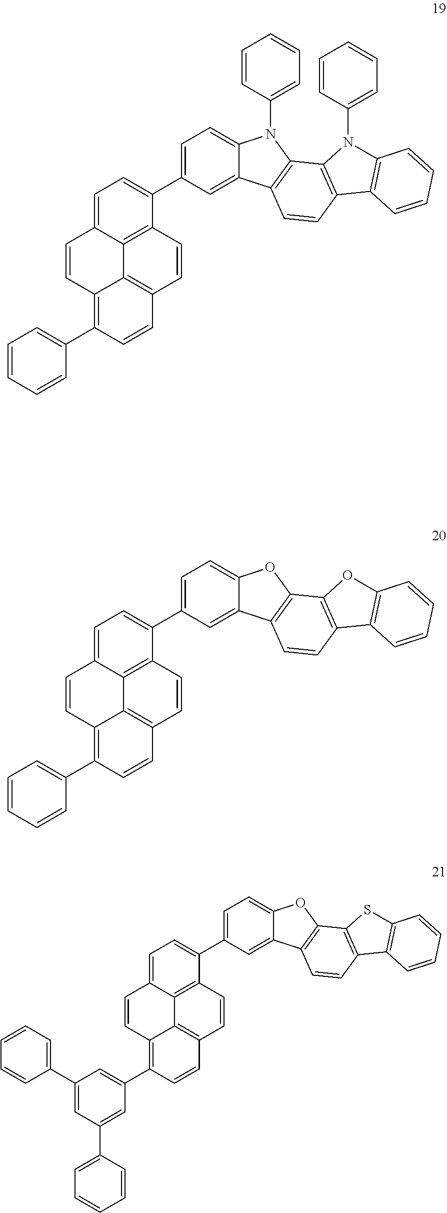 [SMILES: c1ccc(-c2ccc3ccc4c(-c5ccc6c(c5)c5ccc7c8ccccc8n(-c8ccccc8)c7c5n6-c5ccccc5)ccc5ccc2c3c54)cc1 ; c1ccc(-c2ccc3ccc4c(-c5ccc6oc7c(ccc8c9ccccc9oc87)c6c5)ccc5ccc2c3c54)cc1 ; c1ccc(-c2cc(-c3ccccc3)cc(-c3ccc4ccc5c(-c6ccc7oc8c(ccc9c%10ccccc%10sc98)c7c6)ccc6ccc3c4c65)c2)cc1]

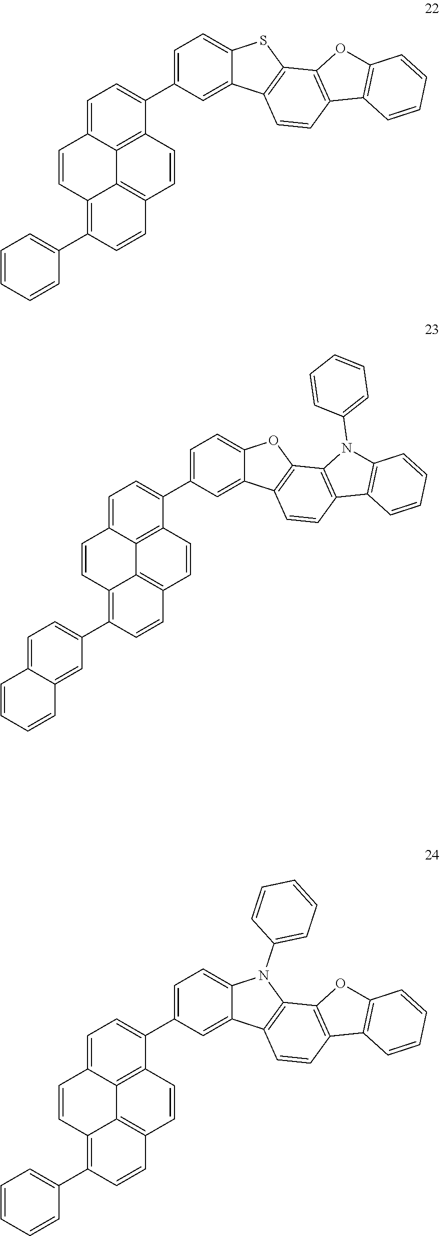 [SMILES: c1ccc(-c2ccc3ccc4c(-c5ccc6sc7c(ccc8c9ccccc9oc87)c6c5)ccc5ccc2c3c54)cc1 ; c1ccc(-n2c3ccccc3c3ccc4c5cc(-c6ccc7ccc8c(-c9ccc%10ccccc%10c9)ccc9ccc6c7c98)ccc5oc4c32)cc1 ; c1ccc(-c2ccc3ccc4c(-c5ccc6c(c5)c5ccc7c8ccccc8oc7c5n6-c5ccccc5)ccc5ccc2c3c54)cc1]

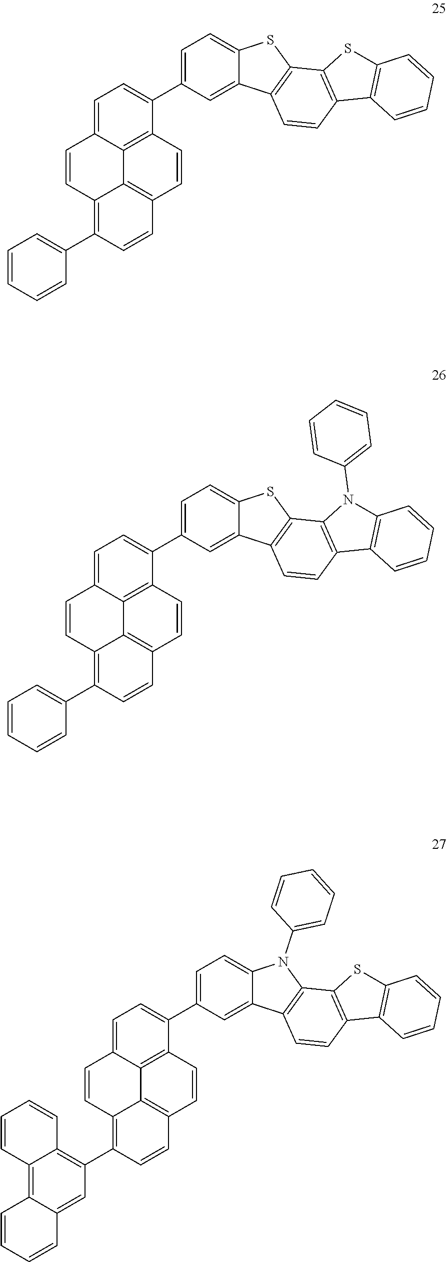 [SMILES: c1ccc(-c2ccc3ccc4c(-c5ccc6sc7c(ccc8c9ccccc9sc87)c6c5)ccc5ccc2c3c54)cc1 ; c1ccc(-c2ccc3ccc4c(-c5ccc6sc7c(ccc8c9ccccc9n(-c9ccccc9)c87)c6c5)ccc5ccc2c3c54)cc1 ; c1ccc(-n2c3ccc(-c4ccc5ccc6c(-c7cc8ccccc8c8ccccc78)ccc7ccc4c5c76)cc3c3ccc4c5ccccc5sc4c32)cc1]

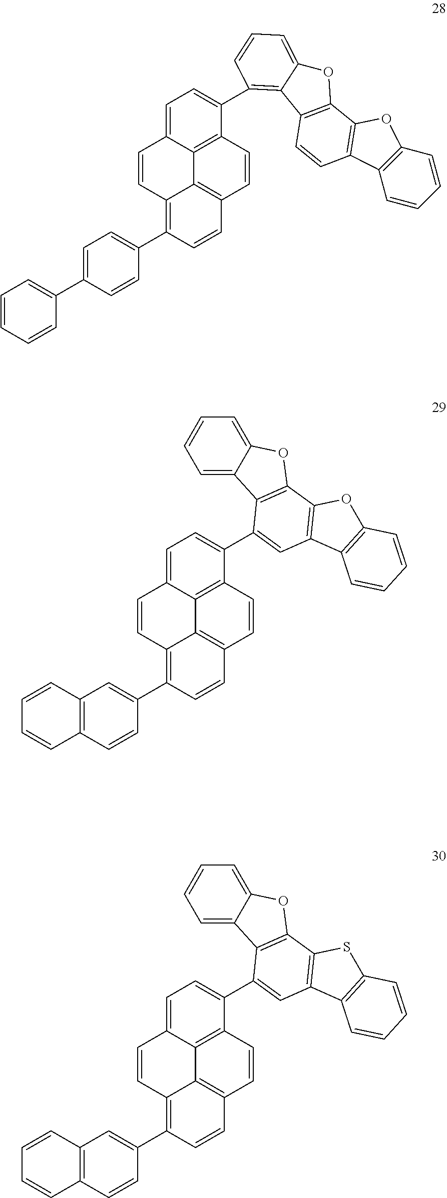 [SMILES: c1ccc(-c2ccc(-c3ccc4ccc5c(-c6cccc7oc8c(ccc9c%10ccccc%10oc98)c67)ccc6ccc3c4c65)cc2)cc1 ; c1ccc2cc(-c3ccc4ccc5c(-c6cc7c8ccccc8oc7c7oc8ccccc8c67)ccc6ccc3c4c65)ccc2c1 ; c1ccc2cc(-c3ccc4ccc5c(-c6cc7c8ccccc8sc7c7oc8ccccc8c67)ccc6ccc3c4c65)ccc2c1]

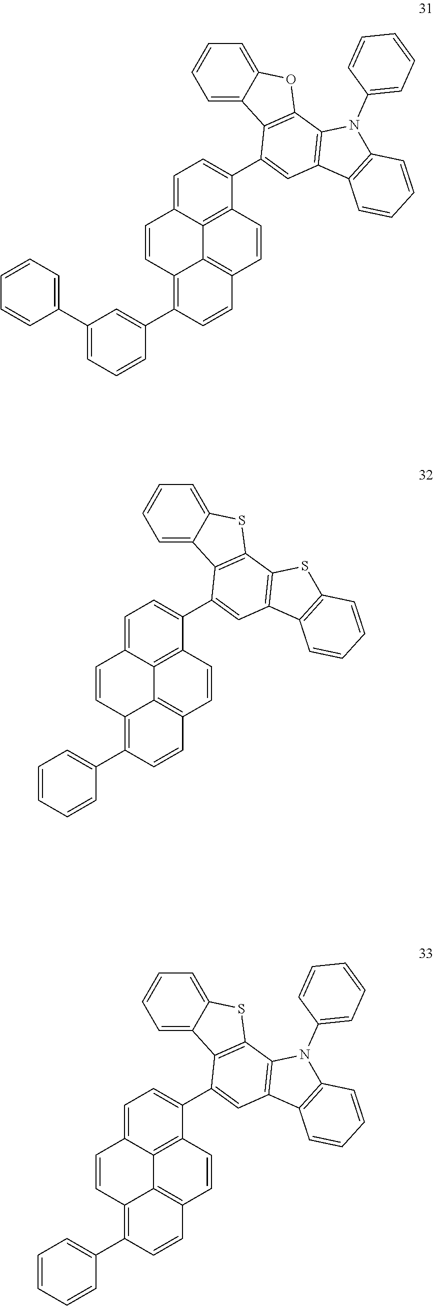 [SMILES: c1ccc(-c2cccc(-c3ccc4ccc5c(-c6cc7c8ccccc8n(-c8ccccc8)c7c7oc8ccccc8c67)ccc6ccc3c4c65)c2)cc1 ; c1ccc(-c2ccc3ccc4c(-c5cc6c7ccccc7sc6c6sc7ccccc7c56)ccc5ccc2c3c54)cc1 ; c1ccc(-c2ccc3ccc4c(-c5cc6c7ccccc7n(-c7ccccc7)c6c6sc7ccccc7c56)ccc5ccc2c3c54)cc1]

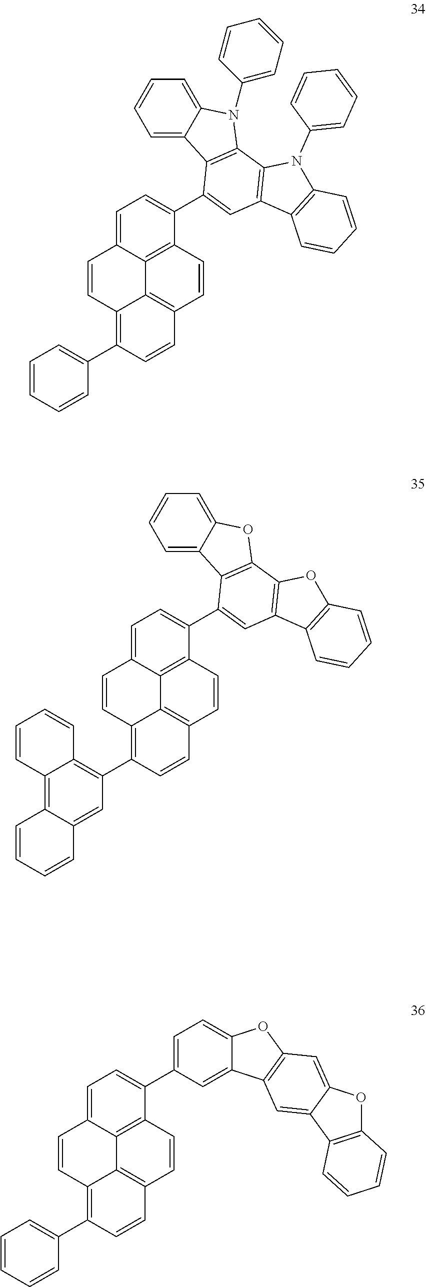 [SMILES: c1ccc(-c2ccc3ccc4c(-c5cc6c7ccccc7n(-c7ccccc7)c6c6c5c5ccccc5n6-c5ccccc5)ccc5ccc2c3c54)cc1 ; c1ccc2c(c1)cc(-c1ccc3ccc4c(-c5cc6c7ccccc7oc6c6oc7ccccc7c56)ccc5ccc1c3c54)c1ccccc12 ; c1ccc(-c2ccc3ccc4c(-c5ccc6oc7cc8oc9ccccc9c8cc7c6c5)ccc5ccc2c3c54)cc1]

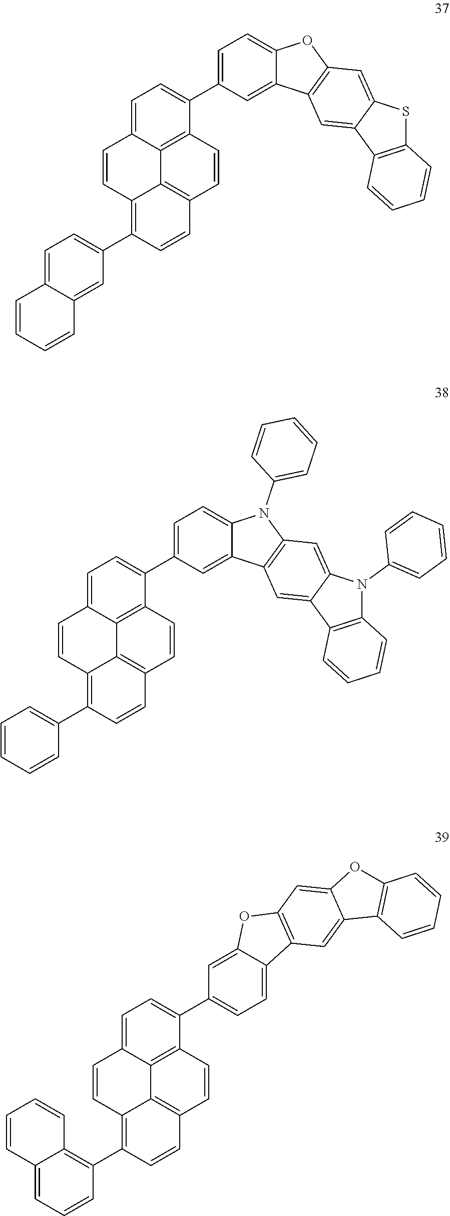 [SMILES: c1ccc2cc(-c3ccc4ccc5c(-c6ccc7oc8cc9sc%10ccccc%10c9cc8c7c6)ccc6ccc3c4c65)ccc2c1 ; c1ccc(-c2ccc3ccc4c(-c5ccc6c(c5)c5cc7c8ccccc8n(-c8ccccc8)c7cc5n6-c5ccccc5)ccc5ccc2c3c54)cc1 ; c1ccc2c(-c3ccc4ccc5c(-c6ccc7c(c6)oc6cc8oc9ccccc9c8cc67)ccc6ccc3c4c65)cccc2c1]

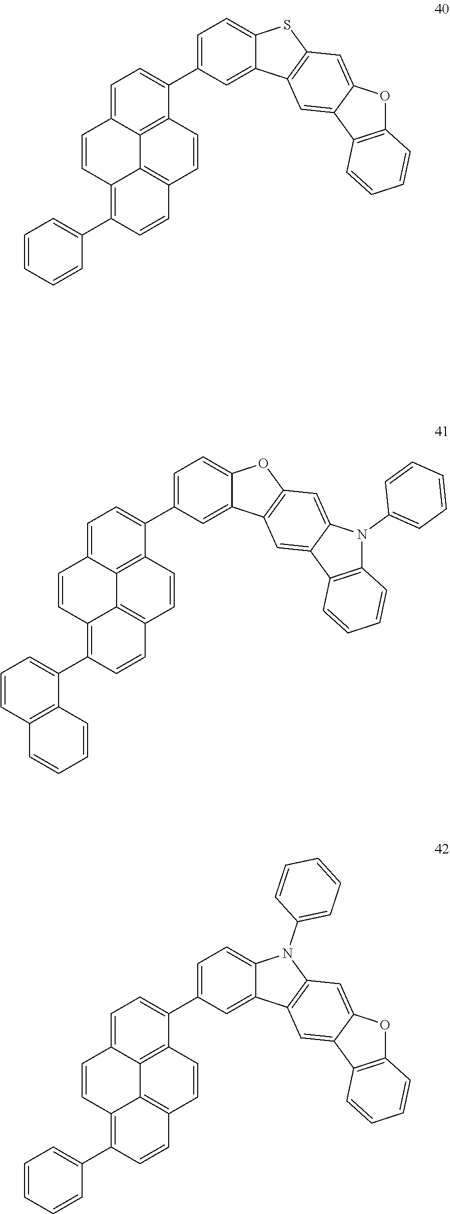 [SMILES: c1ccc(-c2ccc3ccc4c(-c5ccc6sc7cc8oc9ccccc9c8cc7c6c5)ccc5ccc2c3c54)cc1 ; c1ccc(-n2c3ccccc3c3cc4c(cc32)oc2ccc(-c3ccc5ccc6c(-c7cccc8ccccc78)ccc7ccc3c5c76)cc24)cc1 ; c1ccc(-c2ccc3ccc4c(-c5ccc6c(c5)c5cc7c(cc5n6-c5ccccc5)oc5ccccc57)ccc5ccc2c3c54)cc1]

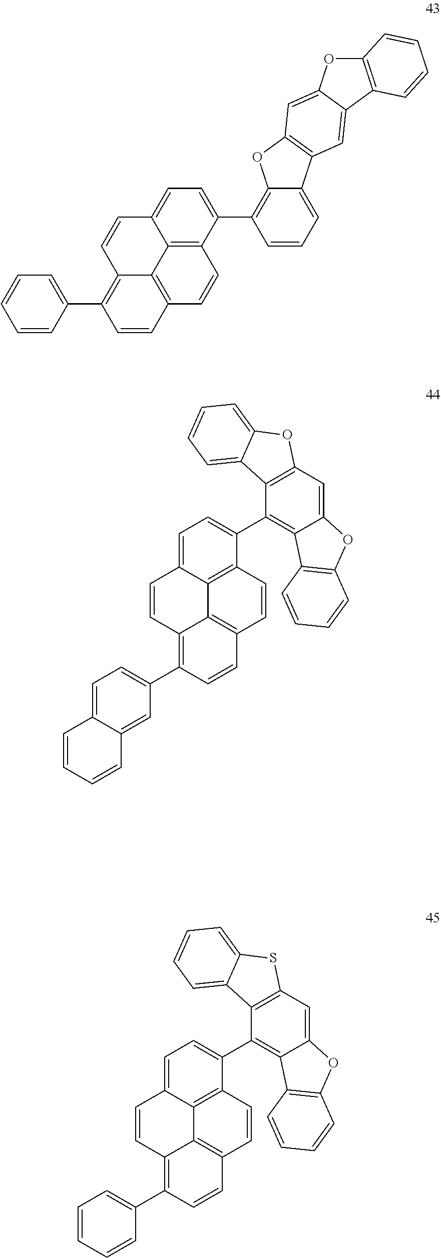 [SMILES: c1ccc(-c2ccc3ccc4c(-c5cccc6c5oc5cc7oc8ccccc8c7cc56)ccc5ccc2c3c54)cc1 ; c1ccc2cc(-c3ccc4ccc5c(-c6c7c(cc8oc9ccccc9c68)oc6ccccc67)ccc6ccc3c4c65)ccc2c1 ; c1ccc(-c2ccc3ccc4c(-c5c6c(cc7sc8ccccc8c57)oc5ccccc56)ccc5ccc2c3c54)cc1]

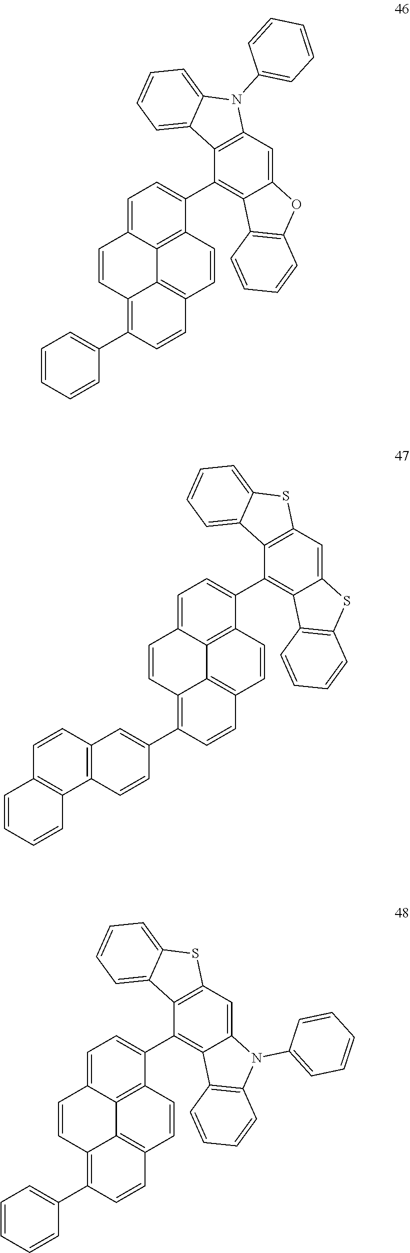 [SMILES: c1ccc(-c2ccc3ccc4c(-c5c6c(cc7c5c5ccccc5n7-c5ccccc5)oc5ccccc56)ccc5ccc2c3c54)cc1 ; c1ccc2c(c1)ccc1cc(-c3ccc4ccc5c(-c6c7c(cc8sc9ccccc9c68)sc6ccccc67)ccc6ccc3c4c65)ccc12 ; c1ccc(-c2ccc3ccc4c(-c5c6c(cc7c5c5ccccc5n7-c5ccccc5)sc5ccccc56)ccc5ccc2c3c54)cc1]

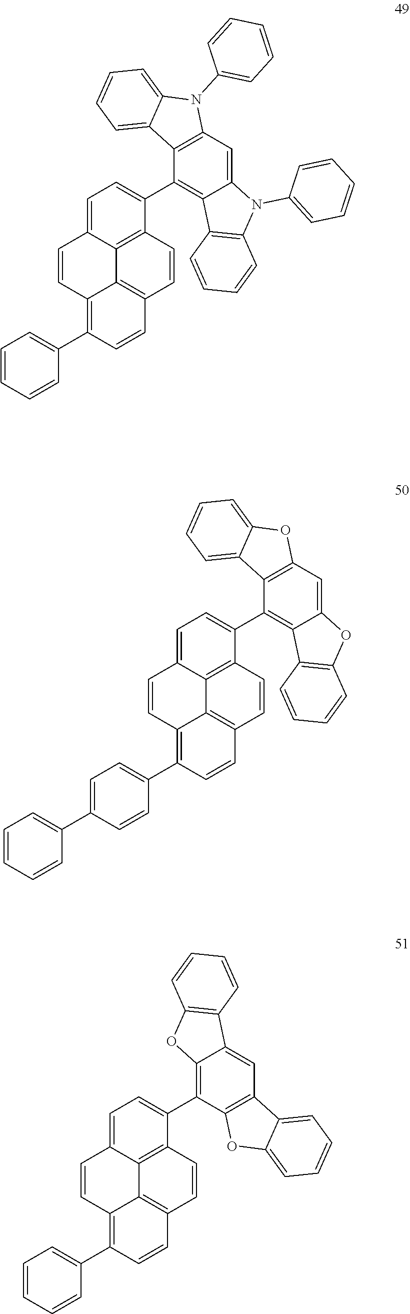 [SMILES: c1ccc(-c2ccc3ccc4c(-c5c6c7ccccc7n(-c7ccccc7)c6cc6c5c5ccccc5n6-c5ccccc5)ccc5ccc2c3c54)cc1 ; c1ccc(-c2ccc(-c3ccc4ccc5c(-c6c7c(cc8oc9ccccc9c68)oc6ccccc67)ccc6ccc3c4c65)cc2)cc1 ; c1ccc(-c2ccc3ccc4c(-c5c6oc7ccccc7c6cc6c5oc5ccccc56)ccc5ccc2c3c54)cc1]

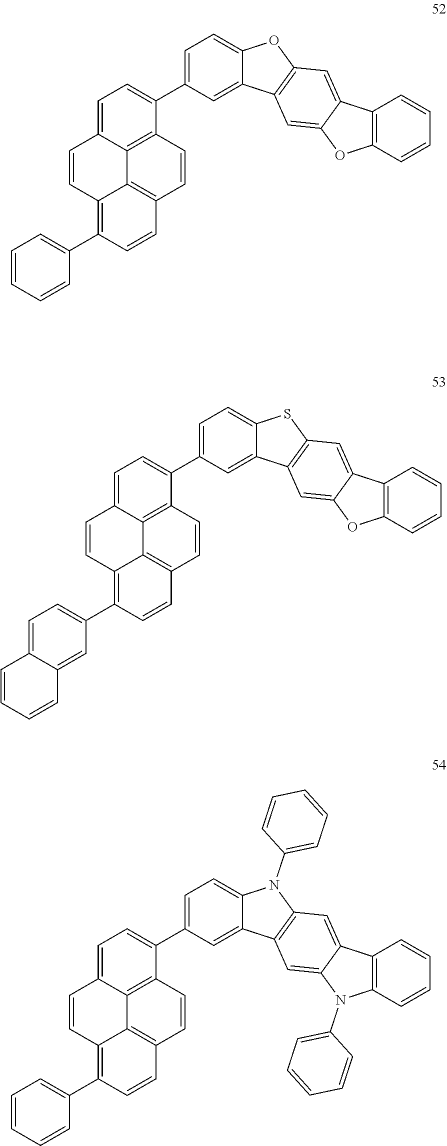 [SMILES: c1ccc(-c2ccc3ccc4c(-c5ccc6oc7cc8c(cc7c6c5)oc5ccccc58)ccc5ccc2c3c54)cc1 ; c1ccc2cc(-c3ccc4ccc5c(-c6ccc7sc8cc9c(cc8c7c6)oc6ccccc69)ccc6ccc3c4c65)ccc2c1 ; c1ccc(-c2ccc3ccc4c(-c5ccc6c(c5)c5cc7c(cc5n6-c5ccccc5)c5ccccc5n7-c5ccccc5)ccc5ccc2c3c54)cc1]

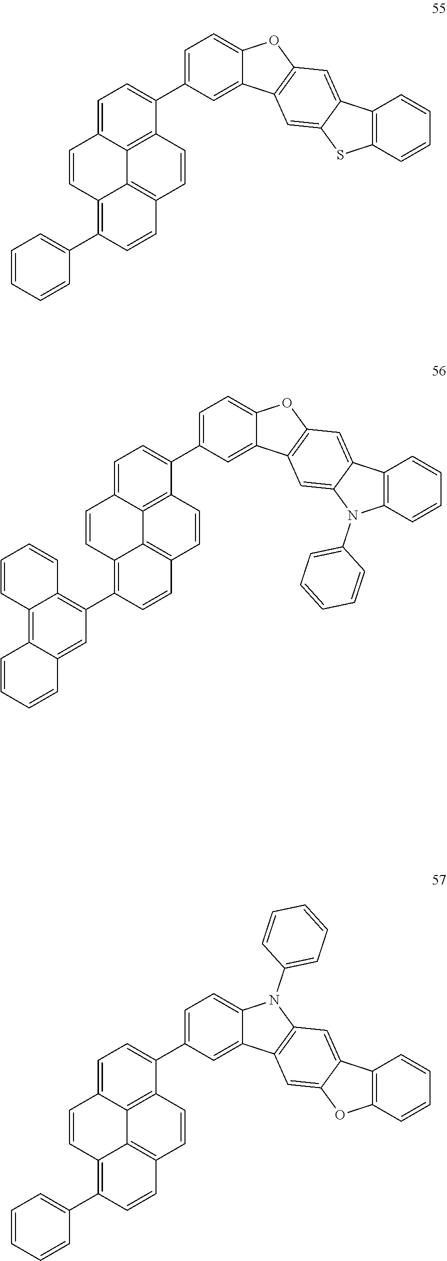 [SMILES: c1ccc(-c2ccc3ccc4c(-c5ccc6oc7cc8c(cc7c6c5)sc5ccccc58)ccc5ccc2c3c54)cc1 ; c1ccc(-n2c3ccccc3c3cc4oc5ccc(-c6ccc7ccc8c(-c9cc%10ccccc%10c%10ccccc9%10)ccc9ccc6c7c98)cc5c4cc32)cc1 ; c1ccc(-c2ccc3ccc4c(-c5ccc6c(c5)c5cc7oc8ccccc8c7cc5n6-c5ccccc5)ccc5ccc2c3c54)cc1]

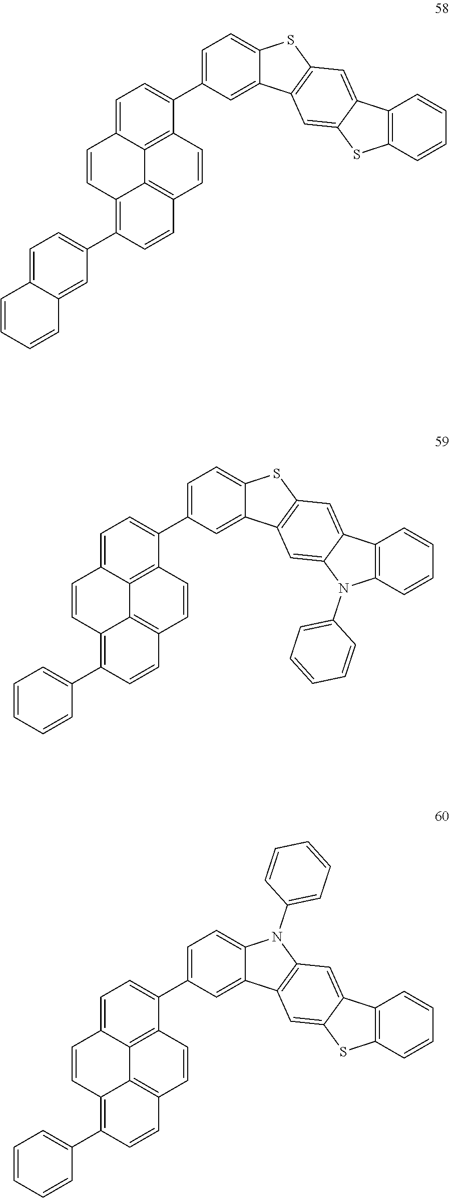 [SMILES: c1ccc2cc(-c3ccc4ccc5c(-c6ccc7sc8cc9c(cc8c7c6)sc6ccccc69)ccc6ccc3c4c65)ccc2c1 ; c1ccc(-c2ccc3ccc4c(-c5ccc6sc7cc8c9ccccc9n(-c9ccccc9)c8cc7c6c5)ccc5ccc2c3c54)cc1 ; c1ccc(-c2ccc3ccc4c(-c5ccc6c(c5)c5cc7sc8ccccc8c7cc5n6-c5ccccc5)ccc5ccc2c3c54)cc1]

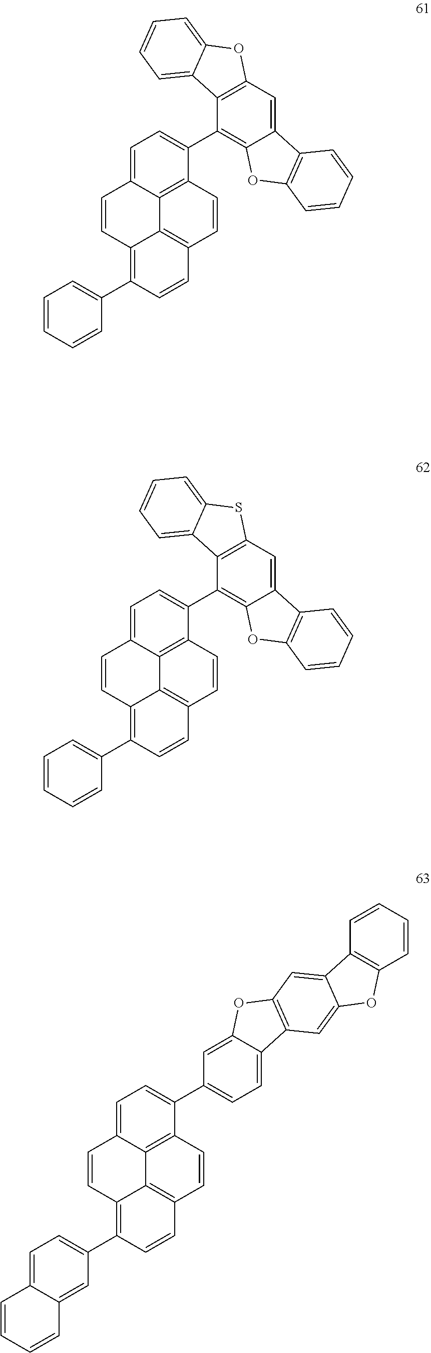 [SMILES: c1ccc(-c2ccc3ccc4c(-c5c6oc7ccccc7c6cc6oc7ccccc7c56)ccc5ccc2c3c54)cc1 ; c1ccc(-c2ccc3ccc4c(-c5c6oc7ccccc7c6cc6sc7ccccc7c56)ccc5ccc2c3c54)cc1 ; c1ccc2cc(-c3ccc4ccc5c(-c6ccc7c(c6)oc6cc8c(cc67)oc6ccccc68)ccc6ccc3c4c65)ccc2c1]

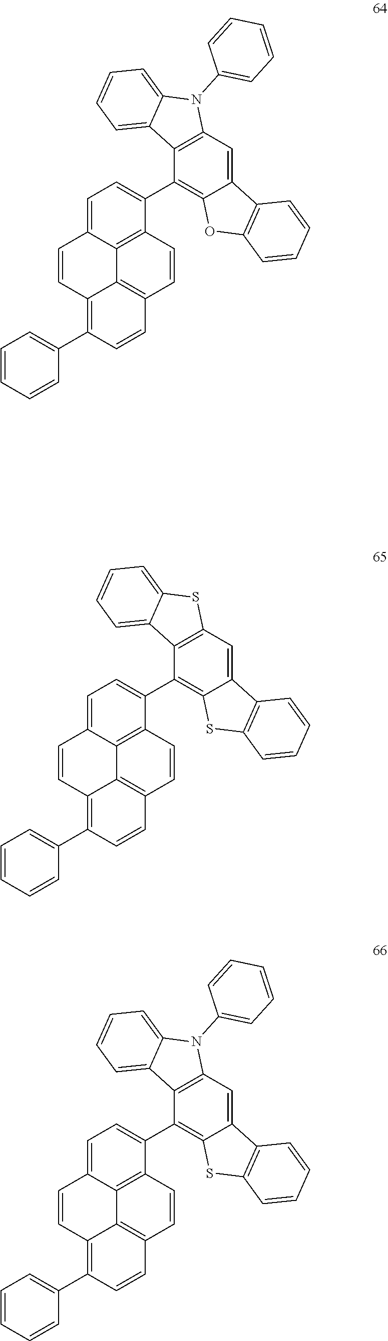 [SMILES: c1ccc(-c2ccc3ccc4c(-c5c6oc7ccccc7c6cc6c5c5ccccc5n6-c5ccccc5)ccc5ccc2c3c54)cc1 ; c1ccc(-c2ccc3ccc4c(-c5c6sc7ccccc7c6cc6sc7ccccc7c56)ccc5ccc2c3c54)cc1 ; c1ccc(-c2ccc3ccc4c(-c5c6sc7ccccc7c6cc6c5c5ccccc5n6-c5ccccc5)ccc5ccc2c3c54)cc1]

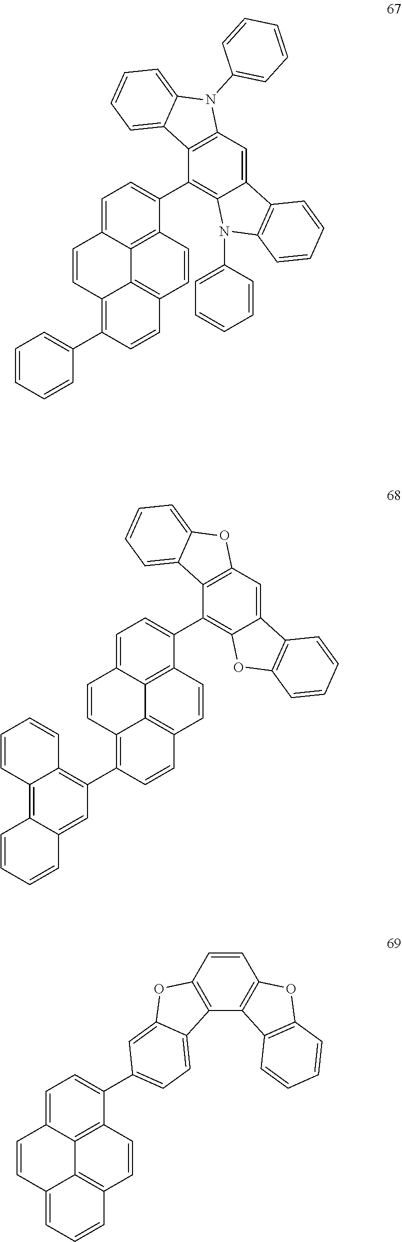 [SMILES: c1ccc(-c2ccc3ccc4c(-c5c6c7ccccc7n(-c7ccccc7)c6cc6c7ccccc7n(-c7ccccc7)c56)ccc5ccc2c3c54)cc1 ; c1ccc2c(c1)cc(-c1ccc3ccc4c(-c5c6oc7ccccc7c6cc6oc7ccccc7c56)ccc5ccc1c3c54)c1ccccc12 ; c1cc2ccc3ccc(-c4ccc5c(c4)oc4ccc6oc7ccccc7c6c45)c4ccc(c1)c2c34]

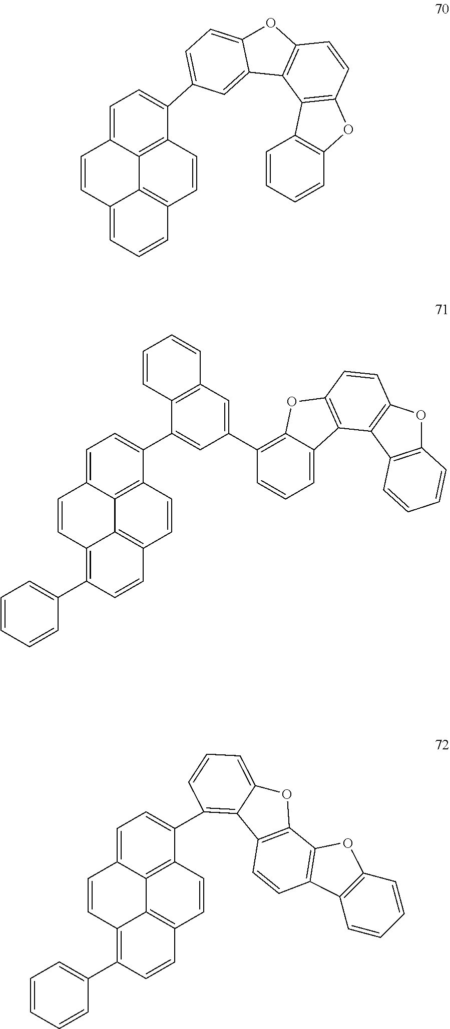 [SMILES: c1cc2ccc3ccc(-c4ccc5oc6ccc7oc8ccccc8c7c6c5c4)c4ccc(c1)c2c34 ; c1ccc(-c2ccc3ccc4c(-c5cc(-c6cccc7c6oc6ccc8oc9ccccc9c8c67)cc6ccccc56)ccc5ccc2c3c54)cc1 ; c1ccc(-c2ccc3ccc4c(-c5cccc6oc7c(ccc8c9ccccc9oc87)c56)ccc5ccc2c3c54)cc1]

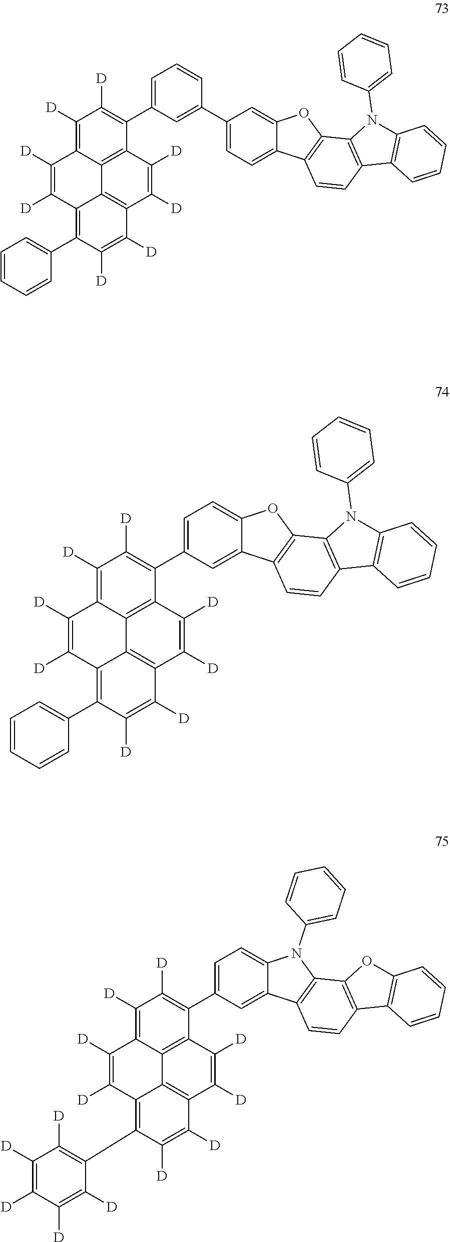 [SMILES: [2H]c1c([2H])c2c([2H])c([2H])c3c(-c4cccc(-c5ccc6c(c5)oc5c6ccc6c7ccccc7n(-c7ccccc7)c65)c4)c([2H])c([2H])c4c([2H])c([2H])c(c1-c1ccccc1)c2c43 ; [2H]c1c([2H])c2c([2H])c([2H])c3c(-c4ccc5oc6c(ccc7c8ccccc8n(-c8ccccc8)c76)c5c4)c([2H])c([2H])c4c([2H])c([2H])c(c1-c1ccccc1)c2c43 ; [2H]c1c([2H])c([2H])c(-c2c([2H])c([2H])c3c([2H])c([2H])c4c(-c5ccc6c(c5)c5ccc7c8ccccc8oc7c5n6-c5ccccc5)c([2H])c([2H])c5c([2H])c([2H])c2c3c54)c([2H])c1[2H]]

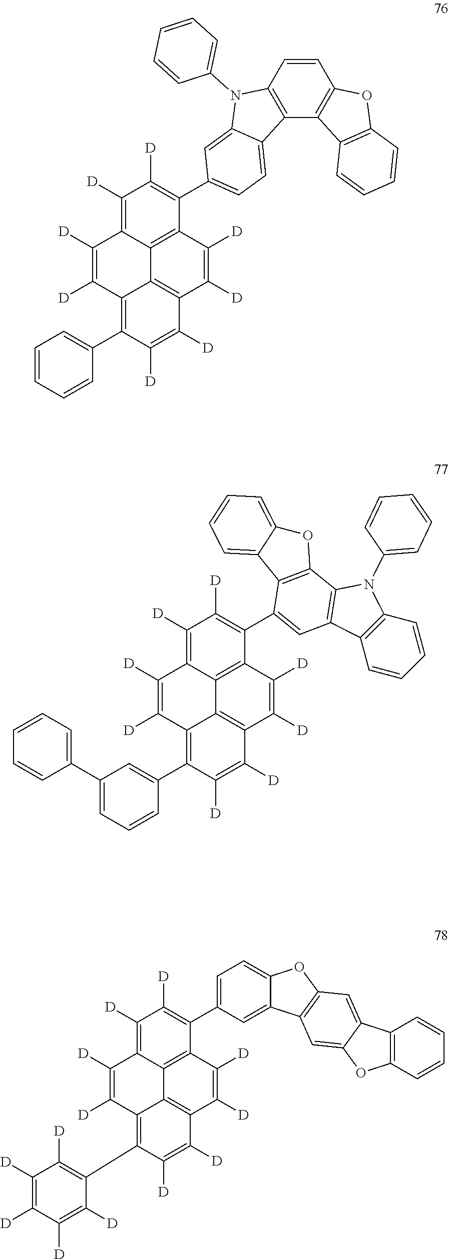 [SMILES: [2H]c1c([2H])c2c([2H])c([2H])c3c(-c4ccc5c6c7c(ccc6n(-c6ccccc6)c5c4)oc4ccccc47)c([2H])c([2H])c4c([2H])c([2H])c(c1-c1ccccc1)c2c43 ; [2H]c1c([2H])c2c([2H])c([2H])c3c(-c4cc5c6ccccc6n(-c6ccccc6)c5c5oc6ccccc6c45)c([2H])c([2H])c4c([2H])c([2H])c(c1-c1cccc(-c5ccccc5)c1)c2c43 ; [2H]c1c([2H])c([2H])c(-c2c([2H])c([2H])c3c([2H])c([2H])c4c(-c5ccc6oc7cc8c(cc7c6c5)oc5ccccc58)c([2H])c([2H])c5c([2H])c([2H])c2c3c54)c([2H])c1[2H]]

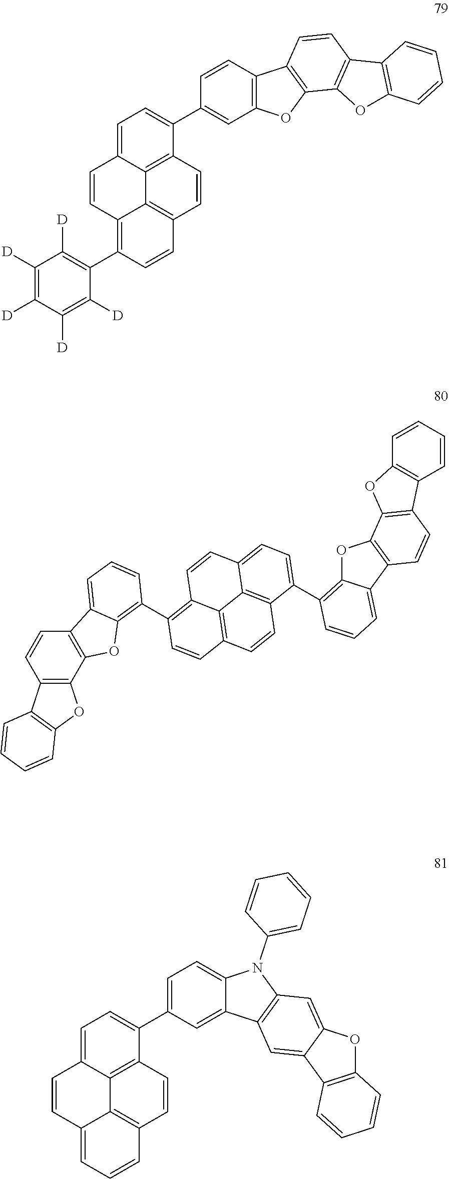 [SMILES: [2H]c1c([2H])c([2H])c(-c2ccc3ccc4c(-c5ccc6c(c5)oc5c6ccc6c7ccccc7oc65)ccc5ccc2c3c54)c([2H])c1[2H] ; c1ccc2c(c1)oc1c2ccc2c3cccc(-c4ccc5ccc6c(-c7cccc8c7oc7c8ccc8c9ccccc9oc87)ccc7ccc4c5c76)c3oc21 ; c1ccc(-n2c3ccc(-c4ccc5ccc6cccc7ccc4c5c67)cc3c3cc4c(cc32)oc2ccccc24)cc1]

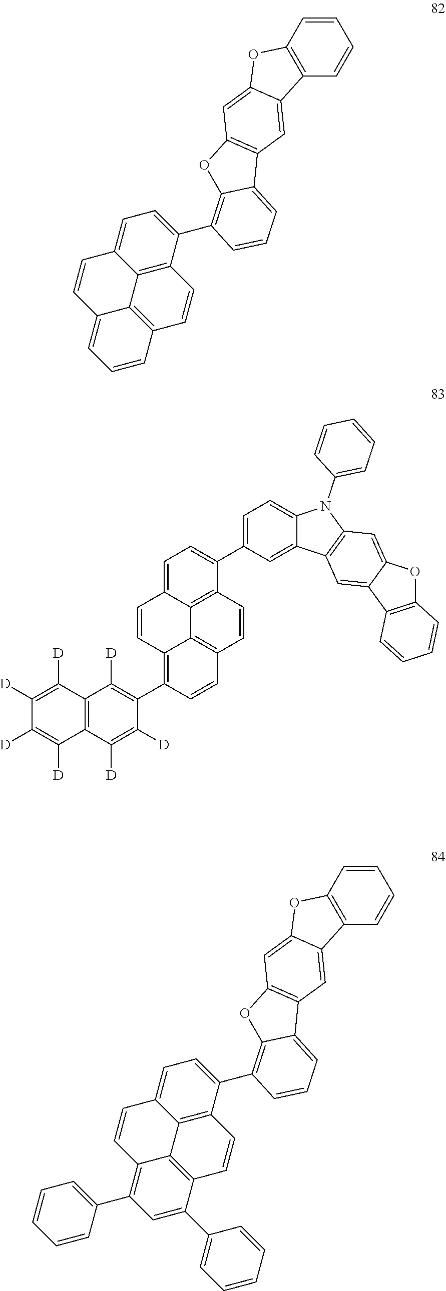 [SMILES: c1cc2ccc3ccc(-c4cccc5c4oc4cc6oc7ccccc7c6cc45)c4ccc(c1)c2c34 ; [2H]c1c([2H])c([2H])c2c([2H])c(-c3ccc4ccc5c(-c6ccc7c(c6)c6cc8c(cc6n7-c6ccccc6)oc6ccccc68)ccc6ccc3c4c65)c([2H])c([2H])c2c1[2H] ; c1ccc(-c2cc(-c3ccccc3)c3ccc4c(-c5cccc6c5oc5cc7oc8ccccc8c7cc56)ccc5ccc2c3c54)cc1]

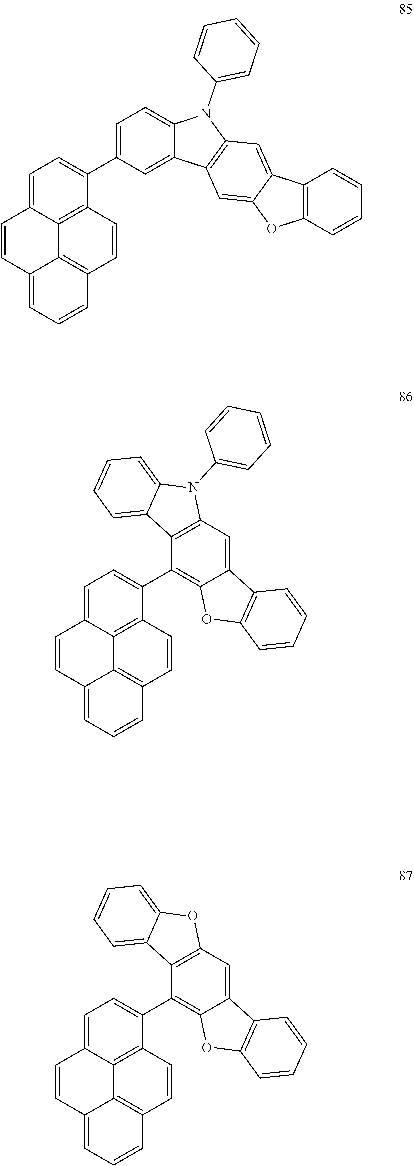 [SMILES: c1ccc(-n2c3ccc(-c4ccc5ccc6cccc7ccc4c5c67)cc3c3cc4oc5ccccc5c4cc32)cc1 ; c1ccc(-n2c3ccccc3c3c(-c4ccc5ccc6cccc7ccc4c5c67)c4oc5ccccc5c4cc32)cc1 ; c1cc2ccc3ccc(-c4c5oc6ccccc6c5cc5oc6ccccc6c45)c4ccc(c1)c2c34]

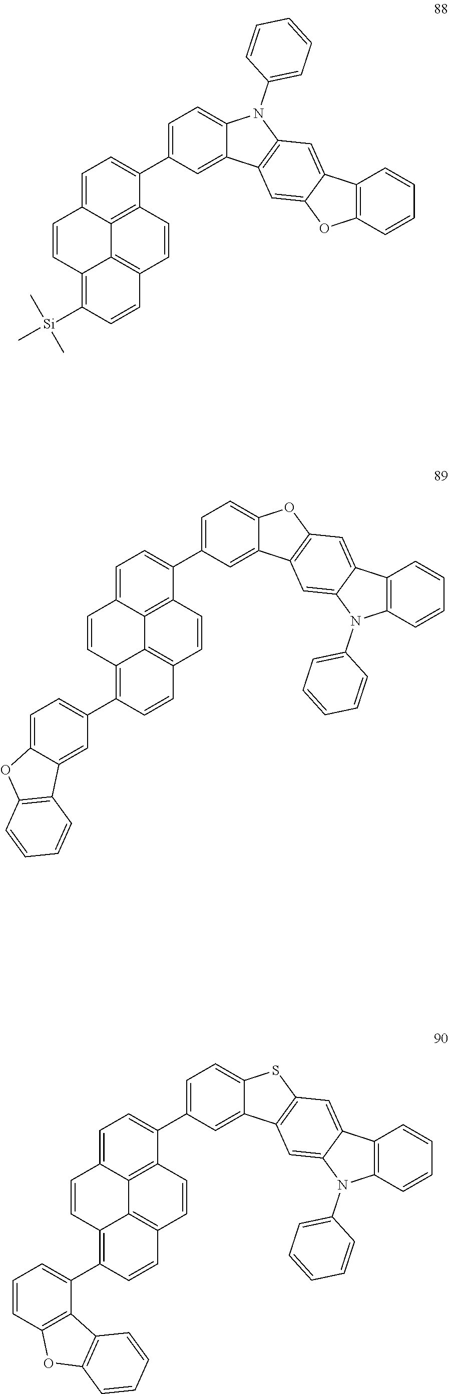 [SMILES: C[Si](C)(C)c1ccc2ccc3c(-c4ccc5c(c4)c4cc6oc7ccccc7c6cc4n5-c4ccccc4)ccc4ccc1c2c43 ; c1ccc(-n2c3ccccc3c3cc4oc5ccc(-c6ccc7ccc8c(-c9ccc%10oc%11ccccc%11c%10c9)ccc9ccc6c7c98)cc5c4cc32)cc1 ; c1ccc(-n2c3ccccc3c3cc4sc5ccc(-c6ccc7ccc8c(-c9cccc%10oc%11ccccc%11c9%10)ccc9ccc6c7c98)cc5c4cc32)cc1]

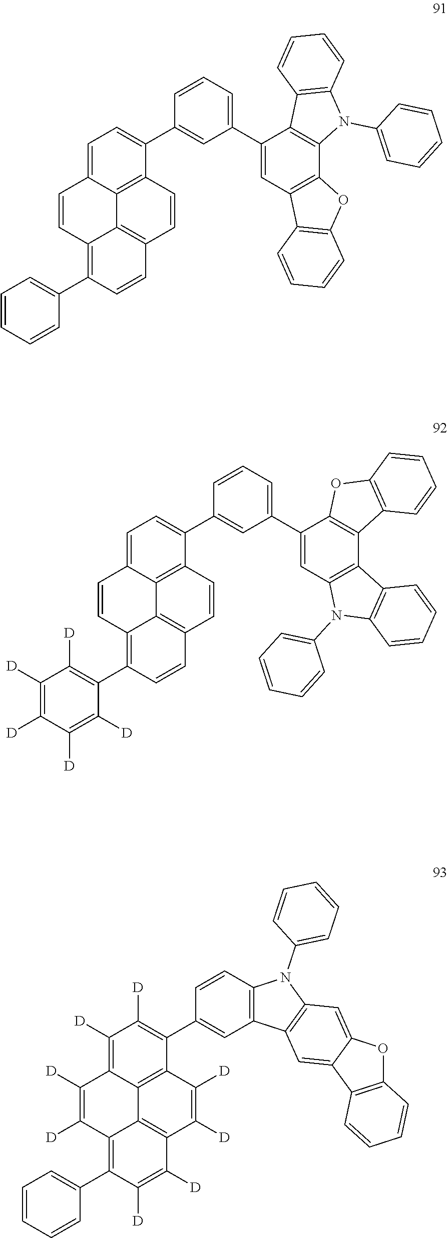 [SMILES: c1ccc(-c2ccc3ccc4c(-c5cccc(-c6cc7c8ccccc8oc7c7c6c6ccccc6n7-c6ccccc6)c5)ccc5ccc2c3c54)cc1 ; [2H]c1c([2H])c([2H])c(-c2ccc3ccc4c(-c5cccc(-c6cc7c(c8ccccc8n7-c7ccccc7)c7c6oc6ccccc67)c5)ccc5ccc2c3c54)c([2H])c1[2H] ; [2H]c1c([2H])c2c([2H])c([2H])c3c(-c4ccc5c(c4)c4cc6c(cc4n5-c4ccccc4)oc4ccccc46)c([2H])c([2H])c4c([2H])c([2H])c(c1-c1ccccc1)c2c43]

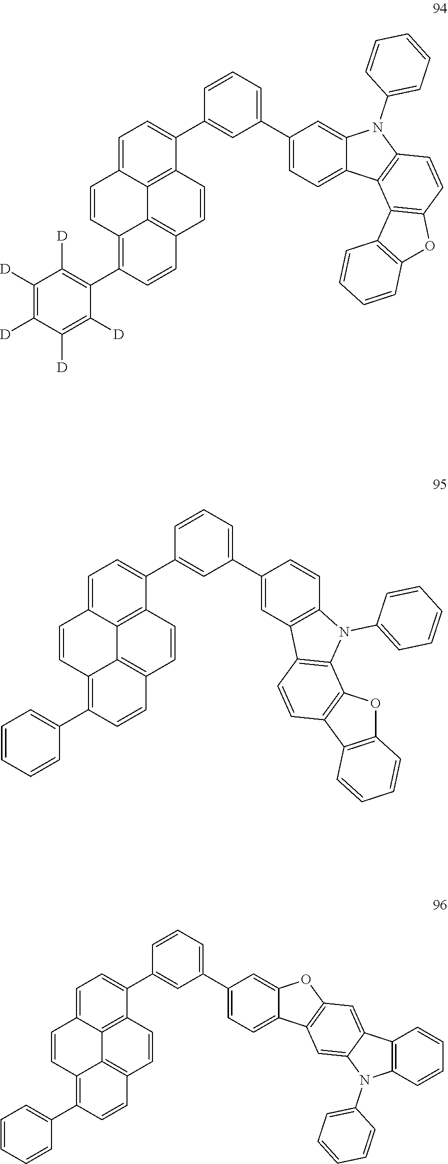 [SMILES: [2H]c1c([2H])c([2H])c(-c2ccc3ccc4c(-c5cccc(-c6ccc7c8c9c(ccc8n(-c8ccccc8)c7c6)oc6ccccc69)c5)ccc5ccc2c3c54)c([2H])c1[2H] ; c1ccc(-c2ccc3ccc4c(-c5cccc(-c6ccc7c(c6)c6ccc8c9ccccc9oc8c6n7-c6ccccc6)c5)ccc5ccc2c3c54)cc1 ; c1ccc(-c2ccc3ccc4c(-c5cccc(-c6ccc7c(c6)oc6cc8c9ccccc9n(-c9ccccc9)c8cc67)c5)ccc5ccc2c3c54)cc1]

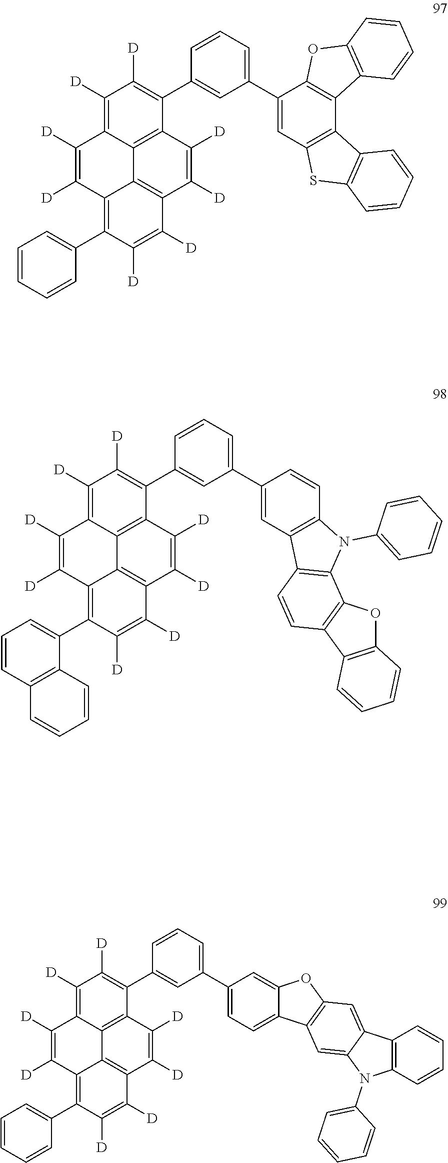 [SMILES: [2H]c1c([2H])c2c([2H])c([2H])c3c(-c4cccc(-c5cc6sc7ccccc7c6c6c5oc5ccccc56)c4)c([2H])c([2H])c4c([2H])c([2H])c(c1-c1ccccc1)c2c43 ; [2H]c1c([2H])c2c([2H])c([2H])c3c(-c4cccc5ccccc45)c([2H])c([2H])c4c([2H])c([2H])c(c1-c1cccc(-c5ccc6c(c5)c5ccc7c8ccccc8oc7c5n6-c5ccccc5)c1)c2c43 ; [2H]c1c([2H])c2c([2H])c([2H])c3c(-c4cccc(-c5ccc6c(c5)oc5cc7c8ccccc8n(-c8ccccc8)c7cc56)c4)c([2H])c([2H])c4c([2H])c([2H])c(c1-c1ccccc1)c2c43]

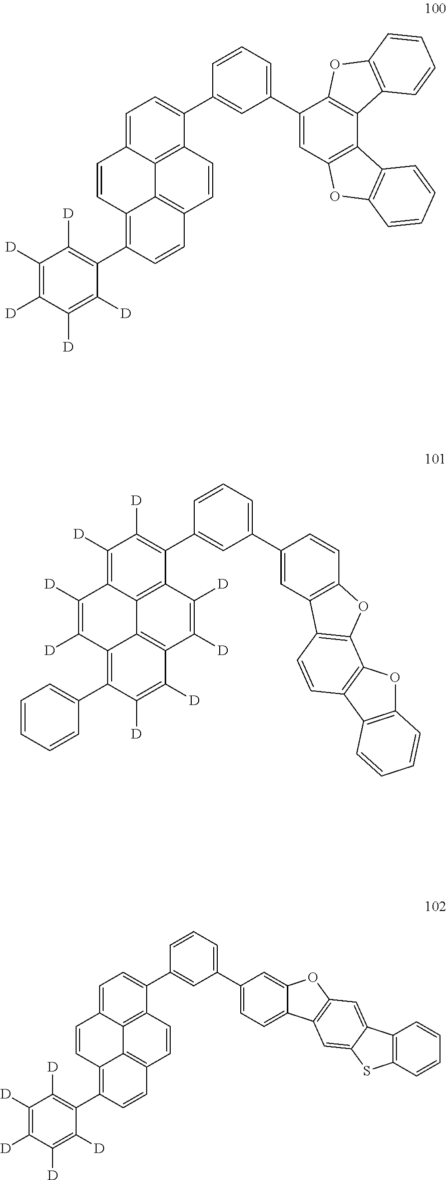 [SMILES: [2H]c1c([2H])c([2H])c(-c2ccc3ccc4c(-c5cccc(-c6cc7oc8ccccc8c7c7c6oc6ccccc67)c5)ccc5ccc2c3c54)c([2H])c1[2H] ; [2H]c1c([2H])c2c([2H])c([2H])c3c(-c4cccc(-c5ccc6oc7c(ccc8c9ccccc9oc87)c6c5)c4)c([2H])c([2H])c4c([2H])c([2H])c(c1-c1ccccc1)c2c43 ; [2H]c1c([2H])c([2H])c(-c2ccc3ccc4c(-c5cccc(-c6ccc7c(c6)oc6cc8c(cc67)sc6ccccc68)c5)ccc5ccc2c3c54)c([2H])c1[2H]]

5. An organic light emitting device comprising a first electrode, a second electrode opposite to the first electrode, and one or more organic layers interposed between the first and second electrodes wherein one of the organic layers comprises the compound according to claim 1.

6. The organic light emitting device according to claim 5, wherein the organic layers comprise a hole injecting layer, a hole transport layer, a functional layer having functions of both hole injection and hole transport, a light emitting layer, an electron transport layer, an electron injecting layer, and/or a functional layer having functions of both electron injection and electron transport.

7. The organic light emitting device according to claim 6, wherein one of the organic layers interposed between the first and second electrodes is a light emitting layer composed of a host and a dopant and wherein the host is the compound represented by Formula I.

8. The organic light emitting device according to claim 6, wherein the dopant comprises a compound represented by one of Formulas D1 to D10: 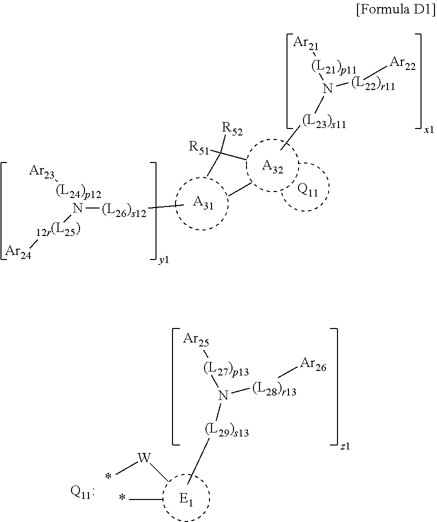
wherein A₃₁, A₃₂, and E₁ are each independently a substituted or unsubstituted C₆-C₅₀ aromatic hydrocarbon ring or a substituted or unsubstituted C₂-C₄₀ aromatic heterocyclic ring, with the proviso that two adjacent carbon atoms in the aromatic ring A₃₁ and two adjacent carbon atoms in the aromatic ring A₃₂ optionally form a 5-membered ring with the carbon atom to which R₅₁ and R₅₂ are attached, to form a fused ring, L₂₁ to L₂₉ are the same as or different from each other and are each independently selected from a single bond, substituted or unsubstituted C₁-C₅₀ alkylene, substituted or unsubstituted C₂-C₅₀ alkenylene, substituted or unsubstituted C₂-C₅₀ alkynylene, substituted or unsubstituted C₃-C₆₀ cycloalkylene, substituted or unsubstituted C₂-C₆₀ heterocycloalkylene, substituted or unsubstituted C₆-C₅₀ arylene, and substituted or unsubstituted C₂-C₅₀ heteroarylene, W is selected from N—R₅₃, CR₅₄R₅₅, SiR₅₆R₅₇, GeR₅₈R₅₉, O, S, and Se, R₅₁ to R₅₉ and Ar₂₁ to Ar₂₆ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₂-C₃₀ alkenyl, substituted or unsubstituted C₂-C₂₀ alkynyl, substituted or unsubstituted C₃-C₃₀ cycloalkyl, substituted or unsubstituted C₅-C₃₀ cycloalkenyl, substituted or unsubstituted C₂-C₅₀ heteroaryl, substituted or unsubstituted C₂-C₃₀ heterocycloalkyl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, substituted or unsubstituted C₁-C₃₀ alkylgermanium, substituted or unsubstituted C₁-C₃₀ arylgermanium, cyano, nitro, and halogen, with the proviso that R₅₁ and R₅₂ are optionally linked to each other to form an alicyclic or aromatic monocyclic or polycyclic ring in which carbon atoms are optionally substituted with one or more heteroatoms selected from the group consisting of N, O, P, Si, S, Ge, Se, and Te, p11 to p13, r11 to r13, and s11 to s13 are each independently an integer from 1 to 3, provided that when p11 is 2 or greater, the linkers L₂₁ are the same as or different from each other, when p12 is 2 or greater, the linkers L₂₄ are the same as or different from each other, when p13 is 2 or greater, the linkers L₂₇ are the same as or different from each other, when r11 is 2 or greater, the linkers L₂₂ are the same as or different from each other, when r12 is 2 or greater, the linkers L₂₅ are the same as or different from each other, when r13 is 2 or greater, the linkers L₂₈ are the same as or different from each other, when s11 is 2 or greater, the linkers L₂₃ are the same as or different from each other, when s12 is 2 or greater, the linkers L₂₆ are the same as or different from each other, and when s13 is 2 or greater, the linkers L₂₉ are the same as or different from each other, x1 is 1, and y1 and z1 are each independently an integer of 0 or 1, with the proviso that Ar₂₁ and Ar₂₂ are optionally linked to each other to form a ring, Ar₂₃ and Ar₂₄ are optionally linked to each other to form a ring, and Ar₂₅ and Ar₂₆ are optionally linked to each other to form a ring, and with the proviso that two adjacent carbon atoms in the ring A₃₂ are optionally bonded to Q₁₁ at the asterisks (*) to form a fused ring; 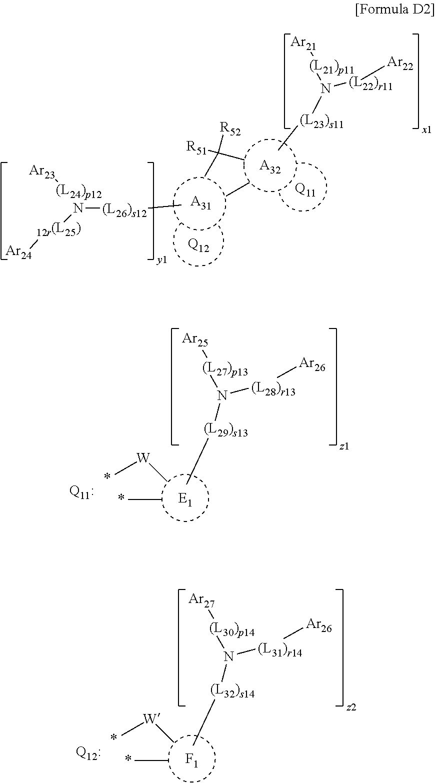
wherein A₃₁, A₃₂, E₁, and F₁ are as defined for A₃₁, A₃₂, and E₁ in Formula D1, L₂₁ to L₃₂ are as defined for L₂₁ to L₂₉ in Formula D1, W and W′ are the same as or different from each other and are each independently as defined for W in Formula D1, R₅₁ to R₅₉ and Ar₂₁ to Ar₂₈ are as defined for R₅₁ to R₅₉ and Ar₂₁ to Ar₂₆ in Formula D1, p11 to p14, r11 to r14, and s11 to s14 are as defined for p11 to p13, r11 to r13, and s11 to s13 in Formula D1, provided that when p14 is 2 or greater, the linkers L₃₀ are the same as or different from each other, when r14 is 2 or greater, the linkers L₃₁ are the same as or different from each other, and when s14 is 2 or greater, the linkers L₃₂ are the same as or different from each other, x1 is as defined in Formula D1, and y1, z1, and z2 are as defined for y1 and z1 in Formula D1, with the proviso that Ar₂₁ and Ar₂₂ are optionally linked to each other to form a ring, Ar₂₃ and Ar₂₄ are optionally linked to each other to form a ring, Ar₂₅ and Ar₂₆ are optionally linked to each other to form a ring, and Ar₂₇ and Ar₂₈ are optionally linked to each other to form a ring, and with the proviso that two adjacent carbon atoms in the ring A₃₁ are optionally bonded to Q₁₂ at the asterisks (*) to form a fused ring and two adjacent carbon atoms in the ring A₃₂ are optionally bonded to Q₁₁ at the asterisks (*) to form a fused ring; 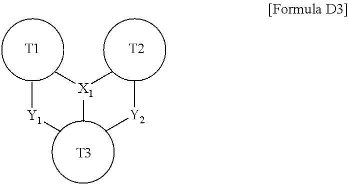
wherein X₁ is selected from B, P, and P═O, T1 to T3 are the same as or different from each other and are each independently a substituted or unsubstituted C₆-C₅₀ aromatic hydrocarbon ring or a substituted or unsubstituted C₂-C₄₀ aromatic heterocyclic ring, Y₁ is selected from N—R₆₁, CR₆₂R₆₃, O, S, and SiR₆₄R₆₅, Y₂ is selected from N—R₆₆, CR₆₇R₆₈, O, S, and SiR₆₉R₇₀, and R₆₁ to R₇₀ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₃-C₃₀ cycloalkyl, substituted or unsubstituted C₂-C₅₀ heteroaryl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, cyano, and halogen, with the proviso that each of R₆₁ to R₇₀ is optionally bonded to one or more rings selected from T1 to T3 to form an alicyclic or aromatic monocyclic or polycyclic ring; 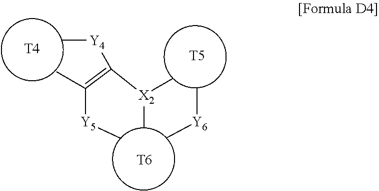
wherein X₂ is selected from B, P, and P═O, T4 to T6 are as defined for T1 to T3 in Formula D3, Y₄ is selected from N—R₆₁, CR₆₂R₆₃, O, S, and SiR₆₄R₆₅, Y₅ is selected from N—R₆₆, CR₆₇R₆₈, O, S, and SiR₆₉R₇₀, Y₆ is selected from N—R₇₁, CR₇₂R₇₃, O, S, and SiR₇₄R₇₅, and R₆₁ to R₇₅ are as defined for R₆₁ to R₇₀ in Formula D3; 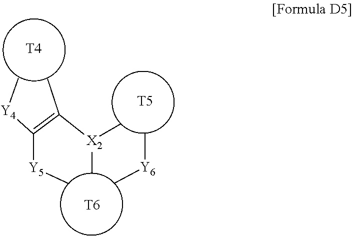
wherein X₂, T4 to T6, and Y₄ to Y₆ are as defined in Formula D4; 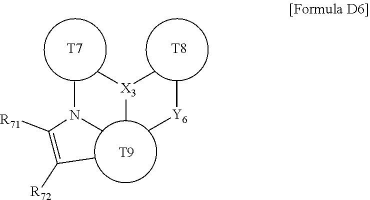
wherein X₃ is selected from B, P, and P═O, T7 to T9 are as defined for T1 to T3 in Formula D3, Y₆ is selected from N—R₆₁, CR₆₂R₆₃, O, S, and SiR₆₄R₆₅, and R₆₁ to R₆₅, R₇₁, and R₇₂ are as defined for R₆₁ to R₇₀ in Formula D3, with the proviso that R₇₁ and R₇₂ are optionally linked to each other to form an alicyclic or aromatic monocyclic or polycyclic ring or are optionally bonded to the ring T7 or T9 to form an alicyclic or aromatic monocyclic or polycyclic ring; 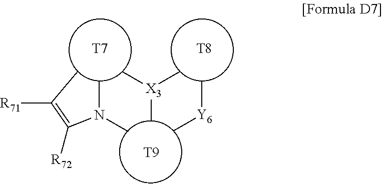
wherein X₃, T7 to T9, Y₆, R₇₁, and R₇₂ are as defined in Formula D6; 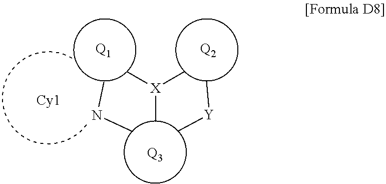
wherein X is selected from B, P, and P═O, Q₁ to Q₃ are as defined for T1 to T3 in Formula D3, Y is selected from N—R₃, CR₄R₅, O, S, and Se, R₃ to R₅ are as defined for R₆₁ to R₇₀ in Formula D3, with the proviso that each of R₃ to R₅ is optionally bonded to the ring Q₂ or Q₃ to form an alicyclic or aromatic monocyclic or polycyclic ring and each of R₄ and R₅ is optionally linked to each other to form an alicyclic or aromatic monocyclic or polycyclic ring, and Cy1 represents a ring formed by a substituted or unsubstituted C₁-C₁₀ alkylene group with the nitrogen (N) atom between the rings Q₁ and Q₃, the aromatic carbon atom in the ring Q₁ to which the nitrogen (N) atom is bonded, and the aromatic carbon atom in the ring Q₁ to which Cy1 is to be bonded; 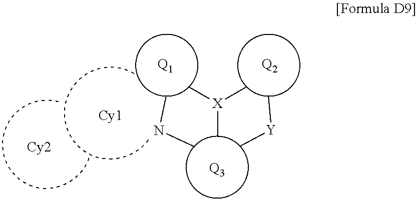
wherein X, Q₁ to Q₃, Y, and Cy1 are as defined in Formula D8 and Cy2 represents a saturated hydrocarbon ring attached to Cy1 and formed by a substituted or unsubstituted C₁-C₁₀ alkylene group with carbon atoms in Cy1; and 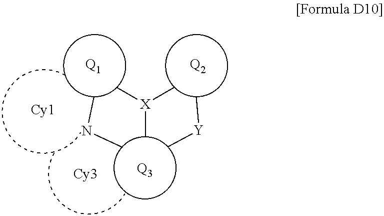
wherein X, Q₁ to Q₃, Y, and Cy1 are as defined in Formula D8 and Cy3 represents a ring formed by a substituted or unsubstituted C₁-C₁₀ alkylene group with the aromatic carbon atom in the ring Q₃ to which Cy3 is to be bonded, the nitrogen (N) atom between the rings Q₁ and Q₃, the aromatic carbon atom in the ring Q₃ to which the nitrogen (N) atom is bonded, and the carbon atom in Cy1 to which the nitrogen (N) atom is bonded.

9. The organic light emitting device according to claim 6, wherein one or more of the layers are formed by a deposition or solution process.

10. The organic light emitting device according to claim 5, wherein the organic light emitting device is used in a display or lighting system selected from flat panel displays, flexible displays, monochromatic flat panel lighting systems, white flat panel lighting systems, flexible monochromatic lighting systems, and flexible white lighting systems.
